(19) United States
(12) Patent Application Publication
(10) Pub. No.: US 2002/0183249 A1
(43) Pub. Date: Dec. 5, 2002

(54) Method of identifying inhibitors of CDC25

(76) Inventors: Neil R. Taylor, Sydney (AU); David Borhani, Worcester, MA (US); David Epstein, Belmont, MA (US); Johannes Rudolph, Durham, NC (US); Kurt Ritter, Newton, MA (US); Taro Fujimori, Shrewsbury, MA (US); Simon Robinson, Stow, MA (US); Jens Eckstein, Arlington, MA (US); Andreas Haupt, Schwetzingen (DE); Nigel Walker, Burlingame, CA (US); Richard W. Dixon, Jefferson, MA (US); Deborah Choquette, Rutland, MA (US); Jill Blanchard, Arlington, MA (US); Arthur Kluge, Lincoln, MA (US); Kollol Pal, Needham, MA (US); Nicholas Bockovich, Malden, MA (US); Jon Come, Cambridge, MA (US); Mark Hediger, Marlboro, MA (US)

Correspondence Address: LAHIVE & COCKFIELD, 28 STATE STREET, BOSTON, MA, 02109, US

(21) Appl. No.: 09/797,500

(22) Filed: Mar. 1, 2001

Related U.S. Application Data
(63) Continuation-in-part of application No. 09/645,750, filed on Aug. 24, 2000.
(60) Provisional application No. 60/172,215, filed on Aug. 31, 1999.

(51) Int. Cl. A61K 38/17; G06F 19/00; G01N 33/48; G01N 33/50; C12N 9/64

(52) U.S. Cl. 514/12; 435/226; 702/19

(57) ABSTRACT

The present invention relates to polypeptides which comprise the ligand binding domain of Cdc25, crystalline forms of these polypeptides and the use of these crystalline forms to determine the three dimensional structure of the catalytic domain of Cdc25. The invention also relates to the use of the three dimensional structure of the Cdc25 catalytic domain in methods of designing and/or identifying potential inhibitors of Cdc25 activity, for example, compounds which inhibit the binding of a native substrate to the Cdc25 catalytic domain.

### RELATED APPLICATION

This application claims the benefit of U.S. Provisional Application No. 60/172,215, filed Aug. 31, 1999, the contents of which are incorporated herein by reference in their entirety.

### BACKGROUND OF THE INVENTION

The application of modem molecular genetics to the study of cancer has established that changes in specific DNA sequences can lead to tumor initiation, growth and progression. Many of these changes occur in genes which alter cellular proliferation, either directly (growth stimulatory factors/receptors such as Ras, ErbB2, bcr/abl) or indirectly (transcriptional proteins such as Rb, myc and p53). Recently, many of these oncogenic changes have been linked to alterations in cell cycle regulation. For example, many oncogenes encode components of the pathways by which growth factor signals feed into the cell cycle to induce cell division. Changes in cell cycle proteins, such as cyclins D1 and E, have also been demonstrated to be oncogenic in some cell types.

Cdc25 is a family of dual specificity phosphatases which dephosphorylate both protein phosphotyrosine and phosphothreonine residues. Cdc25 regulates cell cycle progression by controlling the phosphorylation state of the cyclin dependent kinases. When phosphorylated on Tyr¹⁵ and Thr¹⁴, the cyclin dependent kinases (cdk) are inactive and cell cycle progression is prevented. Dephosphorylation by Cdc25 activates cdk and the cell cycle progresses. The activity of Cdc25 phosphatases is clearly required for cell cycle transition, and these enzymes serve as the rate-limiting mitotic activators. For example, mutation of Cdc25 in yeast produces cells that arrest in G2 phase. Mutation of the Cdc25 homologue in Drosophila results in G2 arrest of cells early in embryogenesis.

Three distinct mammalian Cdc25 homologues have been identified, denoted Cdc25A, Cdc25B and Cdc25C. Each of these appears to target a particular cdk/cyclin complex. In mammalian cells microinjection of antibodies against Cdc25A or C inhibits cell entry into S (Hoffmann et al., EMBO J. 13: 4302-10 (1994)) and M (Millar et al., Proc. Nat. Acad, Sci. USA 88: 10500-4 (1991)) respectively. Recent publications have demonstrated that Cdc25C is central to DNA damage checkpoint arrest, such as an arrest produced by a cytotoxic agent. Damage activates the serine kinase Chk-1 that phosphorylates Cdc25C on serine 216. This phosphorylation makes Cdc25C a binding partner for the family of 14-3-3 binding proteins. Binding to 14-3-3 proteins is believed to prevent Cdc25C from activating the cdk/cyclin complex cdc2/cycB and results in G2 cell cycle arrest (Furnari et al., Science 277: 1495-1497 (1997); Sanchez et al., Science 277: 1497-1501 (1997), Peng et al., Science 277: 1501-1505 (1997)). Additional studies have demonstrated that both Cdc25A and B can act as oncogenes and transform cells when overexpressed (Galaktionov et al., Science 269: 1575-1577 (1995)).

Due to its role in regulating the cell cycle, Cdc25 is a potential target for therapies aimed at controlling proliferative diseases, such as cancer. The development of biochemical assays for Cdc25 has enabled drug discovery to proceed along the pathways of identifying lead Cdc25 inhibitors by high-throughput screening of compound libraries and by testing compounds that mimic substrate structure; however, rational, structure-based design has not been possible up to this point because of the lack of accurate three-dimensional data.

### SUMMARY OF THE INVENTION

The present invention relates to polypeptides which comprise the ligand binding domain of Cdc25, crystalline forms of these polypeptides and the use of these crystalline forms to determine the three dimensional structure of the catalytic domain of Cdc25. The invention also relates to the use of the three dimensional structure of the Cdc25 catalytic domain in methods of designing and/or identifying potential inhibitors of Cdc25 activity, for example, compounds which inhibit the binding of a native substrate to the Cdc25 catalytic domain.

In one embodiment, the present invention provides polypeptides comprising the ligand binding domain of Cdc25, crystalline forms of these polypeptides, optionally complexed with a ligand, and the three dimensional structure of the polypeptides, including the three dimensional structure of the Cdc25 catalytic domain. The polypeptide, preferably, has the catalytic activity of a Cdc25.

In another embodiment, the invention provides a method of determining the three dimensional structure of a crystalline polypeptide comprising the Cdc25 catalytic domain. The method comprises the steps of (1) obtaining a crystal of the polypeptide comprising the catalytic domain of Cdc25; (2) obtaining x-ray diffraction data for said crystal; and (3) solving the crystal structure of said crystal by using said x-ray diffraction data and the atomic coordinates for the Cdc25 binding domain of a second polypeptide. The method optionally comprises the additional step of obtaining the polypeptide prior to obtaining the crystal.

The invention further relates to a method of identifying a compound which is a potential inhibitor of Cdc25. The method comprises the steps of (1) obtaining a crystal of a polypeptide comprising the catalytic domain of Cdc25; (2) obtaining the atomic coordinates of the polypeptide in said crystal; (3) using said atomic coordinates to define the catalytic domain of Cdc25; and (4) identifying a compound which fits the catalytic domain. The method can further include the steps of obtaining or synthesizing the compound identified in step 4, and assessing the ability of the identified compound to inhibit at least one biological activity of Cdc25, such as enzymatic activity.

In another embodiment, the method of identifying a potential inhibitor of Cdc25 comprises the step of determining the ability of one or more functional groups and/or moieties of the compound, when present in, or bound to, the Cdc25 catalytic domain, to interact with one or more subsites of the Cdc25 catalytic domain. Generally, the Cdc25 catalytic domain is defined by the atomic coordinates of a polypeptide comprising the Cdc25 catalytic domain. If the compound is able to interact with a preselected number or set of subsites, or has a calculated interaction energy within a desired or preselected range, the compound is identified as a potential inhibitor of Cdc25.

The invention further provides a method of designing a compound which is a potential inhibitor of Cdc25. The method includes the steps of (1) identifying one or more functional groups capable of interacting with one or more subsites of the Cdc25 catalytic domain; and (2) identifying a scaffold which presents the functional group or functional groups identified in step 1 in a suitable orientation for interacting with one or more subsites of the Cdc25 catalytic domain. The compound which results from attachment of the identified functional groups or moieties to the identified scaffold is a potential inhibitor of Cdc25. The Cdc25 catalytic domain is, generally, defined by the atomic coordinates of a polypeptide comprising the Cdc25 catalytic domain.

In yet another embodiment, the invention provides compounds which are inhibitors of Cdc25 and which fit, or bind to, the Cdc25 catalytic domain. Such compounds typically comprise one or more functional groups which, when the compound is bound in the Cdc25 catalytic domain, interact with one or more subsites of the catalytic domain. Generally, the Cdc25 catalytic domain is defined by the atomic coordinates of a polypeptide comprising the Cdc25 catalytic domain. In a particular embodiment, the Cdc25 inhibitor is a compound which is identified or designed by a method of the present invention.

The present invention further provides a method for treating a condition mediated by Cdc25 in a patient. The method comprises administering to the patient a therapeutically or prophylactically effective amount of a Cdc25 inhibitor, such as a Cdc25 inhibitor of the invention, for example, a compound identified as a Cdc25 inhibitor or designed to inhibit Cdc25 by a method of the present invention.

The present invention provides several advantages. For example, the invention provides the first detailed three dimensional structure of the catalytic domain of a Cdc25 protein. This structure enables the rational development of inhibitors of Cdc25 by permitting the design and/or identification of molecular structures having features which facilitate binding to the Cdc catalytic domain. The methods of use of this structure disclosed herein, thus, permit more rapid discovery of compounds which are potentially useful for the treatment of conditions which are mediated, at least in part, by Cdc25 activity.

### BRIEF DESCRIPTION OF THE DRAWINGS

FIG. 1 presents the amino acid sequence of human Cdc25A (SEQ ID NO: 1).

FIG. 2 presents the amino acid sequence of human Cdc25B (SEQ ID NO: 2).

FIG. 3 presents the amino acid sequence of human Cdc25C (SEQ ID NO: 3).

FIG. 4 presents the amino acid sequence of polypeptide Cdc25A ΔN1A (SEQ ID NO: 4).

FIG. 5 presents the amino acid sequence of polypeptide Cdc25B ΔN1B (SEQ ID NO: 5).

FIG. 6 presents the amino acid sequence of polypeptide Cdc25A ΔN5A (SEQ ID NO: 6).

FIG. 7 presents the amino acid sequence of polypeptide Cdc25C ΔN1C (SEQ ID NO: 7).

FIG. 8 presents the amino acid sequence of polypeptide Cdc25A ΔN8A (SEQ ID NO: 8).

FIG. 9 presents the amino acid sequence of polypeptide Cdc25A ΔN8A-c17 (SEQ ID NO: 9).

FIG. 10 presents the amino acid sequence of polypeptide Cdc25B ΔN5B (SEQ ID NO: 10).

FIG. 11 presents the amino acid sequence of polypeptide Cdc25B ΔN8B (SEQ ID NO: 11).

FIG. 12 presents the amino acid sequence of polypeptide Cdc25B ΔN8B-c17 (SEQ ID NO: 12).

FIG. 13 presents the amino acid sequence of polypeptide Cdc25B ΔN8B-c18 (SEQ ID NO: 13).

FIG. 14 presents the amino acid sequence of polypeptide Cdc25C ΔN9C (SEQ ID NO: 14).

FIG. 15A to 15PPP present the atomic coordinates for dc25B(ΔN8B)/cdc1249 complex (crystal 19).

FIG. 16A to 16I present the atomic coordinates for Cdc25A(ΔN1A).

FIG. 17A to 17EE present the atomic coordinates for Cdc25B(ΔN1B)/cdc1249 complex (crystal 5).

FIG. 18A to 18X present the atomic coordinates for Cdc25A(ΔN8A) (crystal 3).

FIG. 19A to 19I present the atomic coordinates for Cdc25B(ΔN1B)/cdc1671 complex (crystal 15).

FIG. 20 illustrates the complex of the Cdc25B catalytic domain and the pentapeptide cdc1249 showing the protein secondary structure, the ligand bound at the catalytic loop (thick bonds), and the ligand bound at the distal site (thin bonds)

FIG. 21 shows the complex of the Cdc25B catalytic domain and the pentapeptide cdc1249 showing two symmetry related protein molecules interacting with the ligand bound at the catalytic site; water molecules and ions are not shown

FIG. 22 shows the complex of the Cdc25B catalytic domain and the pentapeptide cdc1249 showing a top view of the molecular surface around the ligand binding area.

FIG. 23 shows a side view of the complex of the Cdc25B catalytic domain and the pentapeptide cdc1249.

FIG. 24 shows a top view of the complex of the Cdc25B catalytic domain and the pentapeptide cdc1249 with protein residues labeled.

FIG. 25 shows a side view of the complex of the Cdc25B catalytic domain and the pentapeptide cdc1249.

FIG. 26 illustrates the complex of the Cdc25B catalytic domain and the pentapeptide cdc1249 showing a top view of the molecular surface around the ligand binding area, with each subsite labeled.

FIG. 27 shows the complex of the Cdc25B catalytic domain and the pentapeptide cdc1249 showing a side view with subsites 1-6 labeled

FIG. 28 is a side view of a potential tight-binding inhibitor complexed to the Cdc25B catalytic domain.

### DETAILED DESCRIPTION OF THE INVENTION

The present invention relates to the x-ray crystallographic study of polypeptides comprising the catalytic domains of Cdc25. The atomic coordinates which result from this study are of use in identifying compounds which fit in the catalytic domain and are, therefore, potential inhibitors of Cdc25. These Cdc25 inhibitors are of use in methods of treating a patient having a condition which is modulated by Cdc25 activity, for example, a condition characterized by excessive, inappropriate or undesirable cellular proliferation. Recent evidence indicates that Cdc25 plays a role in the development of cancer. For example, studies have suggested that overexpression of Cdc25B in transgenic mice under the MMTV promoter make them more susceptible to DMBA induced mammary tumors (Slosberg et al., Proc. Am. Assoc. Cancer Res. 39: 255 (1998)). Both Cdc25A and Cdc25B are frequently overexpressed in breast cancer (Galaktionov et al., Science 269: 1575-1577 (1995)), head and neck cancer (Gasparotto et al., Cancer Res 57: 2366-2368 (1997)), gastric carcinoma (Kudo et al., Jpn J. Cancer Res 88: 947-952 (1997)), and Non Hodgkin's lymphomas (Hernandez et al., Cancer Res 58: 1762-1767 (1998)).

Studies in cell lines lacking the cdk inhibitor p15 indicate TGF-β can inhibit cell progression by modulating levels of Cdc25A (Iavarone et al., Nature 387: 417-422 (1997)). Similarly, levels of Cdc25A and growth of a tumor cell line has been shown to be modulated by α-interferon (Tiefenbrun et al., Mol Cell Biol 16: 3934-3944 (1996)). Further, it has recently been shown that antisense oligonucleotides against Cdc25B inhibit the growth of a tumor cell line (Garnerhamrick et al., Int. J. Cancer 76: 729-728 1998). These results support the idea that Cdc25 inhibitors may block one or more pathways involved in cell transformation.

The x-ray crystal structure of human Cdc25A was reported by Saper et al. in 1998 (Saper et al., Cell 93: 617-625 (1998)). The structure does not provide atomic-level details of the catalytic loop or the amino acid residues at the carboxyl terminus. Further, the structure does not include a bound inhibitor of Cdc25A.

The Examples describe the preparation of polypeptides comprising the catalytic domains of human Cdc25A, Cdc25B and Cdc25C and the crystallization of the Cdc25A and Cdc25B polypeptides. As used herein, the term “catalytic domain” refers to any or all of the following sites in Cdc25: the substrate binding site; the site where the pentapeptide inhibitor described below binds and the site where the cleavage of a substrate occurs. For Cdc25A, the catalytic domain is defined by amino acid residues from about residue 336 to about residue 523 of SEQ ID NO: 1. For Cdc25B, the catalytic domain is defined by residues from about residue 378 to about residue 566 of SEQ ID NO: 2 (Xu et al., J. Biol. Chem., 271: 5118-5124 (1996)).

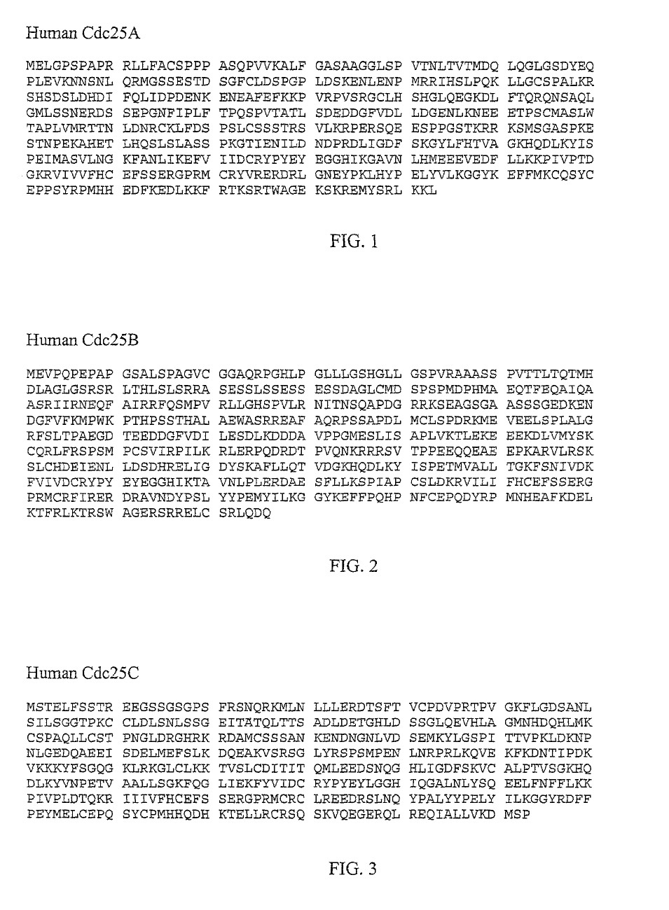

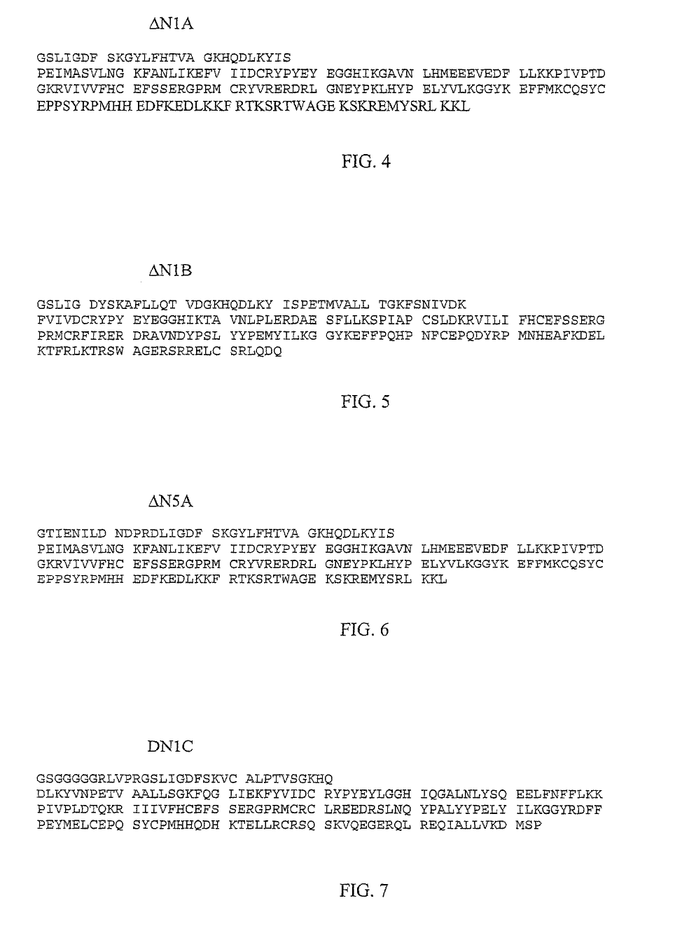

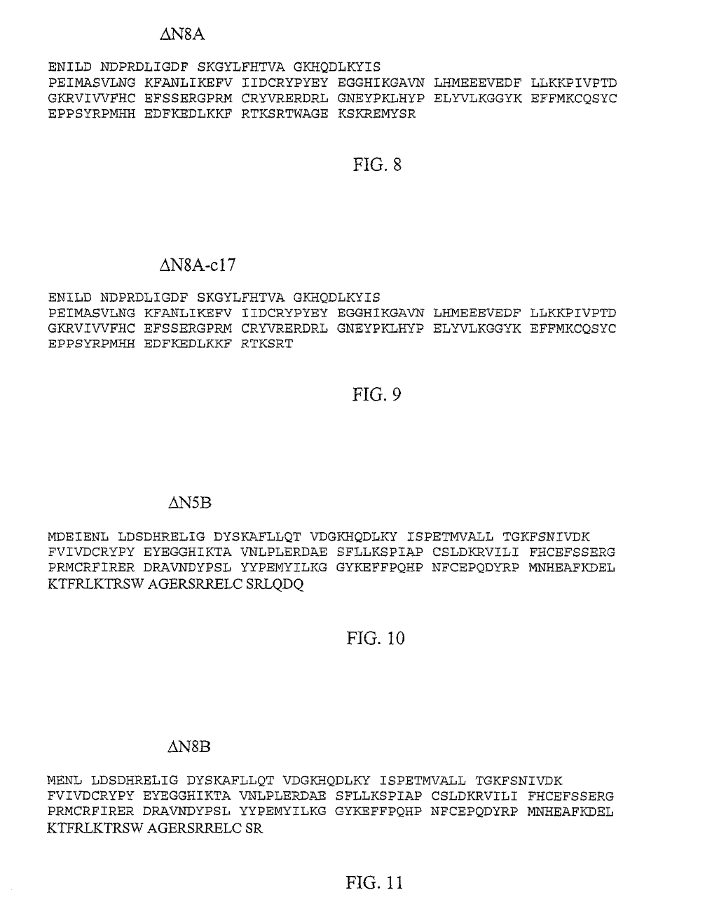

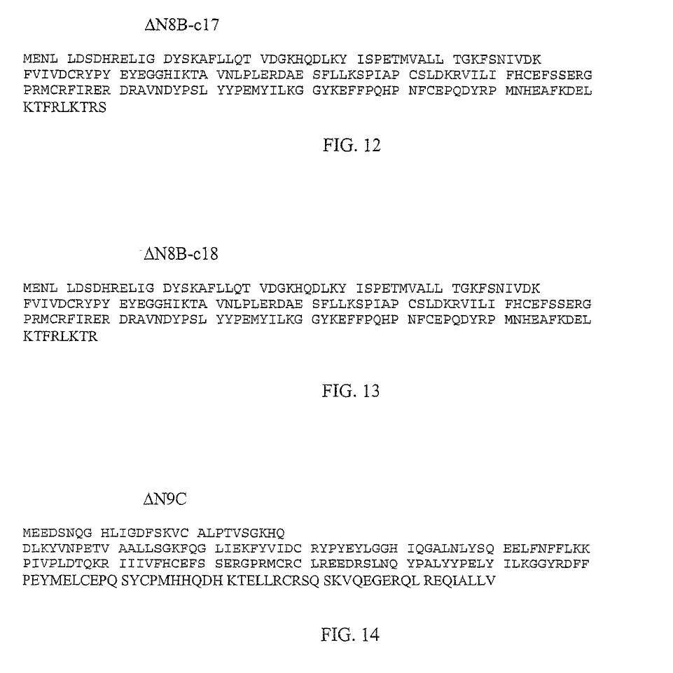

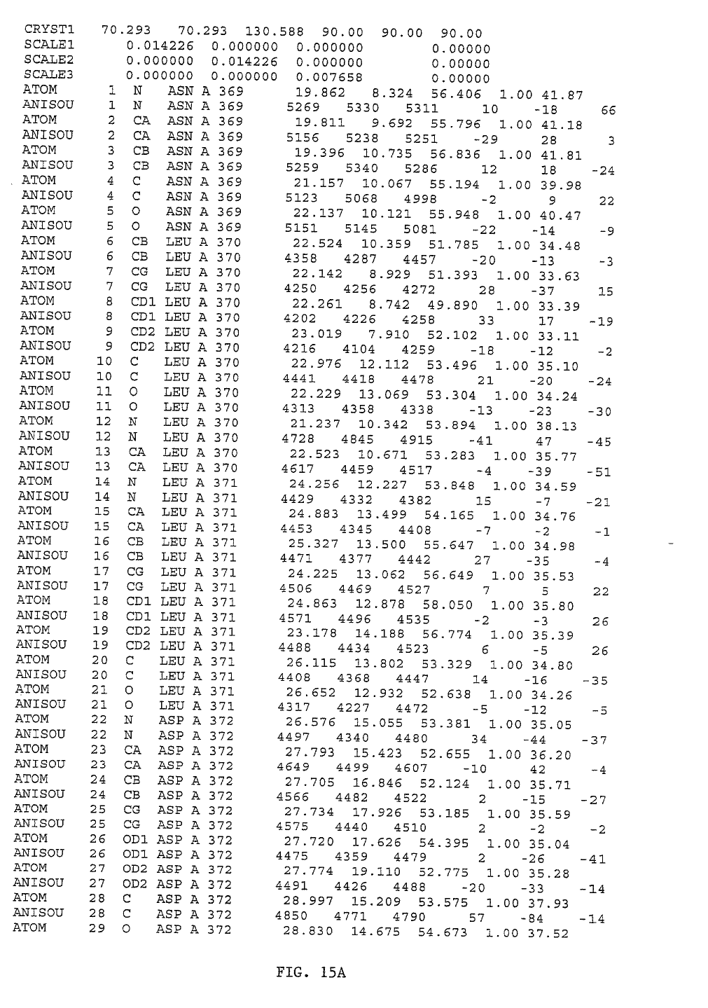

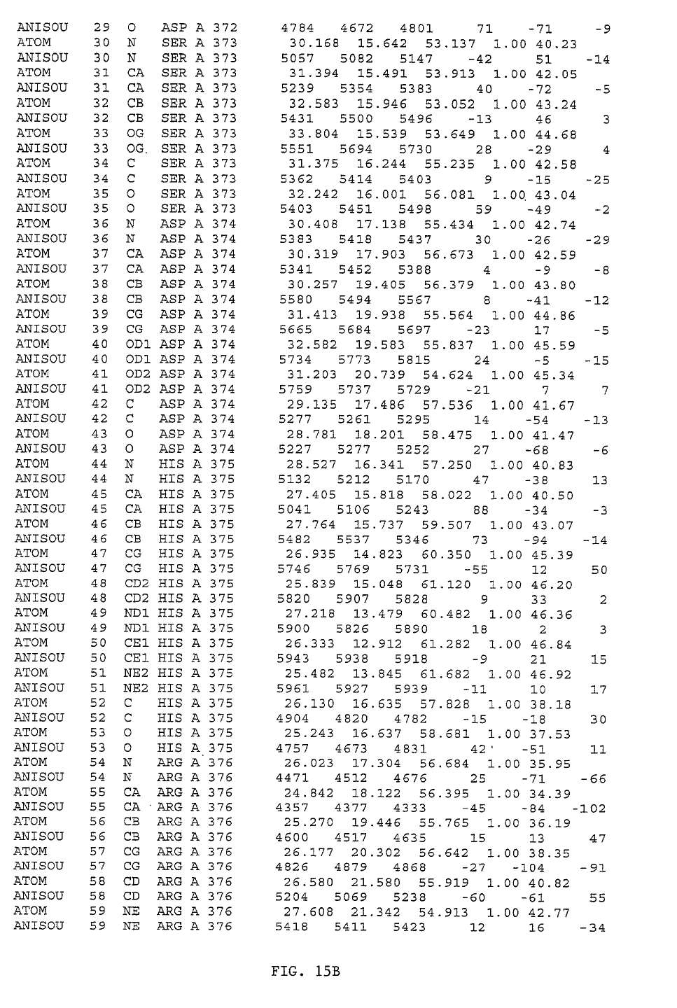

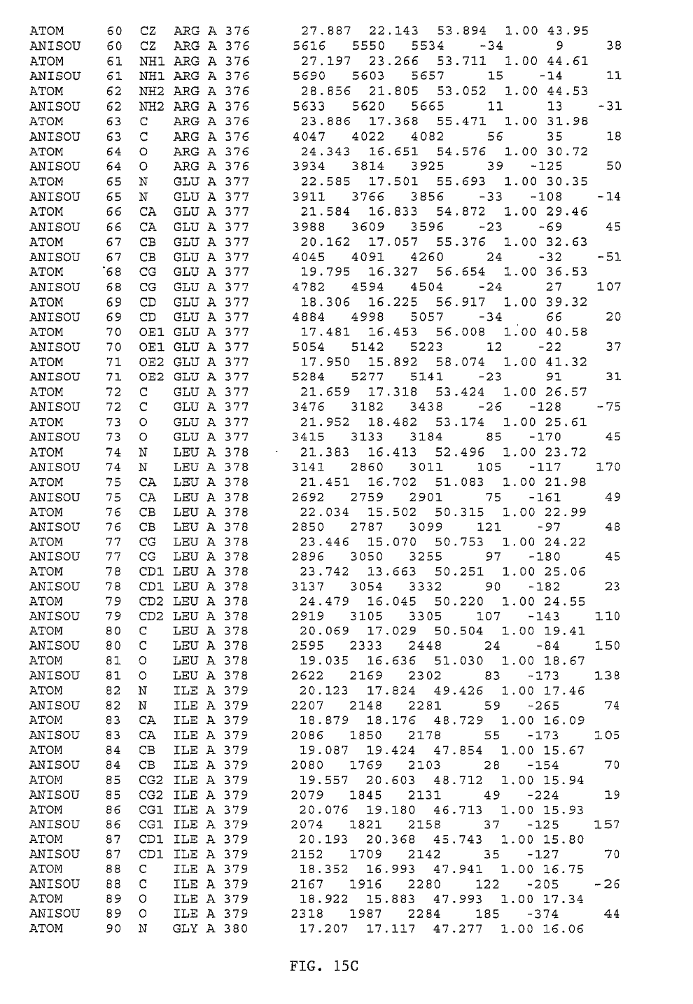

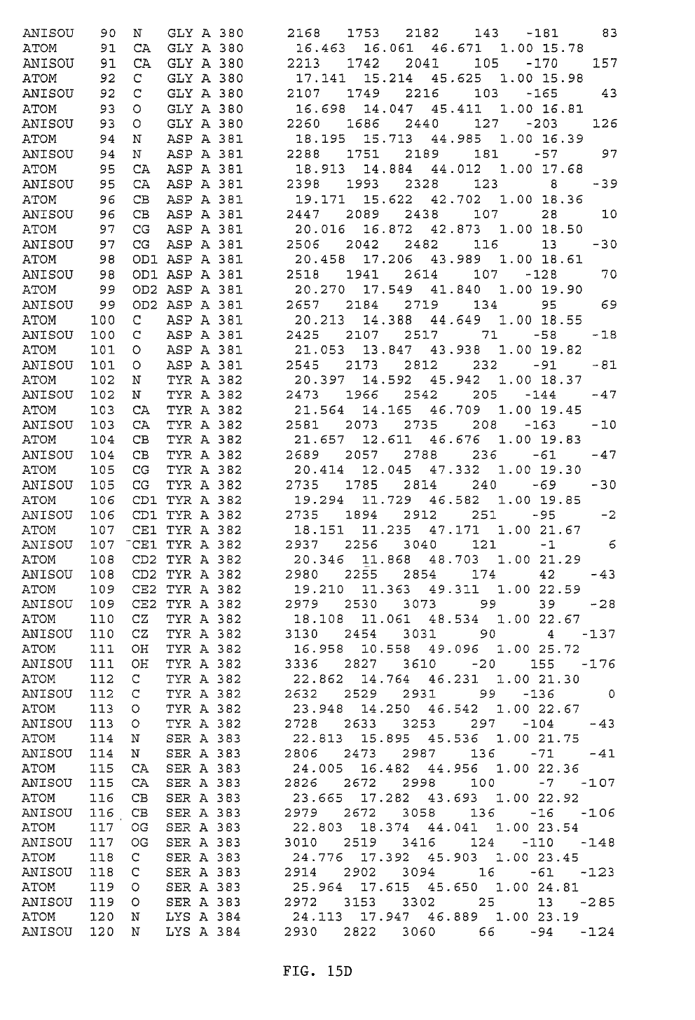

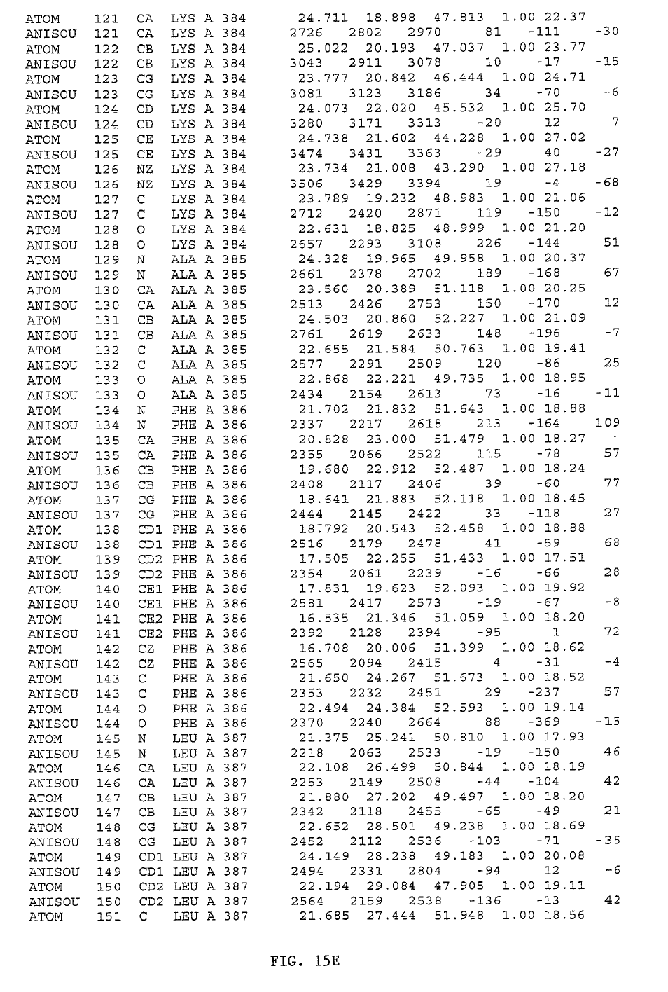

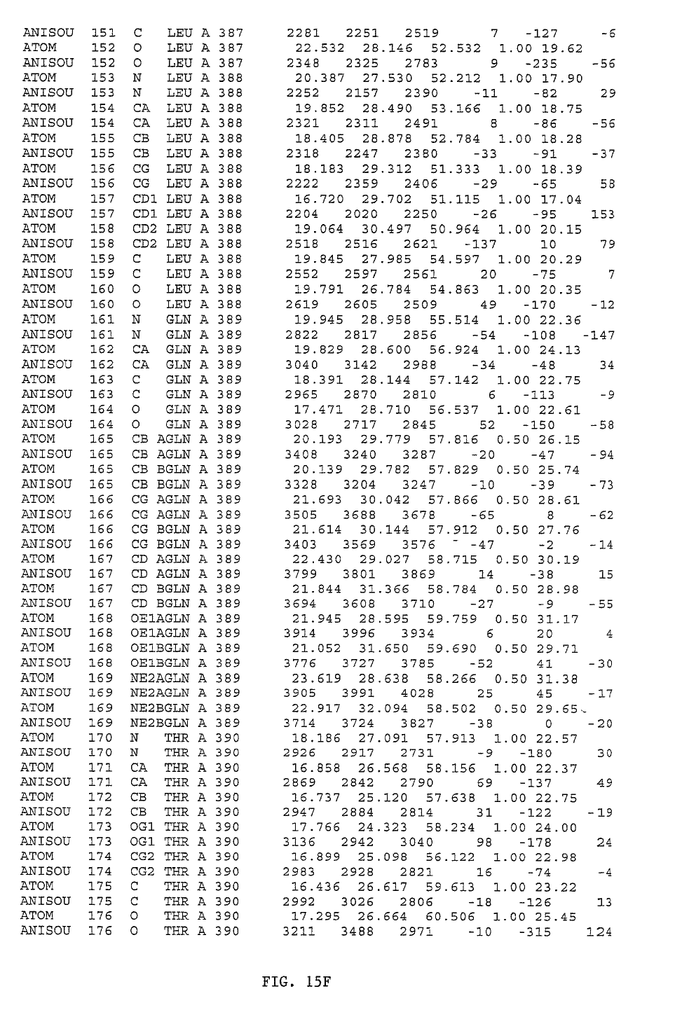

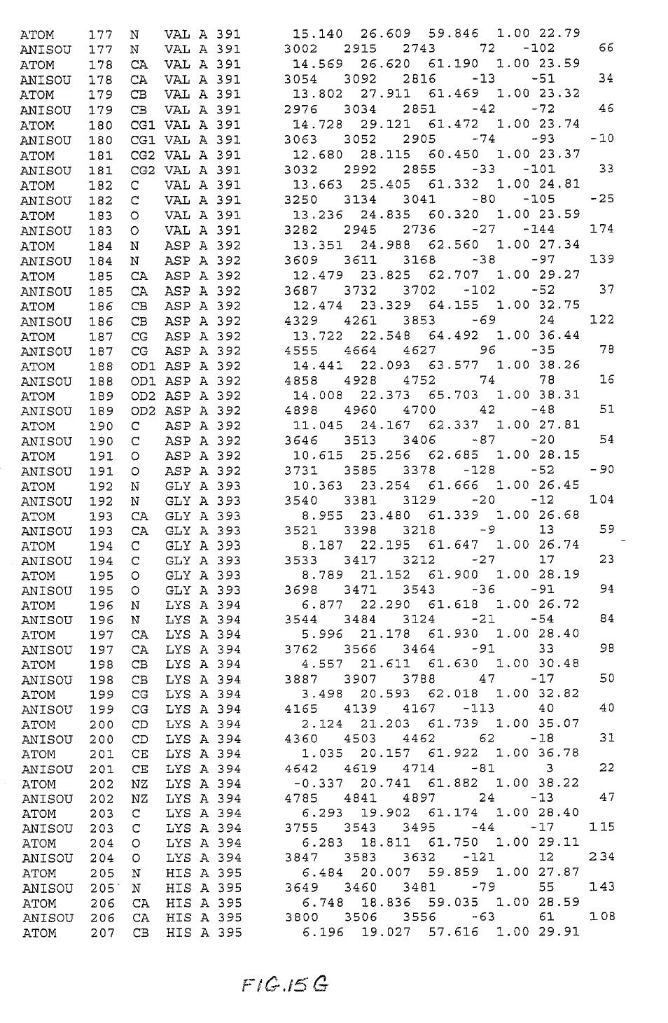

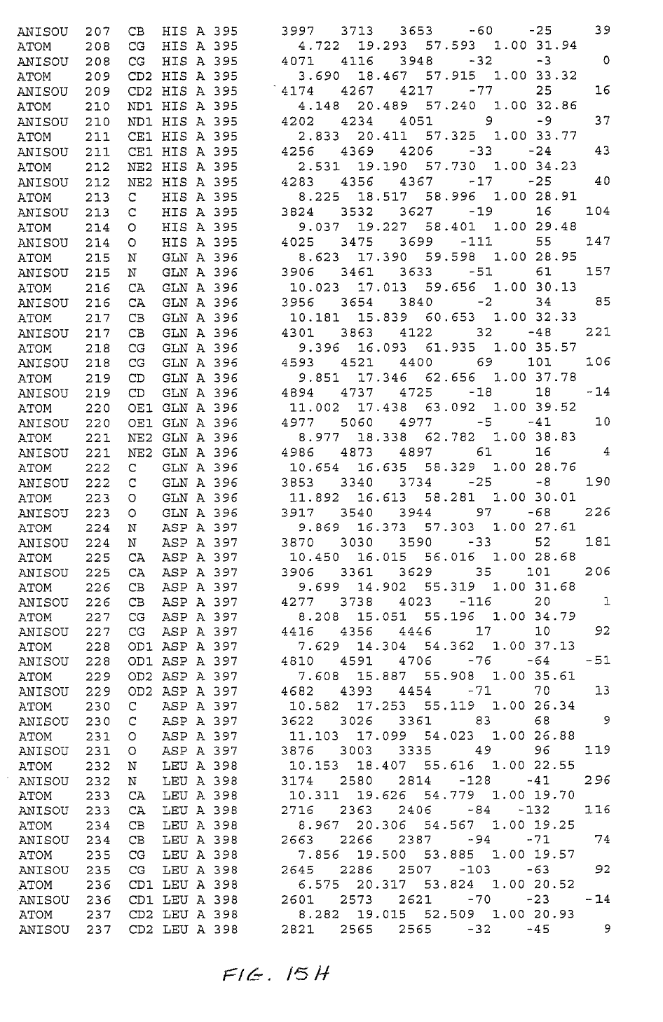

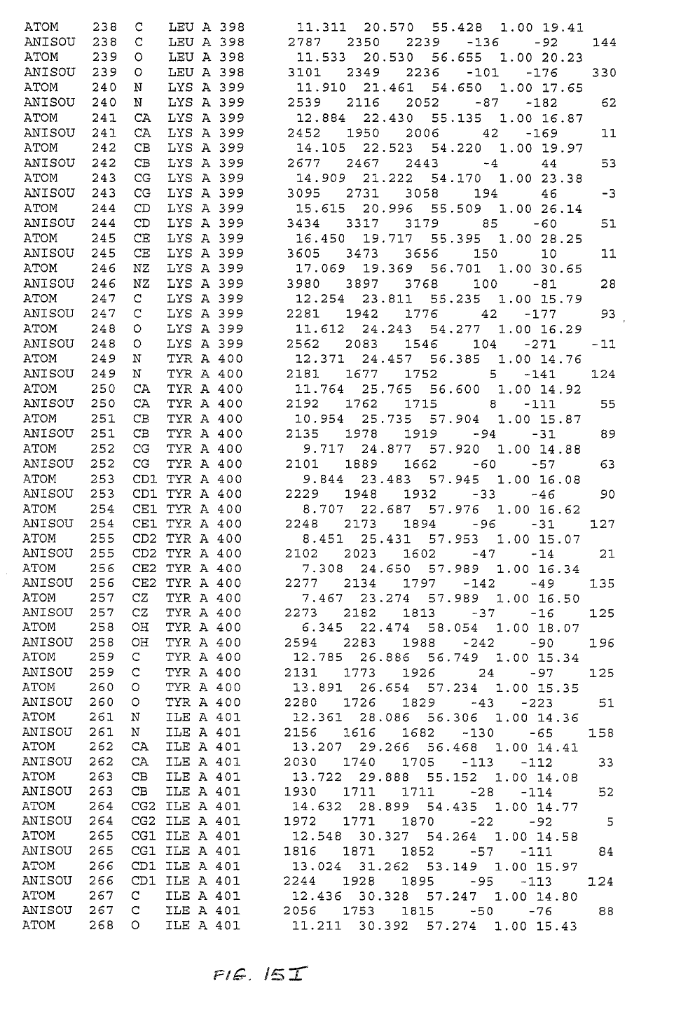

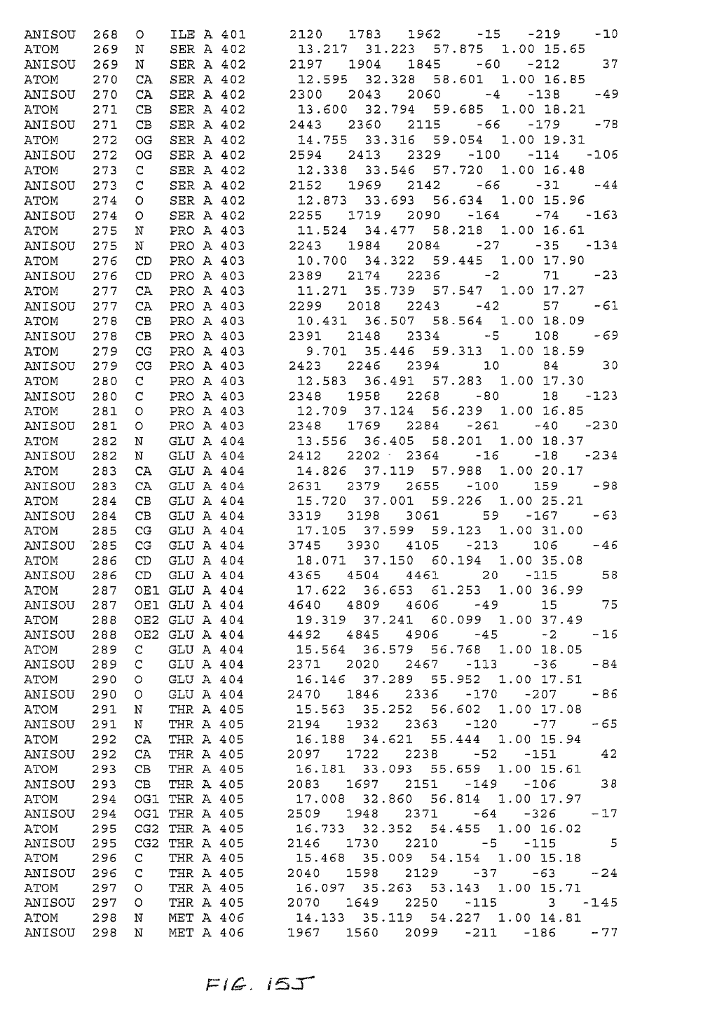

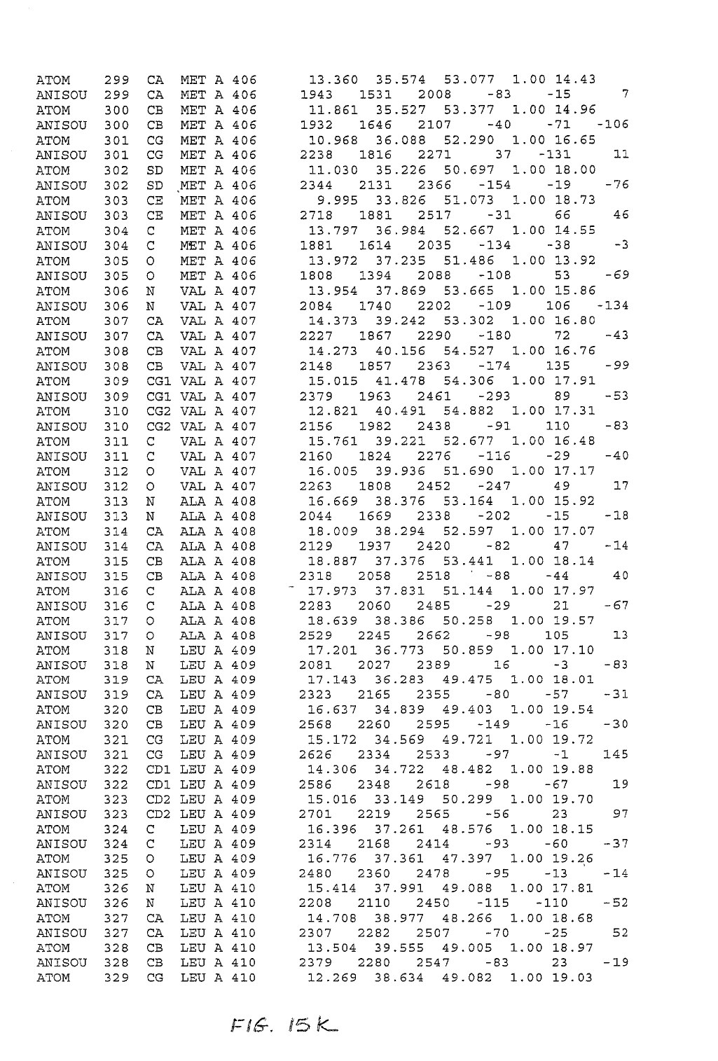

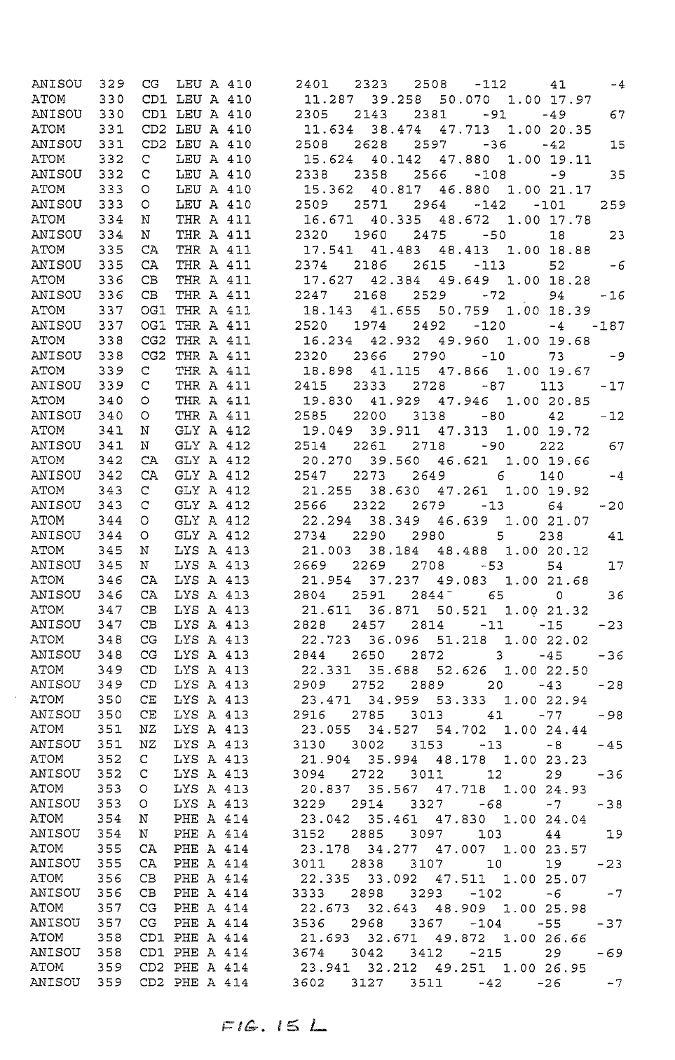

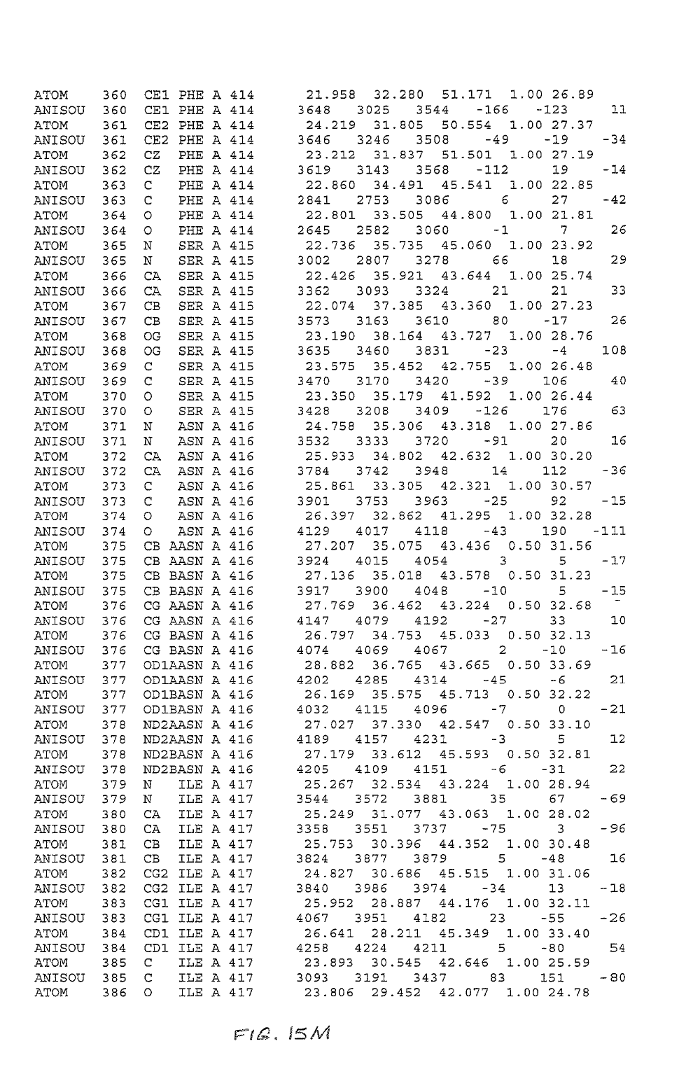

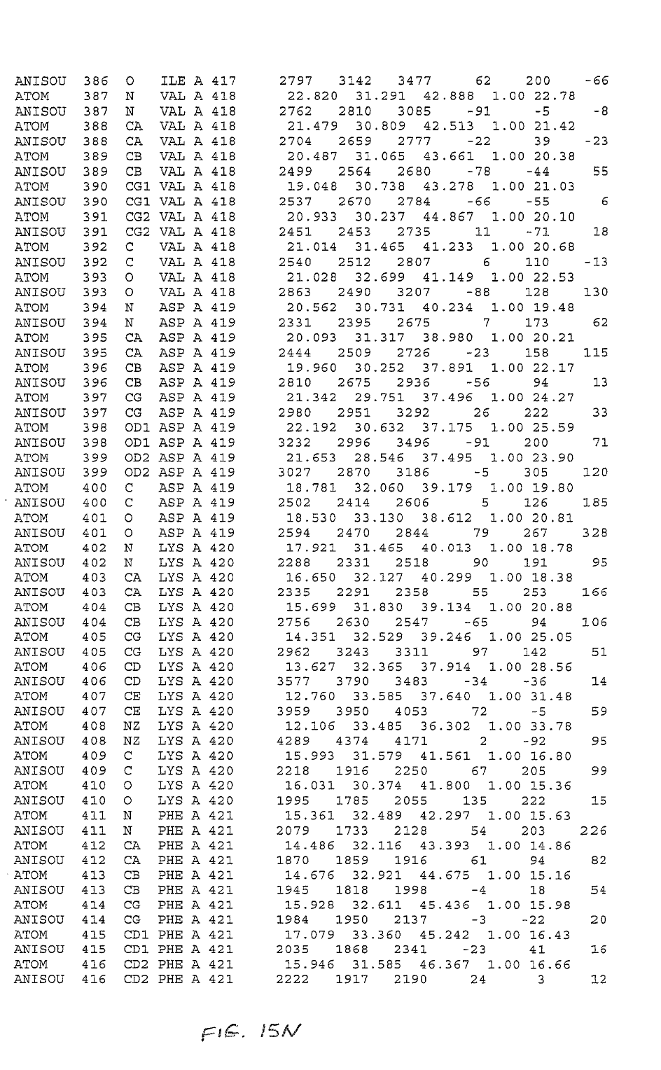

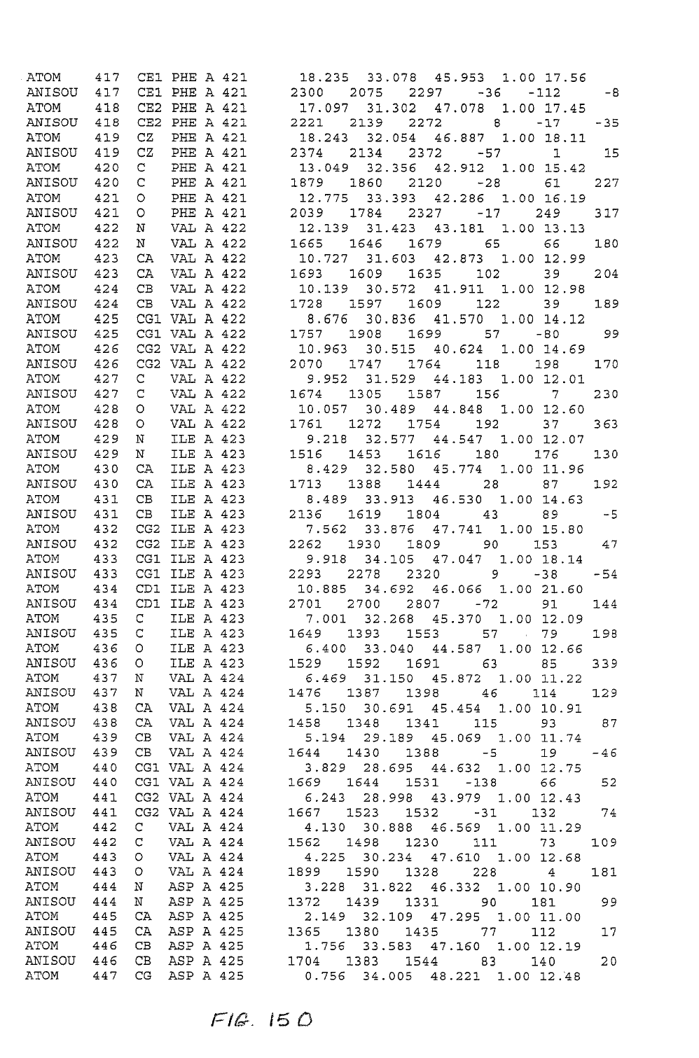

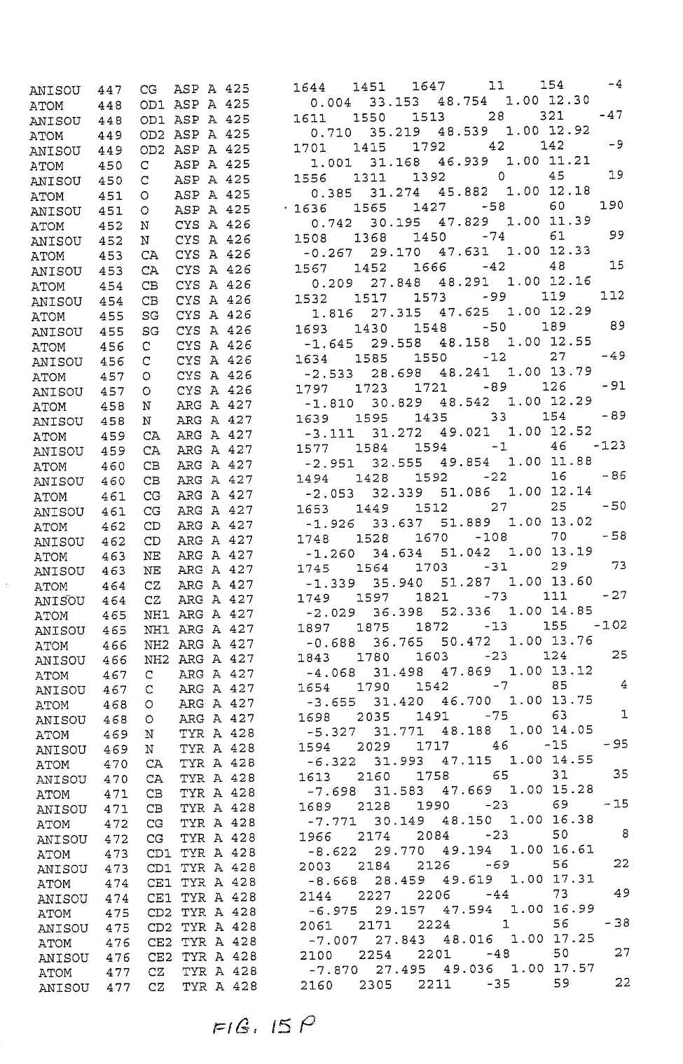

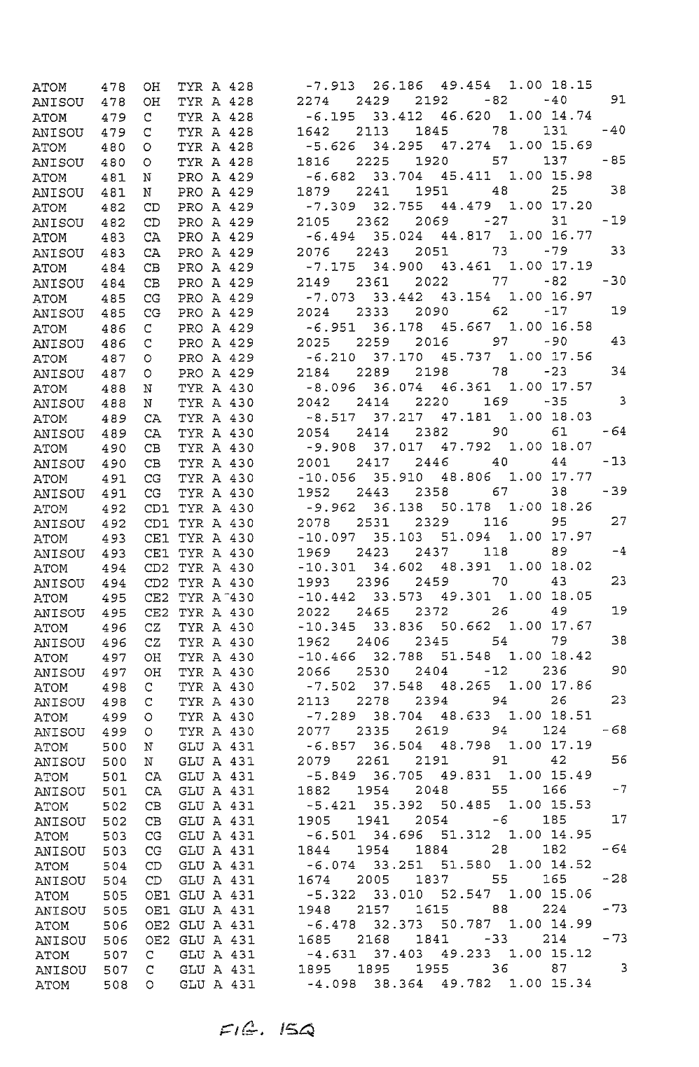

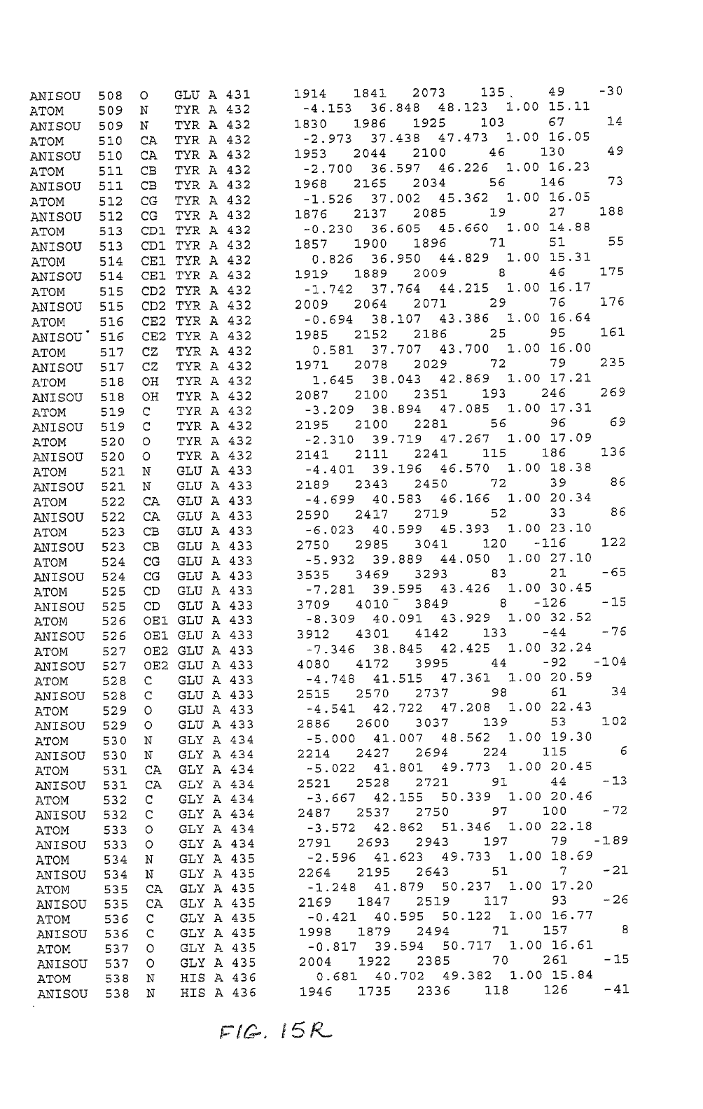

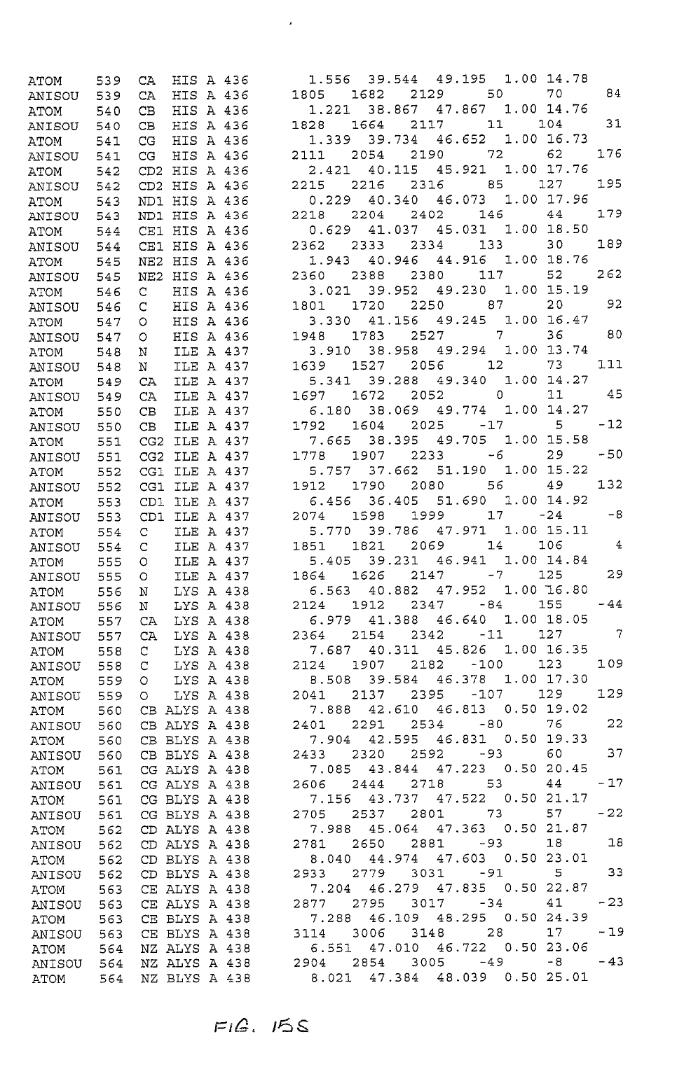

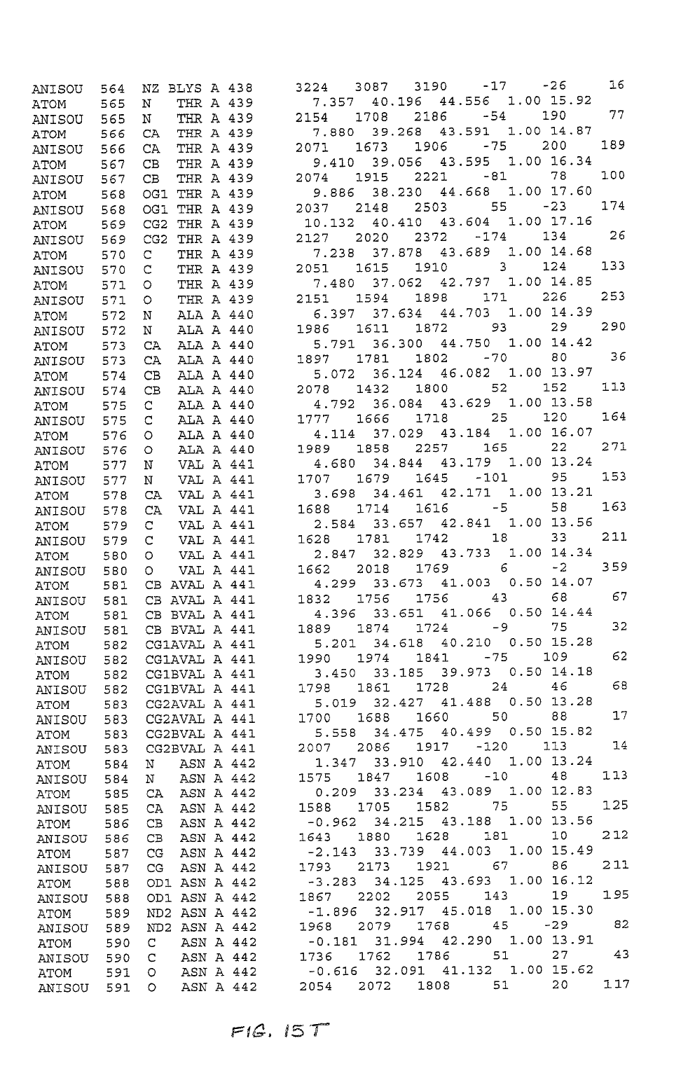

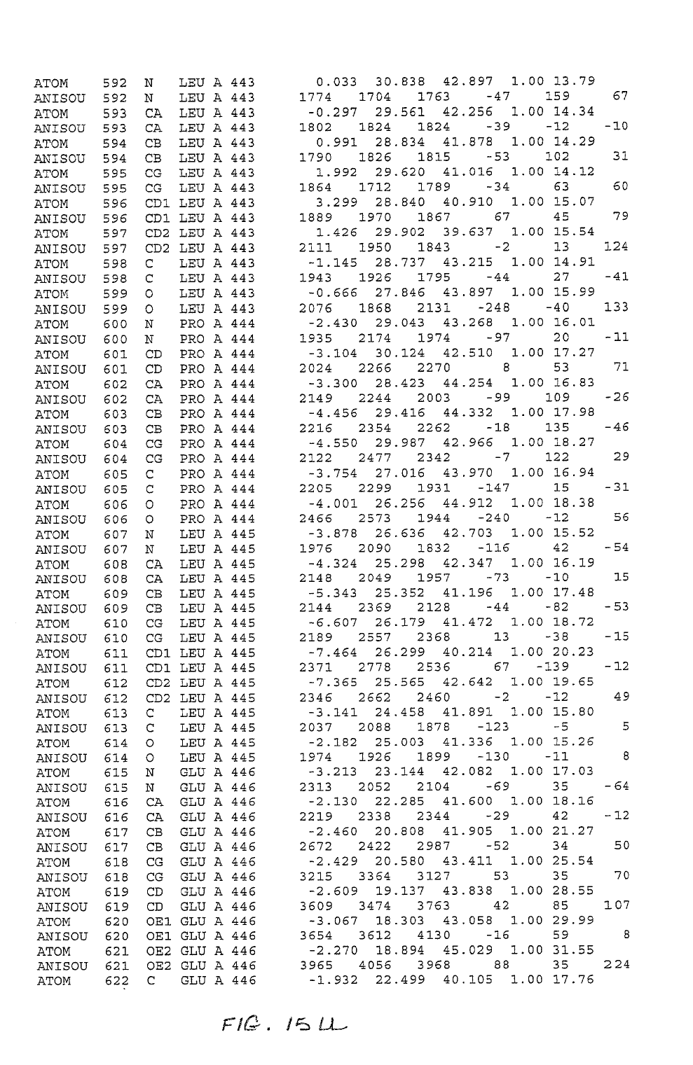

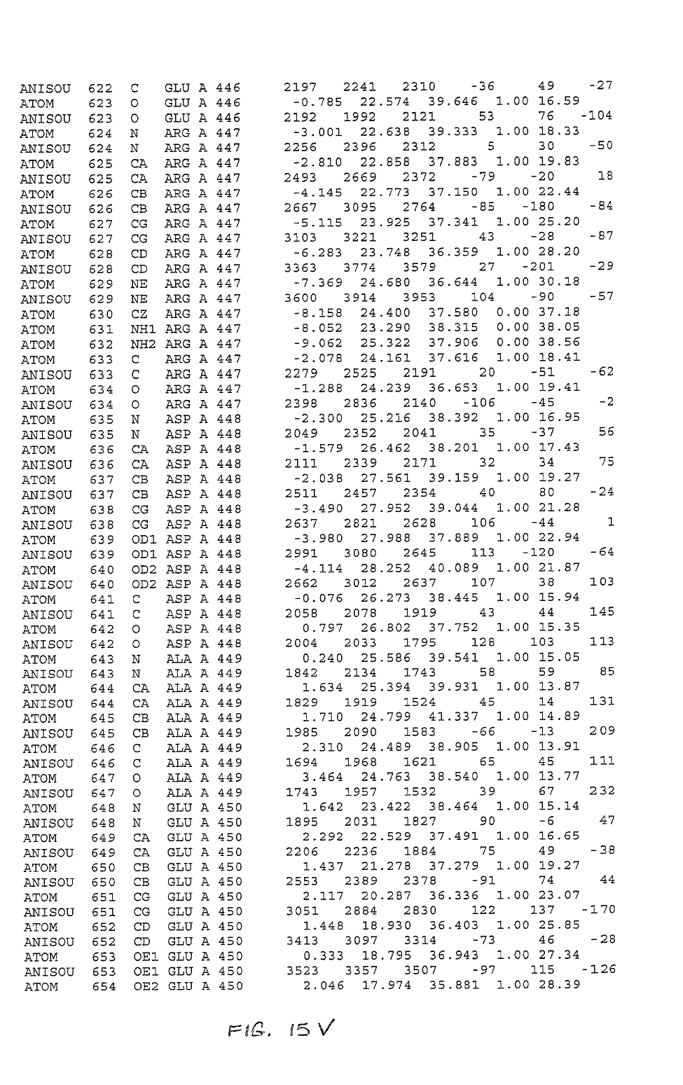

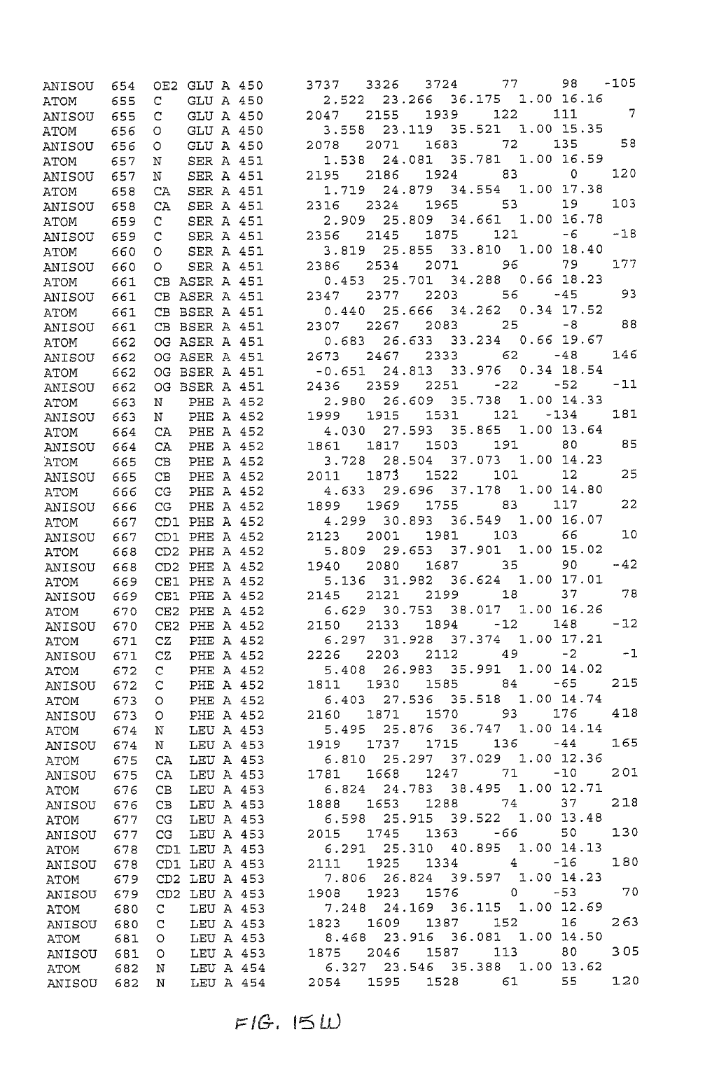

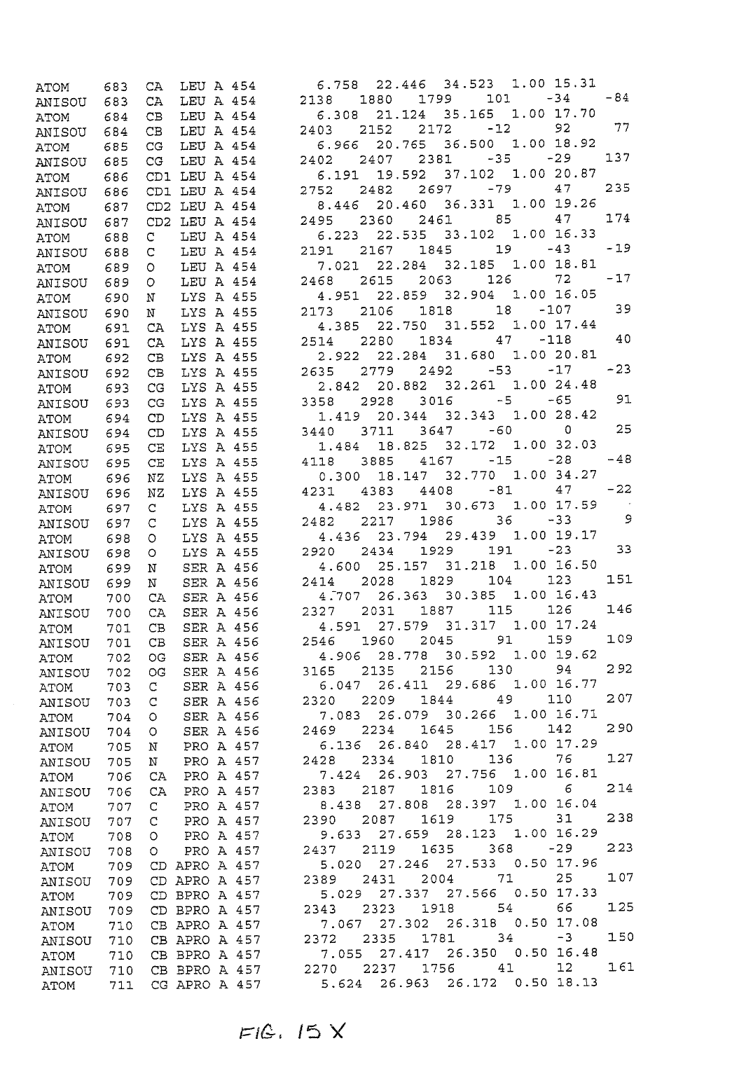

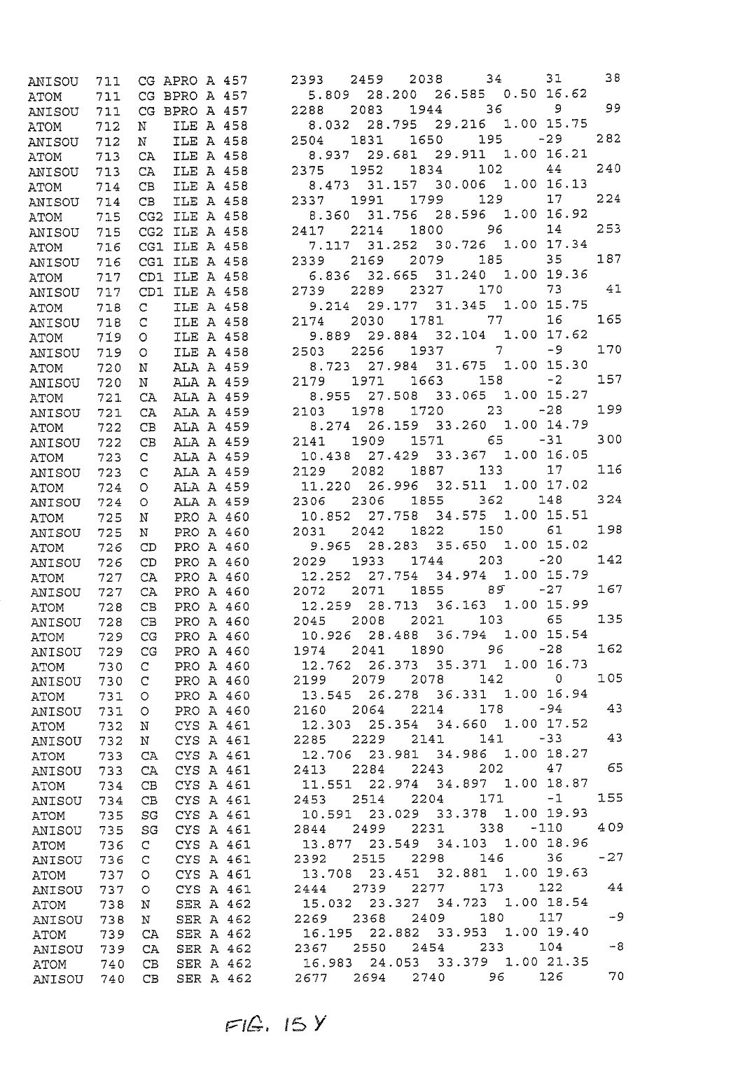

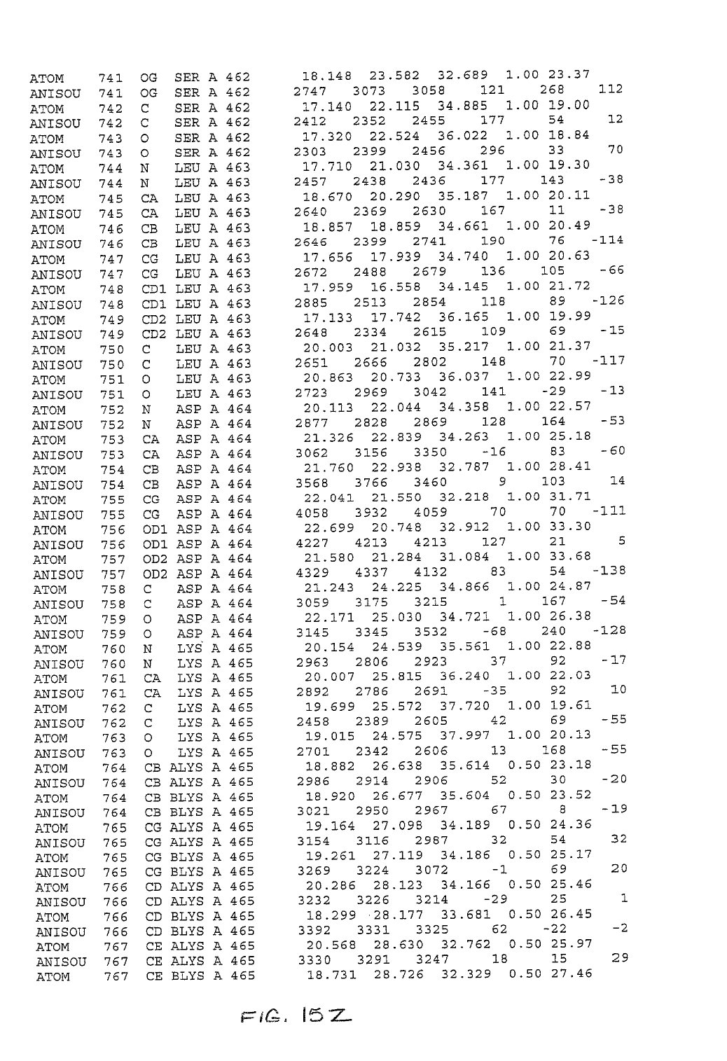

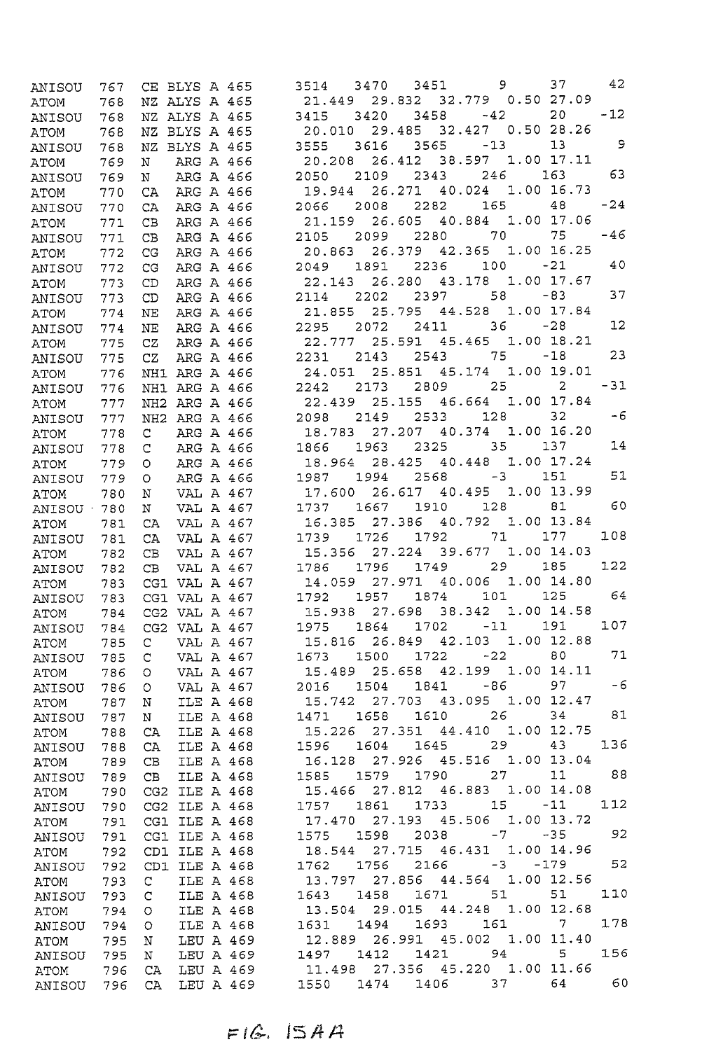

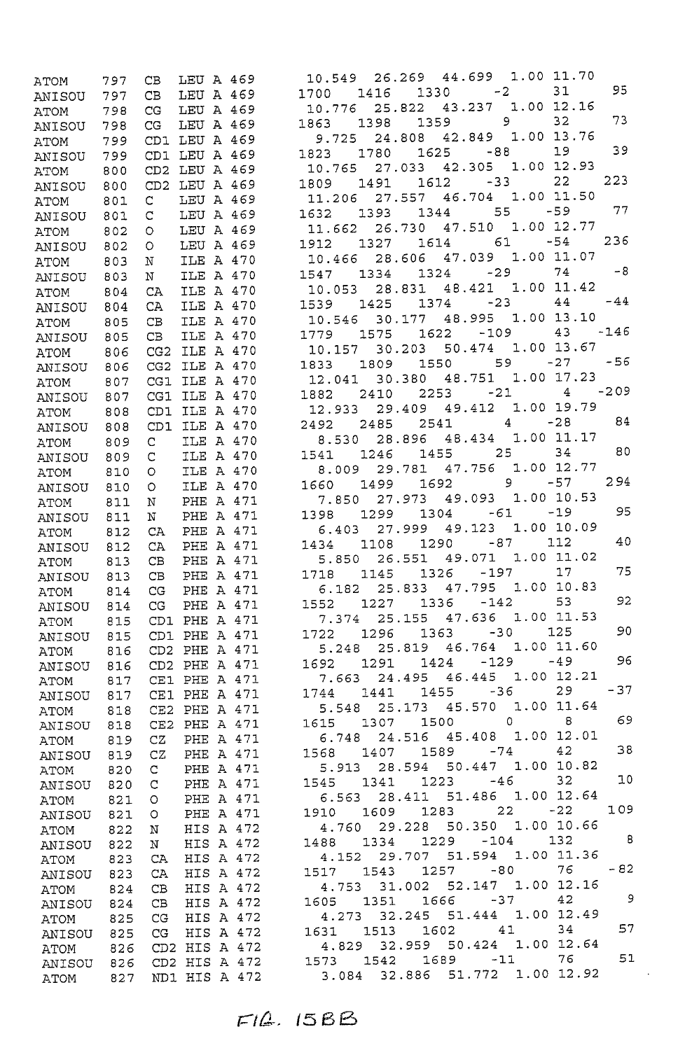

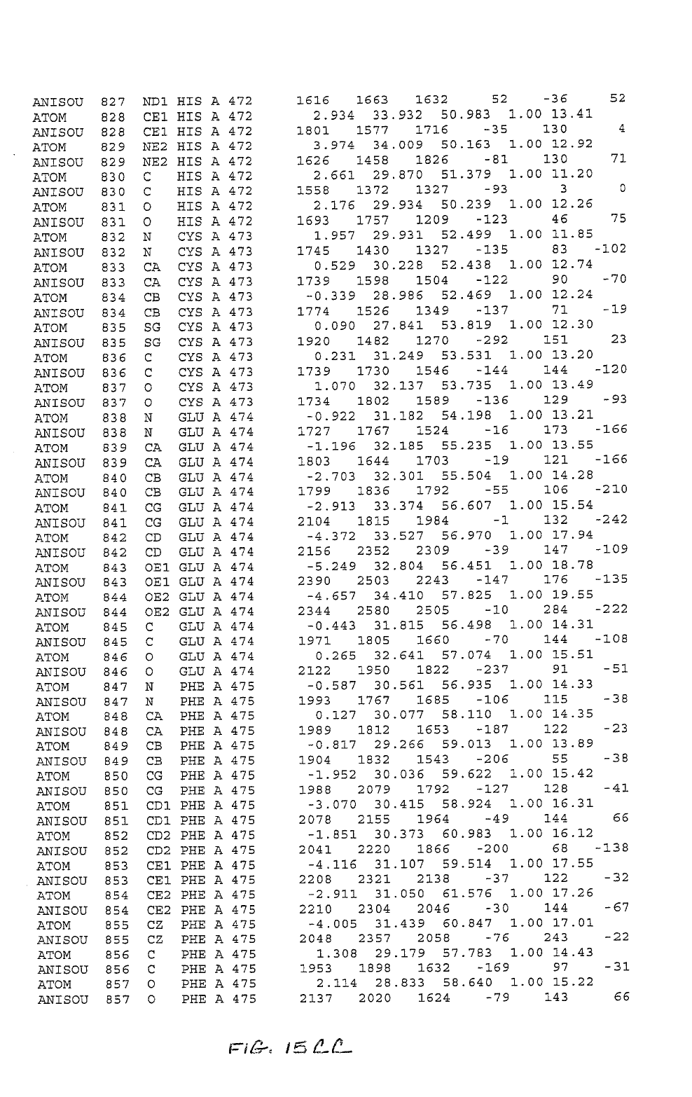

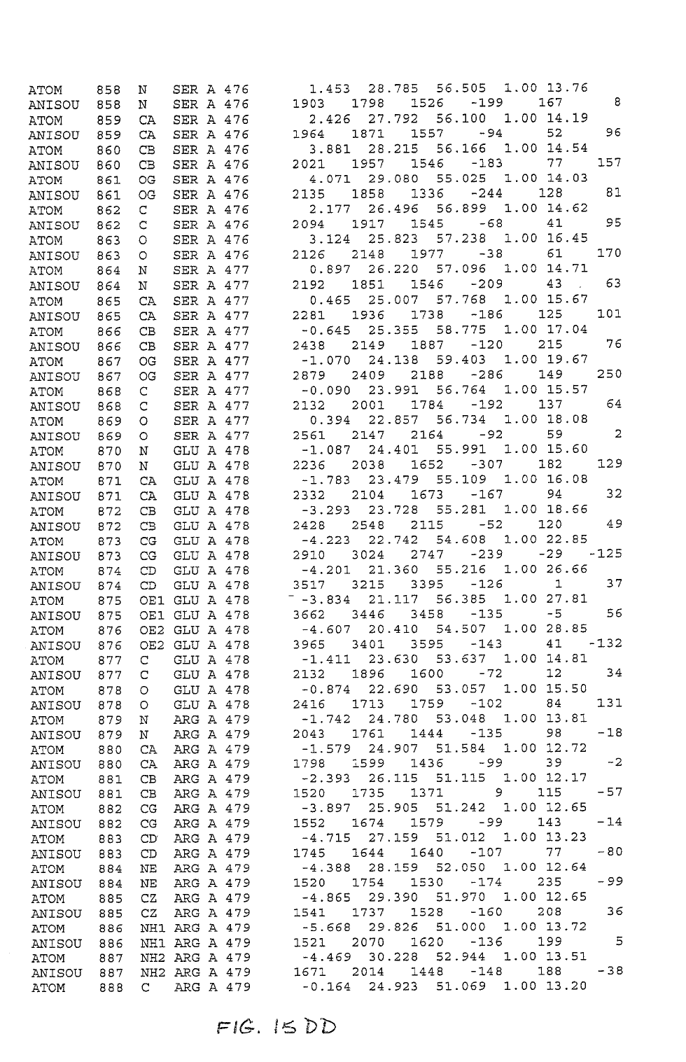

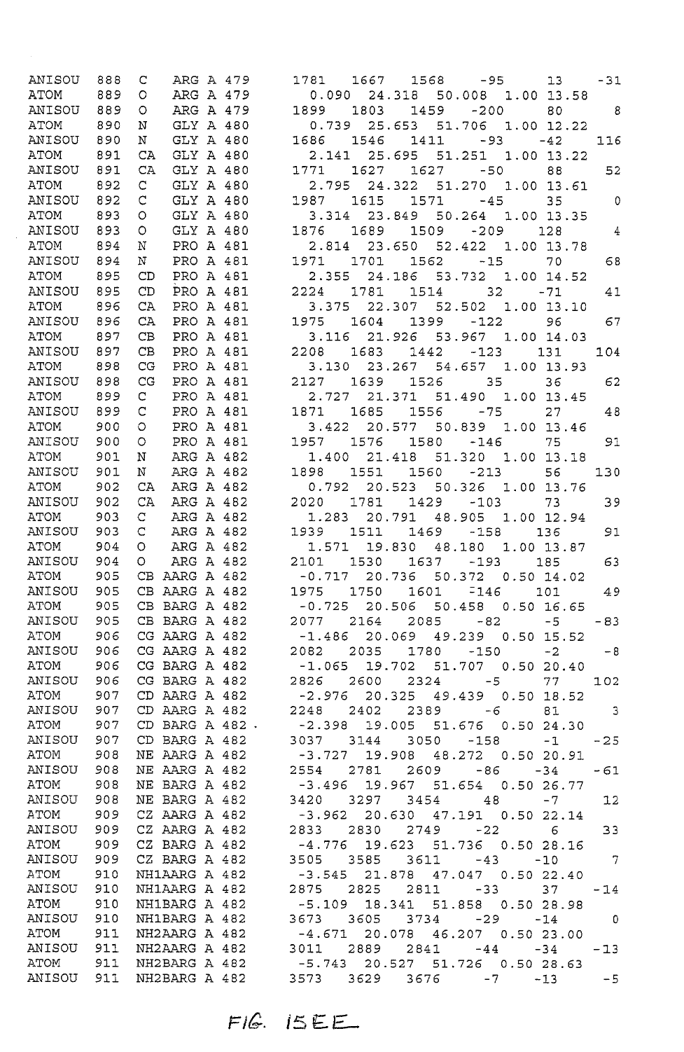

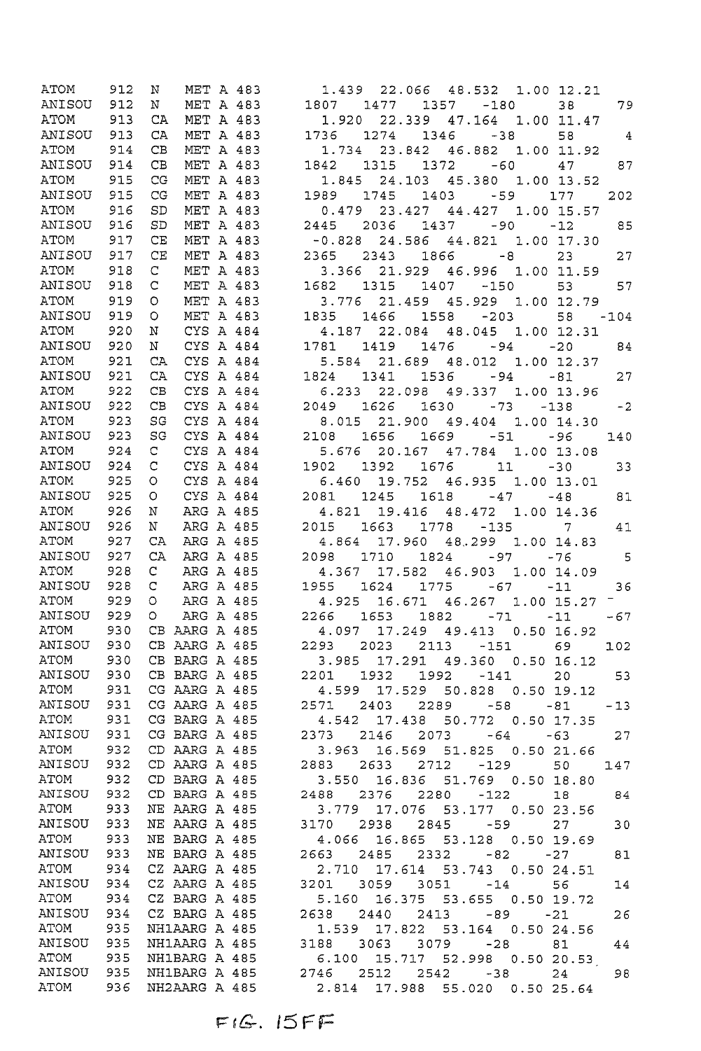

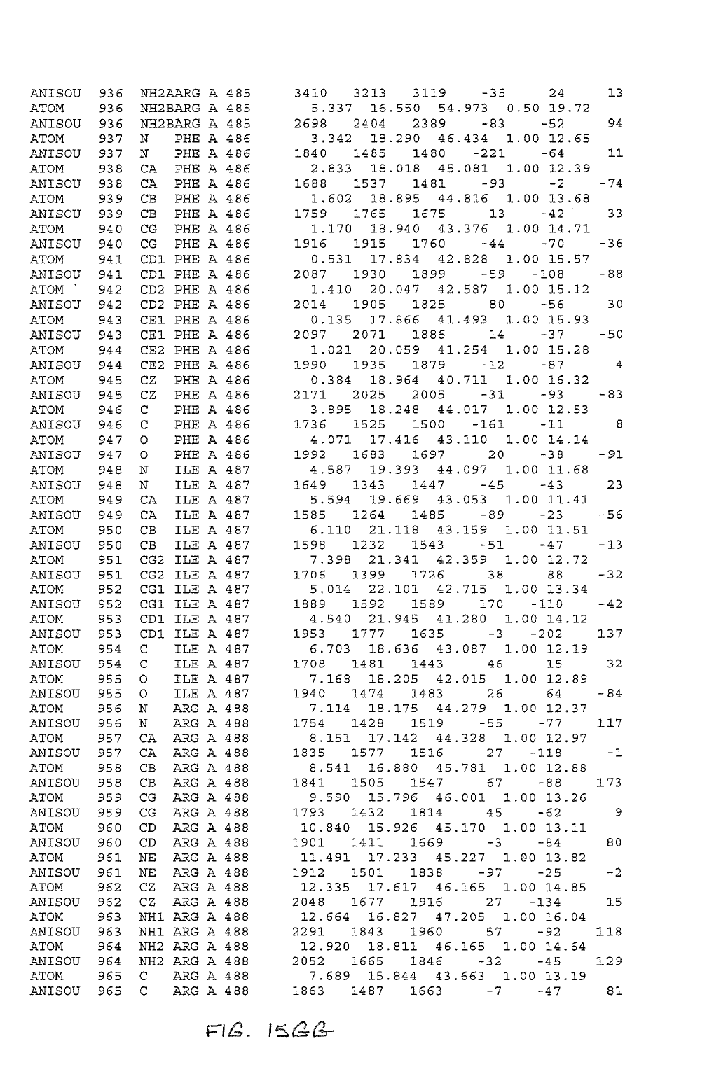

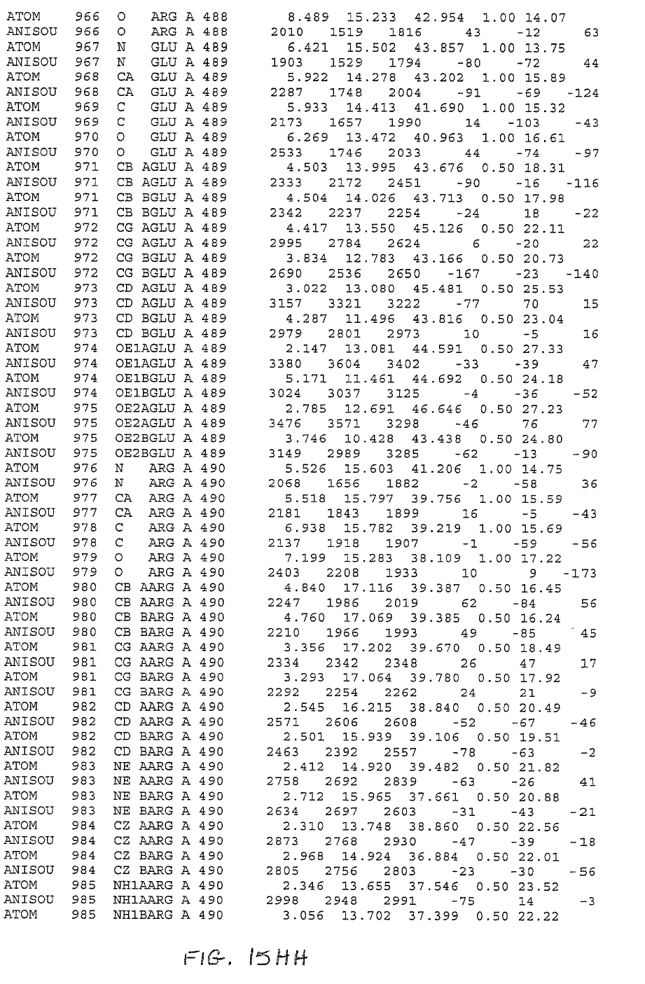

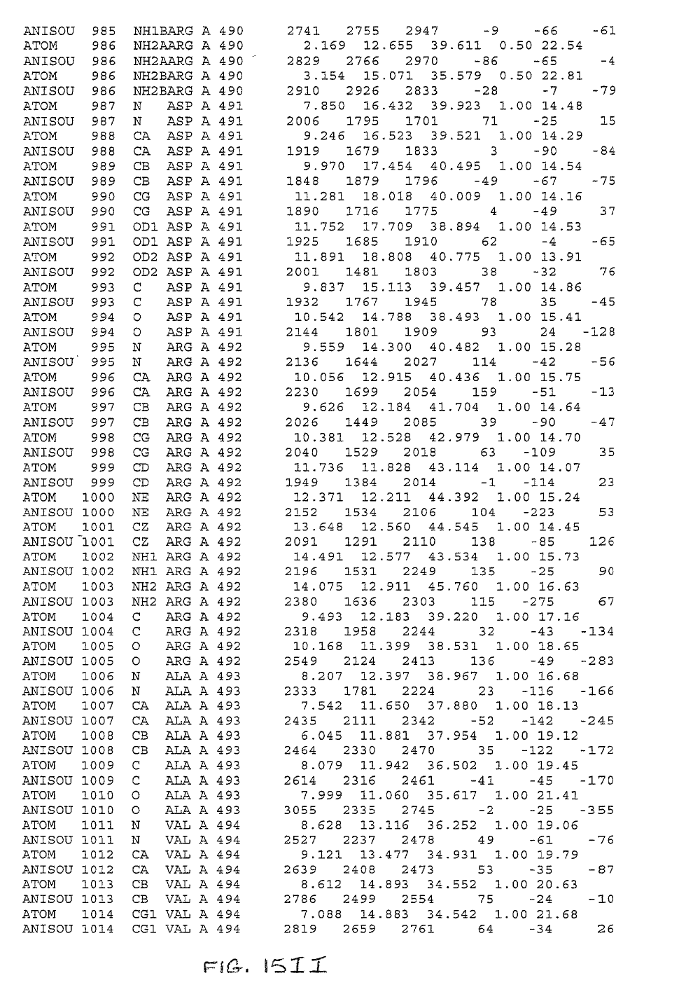

The polypeptides prepared are listed in Table 9, together with their N-terminal and C-terminal amino acid residues. The amino acid sequences of these polypeptides are presented in FIGS. 4-14. The numbering of the residues in Table 9 refers to the appropriate residue in the amino acid sequence (SEQ ID NO: 1, 2 or 3) of the corresponding native protein, as presented in FIGS. 1, 2 and 3. The amino acid sequences of the native proteins (SEQ ID NOs: 1, 2 and 3) are taken as defined in SWISS-PROT (Bairoch et al. Nucleic Acid Res. 22:3578 (1994)). As described in the Examples, certain of these crystals were examined by x-ray crystallography and atomic coordinates for the peptide were obtained. In certain cases, the polypeptide was unligated, that is, not complexed with a ligand. In other cases, the polypeptide was complexed with a ligand and the atomic coordinates of the ligand bound to the Cdc25 catalytic domain were also obtained.

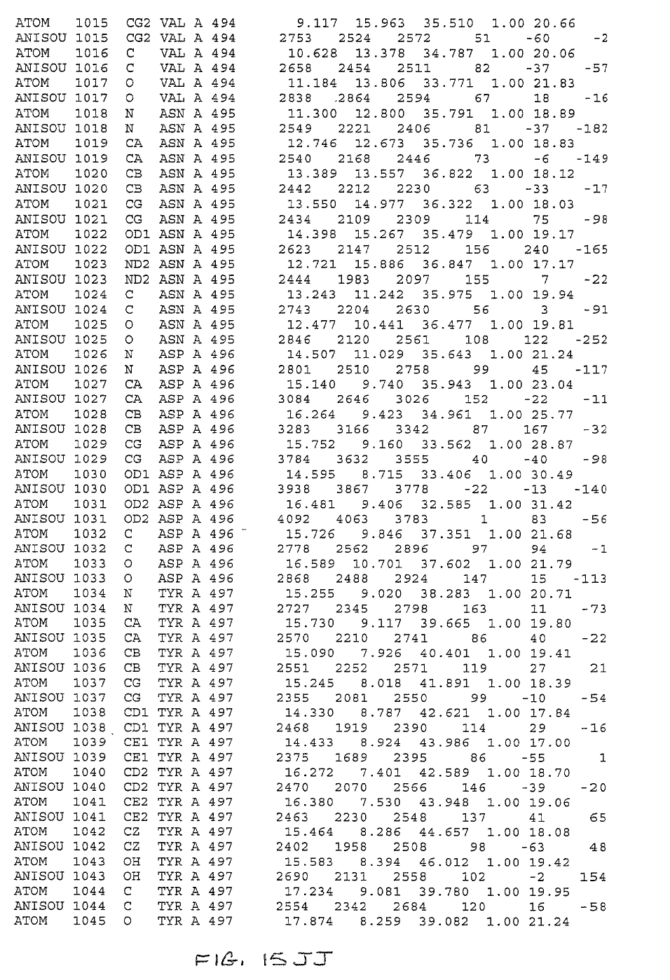

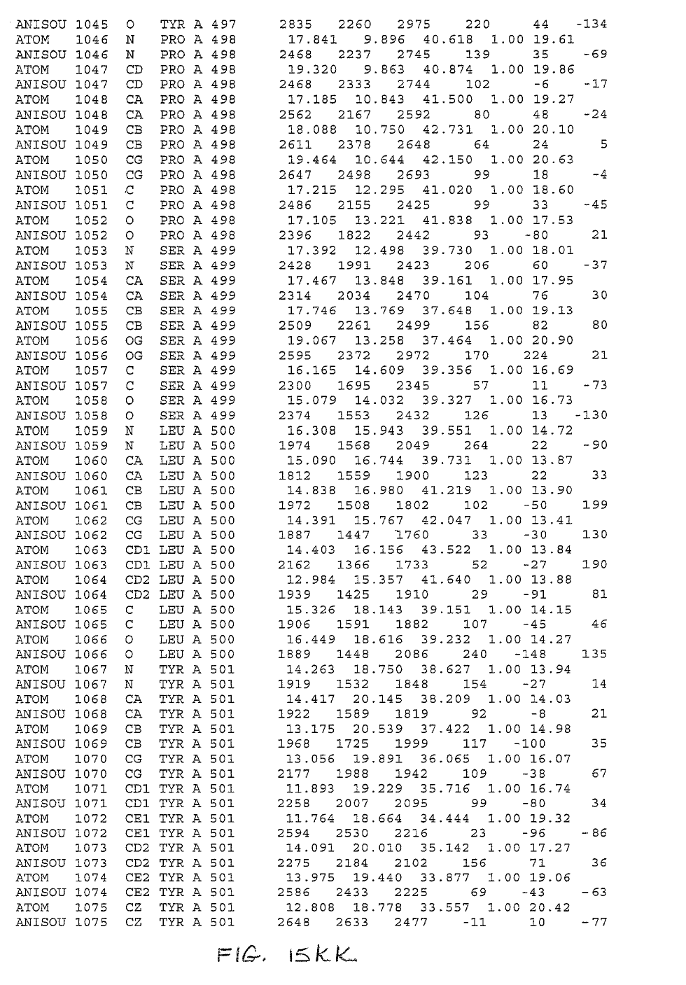

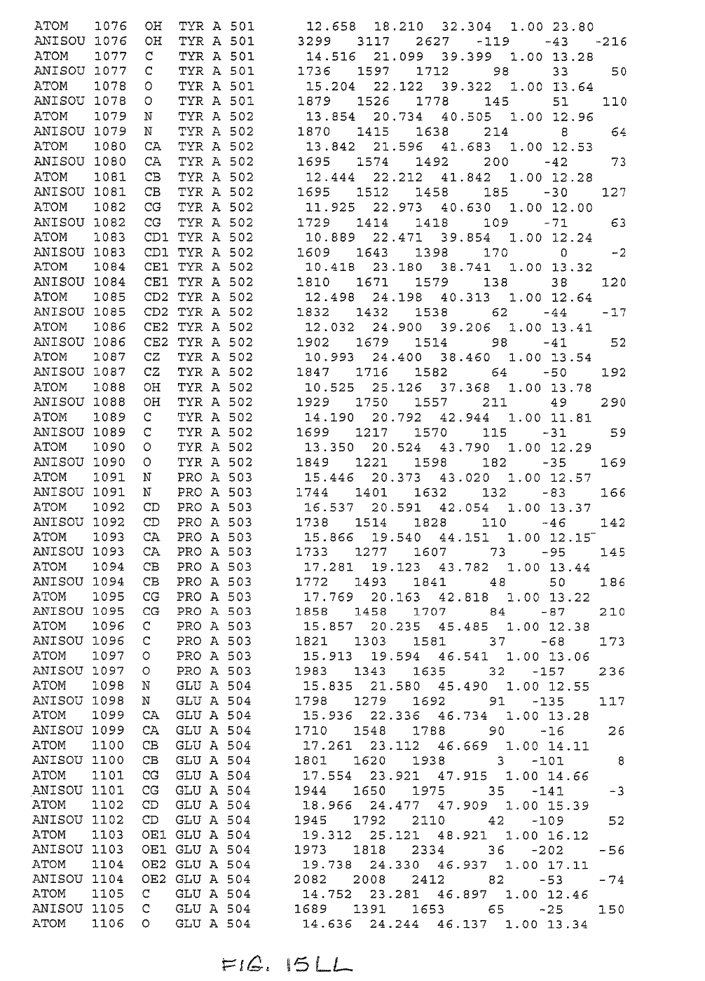

The atomic coordinates for five crystals examined by x-ray crystallography are presented in FIGS. 15A-15PPP, 16A-16I, 17A-17EE, 18A-18X and 19A-19I. The term “atomic coordinates” (or “structural coordinates”) refers to mathematical coordinates derived from mathematical equations related to the patterns obtained on diffraction of x-rays by atoms (scattering centers) of a crystalline polypeptide comprising a Cdc25 catalytic domain molecule. The diffraction data are used to calculate an electron density map of the repeating unit of the crystal. The electron density maps are used to establish the positions of the individual atoms within the unit cell of the crystal. Atomic coordinates can be transformed as is known in the art to different coordinate systems without affecting the relative positions of the atoms.

In particular, a high resolution crystal structure was obtained for the polypeptide denoted Cdc25B (ΔN8B) in Table 9, complexed with the pentapeptide inhibitor shown below, denoted “cdc1249” herein.

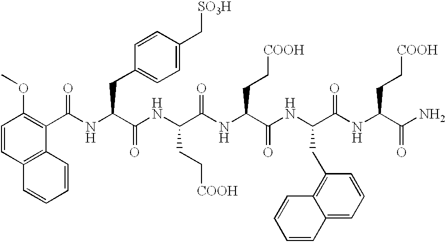

Polypeptide Cdc25B (ΔN8B) includes residues Leu 368 to Arg 562 of human Cdc25B (SEQ ID NO: 2). The complex of this polypeptide and cdc1249 crystallized in space group P4₃2₁2, a=70.29, c=130.59. The term “space group” is a term of art which refers to the collection of symmetry elements of the unit cell of a crystal. The results of the x-ray crystal structure determination for Cdc25B (ΔN1B) indicated that the unit cell includes eight polypeptide molecules. Atomic coordinates for the non-hydrogen atoms in the protein, the inhibitor in active site; a second molecule of the inhibitor (distal to the catalytic domain); water molecules; and sodium and chloride counter ions were determined and are provided in FIG. 15A-15PPP. For the inhibitor molecule in the active site, all heavy (non-hydrogen) atoms were observed except for the second Glu residue beyond CB and the C-terminal Glu-amide. For the second inhibitor molecule, all non-hydrogen atoms were observed. For the present purposes, the carbon atoms in an amino acid side chain are designated CB, CG, CD, and so forth, where CB is the carbon atom bonded to the α-carbon, CG is the side chain carbon atom bonded to CB and so forth. The letters designating the carbon atoms are ordered according to the corresponding Greek letters.

The structures determined for polypeptides comprising the Cdc25B catalytic domain complexed with a ligand differ significantly from the structure determined by Saper et al. for Cdc25A. Most importantly, the Cdc25B protein structure has a ligand bound at the catalytic site, and all protein atoms in proximity to the ligand are well defined. In the Cdc25A structure of Saper et al. there is no bound ligand and the catalytic loop is very poorly resolved (the residues composing the catalytic loop are disordered). One particular residue of the catalytic loop, Arg 479, appears in the Cdc25A structure to be misplaced when compared to the structures determined for polypeptides comprising the Cdc25B catalytic domain and the structures of other known phosphatases. Consequently, no reliable information with regard to ligand binding can be directly obtained from the Cdc25A protein structure, and the lack of atomic resolution around the binding site means that molecular modeling techniques can not be reliably used to predict ligand binding modes or for ligand design. Another major difference between the Cdc25B catalytic domain structure described herein and the Cdc25A structure of Saper et al. is in the C-terminal region (residues 531-547, Cdc25B numbering). This region of Cdc25B, which is well resolved in the present structures, contains an alpha-helix which is positioned against the bulk of the protein, and several residues of the helix, such as Met 531 and Arg 544, interact with the bound ligand. In contrast, in the Cdc25A structure of Saper et al., this region is undefined beyond Asp 492 (Cdc25A numbering scheme), and the few residues that are observed appear to be misplaced. For example, the sequence is directed away from the bulk of the protein and towards a symmetry related molecule in the crystal. The position of the C-terminus in the Cdc25A structure, thus, appears to be determined by packing forces within the crystal.

The structure of the Cdc25B(ΔN8B)/cdc1249 complex shows that the phenyl group of the ligand (HO₃SCH₂)Phe residue is completely surrounded by hydrophobic groups including: Phe 475, Ser 477, and Glu 478 in the catalytic loop; by Met 531 on the C-terminus; by the naphthyl group of the ligand; and by Pro 457 and Ile 458 of a symmetry-related polypeptide molecule (crystal contacts). There is also a hydrogen bond from the ligand NH between PheCH₂SO₃H and 2-OMe-naphth, to the carbonyl oxygen atom of Pro 457. The naphthyl ring of the 2-OMe-naphth group also makes van der Waals contact with the symmetry-related molecule at Pro 457 and with with the backbone at Lys 455 and Ser 456. The methyl group of the 2-O-Me-naphth group sits in a groove on the polypeptide molecule. This naphthyl group also contacts Met 531 and Leu 540 on the C-terminus, and the Nal residue of the ligand. There is an important interaction, possibly a cation-pi, pi-pi or van der Waals, interaction, between the inhibitor Nal and Arg 544 (the sidechain of Arg 544 is hydrogen-bonded to Tyr 428 and it has one unfavorable torsion angle at CB-CG). This Nal residue also makes van der Waals contacts with the sidechains of Glu 478 and Arg 479 in the catalytic loop, and Met 531 in the C-terminus.

The pentapeptide inhibitor adopts a helix-like conformation with a mixture of 3₁₀/α properties. The inhibitor exhibits two intramolecular H-bonds: amide O, from between PheCSO₃H and 2-OMe-Naphth, to backbone NH between the two Glu residues, and to backbone NH between the second Glu and Nal. The hydrophobic groups in the ligand are close to one another, a situation which can be described as hydrophobic collapse.

As mentioned above, no electron density was observed for the terminal carboxylic acid in the ligand, a result which could indicate that there are a number of possible binding modes for this portion of the ligand.

A second molecule of the inhibitor is observed in the crystal structure, binding to the protein at a site distal to the catalytic site. This molecule appears to stabilize the crystal by forming a number of favorable interactions at the interface of two symmetry related protein molecules. The conformation of the ligand molecule at the distal site is very similar to the conformation of the ligand at the binding site. This indicates that the ligand is in a low-energy conformation, one that is not significantly biased by interactions with the protein. This result has been confirmed by molecular modeling and conformational analysis.

The C-terminal region of the Cdc25B catalytic domain is helical and plays a significant role in ligand binding. This region was not observed in the structure of Saper et al. This part of the protein may be highly flexible, with a geometry dependent upon such factors as salt concentration, length of the construct, protein-protein interactions (CDK/cyclin), bound ligand, and pH, among others.

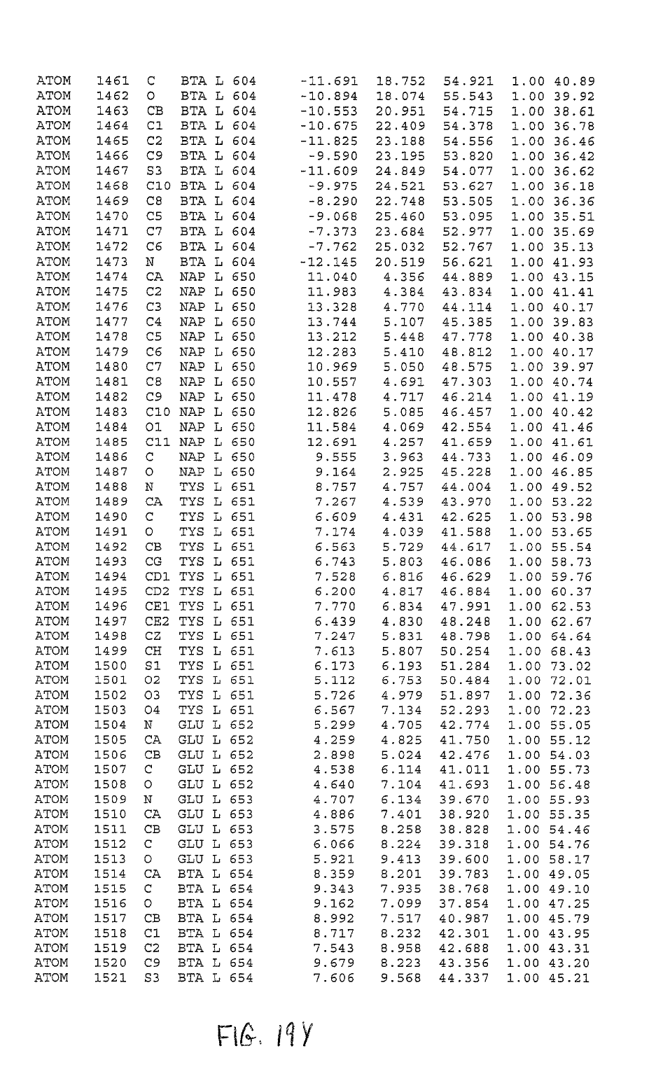

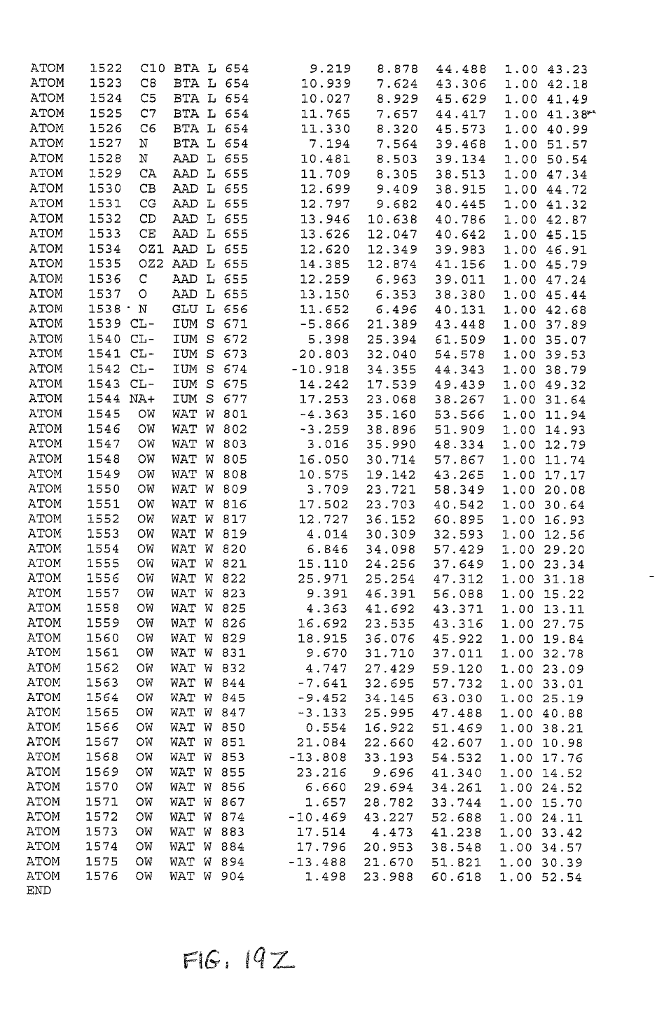

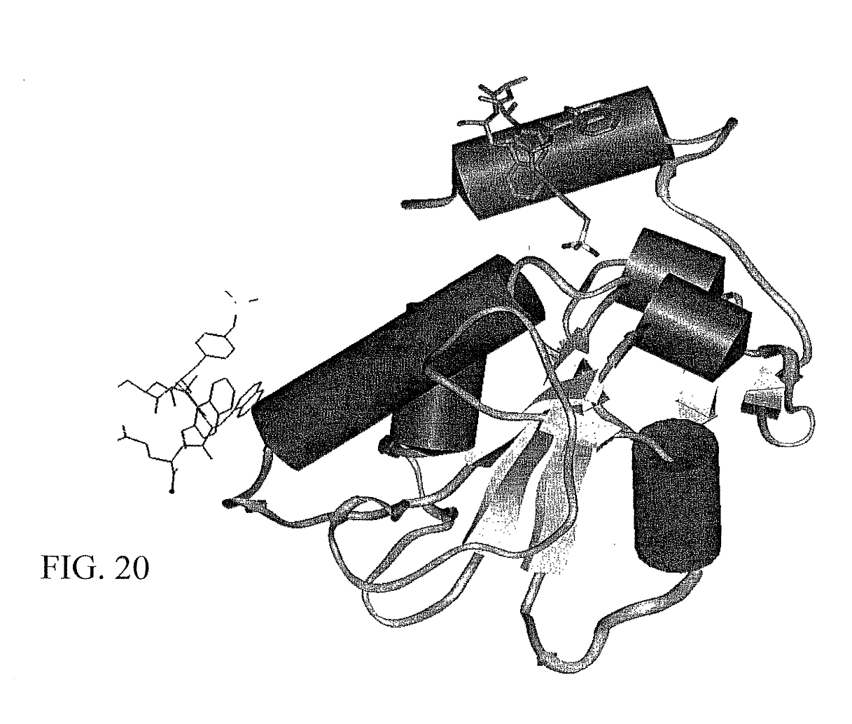

Analysis of the three dimensional structure of the Cdc25B catalytic domain has indicated the presence of a number of subsites, each of which includes molecular functional groups capable of interacting with complementary moieties of an inhibitor. Subsites 1-16 of the Cdc25B catalytic domain are defined below. The catalytic domain consists of the catalytic loop and surrounding area. Sixteen subsites are defined; subsites 1-9 correspond to pockets, clefts, grooves, etc., and the remaining seven are bumps, that is, the solvent exposed tops of amino acid side chains. FIGS. 20 (top view) and 21 (side view) illustrate the binding site region with the subsites labeled.

Subsites are characterized below according to the properties of chemical moieties with which they are complementary, or with which they can interact. Such moieties can include hydrogen bond acceptors (“HA”), such as hydroxyl, amino, and carbonyl groups, halogen atoms, such as fluorine, chlorine, bromine and iodine atoms; and other groups including a heteroatom having at least one lone pair of electrons, such as groups containing trivalent phosphorous, di- and tetravalent sulfur, oxygen and nitrogen atoms; hydrogen bond donors (“HD”), such as hydroxyl, amino, carboxylic acid groups and any of the groups listed under hydrogen atom acceptors to which a hydrogen atom is bonded; hydrophobic groups (“H”), such as linear, branched or cyclic alkyl groups; linear, branched or cyclic alkenyl groups; linear, branched or cyclic alkynyl groups; aryl groups, such as mon- and polycyclic aromatic hydrocarbyl groups and mono- and polycyclic heteroaryl groups; positively charged groups (“P”), such as primary, secondary, tertiary and quatemary ammonium groups, substituted and unsubstituted guanidinium groups, sulfonium groups and phosphonium groups; and negatively charged groups (“N”), such as carboxylate, sulfonamide, sulfamate, boronate, vanadate, sulfonate, sulfinate and phosphonate groups. A given chemical moiety can contain one or more of these groups.

Subsite 1: Catalytic loop; interacting chemical moieties: HA, H, N;

Residues involved: Cys 473; Glu 474; Phe 475; Ser 476; Ser 477; Glu 478; Arg 479;

Non-hydrogen atoms which interact with HA and N: Cys 473 S; Glu 474 N; Phe 475 N; Ser 476 N; Ser 477 N; Glu 478 N; Arg 479 N, NE, NH₂

Non-hydrogen atoms which interact with H: Glu 474 CA, CB, CG, CD; Phe 475 CB, CG, CD1, CD2, CE1, CE2, CZ; Ser 477 CB; Glu 478 CB, CG, CD

Subsite 2: Swimming pool

Interacting chemical moieties: HA, HD, H, N, P

Residues involved: Cys 426; Tyr 428; Pro 444; Leu 445; Glu 446; Glu 478; Arg 479; Arg 482; Met 483; Arg 544; Thr 547; Arg 548

Non-hydrogen atoms which interact with HA and N: Tyr 428 OH; Arg 482 NH1 or NH2; Arg 544 NH1

Non-hydrogen atoms which interact with HD and P: Cys 426 O; Tyr 428 OH; Pro 444 O; Glu 446 OE1, OE2; Thr 547 OG1.

Non-hydrogen atoms which interact with H: Leu 445 CA, CB, CD1; Glu 446 CA, CB, CG, CD; Arg 479 CA, CB, CG, CD; Met 483 CA, CB, CG, SD, CE; Thr 547 CB, CG2; Arg 548 CA, CB, CG, CD, CE

Subsite 3: Anion binding site

Interacting chemical moieties: HA, N

Residues involved: Arg 482; Arg 544

Non-hydrogen atoms which interact with HA and N: Arg 482 NH1, NH2; Arg 544 NH1, NH2

Subsite 4: Groove between catalytic loop and swimming pool

Interacting chemical moieties: H

Residues involved: Glu 478; Arg 479; Met 531; Arg 544.

Non-hydrogen atoms which interact with H: Glu 478 CA, CB, CG, CD; Arg 479 CA, CB, CG, CD, CZ; Met 531 CB, CG, SD, CE; Arg 544 CG, CD, CZ.

Subsite 5: Na1 binding region

Interacting chemical moieties: HA, HD, H, N

Residues involved: Tyr 428; Glu 478; Arg 479; Met 531; Leu 540; Arg 544

Non-hydrogen atoms which interact with HA and HD: Tyr 428 OH

Non-hydrogen atoms which interact with H: Tyr 428 CD1,CE1; Glu 478 CA, CB, CG, CD; Arg 479 CA, CB, CG, CD, CZ; Met 531 CG, SD, CE; Leu 540 CB, CG, CD1, CD2; Arg 544 CG, CD, CZ

Non-hydrogen atoms which interact with N: Arg 544 NE, NH1, NH2

Subsite 6: 2-MeO-Na1 binding region

Interacting chemical moieties: H

Residues involved: Phe 475; Met 531; Asn 532; Leu 540

Non-hydrogen atoms which interact with H: Phe 475 CG, CD1, CD2, CE1, CE2, CZ; Met 531 CB, CG, SD, CE; Asn 532 CB, CG; Leu 540 CB, CG, CD1, CD2

Subsite 7: Interactions involving Ile 458 of the symmetry related polypeptide

Interacting chemical moieties: H

Residues involved: Phe 475; Ser 477

Non-hydrogen atoms which interact with H: Phe 475 C, CB, CG, CD1, CD2, CE1, CE2, CZ; Ser 477 CB

Subsite 8: Interactions involving Pro 457 of the symmetry related polypeptide

Interacting chemical moieties: HA, HD, H

Residues involved: Glu 474; Phe 475; Met 531; Asn 532

Non-hydrogen atoms which interact with HA: Asn 532 ND2

Non-hydrogen atoms which interact with HD: Glu 474 OE1, OE2; Asn 532 OD1

Non-hydrogen atoms which interact with H: Glu 474 CB, CG, CD; Phe 475 CG, CD1, CD2, CE1, CE2, CZ; Met 531 C, CA, CB, CG, SD, CE; Asn 532 CA, CB, CG

Subsite 9: Region around Leu 540

Interacting chemical moieties: H

Residues involved: Tyr 428; Met 531; Lys 537; Leu 540; Lys 541; Arg 544

Non-hydrogen atoms which interact with H: Tyr 428 CD1, CE1; Met 531 CB, CG, SD, CE; Lys 537 CA, CB, CG, CD, CE; Leu 540 CB, CC, CD1, CD2; Lys 541 CA, CB, CG, CD, CE; Arg 544 CB, CG, CD

Subsite 10: Ser477

Interacting chemical moieties: HA, HD

Residues involved: Ser 477

Non-hydrogen atoms which interact with HA and HD: Ser 477 OG

Subsite 11: Glu 478

Interacting chemical moieties: HD, P

Residues involved: Glu 478;

Non-hydrogen atoms which interact with HD and P: Glu 478 OE1, OE2

Subsite 12: Lys 394

Interacting chemical moieties: HA, N

Non-hydrogen atoms which interact with HA and N: Lys 394 NZ

Subsite 13: Arg 482

Interacting chemical moieties: HA, N

Non-hydrogen atoms which interact with HA and N: Arg 482 NE, NH1 or NH2

Subsite 14: Arg 544

Interacting chemical moieties: HA, N

Non-hydrogen atoms which interact with HA and N: Arg 544 NE, NH2

Subsite 15: Phe 475

Interacting chemical moieties: H

Non-hydrogen atoms which interact with H: Phe 475 CB, CG, CD1, CD2, CE1, CE2, CZ

Subsite 16: Asn 532

Interacting chemical moieties: HA, HD, H

Non-hydrogen atoms which interact with HA: Asn 532 ND2

Non-hydrogen atoms which interact with HD: Asn 532 OD1

Non-hydrogen atoms which interact with H: Asn 532 CB, CG

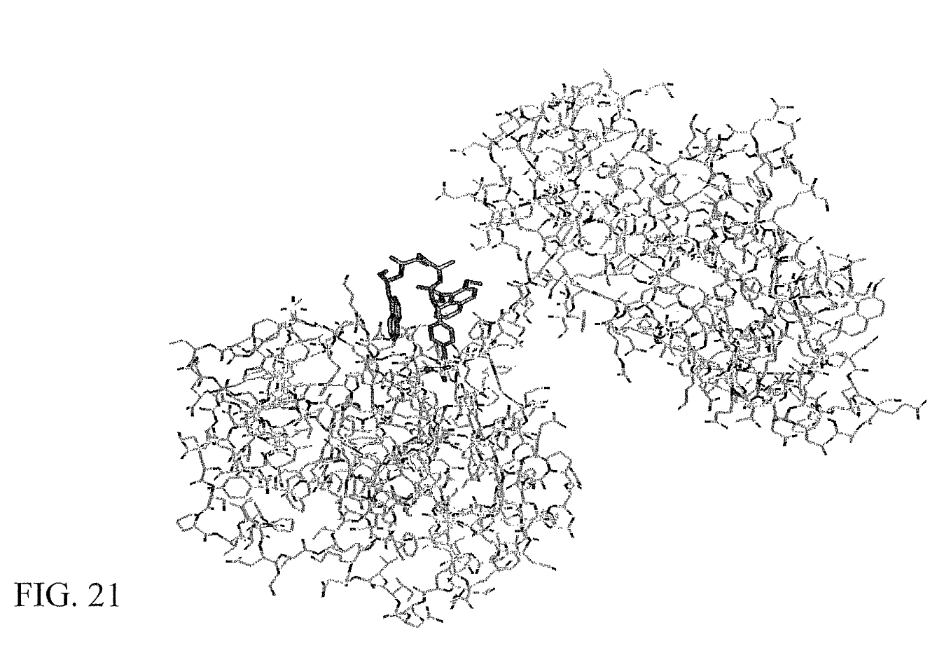

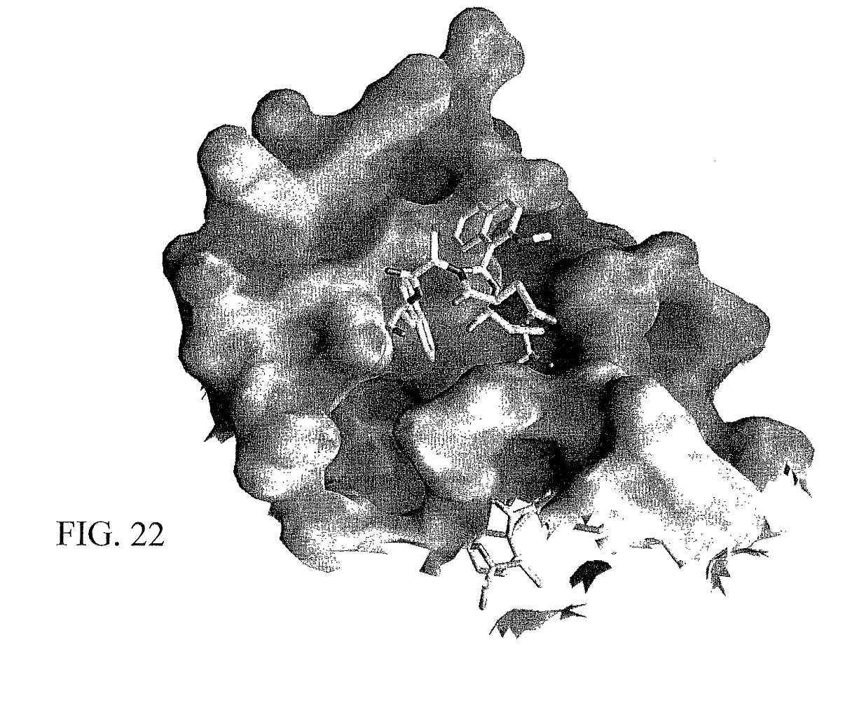

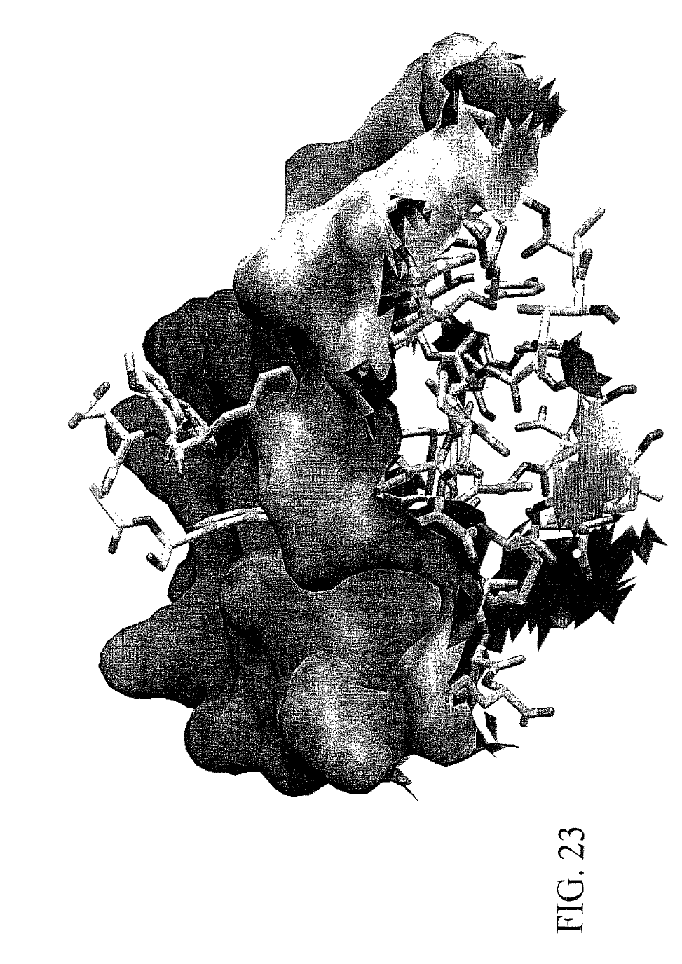

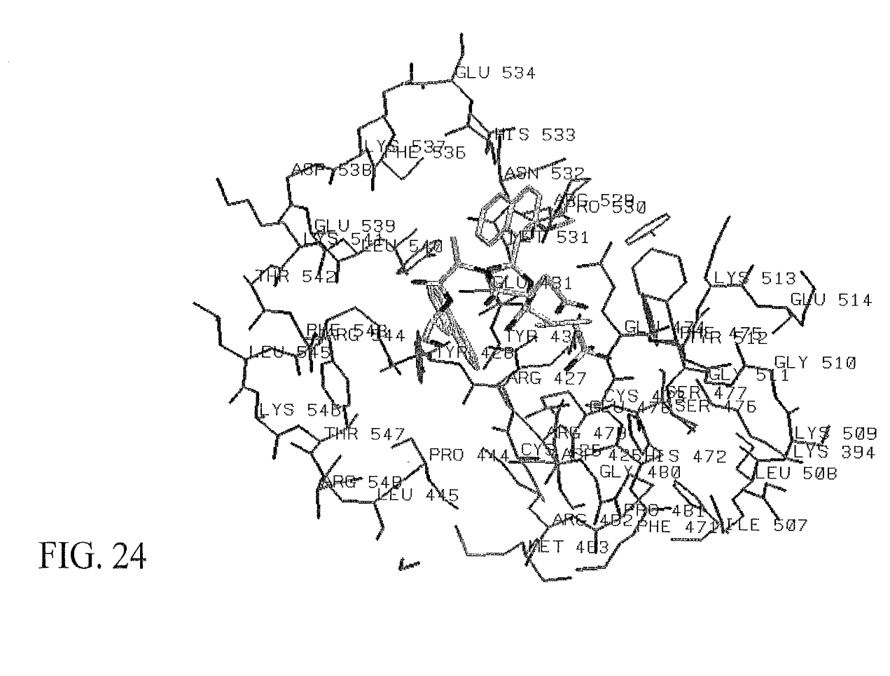

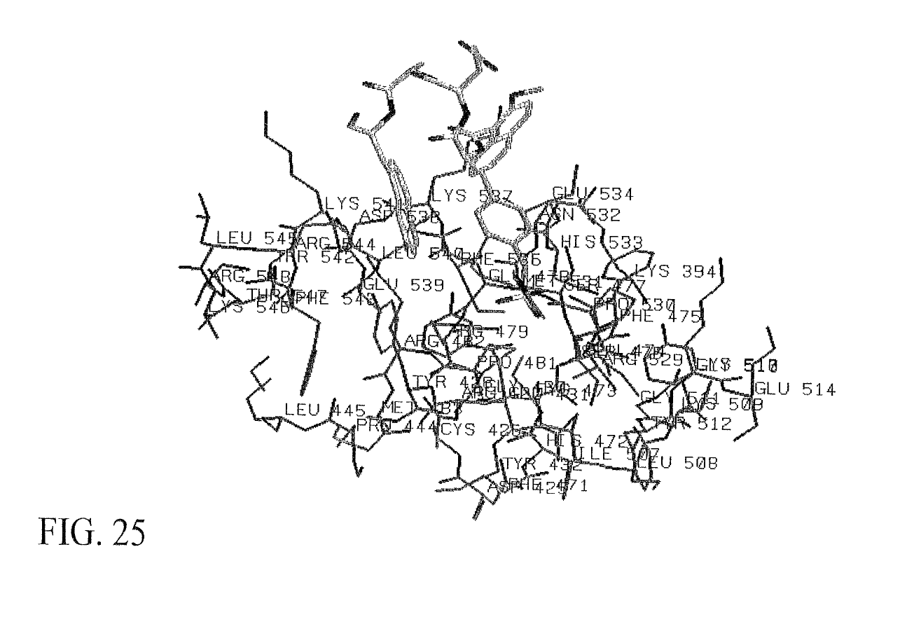

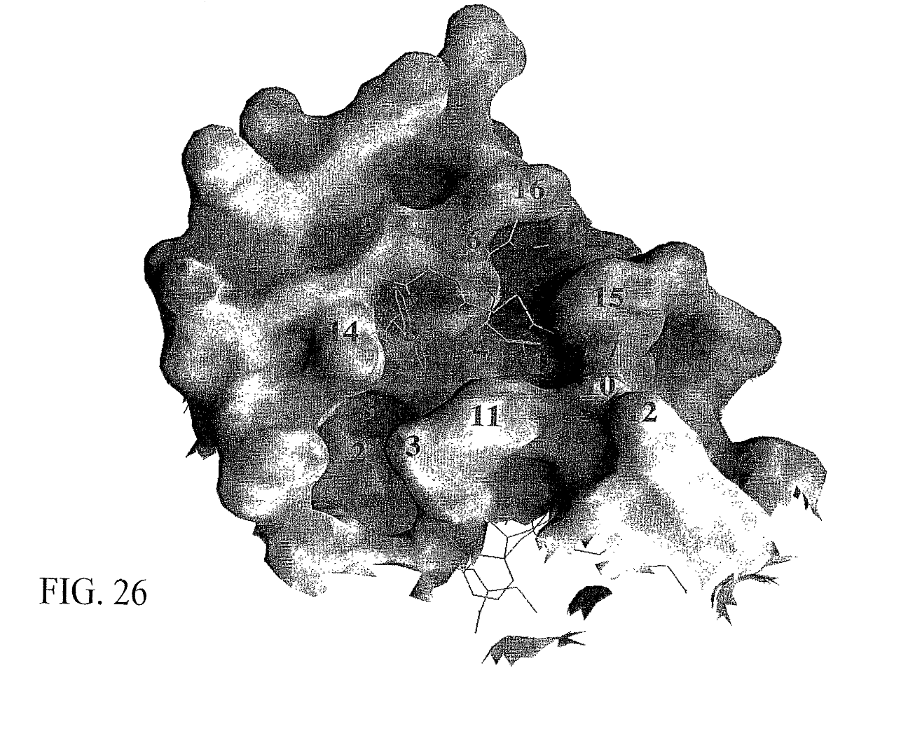

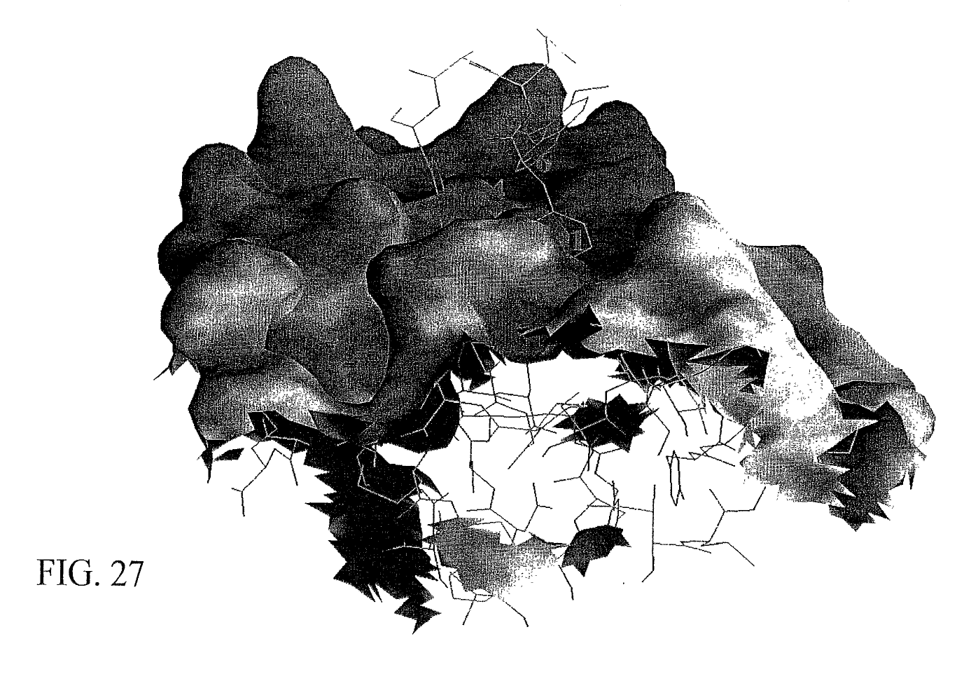

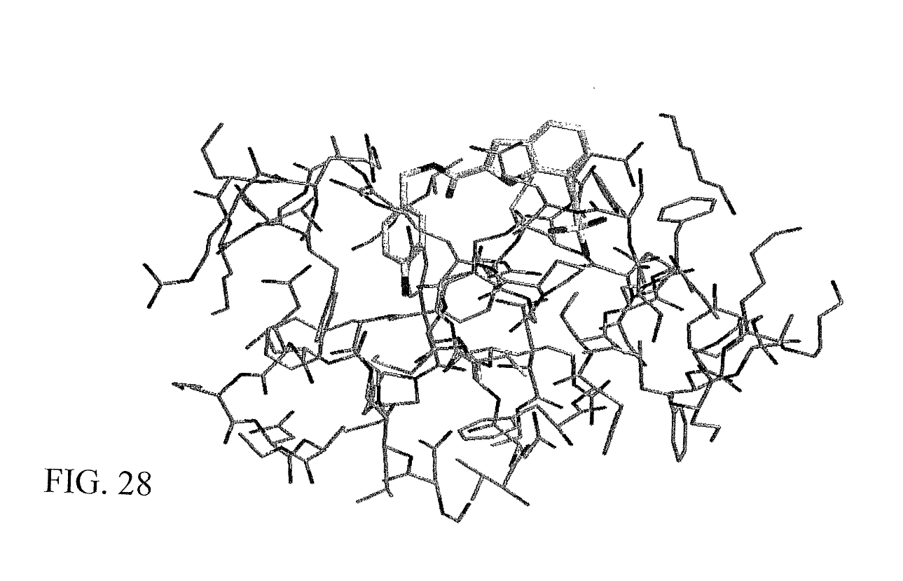

FIGS. 20-28 provide different views of the Cdc25 catalytic domain structure and the interaction of cdc1249 with the polypeptide. For example, FIG. 20 provides a view of the complex of Cdc25B and cdc1249 showing the protein secondary structure, the ligand bound at the catalytic loop (thick bonds), and the ligand bound at the distal site (thin bonds). FIG. 21 is another view of this complex showing two symmetry related protein molecules interacting with the ligand bound at the catalytic site. Water molecules and ions are not shown. FIG. 22 shows a top view of the molecular surface around the ligand binding area. The terminal atoms of Arg 482 have been removed so that the swimming pool can be clearly observed. Water molecules and ions are not shown. The view of the complex presented in FIG. 23 is a side view relative to the view in FIG. 22. FIG. 24 shows a top view of the complex of Cdc25B and cdc1249 with protein residues labeled. Water molecules and ions are not shown. FIG. 25 shows a side view of the complex relative to the view presented in FIG. 23. FIG. 26 presents a top view of the complex, showing the molecular surface around the ligand binding area, with each subsite labeled. The terminal atoms of Arg 482 have been removed so that the swimming pool can be clearly observed. Water molecules and ions are not shown. FIG. 27 shows a side view of the complex relative to the view in FIG. 26, and only subsites 1-6 are labeled. catalytic loop of Cdc25A is not shown and the well-defined catalytic loop of Cdc25B is shown in purple. FIG. 28 presents a side view of a potential tight-binding inhibitor complexed to Cdc25B. The designed ligand binds in the catalytic loop and swimming pool, and spans the groove between the two.

In one embodiment, the present invention provides polypeptides comprising the catalytic domain of Cdc25, crystalline forms of these polypeptides, optionally complexed with a ligand, and the three dimensional structure of the polypeptides, including the three dimensional structure of the Cdc25 catalytic domain. In general, these three dimensional structures are defined by atomic coordinates derived from x-ray crystallographic studies of the polypeptides. The polypeptides can include the catalytic domain of Cdc25 from any species, such as a yeast or other unicellular organism, an invertebrate or a vertebrate. Preferably, the polypeptide includes the binding domain of a mammalian Cdc25, such as a mammalian Cdc25A, Cdc25B or Cdc25C. More preferably, the polypeptide includes the catalytic domain of human Cdc25A, Cdc25B or Cdc25C. In one embodiment, the polypeptide includes amino acids Leu 336 to Thr 506 of SEQ ID NO: 1, amino acids Leu 378 to Arg 548 of SEQ ID NO: 2 or amino acids Leu 282 to Val 453 of SEQ ID NO: 3. In particular embodiments, the polypeptides can include amino acids Leu 336 to Leu 523; Gly 323 to Leu 523; Glu 326 to Arg 519; or Glu 326 to Thr 506 of SEQ ID NO: 1; Leu 378 to Gln 566; Asp 365 to Gln 566; Glu 368 to Arg 562; Glu 368 to Ser 549 or Glu 368 to Arg 548 of SEQ ID NO: 2; or amino acids Leu 282 to Pro 473 or Gly 280 to Val 453 of SEQ ID NO: 3.

The crystalline polypeptide, preferably, further includes a ligand bound to the Cdc25 catalytic domain. The ligand is, preferably, a small (less than about 1500 molecular weight) organic molecule, for example, a peptide, such as a pentapeptide.

In one embodiment, the invention relates to a method of determining the three dimensional structure of a first polypeptide comprising the catalytic domain of a CdC25 protein. The method includes the steps of (1) obtaining a crystal comprising the first polypeptide; (2) obtaining x-ray diffraction data for said crystal; and (3) using the x-ray diffraction data and the atomic coordinates of a second polypeptide comprising the catalytic domain of a Cdc25 protein to solve the crystal structure of the first polypeptide, thereby determining the three dimensional structure of the first polypeptide. The second polypeptide can include the same Cdc25 catalytic domain as the first polypeptide, or a different Cdc25 catalytic domain. Either or both of the first and second polypeptides can, optionally, be complexed with a ligand. That is, the crystal of the first polypeptide can comprise a complex of the first polypeptide with a ligand. The atomic coordinates of the second polypeptide can, optionally, include the atomic coordinates of a ligand molecule bound to the second polypeptide. The atomic coordinates of the second polypeptide, generally, have been previously obtained, for example, by x-ray crystallographic analysis of a crystal comprising the second polypeptide or a complex of the second polypeptide with a ligand. The atomic coordinates of the second polypeptide can be used to solve the crystal structure using methods known in the art, for example, molecular replacement or isomorphous replacement. Preferably, the second polypeptide comprises the catalytic domain of a mammalian Cdc25, more preferably a mammalian Cdc25B, and, most preferably, human Cdc25B. For example the atomic coordinates which can be used include the atomic coordinates presented herein, preferably the atomic coordinates presented in FIG. 15A to 15PPP.

The invention also provides a method of identifying a compound which is a potential inhibitor of Cdc25. The method comprises the steps of (1) obtaining a crystal of a polypeptide comprising the catalytic domain of Cdc25; (2) obtaining the atomic coordinates of the polypeptide by x-ray diffraction studies using said crystal; (3) using said atomic coordinates to define the catalytic domain of Cdc25; and (4) identifying a compound which fits the catalytic domain. The method can further include the steps of obtaining, for example, from a compound library, or synthesizing the compound identified in step 4, and assessing the ability of the identified compound to inhibit Cdc25 enzymatic activity.

The polypeptide preferably comprises the catalytic domain of a mammalian Cdc25, such as a mammalian Cdc25A, Cdc25B or Cdc25C. More preferably the polypeptide comprises the catalytic domain of human Cdc25A, Cdc25B or Cdc25C. In a preferred embodiment, the polypeptide is a polypeptide of the present invention, as described above.

The polypeptide can be crystallized using methods known in the art, such as the methods described in the Examples, to afford polypeptide crystals which are suitable for x-ray diffraction studies. A crystalline polypeptide/ligand complex can be produced by soaking the resulting crystalline polypeptide in a solution including the ligand. Preferably, the ligand solution is in a solvent in which the polypeptide is insoluble.

The atomic coordinates of the polypeptide (and ligand) can be determined, for example, by x-ray crystallography using methods known in the art. The data obtained from the crystallography can be used to generate atomic coordinates, for example, of the atoms of the polypeptide and ligand, if present. As is known in the art, solution and refinement of the x-ray crystal structure can result in the determination of coordinates for some or all of the non-hydrogen atoms. The atomic coordinates can be used, as is known in the art, to generate a three-dimensional structure of the Cdc25 catalytic domain. This structure can then be used to assess the ability of any given compound, preferably using computer-based methods, to fit into the catalytic domain.

A compound fits into the catalytic domain if it is of a suitable size and shape to physically reside in the catalytic domain, that is, if it has a shape which is complementary to the catalytic domain and can reside in the catalytic domain without significant unfavorable steric or van der Waals interactions. Preferably, the compound includes one or more functional groups and/or moieties which interact with one or more subsites within the catalytic domain. Computational methods for evaluating the ability of a compound to fit into the catalytic domain, as defined by the atomic coordinates of the polypeptide, are known in the art, and representative examples are provided below.

In another embodiment, the method of identifying a potential inhibitor of Cdc25 comprises the step of determining the ability of one or more functional groups and/or moieties of the compound, when present in the Cdc25 catalytic domain, to interact with one or more subsites of the Cdc25 catalytic domain. Preferably, the Cdc25 catalytic domain is defined by the atomic coordinates of a polypeptide comprising the Cdc25 catalytic domain. If the compound is able to interact with a preselected number or set of subsites, the compound is identified as a potential inhibitor of Cdc25.

A functional group or moiety of the compound is said to “interact” with a subsite of the Cdc25 catalytic domain if it participates in an energetically favorable, or stabilizing, interaction with one or more complementary moieties within the subsite. Two chemical moieties are “complementary” if they are capable, when suitably positioned, of participating in an attractive, or stabilizing, interaction, such as an electrostatic or van der Waals interaction. Typically, the attractive interaction is an ion-ion (or salt bridge), ion-dipole, dipole-dipole, hydrogen bond, pi-pi or hydrophobic interaction. For example, a negatively charged moiety and a positively charged moiety are complementary because, if suitably positioned, they can form a salt bridge. Likewise, a hydrogen bond donor and a hydrogen bond acceptor are complementary if suitably positioned.

Typically, the assessment of interactions between the test compound and the Cdc25 catalytic domain employs computer-based computational methods, such as those known in the art, in which possible interactions of a compound with the protein, as defined by atomic coordinates, are evaluated with respect to interaction strength by calculating the interaction energy upon binding the compound to the protein. Compounds which have calculated interaction energies within a preselected range or which otherwise, in the opinion of the computational chemist employing the method, have the greatest potential as Cdc25 inhibitors, can then be provided, for example, from a compound library or via synthesis, and assayed for the ability to inhibit Cdc25. The interaction energy for a given compound generally depends upon the ability of the compound to interact with one or more subsites within the protein catalytic domain.

In one embodiment, the atomic coordinates used in the method are the atomic coordinates set forth in FIGS. 15A to 15PPP, 16A to 16I, 17A to 17EE or 18A to 18X. Preferably the atomic coordinates are the coordinates set forth in FIG. 15A to 15PPP. It is to be understood that the coordinates set forth in FIGS. 15A to 15PPP, 16A to 16I, 17A to 17EE and 18A to 18X can be transformed, for example, into a different coordinate system, in ways known to those of skill in the art without substantially changing the three dimensional structure represented thereby.

In certain cases a moiety of the compound can interact with a subsite via two or more individual interactions. A moiety of the compound and a subsite can interact if they have complementary properties and are positioned in sufficient proximity and in a suitable orientation for a stabilizing interaction to occur. The possible range of distances for the moiety of the compound and the subsite depends upon the distance dependence of the interaction, as is known in the art. For example, a hydrogen bond typically occurs when a hydrogen bond donor atom, which bears a hydrogen atom, and a hydrogen bond acceptor atom are separated by about 2.5 Å and about 3.5 Å. Hydrogen bonds are well known in the art (Pimentel et al., The Hydrogen Bond, San Francisco: Freeman (1960)). Generally, the overall interaction, or binding, between the compound and the Cdc25 catalytic domain will depend upon the number and strength of these individual interactions.

The ability of a test compound to interact with one or more subsites of the catalytic domain of Cdc25 can be determined by computationally evaluating interactions between functional groups, or moieties, of the test compound and one or more amino acid side chains in a particular protein subsite, such as subsites 1 to 16 above. Typically, a compound which is capable of participating in stabilizing interactions with a preselected number of subsites, preferably without simultaneously participating in significant destabilizing interactions, is identified as a potential inhibitor of Cdc25. Such a compound will interact with one or more subsites, preferably with two or more subsites and, more preferably, with three or more subsites.

The invention further provides a method of designing a compound which is a potential inhibitor of Cdc25. The method includes the steps of (1) identifying one or more functional groups capable of interacting with one or more subsites of the Cdc25 catalytic domain; and (2) identifying a scaffold which presents the functional group or functional groups identified in step 1 in a suitable orientation for interacting with one or more subsites of the Cdc25 catalytic domain. The compound which results from attachment of the identified functional groups or moieties to the identified scaffold is a potential inhibitor of Cdc25. The Cdc25 catalytic domain is, generally, defined by the atomic coordinates of a polypeptide comprising the Cdc25 catalytic domain, for example, the atomic coordinates set forth in FIGS. 15A-15PPP, 16A-16I, 17A-17EE, 18A-18X or 19A-19I. Preferably, the Cdc25 catalytic domain is defined by the atomic coordinates set forth in FIG. 15A-15PPP.

Suitable methods, as are known in the art, can be used to identify chemical moieties, fragments or functional groups which are capable of interacting favorably with a particular subsite or set of subsites. These methods include, but are not limited to: interactive molecular graphics; molecular mechanics; conformational analysis; energy evaluation; docking; database searching; pharmacophore modeling; de novo design and property estimation. These methods can also be employed to assemble chemical moieties, fragments or functional groups into a single inhibitor molecule. These same methods can also be used to determine whether a given chemical moiety, fragment or functional group is able to interact favorably with a particular subsite or set of subsites.

In one embodiment, the design of potential human Cdc25 inhibitors begins from the general perspective of three-dimensional shape and electrostatic complementarity for the catalytic domain, encompassing subsites 1-16, and subsequently, interactive molecular modeling techniques can be applied by one skilled in the art to visually inspect the quality of the fit of a candidate inhibitor modeled into the binding site. Suitable visualization programs include INSIGHTII (Molecular Simulations Inc., San Diego, Calif.), QUANTA (Molecular Simulations Inc., San Diego, Calif.), SYBYL (Tripos Inc., St Louis, Mo.), RASMOL (Roger Sayle et al., Trends Biochem. Sci. 20: 374-376 (1995)), GRASP (Nicholls et al, Proteins 11: 281-289 (1991)), and MIDAS (Ferrin et al., J. Mol. Graphics 6:13-27(1988)).

A further embodiment of the present invention utilizes a database searching program which is capable of scanning a database of small molecules of known three-dimensional structure for candidates which fit into the target protein site. Suitable software programs include CATALYST (Molecular Simulations Inc., San Diego, Calif.), UNITY (Tripos Inc., St Louis, Mo.), FLEXX (Rarey et al., J. Mol. Biol. 261: 470-489 (1996)), CHEM-3DBS (Oxford Molecular Group, Oxford, UK), DOCK (Kuntz et al, J. Mol. Biol 161: 269-288 (1982)), and MACCS-3D (MDL Information Systems Inc., San Leandro, Calif.). It is not expected that the molecules found in the search will necessarily be leads themselves, since a complete evaluation of all interactions will necessarily be made during the initial search. Rather, it is anticipated that such candidates might act as the framework for further design, providing molecular skeletons to which appropriate atomic replacements can be made. Of course, the chemical complimentary of these molecules can be evaluated, but it is expected that the scaffold, functional groups, linkers and/or monomers may be changed to maximize the electrostatic, hydrogen bonding, and hydrophobic interactions with the enzyme. Goodford (Goodford J Med Chem 28:849-857 (1985)) has produced a computer program, GRID, which seeks to determine regions of high affinity for different chemical groups (termed probes) on the molecular surface of the binding site. GRID hence provides a tool for suggesting modifications to known ligands that might enhance binding.

A range of factors, including electrostatic interactions, hydrogen bonding, hydrophobic interactions, desolvation effects, conformational strain, and cooperative motions of ligand and enzyme, all influence the binding effect and should be taken into account in attempts to design bioactive inhibitors.

Yet another embodiment of a computer-assisted molecular design method for identifying inhibitors comprises searching for fragments which fit into a binding region subsite and link to a pre-defined scaffold. The scaffold itself may be identified in such a manner. Programs suitable for the searching of such functional groups and monomers include LUDI (Boehm, J Comp. Aid. Mol. Des. 6:61-78 (1992)), CAVEAT (Bartlett et al. in “Molecular Recognition in Chemical and Biological Problems”, special publication of The Royal Chem. Soc., 78:182-196 (1989)) and MCSS (Miranker et al. Proteins 11: 29-34 (1991)).

Yet another embodiment of a computer-assisted molecular design method for identifying inhibitors of the subject phosphatase comprises the de novo synthesis of potential inhibitors by algorithmic connection of small molecular fragments that will exhibit the desired structural and electrostatic complementarity with the active site of the enzyme. The methodology employs a large template set of small molecules with are iteratively pieced together in a model of the Cdc25 active site. Programs suitable for this task include GROW (Moon et al. Proteins 11:314-328 (1991)) and SPROUT (Gillet et al. J Comp. Aid. Mol. Des. 7:127 (1993)).

In yet another embodiment, the suitability of inhibitor candidates can be determined using an empirical scoring function, which can rank the binding affinities for a set of inhibitors. For an example of such a method see Muegge et al. and references therein (Muegge et al., J Med. Chem. 42:791-804 (1999)).

Other modeling techniques can be used in accordance with this invention, for example, those described by Cohen et al. (J. Med. Chem. 33: 883-894 (1994)); Navia et al. (Current Opinions in Structural Biology 2: 202-210 (1992)); Baldwin et al. (J. Med. Chem. 32: 2510-2513 (1989)); Appelt et al. (J. Med. Chem. 34: 1925-1934(1991)); and Ealick et al. (Proc. Nat. Acad. Sci. USA 88: 11540-11544 (1991)).

A compound which is identified by one of the foregoing methods as a potential inhibitor of Cdc25 can then be obtained, for example, by synthesis or from a compound library, and assessed for the ability to inhibit Cdc25 in vitro. Such an in vitro assay can be performed as is known in the art, for example, by contacting Cdc25 in solution with the test compound in the presence of a substrate for Cdc25. The rate of substrate transformation can be determined in the presence of the test compound and compared with the rate in the absence of the test compound. Suitable assays for Cdc25 biological activity are described in U.S. Pat. Nos. 5,861,249; 5,695,950; 5,443,962; and 5,294,538, the teachings of each of which are hereby incorporated by reference herein in their entirety.

An inhibitor identified or designed by a method of the present invention can be a competitive inhibitor, an uncompetitive inhibitor or a noncompetitive inhibitor. A “competitive” inhibitor is one that inhibits Cdc25 activity by binding to the same kinetic form of Cdc25, as its substrate, thereby directly competing with the substrate for the active site of Cdc25. Competitive inhibition can be reversed completely by increasing the substrate concentration. An “uncompetitive” inhibitor inhibits Cdc25 by binding to a different kinetic form of the enzyme than the substrate. Such inhibitors bind to Cdc25 already bound with the substrate and not to the free enzyme. Uncompetitive inhibition cannot be reversed completely by increasing the substrate concentration. A “non-competitive” inhibitor is one that can bind to either the free or substrate bound form of Cdc25.

In another embodiment, the present invention provides Cdc25 inhibitors, and methods of use thereof, which are capable of binding to the catalytic domain of Cdc25, for example, compounds which are identified as inhibitors of at least one biological activity of Cdc25 or which are designed by the methods described above to inhibit at least one biological activity of Cdc25. For example, the invention includes compounds which interact with one or more, preferably two or more, and more preferably, three or more of Cdc25 subsites 1 to 16.

In one embodiment, the Cdc25 inhibitor of the invention comprises a moiety or moieties positioned to interact with subsite 1, subsite 2 and at least one other subsite when present in the Cdc25 catalytic domain. For example, a functional group which can interact with subsite 1 can be a hydrogen bond acceptor, a hydrophobic moiety or a negatively charged group. Preferably, the functional group includes both a negatively charged group and a hydrophobic group. A functional group which can interact with subsite 2 can be a hydrogen bond donor, a hydrogen bond acceptor, a hydrophobic moiety, a negatively charged group or a positively charged group.

In another embodiment, the Cdc25 inhibitor of the invention comprises functional groups positioned to interact with subsites 1, 2 and 3, and, optionally, one or more additional subsites.

The Cdc25 inhibitors of the invention also include compounds having functional groups positioned to interact with subsite 1, subsite 3 and, optionally, one or more additional subsites. In another embodiment, the inhibitor has functional groups positioned to interact with subsite 1, subsite 3, subsite 4, and, optionally, one or more additional subsites.

In other embodiments, the Cdc25 inhibitors of the invention include compounds which have functional groups positioned to interact with the following groups of subsites, each of which can, optionally, include one or more additional subsites: subsites 1 and 5; subsites 1, 4 and 5; subsites 1, 5 and 6; subsites 1, 7 and/or 8; subsites 1, 2 and 9; subsites 1, 2, 4 and 9; subsites 1, 3 and 9; subsites 1, 3, 4 and 9.

A moiety of the inhibitor compound is “positioned to interact” with a given subsite, if, when placed within the Cdc25 catalytic domain, as defined by the atomic coordinates presented in FIG. 15A to 15EE, the moiety is close enough to, and oriented properly relative to, the appropriate amino acid side chains within the subsite.

As indicated in the description of the subsites above, several of subsites 1-16 can potentially interact with two or more types of moieties. For each of the subsites listed below the preferred type of interacting moiety possessed by the potential inhibitor is indicated.

Subsite 1: negative charged (Arg 479) and hydrophobic moiety (Glu 474, Phe 475, Ser 477, Glu 478)

Subsite 3: negative charged moiety (Arg 482; Arg 544)

Subsite 5: hydrophobic, preferably aromatic, moiety (Tyr 428; Glu 478; Arg 479; Met 531; Leu 540; Arg 544)

Subsite 8: hydrophobic, preferably alkyl, moiety (Glu 474; Phe 475; Met 531; Asn 532)

Subsite 11: positive charged moiety (Glu 478)

Subsite 12: negative charged moiety (Lys 394)

Subsite 13: negative charged moiety (Arg 482)

Subsite 14: negative charged moiety (Arg 544)

Subsite 16: hydrophobic and hydrogen donor/acceptor (Asn 532)

A preferred Cdc25 inhibitor of the invention inhibits Cdc25 enzymatic activity with a Ki of at least about 1 mM, preferably at least about 100 μM and more preferably at least about 10 μM.

In a preferred embodiment, the Cdc25 inhibitor of the invention comprises two or more of the following when present at, or bound to, the Cdc25 catalytic domain: (a) a negatively charged functional group positioned to interact with Arg 479 of human Cdc25B; (b) a hydrogen bond donor or positively charged functional group positioned to interact with one or more of Cys 426, Tyr 428, Pro 444, Glu 446 and Thr 547 of human Cdc25B; (c) a hydrogen bond acceptor or a negatively charged functional group positioned to interact with one or more of Tyr 428, Arg 482 and Arg 544 of human Cdc25B; (d) a hydrophobic moiety positioned to interact with one or more of Leu 445, Glu 446, Arg 479, Met 483, Thr 547 and Arg 548; (e) a negatively charged functional group positioned to interact with one or more of Arg 482 and Arg 544 of human Cdc25B; (f) a hydrophobic moiety positioned to interact with one or more of Glu 478, Arg 479, Met 531 and Arg 544 of human Cdc25B; (g) a hydrophobic moiety positioned to interact with one or more of Tyr 428, Glu 478, Arg 479, Met 531, Leu 540, and Arg 544 of human Cdc25B; (h) a hydrophobic moiety positioned to interact with one or more of Phe 475, Met 531, Asn 532 and Leu 540 of human Cdc25B; (i) a hydrophobic moiety positioned to interact with one or more of Phe 475 and Ser 477 of human Cdc25B; (j) a hydrophobic moiety positioned to interact with one or more of Glu 474, Phe 475, Met 531 and Asn 532 of human Cdc25B; (k) a hydrophobic moiety positioned to interact with one or more of Tyr 428, Met 531, Lys 537, Lys 541, Leu 540 and Arg 544 of human Cdc25B; (1) a hydrogen bond donor or hydrogen bond acceptor positioned to interact with Ser 477 of human Cdc25B; (m) a hydrogen bond donor or positively charged functional group positioned to interact with Glu 478 of human Cdc25B; (n) a negatively charged functional group positioned to interact with Lys 394 of human Cdc25B; (o) a negatively charged functional group positioned to interact with Arg 482 of human Cdc25B; (p) a negatively charged functional group positioned to interact with Arg 544 of human Cdc25B; and (q) a hydrophobic moiety and a hydrogen bond donor or hydrogen bond acceptor positioned to interact with Asn 532 of human Cdc25B.

In preferred embodiments, the Cdc25 inhibitors of the invention comprise (a) and (e); (a) and at least one of (b), (c) and (d); (a), (e) and at least one of (b), (c) and (d); (a), (e) and (f); (a) and (g); (a), (f) and (g); (a), (g) and (h); (a) and at least one of (i) and (j); (a), (k) and at least one of (b), (c) and (d).

Preferred Cdc25 inhibitors of the invention comprise a peptide, peptide mimetic or other molecular scaffold or framework, to which the moieties and/or functional groups which interact with the Cdc25 subsites are attached, either directly or via an intervening moiety. The scaffold can be, for example, a peptide or peptide mimetic backbone, a cyclic or polycyclic moiety, such as a monocyclic, bicyclic or tricyclic moiety, and can include one or more hydrocarbyl or heterocyclic rings. The molecular scaffold presents the attached interacting moieties in the proper configuration or orientation for interaction with the appropriate residues of Cdc25.

Information derived from the Cdc25B(ΔN8B)/cdc1249 crystal structure described above has been successfully used to produce a potent inhibitor of Cdc25A. Compound cdc1671, shown below, inhibits Cdc25A with an IC₅₀ of 5 μM.

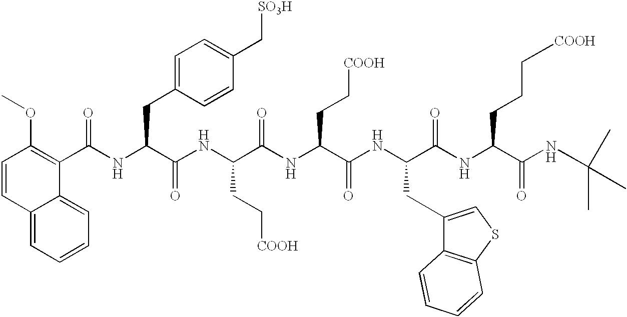

As discussed above, the cdc1249 molecule at the catalytic domain of Cdc25B adopts a “turn” conformation, in which the peptide backbone has a helical turn. Further, the structure shows that the two internal glutamyl residues of cdc1249 do not interact significantly with residues of the catalytic domain. It was therefore reasoned that replacement of one glutamyl residue with a substituted or unsubstituted prolyl or dehydroprolyl residue, which would stabilize the “turn” conformation, would result in a more potent inhibitor. To test this idea, compound cdc1763, shown below, was synthesized. This compound inhibits Cdc25A with an IC₅₀ of 2.2 μM, a two-fold increase in potency compared to cdc1671.

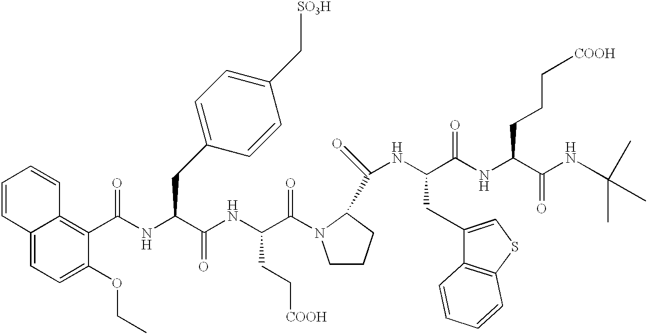

Furthermore, the first glutamyl residue could be replaced by a neutral amino acid such as the tert butyl ester of glutamic acid itself or neutral amino acids such as norvaline or norleucine, thus changing the physicochemical properties of the peptide. The following pentapeptides, cdc1719, cdc1748 and cdc1749 were prepared and they have IC₅₀ values comparable to or better than cdc1679 (1.1 μM, 2.5 μM, 1.5 μM against Cdc25A respectively).

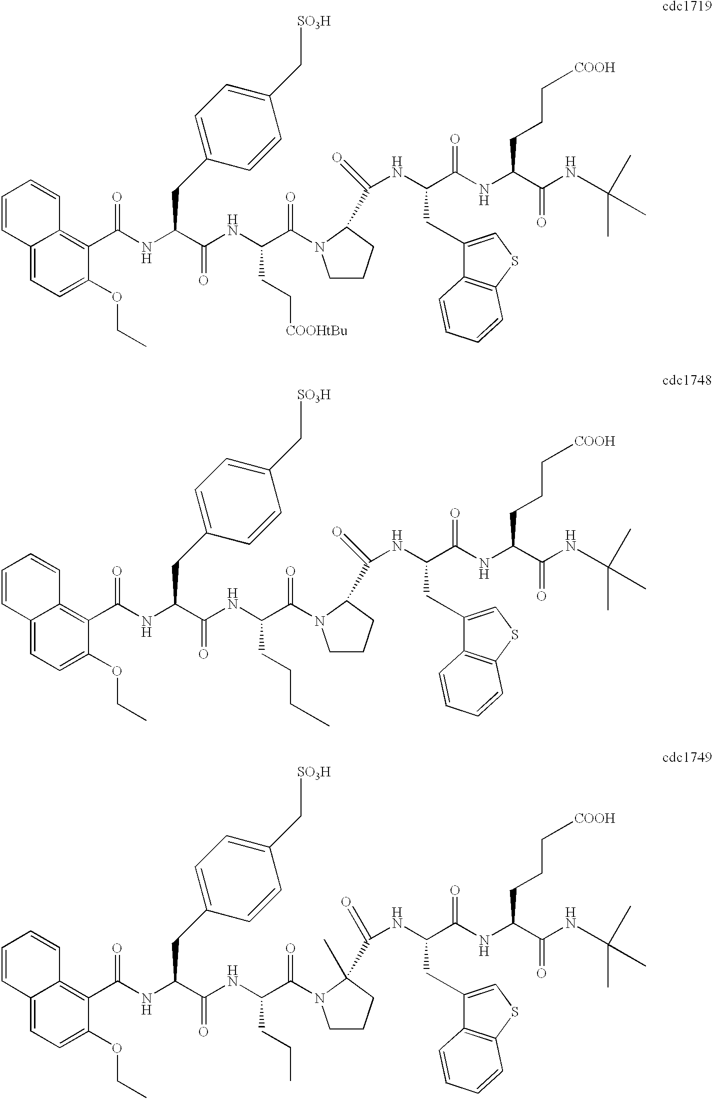

In a preferred embodiment, the Cdc25 inhibitor of the invention is of Formula I,

R₁—A1-A2-A3-A4-R₂ (I)

or a pharmaceutically acceptable salt or prodrug thereof, or a combination thereof, where R₁ is R₃-CO, R₄R₅N-CO, R₆-SO_{2,} R₇R₈NSO₂, wherein R₃, R₄, R₅, R_{6,} R₇ and R_{8,} are independently of each other, hydrogen, substituted or unsubstituted alkyl, substituted or unsubstituted aryl, substituted or unsubstituted cycloalkyl, substituted or unsubstituted heterocycloalkyl, E- or Z-aryl-C₂-C₄-alkenyl or aryl-C₂-C₄-alkinyl.

Suitable alkyl substituents include hydrogen, hydroxy, C₁₋₆alkoxy, phenoxy, benzyloxy, halogen, amino, C₁₋₆alkylamino, di-C₁₋₆alkylamino, C₁₋₆alkyl-CO—NH, substituted and unsubstituted cycloalkyl, substituted and unsubstituted heterocycloalkyl, and substituted or unsubstituted aryl.

An aryl group can be, for example, a phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzodihydrofuranyl, benzothienyl, pyrazolyl, indolyl, purinyl, isoxazolyl, oxazolyl or dibenzofuranyl group. Substituted aryl groups can be, for example, mono-, di- or trisubstituted and suitable substituents can be independently selected from C₁₋₆alkyl, halo, hydroxy, C₁₋₆alkyl amino, di-C₁₋₆alkyl amino, C₁₋₆alkoxy, C₁₋₆alkylthio, C₁₋₆alkylcarbonyl, phenylcarbonyl, benzylcarbonyl, C₁₋₆alkyl-sulfonyl, C₁₋₆alkyl-sulfonyl-amino, C₁₋₆alkyl-carbonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl and oxytrifluoromethyl.

Suitable cycloalkyl groups include substituted and unsubstituted C₃₋₈-cycloalkyl, adamantyl, bicyclooct[3.3.0]-yl. Examples of suitable heterocycloalkyl groups include substituted and unsubstituted pyrrolidinyl, piperazinyl, tetrahydropyranyl, tetrahydrofuranyl, pyrrolidinonyl and morpholinyl. Suitable substituents on the cycloalkyl or heterocycloalkyl group include one or more of, for example, C₁₋₆alkyl, halo, hydroxy, C₁₋₆alkyl amino, di-C₁₋₆alkyl amino, C₁₋₆alkoxy, C₁₋₆alkylthio and C₁₋₆alkylcarbonyl.

R₄ and R₅ or R₇ and R₈ can also form together a four to seven-membered ring. R₃—CO can be further an amino acid residue of the formula R₉-CO-G where R₉ is hydrogen, C₁₋₆alkyl, phenyl, benzyl, naphthyl, benzyloxy or C₁₋₆alkoxy and G is an Asp, Asn, Pro, Ala, Val, Lys, Gly, Arg, Ile, Ser, Thr, Leu, Trp, Cys, Tyr, Met, Gln, Glu, Phe or His residue.

A1 is an amino acid residue of the general formula II

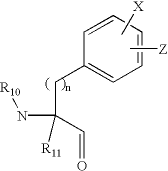

where R₁₀ is hydrogen or C₁₋₆alkyl; R₁₁ is hydrogen or C₁₋₆alkyl; n is 0, 1 or 2; and X is SO₃H, SO₂NR₁₂R₁₃, CH₂—SO₃H, CF₂—SO₃H, CH₂—SO₂NR₁₂R₁₃, CF₂—SO₂NR₁₂R₁₃, where R₁₂ and R₁₃ are independently hydrogen, C₁₋₆alkyl or substituted or unsubstituted phenyl, benzyl, furanyl, thiophenyl, thiazolyl, isothiazolyl, pyrazolyl, isoxazolyl or oxazolyl; or where R₁₂ is hydrogen, R₁₃ can also be hydroxy, C₁₋₆-alkoxy, C₁₋₆-alkylcarbonyl or substituted or unsubstituted benzoyl; or X is PO₃H₂, CH₂PO₃H₂, CF₂—PO₃H₂, OCH₂PO₃H₂, COOH, CH₂COOH, CF₂CO₂H, OCH₂CO₂H, OCF₂CO₂H, OCH(CO₂H)₂, O—CF(CO₂H)_{2,} NH—SO₂—R_{14,} wherein R₁₄ is C₁₋₆alkyl, benzyl or phenyl; or X is NH—CO—COO-R₁₅, wherein R₁₅ is C₁₋₆alkyl, benzyl or phenyl. Preferably, X is at the 3 or 4 position of the phenyl ring. Z is hydrogen, C₁₋₆alkyl, halo, hydroxy, C₁₋₆alkyl amino, di-C₁₋₆alkylamino, C₁₋₆alkoxy, C₁₋₆alkylthio, C₁₋₆alkylcarbonyl, halogen-substituted C₁₋₆alkylcarbonyl, formyl, phenylcarbonyl, benzylcarbonyl, C₁₋₆alkyl-sulfonyl, C₁₋₆alkyl-sulfonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl, oxytrifluoromethyl, or —(CH₂)ₘ—NR₁₆R₁₇, wherein m is 0, 1 or 2 and R₁₆ and R₁₇ are independently of each other selected from hydrogen, C₁₋₆alkyl, C₁₋₆alkyl-carbonyl, amino-C₂₋₆alkyl, C₁₋₆alkyl-amino-C₂₋₆alkyl, di-C₁₋₆alkyl-amino-C₂₋₆alkyl, hydroxy-C₂₋₆alkyl, C₂₋₆alkoxy-C₁₋₆alkyl, aryl-C₀₋₆alkyl, C₃₋₈cycloalkyl-C₀₋₆alkyl and heterocycloalkyl-C₀₋₆alkyl. Aryl, cycloalkyl and heterocycloalkyl are as described above for R₃, R₄ and R₅.

A2 is an amino acid residue of the formula III

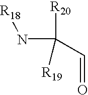

where R₁₈ is hydrogen or C₁₋₆alkyl; R₁₉ is hydrogen or C₁₋₆alkyl; and R₂₀ is the side chain of the amino acid Gly, Ala, Val, Leu, Ile, Nva, Nle, Asp, Glu, Lys, Asn, Gln, Phe, His, homoleucine, Glu(C₁₋₆alkyl), Asp(C₁₋₆alkyl), Lys(Boc). R₂₀ can also be—(CH₂)ₒ—COOR₂₁ with o=3-5 and R₂₁ is hydrogen or C₁₋₆akyl. R₁₉ and R₂₀ can also form, together with the α-carbon, a three to seven-membered carbocyclic ring system. R₁₈ and R₂₀ can also form, together with the nitrogen atom and α-carbon, a four to seven-membered heterocyclic ring system. A2 can, for example, be thioprolyl, dehydroprolyl or substituted or unsubstituted prolyl, for example, mono- or disubstituted prolyl, wherein the substituents are independently of each other hydrogen, C₁₋₆alkyl, phenyl, hydroxy and C₁₋₆alkoxy. R₁₈ and R₂₀ can also form a bicyclic eight to twelve-membered nitrogen-containing ring system such as isoindolinyl, octahydroindolyl or dihydroindolyl.

In preferred embodiments, A2 is aspartyl or an ester thereof; glutamyl or an ester thereof; α-amino adipic acid or an ester thereof; valyl, norvalyl or leucyl.

A3 is an amino acid of the general formula IV,

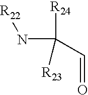

where R₂₂ has the meaning stated above for R₁₈ in Formula III, R₂₃ has the meaning stated for R₁₉ in Formula III and R₂₄ has the meaning stated above for R₂₀ in Formula III.

In preferred embodiments, A3 is aspartyl or an ester thereof; glutamyl or an ester thereof; α-amino adipic acid or an ester thereof; valyl, norvalyl or leucyl; or R₂₃ and R₂₄ together form a three to seven-membered ring; or R₂₂ and R₂₄, together with the nitrogen atom, form a substituted or unsubstituted heterocycle. For example R₃ can be prolyl or substituted prolyl, such as 2-methylprolyl, 3-methylprolyl, 5-phenylprolyl, 3-hydroxyprolyl, 3-tert-butoxyprolyl, 3,3-dimethylprolyl; dehydroprolyl, isoindolyl, octahydroindolyl or dihydroindolyl.

A4 is an amino acid of the general formula V

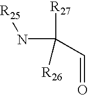

where R₂₅ is hydrogen or C₁₋₆alkyl; R₂₆ is hydrogen or C₁₋₆alkyl; R₂₇ is —(CH₂)ₚ—(CH(R₂₈))_{q}-aryl; p is 0, 1 or 2; q is 0, 1 or 2 and R₂₈ is hydrogen or methyl.

Suitable aryl groups include substituted and unsubstituted phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, tetrahydronaphthyl, benzodihydrofuranyl, quinazoline, benzothienyl, pyrazolyl, indolyl, purinyl, isoxazolyl, oxazolyl, dibenzofuranyl.

Suitable substituents for the aryl groups include hydrogen, C₁₋₆alkyl, halo, hydroxy, C₁₋₆akyl amino, di-C₁₋₆alkyl amino, C₁₋₆alkoxy, C₁₋₆alkylthio, C₁₋₆alkylcarbonyl, halogen-substituted C₁₋₆alkylcarbonyl, formyl-, phenylcarbonyl, benzylcarbonyl, C₁₋₆alkyl-sulfonyl, C₁₋₆alkyl-sulfonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl, oxytrifluoromethyl, aryl, Y—(CH₂)ₛ-C₃₋₈cycloalkyl, Y—(CH₂)ₛ-aryl, where Y is O, S, NH and s is 0, 1, 2 or 3; Y—(CH₂)ᵤ-R₂₉ where Y is O, NH, or S, u is 2 to 6 and R₂₉ is OH, CH₂—OH, NH2 or NH(C═NH)NH₂; Y—(CH₂)ᵥ—R₃₀ where Y is O, NH or S, v is 1-6 and R₃₀ is COC₁₋₆alkyl, COOH or CONH₂; Y—(CH═CH)—R₃₁, where R₃₁ is COC₁₋₆alkyl, COOH, CONH₂ or phenyl. Suitable aryl groups within the foregoing substituents include substituted and unsubstituted phenyl, pyridyl, furanyl, thienyl, thiazolyl, isothiazolyl, imidazolyl, pyrazinyl, pyrimidyl, pyrazolyl, isoxazolyl and oxazolyl, which can be independently substituted by one or more of hydroxy, amino, carboxyl, carboxamide, halo, hydroxy, C₁₋₆alkyl amino, di-C₁₋₆alkyl amino, C₁₋₆alkoxy, C₁₋₆alkylthio, C₁₋₆alkylcarbonyl, Y—(CH₂)ₜ-heterocycloalkyl, where Y is O, S or NH and t is 0, 1, 2 or 3, and the heterocycloalkyl group is selected from the group consisting of morpholinyl, pyrrolidinyl, piperazinyl, N-substituted piperazinyl, piperidinyl, tetrahydropyranyl, tetrahydrofuranyl, and pyrrolidinonyl. In a preferred embodiment, R₂₇ is an aryl group selected from substituted and unsubstituted phenyl, naphthyl, such as 1-naphthyl, and benzothienyl, such as 3-benzothienyl.

R₂ is NR₃₂R₃₃, where R₃₂ is hydrogen or C₁₋₆alkyl; and R₃₃ is (CH₂)_{w}—W—(CH₂)ₓ—V, where W is a single bond, wherein the sum of w and x is 1 to 6, or, where w is 0, 1, 2 or 3 and x is 0, 1, 2 or 3, W can be aryl or aryl-T, where T is O, S or NH. Suitable aryl groups include substituted and unsubstituted phenyl, naphthyl, pyridyl, furanyl, thienyl and pyrimidyl. W can also be C₃₋₈cycloalkyl, where w is 0, 1, 2 or 3 and x is 0, 1, 2 or 3. V is COOR₃₄ where R34 is hydrogen or C₁₋₆alkyl; or V is COC₁₋₆alkyl, CONH₂, SO₃H or NO₂.

R₂ can also be an amino acid A5 of Formula VI

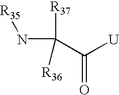

where R₃₅ is hydrogen or C₁₋₆alkyl; R₃₆ is hydrogen or C₁₋₆alkyl; R₃₇ is the side chain of the amino acid Asp, Asn, Glu, Gln, Asp (C₁₋₆alkyl), Glu(C₁₋₆alkyl) or (CH₂)_{y}—COOR₄₂ where y is 3, 4 or 5; and R₄₂ is hydrogen or C₁₋₆alkyl; or R₃₇ is (CH₂)_{z}—CONR₄₀R₄₁, where z is 1 to 5 and R₄₀ and R₄₁ are independently, hydrogen or C₁₋₆-alkyl, or R_{40,} R₄₁ and the nitrogen atom together form a 5- to 8-member heterocycle; or R₃₇ is (CH₂)ₐ—SO₃H, where a is 1, 2, 3, 4 or 5; or (CH₂)_{b}-tetrazolyl where b is 1, 2, 3, 4 or 5; or R₃₇ is (CH₂)_{d}-phenyl-(CH₂)ₑ—COOR43 where d is 0 to 2, e is 0 to 2 and R₄₃ is hydrogen or C₁₋₆alkyl; or R₃₇ is (CH₂)_{d}-phenyl-(CH₂)ₑ—CONR₄₄R₄₅ wherein d is 0 to 2 and e is 0 to 2 and R₄₄ and R₄₅ are independently hydrogen, C₁₋₆alkyl or R₄₄ and R₄₅ and the nitrogen atom together form a 5 to 8-member heterocycle. Preferably, R₃₇ is the side chain of aspartic acid or glutamic acid; (CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl. U is hydroxy, C₁₋₆alkoxy or NR₃₈R₃₉, where R₃₈ and R₃₉ are, independently of each other, hydrogen; substituted or unsubstituted C₁₋₁₀-alkyl, substituted or unsubstituted aryl or substituted or unsubstituted cycloalkyl or bicycloalkyl. Suitable alkyl substituents include hydrogen, hydroxy, halogen, substituted and unsubstituted aryl and substituted and unsubstituted cycloalkyl. The aryl group can be selected from substituted and unsubstituted phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzothienyl, pyrazolyl, indolyl, purinyl, isoxazolyl, oxazolyl, dibenzofuranyl. Suitable aryl substituents are independently, C₁₋₆alkyl, halo, hydroxy, C₁₋₆alkyl amino, di-C₁₋₆alkyl amino, C₁₋₆alkoxy, C₁₋₆alkylthio, C₁₋₆alkylcarbonyl, phenylcarbonyl, benzylcarbonyl, C₁₋₆alkyl-sulfonyl, C₁₋₆alkyl-sulfonyl-amino, C₁₋₆alkyl-carbonyl-amino, carboxyl, O-C₁₋₆alkylcarboxyl, carboxylalkenyl, O-C₁₋₆alkyl carboxyl alkenyl, C₁₋₆alkylcarbamoyl, cyano, nitro, trifluoromethyl and oxytrifluoromethyl. Suitable cycloalkyl groups include C₃₋₈-cycloalkyl, adamantyl and bicyclooctyl. Preferably, U is OH or NHR_{38,} wherein R₃₈ is tert. butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

In one subset of compounds of Formula I, R₂ is (CH₂)_{w}—W—(CH₂)ₓ—COOR₃₄, wherein W is a single bond, phenyl or C₆-cycloalkyl.

By the terms “amino acid residue” and “peptide residue” is meant an amino acid or peptide molecule without the —OH of its carboxyl group (C-terminally linked) or the proton of its amino group (N-terminally linked). In general the abbreviations used herein for designating the amino acids and the protective groups are based on recommendations of the IUPAC-IUB Commission on Biochemical Nomenclature (see Biochemistry (1972) 11:1726-1732). For instance Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the H portion of the α-amino group. The term “amino acid side chain” is that part of an amino acid exclusive of the —CH(NH₂)COOH portion, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH(CH₃)—CH₂CH₃ (the side chain of isoleucine), —CH₂CH(CH₃)₂ (the side chain of leucine) or H-(the side chain of glycine).

For the most part, the amino acids used in the application of this invention are those naturally occurring amino acids found in proteins, or the naturally occurring anabolic or catabolic products of such amino acids which contain amino and carboxyl groups. Particularly suitable amino acid side chains include side chains selected from those of the following amino acids: glycine, alanine, valine, cysteine, leucine, isoleucine, serine, threonine, methionine, glutamic acid, aspartic acid, glutamine, asparagine, lysine, arginine, proline, histidine, phenylalanine, tyrosine, and tryptophan.

However, the term amino acid residue further includes analogs, derivatives and congeners of any specific amino acid referred to herein. For example, the present invention contemplates the use of amino acid analogs wherein a side chain is lengthened or shortened while still providing a carboxyl, amino or other reactive precursor functional group for cyclization, as well as amino acid analogs having variant side chains with appropriate functional groups). For instance, the subject peptidomimetic can include an amino acid analog as for example, α-cyanoalanine, canavanine, djenkolic acid, norleucine, 3-phosphoserine, homoserine, dihydroxyphenylalanine, 5-hydroxytryptophan, 1-methylhistidine, or 3-methylhistidine. Other naturally occurring amino acid metabolites or precursors having side chains which are suitable herein will be recognized by those skilled in the art and are included in the scope of the present invention.

Also included are the D and L stereoisomers of such amino acids when the structure of the amino acid admits of stereoisomeric forms. The configuration of the amino acids and amino acid residues herein are designated by the appropriate symbols D, L or DL, furthermore when the configuration is not designated the amino acid or residue can have the configuration D, L or DL. It will be noted that the structure of some of the compounds of this invention includes asymmetric carbon atoms. It is to be understood accordingly that the isomers arising from such asymmetry are included within the scope of this invention. Such isomers are obtained in substantially pure form by classical separation techniques and by sterically controlled synthesis and have arbitrarily been named, for example, as isomers #1 or #2. For the purposes of this application, unless expressly noted to the contrary, a named amino acid shall be construed to include both the D or L stereoisomers, preferably the L stereoisomer.

The phrase “protecting group” as used herein means temporary substituents which protect a potentially reactive functional group from undesired chemical transformations. Examples of such protecting groups include esters of carboxylic acids, silyl ethers of alcohols, and acetals and ketals of aldehydes and ketones, respectively. The field of protecting group chemistry has been reviewed (Greene, T. W.; Wuts, P. G. M. Protective Groups in Organic Synthesis, 3^{nd} ed.; Wiley: New York, 1999; and Kocienski, P. J. Protecting Groups, Georg Thieme Verlag: New York, 1994).

The phrase “N-terminal protecting group” or “amino-protecting group” as used herein refers to various amino-protecting groups which can be employed to protect the N-terminus of an amino acid or peptide against undesirable reactions during synthetic procedures. Examples of suitable groups include acyl protecting groups such as, to illustrate, formyl, dansyl, acetyl, benzoyl, trifluoroacetyl, succinyl and methoxysuccinyl; aromatic urethane protecting groups as, for example, carbonylbenzyloxy (Cbz); and aliphatic urethane protecting groups such as t-butyloxycarbonyl (Boc) or 9-Fluorenylmethoxycarbonyl (FMOC). Peptidomimetics of the present invention which have sidechain or azepine ring substituents which include amino groups —such as where R₃ is a lysine or arginine, or where R₈, R₁, R₂ or Y comprise a free amino group, can optionally comprise suitable N-terminal protecting groups attached to the sidechains.

The phrase “C-terminal protecting group” or “carboxyl-protecting group” as used herein refers to those groups intended to protect a carboxylic acid group, such as the C-terminus of an amino acid or peptide. Benzyl or other suitable esters or ethers are illustrative of C-terminal protecting groups known in the art.

In addition to a variety of sidechain replacements which can be carried out to generate the subject peptidomimetics, the present invention specifically contemplates the use of conformationally restrained mimics of peptide secondary structure or mimetics, which are not-cleavable by hydrolytic enzymes. Numerous surrogates have been developed for the amide bond of peptides. Frequently exploited surrogates for the amide bond include the following groups (i) trans-olefins, (ii) fluoroalkene, (iii) methylene-oxy, (iv) methylene-amino, (v) methylene-thio, (vi) dihydroxyethylene, (vii) phosphonamides, (viii) sulfonamides and (ix) ketomethylene.

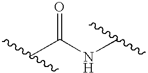

amide bond

| | | |
|---|---|---|
| 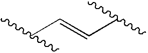 | 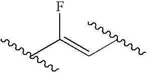 | 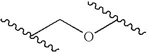 |
| trans-olefine | fluoro-olefine | methylene-oxy |
| 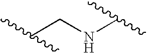 | 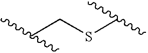 | 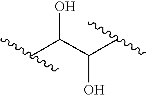 |
| methylene-amino | methylene-thio | dihydroxyethylene |
| 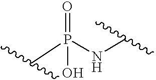 | 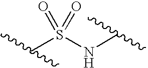 | 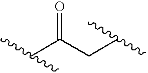 |
| phosphoramidone | sulfonamide | ketomethylene |

Additionally, peptidomimietics based on more substantial modifications of the backbone of the peptide can be used. Peptidomimetics which fall in this category include (i) retro-inverso analogs, and (ii) N-alkyl glycine analogs (so-called peptoids).

Furthermore, the methods of combinatorial chemistry are being brought to bear, e.g., by G. L. Verdine at Harvard University, on the development of new peptidomimetics. For example, one embodiment of a so-called “peptide morphing” strategy focuses on the random generation of a library of peptide analogs that comprise a wide range of peptide bond substitutes.

In an exemplary embodiment, the peptidomimetic can be derived as a retro-inverso analog of the peptide. Such retro-inverso analogs can be made according to the methods known in the art, such as that described by the Sisto et al. U.S. Pat. No. 4,522,752.

Retro-enantio analogs such as this can be synthesized from commercially available D-amino acids (or analogs thereof) and standard solid- or solution-phase peptide-synthesis techniques. For example, in a preferred solid-phase synthesis method, a suitably amino-protected (fluorenyl-methoxycarbonyl, Fmoc) D-_{A1} residue (or analog thereof) is covalently bound to a solid support such as chlortrityl chloride resin. The resin is washed with diemthylformamide, dichloromethane (DCM) and methanol, and the Fmoc protecting group removed by treatment with piperidine in DCM. The resin is washed and neutralized, and the next Fmoc-protected D-amino acid (D-A2) is introduced by coupling with diisopropylcarbodiimide. The resin is again washed, and the cycle repeated for each of the remaining amino acids in turn (D-A3, D-A4, etc). When synthesis of the protected retro-enantio peptide is complete, the protecting groups are removed and the peptide cleaved from the solid support by treatment with trifluoroacetic acid. The final product is purified by HPLC to yield the pure retro-enantio analog.

In still another illustrative embodiment, trans-olefin derivatives can be made for the subject polypeptide. For example, an exemplary olefin analog is derived for the illustrative pentapeptide:

### 2-EtO-Naphthyl-CO—Smp—NH—CH(CH3)—CH═CH—CH(CH₃)—CO—Phe—Glu—NHtBu

The trans olefin analog of the pentapeptide can be synthesized according to the method of Y. K. Shue et al (Tetrahedron Letters, 28: 3225 (1987)). Referring to the illustrated example, Boc-amino L-Ala is converted to the corresponding α-amino aldehyde, which is treated with a vinylcuprate to yield a diastereomeric mixture of alcohols, which are carried on together. The allylic alcohol is acetylated with acetic anhydride in pyridine, and the olefin is cleaved with osmium tetroxide/sodium periodate to yield the aldehyde, which is condensed with the Wittig reagent derived from a protected alanine precursor, to yield the allylic acetate. The allylic acetate is selectively hydrolyzed with sodium carbonate in methanol, and the allylic alcohol is treated with triphenylphosphine and carbon tetrabromide to yield the allylic bromide. This compound is reduced with zinc in acetic acid to give the transposed trans olefin as a mixture of diastereomers at the newly formed center. The diastereomers are separated and the pseudodipeptide is obtained by selective transfer hydrogenolysis to unveil the free carboxylic acid. Other synthetic approaches to trans olefin building block are described by J. S. Wai et al., (Tetrahedron Letters 36: 3461 (1995)), T. Ikuba et al (J. Org. Chem. 56: 4370 (1991)) and J. A. McKinney (Tetrahedron Letters 35: 5985 (1994)).

The pseudodipeptide in its Fmoc-protected form is then coupled instead of A2 and A3 in the sequence. Other pseudodipeptides can be made by the method set forth above merely by substitution of the appropriate starting Boc amino acid and Wittig reagent. Variations in the procedure may be necessary according to the nature of the reagents used, but any such variations will be purely routine and will be obvious to one of skill in the art.

It is further possible to couple the pseudodipeptides synthesized by the above method to other pseudodipeptides, to make peptide analogs with several olefinic functionalities in place of amide functionalities. For example, pseudodipeptides corresponding to Fmoc-protected Glu—Ala or Tyr—Glu, etc. could be made and then coupled together by standard techniques to yield an analog of the pentapeptide which has alternating olefinic bonds between residues.

Still another class of peptidomimetic derivatives includes the phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Other peptidomimetic structures are known in the art and can be readily adapted for use in the subject peptidomimetics. They would replace two adjacent amino acids in the general formula, preferrably the amino acid sequence A2-A3. The synthetic procedures to incorporate this peptidomimetics are similar to the usual peptide synthesis. The Fmoc-protected form of the peptidomimetic is used instead of the corresponding two amino acid in the build-up of the sequence from the C-terminus. Peptidomimetics PM-1 to PM-18, shown below, can be coupled under the usual peptide coupling conditions providing structures such as R1-A1-(PM-x)-A4-R2 with x is 1 to 18

In one embodiment, the invention provides compounds of Formula I in which A2 and A3 together form a peptidomimetic residue selected from (a) 6-amino-5-oxoperhydropyrido[2,1-b][1,3]thiazole-3-carboxylic acid, preferably (R,S,S)-6-amino-5-oxoperhydropyrido[2,1-b][1,3]thiazole-3-carboxylic acid (PM-1); (b) 6-amino-5-oxoperhydro-3-indolizinecarboxylic acid, preferably (S,S,S)-6-amino-5-oxoperhydro-3-indolizinecarboxylic acid (PM-2); (c) (S, R)-6-amino-5-oxoperhydro-8a-indolizinecarboxylic acid or (R,R)-6-amino-5-oxoperhydro-8a-indolizinecarboxylic acid (PM-3); (d) (R, S)-6-amino-5-oxoperhydro-8a-indolizinecarboxylic acid or (S,S)-6-amino-5-oxoperhydro-8a-indolizinecarboxylic acid (PM-4); (e) 2-(3-amino-2-oxo-1,2-dihydro-1-pyridinyl)acetic acid (PM-5); (f) 2-(3-amino-2-oxo-6-phenyl-1,2-dihydro-1-pyridinyl)acetic acid (PM-6); (g) 3-amino benzoic acid (PM-7); (h) 4-aminobenzoic acid (PM-8); (i) 3-aminomethyl benzoic acid (PM-9-1); (j) (S)-3-(1-aminoethyl)benzoic acid or (R)-3-(1-aminoethyl)benzoic acid (PM-9-2); (k) (S)-3-(1-aminopropyl) benzoic acid or (R)-3-(1-aminopropyl) benzoic acid PM-9-3); (l) (S)-3-(1-aminobutyl) benzoic acid or (R)-3-(1-aminobutyl) benzoic acid (PM-9-4); (m) 2-(3-amino-2-oxo-1-azepanyl)acetic acid, preferably (S)-2-(3-amino-2-oxo-1-azepanyl)acetic acid (PM-10); (n) 2-[8-(aminomethyl)-3,6-dimethyl-9,10,10-trioxo-9,10-dihydro-10λ⁶-thioxanthen-1-yl]acetic acid (PM-11); (o) 2-(2-oxopiperazino)acetic acid (PM-12); (p) 2-[8-(aminomethyl)-2-oxo-5-phenyl-2,3-dihydro-1H-1,4-benzodiazepin-1-yl]acetic acid (PM-13-1); (q) 2-[8-(aminomethyl)-2-oxo-5-methyl-2,3-dihydro-1H-1,4-benzodiazepin-1-yl]acetic acid (PM-13-2); (r) 3-aminopropanoic acid (PM-14); (s)4-aminobutanoic acid (PM-15); (t) 5-aminopentanoic acid (PM-16); (u) 2-[2-(2-aminoethoxy)ethoxy]acetic acid (PM-17); and (v) 2-(3-amino-2-oxo-2,3,4,5-tetrahydro-1H-1-benzazepin-1-yl)acetic acid, preferably (S)-2-(3-amino-2-oxo-2,3,4,5-tetrahydro-1H-1-benzazepin-1-yl)acetic acid.

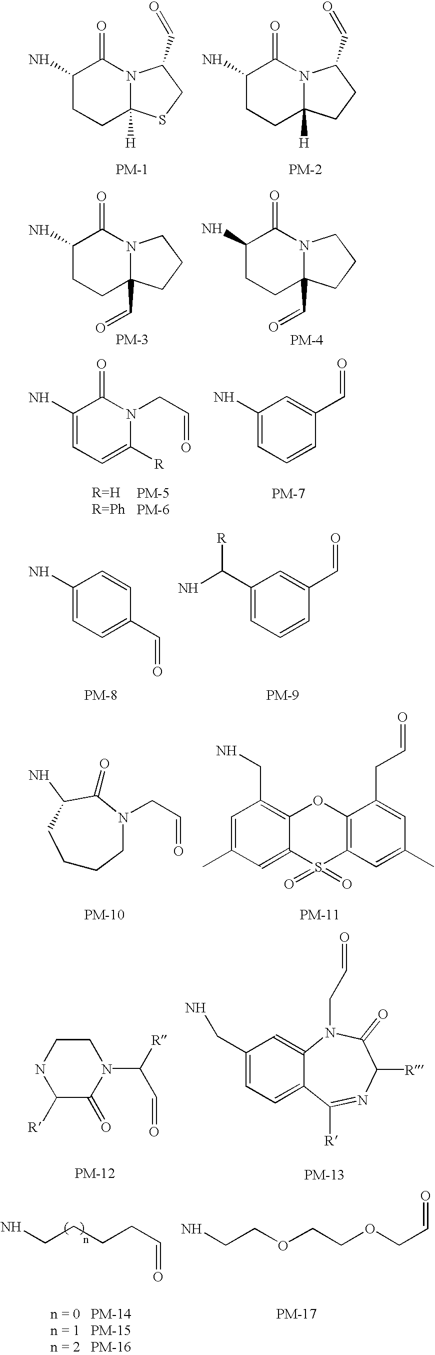

The peptidomimetic residues are shown here in similar way as the “amino acid residue” or “peptide residue” has been defined before. The term “peptidomimetic residue” means without the —OH of its carboxyl group (C-terminally linked) or the proton of its amino group (N-terminally linked). Some of the peptidomimetics are commercially available in their acid form with or without protection of the amino group such as PM-1, PM-7, PM-8, PM-10, PM-14, 15 and 16. Synthesis of the different peptidomimetics are described for PM 5 and 6 by P. D. Edwards et al. (J. Med. Chem. 1996, 39, 1112) and by F. J. Brown et al. (J. Med. Chem. 1994, 37, 1259), for PM 3 and 4 by J. D. Gramberg et al. (Tetrahedron Letters 1994, 35, 861), for PM2 by H. -G. Lombart et al. (J. Org. Chem. 61: 9437 (1996)), for PM-12 by A. Pohlmann et al. (J. Org. Chem. 62: 1016 (1997)), for PM-9 by L. Chen et al. (Tetrahedron Letters 36: 8715 (1995)), for PM-17 by A. M. P. Koskinen (Biorg. & Med. Chem. Lett. 5: 573 (1995)), for PM-13 by W. C. Ripka et al. (Tetrahedron 49: 3593 (1993)), for PM-11 by M. Sato et al. (Biochem. Biophys. Res. Commun. 187: 199 (1992)) and for PM-1 by U. Nagai (Tetrahedron Letters 26: 647 (1985)). The peptidomimetic may incorporate the 1-azabicyclo[4.3.0]nonane surrogate (see Kim et al., J. Org. Chem. 62: 2847 (1997)), or an N-acyl piperazic acid (see Xi et al, J. Am. Chem. Soc. 120: 80 (1998)), or a 2-substituted piperazine moiety as a constrained amino acid analogue (see Williams et al., J. Med. Chem. 39: 1345-1348 (1996)). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject peptidomimetics can be optimized by, e.g., combinatorial synthesis techniques combined with such high throughput screening as described herein.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof.

Numerous efficient methods for the synthesis of racemic unnatural amino acids have been described in literature. The Strecker synthesis involves the reaction of an aldehdye with ammonia or with a substituted primary amine and hydrogen cyanide to form an alpha-amino nitrile which is hydrolyzed to the corresponding amino acid (Houben-Weyl, Methoden der organischen Chemise Vol. 11/2, p. 305 (1958)). The condensation of an amine R₁-NH_{2,} an aldehyde R₂CHO, an acid R₃-COOH and an isocyanide R₄-NC (the Ugi reaction), is a general method of preparing racemic amino acids of the type (R₃—CO)NR₁—CHR₂—CO—NHR₃ (Ugi et al., Liebigs Ann. Chem. 1967, 709, 1; I. Ugi et al. Angew,. Chem. Int. ed. 1962, 1, 8). Solid phase versions of the Ugi reaction have been described (Strocker et al., Tetrahedron Letters 37: 1149 (1996); Zhang et al., Tetrahedron Letters 37: 751 (1996); Tempest et al., Angew. Chem. Int. Ed. 35: 640 (1996); Sutherlin et al., J. Org. Chem. 61: 8350 (1996); Short et al., Tetrahedron Letters 37: 7489 (1996)). Furthermore, racemic amino acids can be prepared by deprotonation of (N-diphenylmethylene)glycine derivatives with strong bases such as sodium hydride or lithium diisopropylaride and reaction of the anion with corresponding alkylating agents, for example substituted bromomethylphenyl-derivatives or bromomethylnaphthyl-derivatives. Protection (Fmoc, Cbz, Boc, Alloc) or acylation of the amino moiety and subsequent hydrolysis affords the corresponding amino acid derivatives. An illustrative example to the building blocks (R₁—CO-(2-Br)—Smp—OH and R₁—CO-(2-CHO)—Smp—OH) is shown in scheme I:

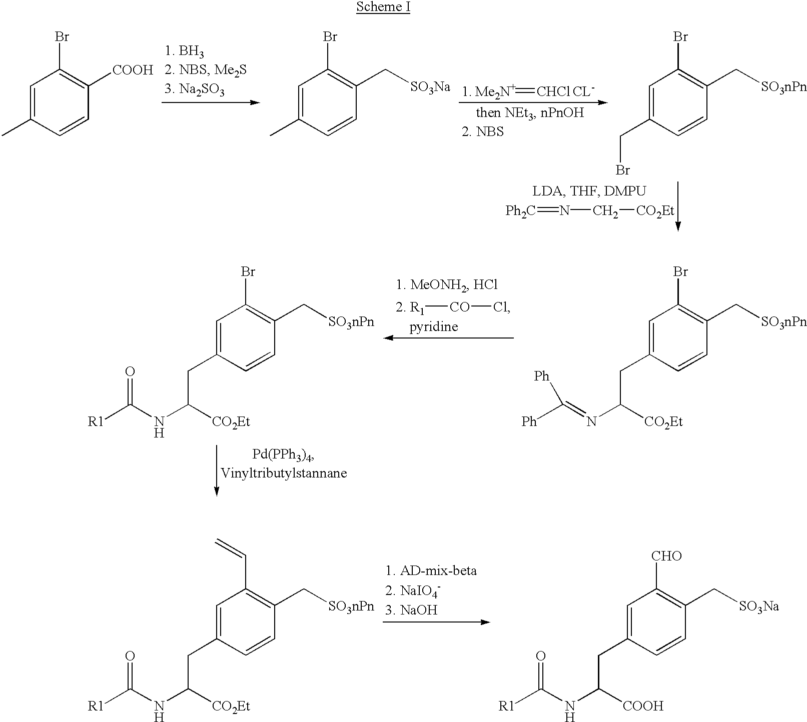

Another route to amino acids consists of reacting the anion of (N-diphenyhnethylene)-glycine derivatives with aldehydes, hydrogenation of thus obtained dehydroamino acids yields the racemic amino acids. Furthermore, hydroxy napthyl alanine derivatives have been prepared by Vela et al. (J. Org. Chem. 55: 2913 (1990)).

The Wittig reaction or its Homer-Emmons-modification of an alpha-phosphoryl-glycine derivatives with aldehydes can be used to synthesize the corresponding dehydroamino acids as described by Ciattini (Ciattini et al., Synthesis 2: 140 (1988)) and Shin (Shin et al., Tetrahedron Lett. 28: 3827 (1987); Shin, et al., Chem. Pharm. Bull. 38: 2020 (1990)).

Hydrogenation of the dehydro amino acid derivatives with metal catalysts such as palladium on carbon, phosphine or amine complexes of rhodium, ruthenium or palladium gives the racemic amino acid derivatives. By using chiral compounds as metal ligands, amino acids can be obtained in high enantiomeric excesses as described in I. Ojima, “Catalytic Asymmetric Synthesis”, Verlag Chemie, 1993, chapter 1, p.6 and R. Noyori, “Asymmetric Catalysis in Organic Synthesis”, John Wiley, 1994, chapter 2, p.16. For example, the synthesis of D- and L-alpha aminoadipate, pimelate and suberate have been described by T. Pham et al. (J. Org. Chem. 59: 3676 (1994)).

Furthermore, methods for preparation of optically active alpha-amino acids can be found in R. M. Williams, “Synthesis of active alpha-amino acids” Pergamon Press, 1989, and in L. M. O'Donnell “alpha-amino acid synthesis”, Tetrahedron symposia-in-print, 44: 5253 (1988). Thus, syntheses of chiral amino acids can be achieved by using chiral glycine derivatives in a similar way as described above. The final chiral amino acid is obtained after removal or cleavage of the chiral auxiliaries. Depending on the chiral auxiliary, either the D or the L-amino acid can be obtained in high enantiomeric excess. Preferred methods are the method described by U. Schoellkopf (Schoellkopf et al, Angew, Chem. Int. Ed. 18: 863 (1979); Schoellkopf et al., Angew, Chem. Int. Ed. 20: 798 (1981); Schoellkopfet al., Synthesis, 969 (1981); Schoellkopf et al., Synthesis, 866 (1982); Schoellkopf et al., Synthesis, 861 (1982); Schoellkopf et al., Synthesis, 37 (1983); Schoellkopf et al., Synthesis, 271 (1984)) the methods described by R. M. Williams using the 5,6-diphenyl-2,3,5,6-tetrahydro-4H-1,4-oxazin-2-one template (Williams et al., J. Am. Chem. Soc. 113: 9276 (1991); Williams et al., Tetrahedron Letters 29: 6075 (1988); Solas et al., J. Org. Chem. 61: 1537 (1996); Williams et al., J. Am. Chem. Soc. 110: 1547 (1988); Williams et al., J. Am. Chem. Soc. 110: 482 (1988); Williams et al., J. Am. Chem. Soc. 108: 1103 (1986)), the methods described by D. Seebach (Fitzi et al., Tetrahedron 44: 5277 (1988), Seebach et al, Liebigs Ann. Chem., 1215 (1989); Schickli et al., Liebigs Ann. Chem., 1323 (1991); Seebach et al., Liebigs Ann. Chem., 1145 (1992); Mueller et al., Helv. Chim. Acta, 75: 855 (1992); Seebach et al., Angew. Chem. 105: 1780 (1993)); and the method described by A. Myers (Myers et al., J. Am. Chem. Soc. 117: 8488 (1995); Myers et al., J. Am. Chem. Soc. 119: 656 (1997)).

An illustrative example, the syntheses of Fmoc-(6-OH)—Nal—OH or Fmoc-(7-OH)Nal—OH, is shown in scheme II.

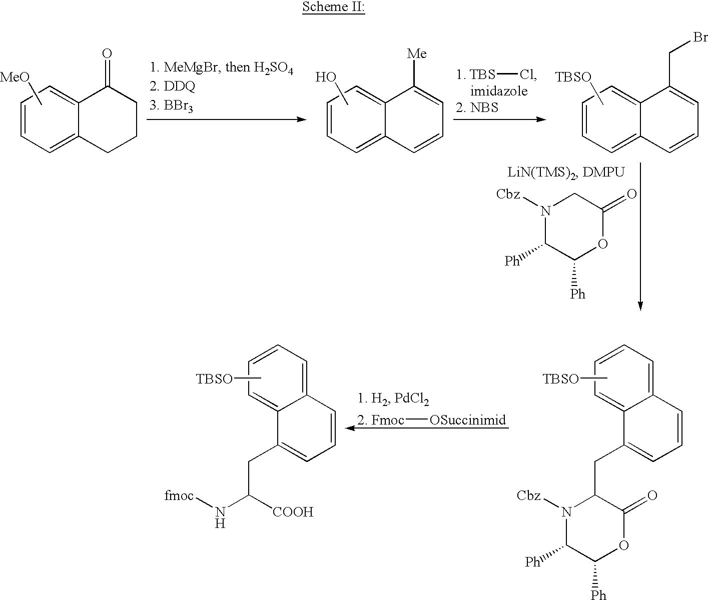

Another route is the electrophilic amination of chiral enolates and subsequent hydrolysis of the chiral auxiliaries as exemplified by D. Evans (D. Evans et al., J. Am. Chem. Soc. 112: 4011 (1990); D. Evans et al., Tetrahedron 44: 5525 (1988)).

Alpha, alpha-disubstituted amino acid derivatives can be obtained by methods described by D. Seebach (Seebach et al., Liebigs Ann. Chem., 217 (1995); Seebach et al., Tetrahedron Letters, 25: 2545 (1984)).

Chiral amino acids are also obtained by resolution of racemic amino acid esters by enzyme-catalyzed acylation as described by Stuermer et al. (BASF AG) Ger. Offen., DE 19727517.

The amino acids obtained by the above methods can be further modified or transformed using standard organic reactions, such as reductive amination, alkylation, esterification, etherification, Mitsunobu reaction, performed with the amino acid in suitable protected form (for example with Boc, Fmoc, Alloc, Cbz) or in a peptidyl environment such as the penta- and tetrapetidyl intermediate, obtained in routes 1 to 4 as described below. The modification or transformation reaction can be carried out in solution or with the amino acid or the peptide derivative bound to a suitable solid phase. For example, the synthesis of Fmoc-N-methyl amino acids has been described by Yang et al., Tetrahedron Letters 42: 7307 (1997), using a solid phase methodology.

An illustrative example for the modification of the pentapeptide is the reductive amination of the 2-formyl-smp-derivative as shown in scheme III:

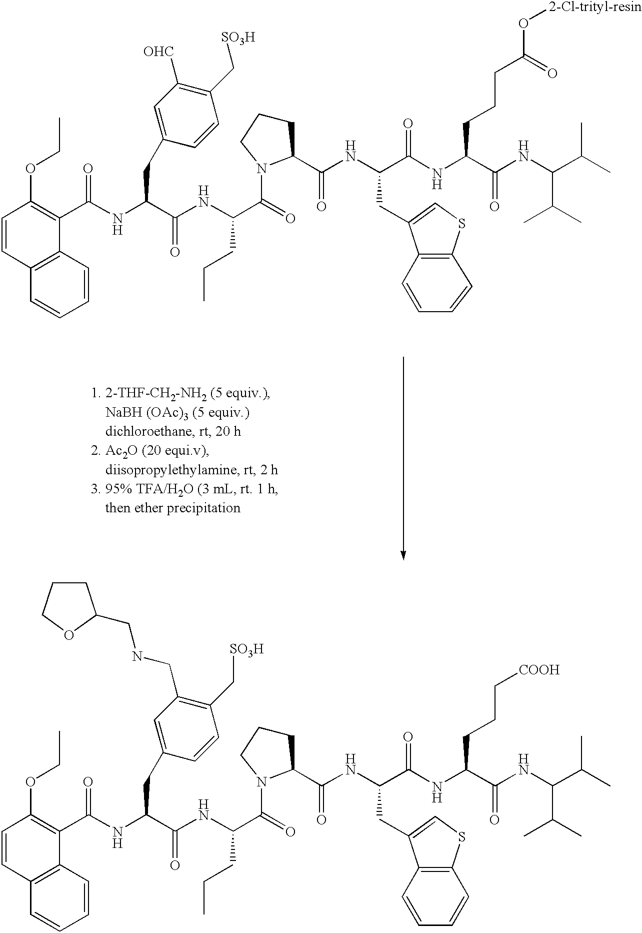

Another illustrative example for modification of a peptidyl intermediate is the Mitsunobu reaction of a Nal-derivative on solid phase as shown in scheme IV:

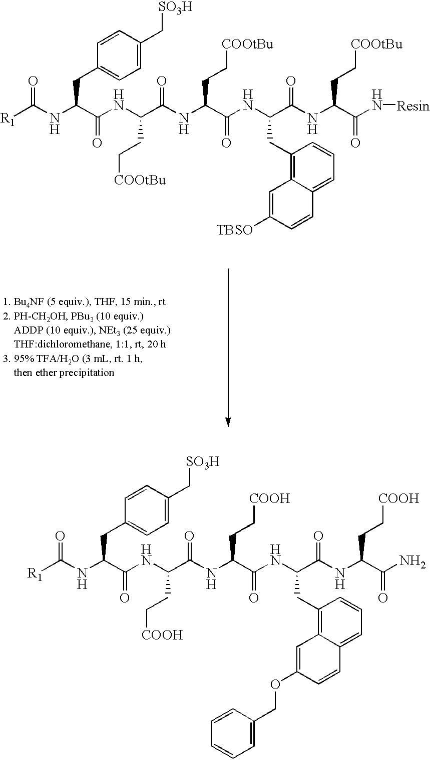

The novel compounds of the general formula I can be prepared by known methods of peptide chemistry. Thus, the peptidyl derivatives can be assembled sequentially from amino acids or by linking suitable small peptide fragments. In the sequential assemblage, starting at the C terminus the peptide chain is extended stepwise by one amino acid each time. In fragment coupling it is possible to link fragments of different lengths, and the fragments in turn can be obtained by sequential assemblage from amino acids or themselves by fragment coupling. Both in the sequential assemblage and in the fragment coupling it is necessary to link the units by forming an amide linkage. Enzymatic and chemical methods are suitable for this. Chemical methods for forming the amide linkage are described in detail by Müller, Methoden der organischen Chemie Vol. XV/2, pp 1 to 364, Thieme Verlag, Stuttgart, 1974; Stewart, Young, Solid Phase Peptide Synthesis, pp 31 to 34, 71 to 82, Pierce Chemical Company, Rockford, 1984; Bodanszky, Klausner, Ondetti, Peptide Synthesis, pp 85 to 128, John Wiley & Sons, New York, 1976 and other standard works on peptide chemistry. Particular preference is given to the azide method, the symmetric and mixed anhydride method, in situ generated or preformed active esters, the use of urethane protected N-carboxy anhydrides of amino acids and the formation of the amide linkage using coupling reagents (activators, especially dicyclohexylcarbodiimide (DCC), diisopropylcarbodiimide (DIC), 1-ethoxycarbonyl-2-ethoxy-1,2-dihydroquinoline (EEDQ), 1-ethyl-3-(3-dimethylaminopropyl) carbodiimide hydrochloride (EDCI), n-propane-phosphonic anhydride (PPA), N,N-bis(2-oxo-3-oxazolidinyl)imido-phosphoryl chloride (BOP-Cl), bromo-tris-pyrrolidinophosphonium hexafluorophosphate (PyBrop), diphenyl-phosphoryl azide (DPPA), Castro's reagent (BOP, PyBop), O-benzotriazolyl-N,N,N′,N′-tetramethyluronium salts (HBTU), diethylphosphoryl cyanide (DEPCN), 2,5-diphenyl-2,3-dihydro-3-oxo-4-hydroxy-thiophene dioxide (Steglich's reagent; HOTDO), 2-(1H-benzotriazole-1-yl)-1,1,3,3-tetramethyluronium tetrafluoroborate (TBTU), O-(7-azabenzotriazole-1-yl)-1,1,3,3-tetramethyluronium hexafluorophosphate (HATU) and 1,1′-carbonyl-diimidazole (CDI). The coupling reagents can be employed alone or in combination with additives such as N,N-dimethyl-4-aminopyridine (DMAP), N-hydroxy-benzotriazole (HOBt), 1-hydroxy-7-azabenzotriazole (HOAt), N-hydroxybenzotriazine (HOOBt), N-hydroxysuccinimide (HOSu) or 2-hydroxypyridine.

Whereas it is normally possible to dispense with protective groups in enzymatic peptide synthesis, reversible protection of reactive groups not involved in formation of the amide linkage is necessary for both reactants in chemical synthesis. Four conventional protective group techniques are preferred for the chemical peptide synthesis: the benzyloxycarbonyl (Cbz), the t-butoxycarbonyl (Boc), the allyloxycarbonyl (Alloc) and the 9-fluorenyhnethoxycarbonyl (Fmoc) techniques. Identified in each case is the protective group on the α-amino group of the chain-extending unit. A detailed review of amino-acid protective groups is given by Müller, Methoden der organischen Chemie Vol. XV/1, pp 20 to 906, Thieme Verlag, Stuttgart, 1974. The units employed for assembling the peptide chain can be reacted in solution, in suspension or by a method similar to that described by Merrifield in J. Amer. Chem. Soc. 85: 2149 (1963). Particularly preferred methods are those in which peptides are assembled sequentially or by fragment coupling using the Cbz, Boc, Alloc or Fmoc protective group technique, with one of the reactants in the said Merrifield technique being bonded to an insoluble polymeric support (also called resin hereinafter). This typically entails the peptide being assembled sequentially on the polymeric support using the Boc, Alloc or Fmoc protective group technique, the growing peptide chain being covalently bonded at the C terminus to the insoluble resin particles. This procedure makes it possible to remove reagents and byproducts by filtration, and thus recrystallization of intermediates is unnecessary. The protected amino acids can be linked to any suitable polymers, which merely have to be insoluble in the solvents used and to have a stable physical form which makes filtration easy. The polymer must contain a functional group to which the first protected amino acid can be firmly attached by a covalent bond. Suitable for this purpose are a wide variety of polymers, eg. cellulose, polyvinyl alcohol, polymethacrylate, sulfonated polystyrene, chloromethylated styrene/divinylbenzene copolymer (Merrifield resin), 4-methylbenz-hydrylamine resin (MBHA-resin), phenylacetamidomethyl-resin (Pam-resin), Rink amide resin, chlorotrityl chloride resin, p-benzyloxy-benzyl-alcohol-resin, benzhydryl-amine-resin (BHA-resin), 4-(hydroxymethyl-)-benzoyl-oxymethyl-resin, the resin of Breipohl et al. (Tetrahedron Letters 28 (1987) 565; supplied by BACHEM), 4-(2,4-dimethoxyphenylaminomethyl)phenoxy-resin (supplied by Novabiochem) or o-chlorotrityl-resin (supplied by Biohellas).

Suitable for peptide synthesis in solution are all solvents which are inert under the reaction conditions, especially water, N,N-dimethylformamide (DMF), dimethyl sulfoxide (DMSO), acetonitrile, dichloromethane (DCM), 1,4-dioxane, tetrahydrofuran (THF), N-methyl-2-pyrrolidinone (NMP) and mixtures of the said solvents.

Peptide synthesis on the polymeric support can be carried out in all inert organic solvents in which the amino-acid derivatives used are soluble; however, preferred solvents additionally have resin-swelling properties, such as DMF, DCM, NMP, acetonitrile and DMSO, and mixtures of these solvents. After synthesis is complete, the peptide is cleaved off the polymeric support. The conditions under which cleavage of the various resin types is possible are disclosed in the literature. The cleavage reactions most commonly used are acid- and palladium-catalyzed, especially cleavage in liquid anhydrous hydrogen fluoride, in anhydrous trifluoromethanesulfonic acid, in dilute or concentrated trifluoroacetic acid, palladium-catalyzed cleavage in THF or THF-DCM-mixtures in the presence of a weak base such as morpholine or cleavage in acetic acid/dichloromethane/trifluoroethanol mixtures. Depending on the chosen protective groups, these may be retained or likewise cleaved off under the cleavage conditions.

More specifically, the following routes can be used to prepare the novel compounds of general formula I.

a) Route 1: Synthesis of R₁—A1-A2-A3-A4-A5-U.

The peptide sequence is built up stepwise from the C-terminus by coupling the corresponding Fmoc-amino acid (Fmoc-A5-OH) to the free amino group on a resin such as Rink amide AM resin, then removal of the Fmoc-protecting group to liberate the amino group for the next coupling, for example with piperidine. The coupling and deprotection is repeated for each of the amino acids in the order Fmoc—A4-OH, Fmo—A3-OH, Fmoc—A2-OH and Fmoc—A1-OH to yield the intermediate NH₂—A1-A2-A3-A4-A5-NH-resin.

The final capping is done by coupling the corresponding acid R₃—COOH to the above resin intermediate or by reacting the corresponding acid chloride R₃—COCl, acid fluoride R₃—COF or anhydride (R₃—CO)₂O in presence of a base such as tertiary amines or alcoholates or inorganic bases with the resin intermediate. In case of the ureas the resin intermediate is treated with the corresponding isocyanate R₄-NCO or with R₄R₅NCOCl, in the case of the sulfonamides with the corresponding sulfonylchloride R₆-SO₂Cl and in the case of the sulfonylurea with R₇R₈SO₂Cl in the presence of a base.

The final cleavage of the compound from the resin is achieved by treating the resin with acids such as hydrochloric acid or trifluoroacetic acid. Depending on the strength of the acid used and the reaction time, simultaneous deprotection of the side chains could be achieved. The resulting compounds could be further purified by standard techniques such as column chromatography.

b) Route 2: Synthesis of R₁—A1-A2-A3-A4-A5-NR₃₈R₃₉.

In the case that the final compound has an amino side chain containing an acid residue such as Glu, Asp or Aad, the corresponding amino acid Fmoc—Xaa(tBu)—OH was coupled in solution with the corresponding amine HNR₃₈R₃₉ using standard peptide coupling techniques to yield Fmoc—Xaa(tBu)—NR₃₈R₃₉ which is then deprotected with 95% trifluoroacetic acid to yield Fmoc—Xaa—NR₃₈R₃₉ with a free carboxylic acid moiety in the side chain. This amino acid is coupled to a resin such as the chlor(tritylchloride resin) to yield the resin-bound ester. The Fmoc group can now be deprotected with bases such as piperidine to yield the resin-bound amine. The coupling and deprotection is repeated for each of the amino acids in the order Fmoc—A4-OH, Fmo—A3-OH, Fmoc—A2-OH and Fmoc—A1-OH to yield the intermediate NH₂—A1-A2-A3-A4-A5(resin)-NR₃₈R₃₉. The final capping is done by coupling the corresponding acid R₃—COOH to the above resin intermediate or by reacting the corresponding acid chloride R₃—COCl, acid fluoride R₃—COF or anhydride (R₃—CO)₂O in presence of a base such as tertiary amines or alcoholates or inorganic bases with the resin intermediate. In case of the ureas the resin intermediate is treated with the corresponding isocyanate R₄—NCO or with R₄R₅NCOCl, in the case of the sulfonamides with the corresponding sulfonylchloride R₆—SO₂Cl and in the case of the sulfonylurea with R₇R₈SO₂Cl in the presence of a base.

The final cleavage of the compound from the resin is achieved by treating the resin with acids such as hydrochloric acid or trifluoroacetic acid. Depending on the strength of the acid used and the reaction time, simultaneous deprotection of the side chains could be achieved. The resulting compounds could be further purified by standard techniques such as column chromatography.

c) Route 3: R1-A1-A2-A3-A4- OH and R1-A1-A2-A3-A4-NR₃₂R₃₃

The tetrapeptide acid sequence is built up stepwise from the C-terminus as described previously by coupling the corresponding Fmoc-amino acid A1 to the chlorotrityl chloride resin, then removal of the Fmoc-protecting group to liberate the amino group for the next coupling. The coupling and deprotection is repeated for each of the amino acids in the order Fmoc—A3-OH, Fmo—A2-OH and Fmoc—A1-OH and Fmoc—A1-OH to yield the intermediate NH₂—A1-A2-A3-A4-resin.

The final capping is done by coupling the corresponding acid R₃—COOH to the above resin intermediate or by reacting the corresponding acid chloride R₃—COCl, acid fluoride R₃—COF or anhydride (R₃—CO)₂O in presence of a base such as tertiary amines or alcoholates or inorganic bases with the resin intermediate. In case of the ureas the resin intermediate is treated with the corresponding isocyanate R₄—NCO or with R₄R₅NCOCl, in the case of the sulfonamides with the corresponding sulfonylchloride R₆—SO₂Cl and in the case of the sulfonylurea with R₇R₈SO₂Cl in the presence of a base.

The final cleavage of the compound from the resin is achieved by treating the resin with acids such as hydrochloric acid or trifluoroacetic acid. Depending on the strength of the acid used and the reaction time, simultaneous deprotection of the side chains could be achieved. The resulting final compounds R₁—A1-A2-A3-A4-OH could be further purified by standard techniques such as column chromatography.

These compounds are also used as intermediate to couple the corresponding amine NR₃₂R₃₃ in solution to yield the final amides R₁—A1-A2-A3-A4-NR₃₂R₃₃ which can be further purified by standard techniques such as column chromatography.

d) Route 4: R₁—A1-A2-A3-A4-A5-OH and R₁—A1-A2-A3-A4-A5-NR₃₈R₃₉

The above described route to the tetrapeptides can also be used to prepare the corresponding pentapeptides by coupling with the amino acid A5 to the resin such as chlorotrityl chloride resin and then proceeded in similar fashion of repeated coupling of the next amino acids and deprotection, final capping and cleavage form the resin to yield the final compounds R₁—A1-A2-A3-A4-A5-OH. These compounds are also used as intermediate to couple the corresponding amine HNR₃₈R₃₉ in solution to yield the final amides R₁—A1-A2-A3-A4-NR₃₈R₃₉ which can be further purified by standard techniques such as column chromatography.

The amino acids used are either commercially available or their syntheses are described in the literature. The amino acid A can be considered a pTyr-mimetic. Examples of non-hydrolizable phosphor-containing pTyr mimetics have been described in literature such as phosphonomethyl phenylalanine (Pmp, I. Marseigne et al., J. Org. Chem. 53: 3621-3624 (1988)) and phosphonodifluoromethyl phenylalanine (F₂Pmp, T. R. Burke Jr. et al., J. Org. Chem. 58: 1336-1340 (1993)).

Examples for non-phophorous containing pTyr mimetics include O-malonyltyrosine (Tyr(Mal), K. H. Kole et al., Biochem. Biophys. Res. Commun. 209: 817-822 (1995); B. Ye et al., J. Med. Chem. 38: 4270-4275 (1995)), fluoro-O-malonyl-tyrosine (Tyr(Fmal), T. R. Burke Jr., J. Med. Chem. 39: 1021-1027 (1996)), O-carboxymethyl-tyrosine (T. R. Burke Jr. et al., Tetrahedron 54: 9981-9994 (1998)), 3-carboxy-4-(O-carboxymethyl)-tyrosine (T. R. Burke Jr. et al., Tetrahedron 54:9981-9994 (1998)), 3,4-Di(O-carboxymethyl)-tyrosine (T. R. Burke Jr. et al., Tetrahedron 54: 9981-9994 (1998)), O-(carboxydifluoromethyl)-tyrosine (H. Fretz, Tetrahedron 54: 4849-4858 (1998)) and hydroxysulfonylmethyl phenylalanine (I. Marseigne et al., J. Med. Chem. 32: 445-449 (1989)).

To improve cellular penetration prodrugs can be used for the different acid functionalities of the compounds with the general structure of formula I. Typical prodrug forms for carboxylic acid residues are described in R. B. Silverman, The Organic Chemistry of Drug Design and Drug Action, Academic Press, 1992, chapter 8. For the phosphonate function in amino acid A1 suitable prodrugs are simple and substituted alkyl and aryl ester, acyloxyalkyl esters as described in the review by J. P. Krise et al. (Advanced Durg Delivery Reviews, 19: 287-310 (1996)), S-acyltifoethyl esters as described by X. Li et al. (Bioorg. Med. Chem. Lett. 8: 57-62 (1998)) or pivaloylmethyl esters as described by C. J. Stankovic et al. (Bioorg. Med. Chem. Lett. 7: 1909 (1997)).

In one embodiment, the present invention relates to a method of treating a Cdc25-mediated condition in a patient. The method comprises the step of administering to the patient a therapeutically effective amount of a Cdc25 inhibitor as described above. The patient can be any animal, and is, preferably, a mammal and, more preferably, a human.

A “Cdc25-mediated condition” is a disease or medical condition in which the catalytic activity of one or more Cdc25 homologues plays a role, for example, in the development of the disease or condition. For example, in one embodiment, the condition is characterized by excessive cellular proliferation.

In one embodiment, the Cdc25-mediated condition is cancer, such as a tumor. For example the condition to be treated can include lymphoma, such as Hodgkin's disease and non-Hodgkin's lymphoma, and tumors of the head, neck, breast, lung, such as non-small cell lung carcinoma, and stomach.

The Cdc25 mediated condition can also be a condition in which hyperproliferation of non-cancer cells plays an important role, such as restinosis and reocclusion of the coronary arteries following angioplasty, both of which result from abnormal proliferation of smooth muscle cells.

In another embodiment, the Cdc25-mediated condition is an inflammatory disease which is characterized by abnormal cell proliferation, such as rheumatoid arthritis, Reiter's disease, systemic lupus.

A therapeutically effective amount, as this term is used herein, is an amount which results in partial or complete inhibition of disease progression or symptoms. Such an amount will depend, for example, on the size and gender of the patient, the condition to be treated, the severity of the symptoms and the result sought, and can be determined by one skilled in the art.

The compound of the invention can, optionally, be administered in combination with one or more additional drugs which, for example, are known for treating and/or alleviating symptoms of the condition mediated by Cdc25. The additional drug can be administered simultaneously with the compound of the invention, or sequentially. For example, the Cdc25 inhibitor can be administered in combination with another anticancer agent, as is known in the art.

The invention further provides pharmaceutical compositions comprising one or more of the Cdc25 inhibitors described above. Such compositions comprise a therapeutically (or prophylactically) effective amount of one or more Cdc25 binding inhibitors, as described above, and a pharmaceutically acceptable carrier or excipient. Suitable pharmaceutically acceptable carriers include, but are not limited to, saline, buffered saline, dextrose, water, glycerol, ethanol, and combinations thereof. The carrier and composition can be sterile. The formulation should suit the mode of administration.

Suitable pharmaceutically acceptable carriers include but are not limited to water, salt solutions (e.g., NaCl), alcohols, gum arabic, vegetable oils, benzyl alcohols, polyethylene glycols, gelatin, carbohydrates such as lactose, amylose or starch, cyclodextrin, magnesium stearate, talc, silicic acid, viscous paraffin, perfume oil, fatty acid esters, hydroxymethylcellulose, polyvinyl pyrolidone, etc. The pharmaceutical preparations can be sterilized and if desired, mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds.

The composition, if desired, can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulation can include standard carriers such as pharmaceutical grades of mannitol, lactose, starch, magnesium stearate, polyvinyl pyrollidinone, sodium saccharine, cellulose, magnesium carbonate, etc.

The composition can be formulated in accordance with the routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic to ease pain at the site of the injection. Generaly, the ingredients are supplied either separately or mixed together in unit dosage form, for example, as a dry lyophilized powder or water free concentrate in a hermetically sealed container such as an ampoule or sachet indicating the quantity of active agent. Where the composition is to be administered by infusion, it can be dispensed with an infusion bottle containing sterile pharmaceutical grade water, saline or dextrose/water. Where the composition is administered by injection, an ampoule of sterile water for injection or saline can be provided so that the ingredients may be mixed prior to administration.

The pharmaceutical compositions of the invention can also include an agent which controls release of the Cdc25 inhibitor compound, thereby providing a timed or sustained release composition.

The Cdc25 inhibitor can be administered subcutaneously, intravenously, parenterally, intraperitoneally, intradermally, intramuscularly, topically, enteral (e.g., orally), rectally, nasally, buccally, sublingually, vaginally, by inhalation spray, by drug pump or via an implanted reservoir in dosage formulations containing conventional non-toxic, physiologically acceptable carriers or vehicles. The preferred method of administration is by oral delivery. The form in which it is administered (e.g., syrup, elixir, capsule, tablet, solution, foams, emulsion, gel, sol) will depend in part on the route by which it is administered. For example, for mucosal (e.g., oral mucosa, rectal, intestinal mucosa, bronchial mucosa) administration, nose drops, aerosols, inhalants, nebulizers, eye drops or suppositories can be used. The compounds and agents of this invention can be administered together with other biologically active agents, such as analgesics, anti-inflammatory agents, anesthetics and other agents which can control one or more symptoms or causes of a Cdc25-mediated condition.

In a specific embodiment, it may be desirable to administer the agents of the invention locally to a localized area in need of treatment; this may be achieved by, for example, and not by way of limitation, local infusion during surgery, topical application, transdermal patches, by injection, by means of a catheter, by means of a suppository, or by means of an implant, said implant being of a porous, non-porous, or gelatinous material, including membranes, such as sialastic membranes or fibers. For example, the agent can be injected into the joints.

### EXAMPLES

### Example 1

### Synthesis of Peptidic Cdc25 Inhibitors

General Materials and Methods

The following amino acid abbreviations are used herein, for the natural amino acids the three letter code:

Asp=asp artic acid; Asn=asparagine; Pro=proline; Ala=alanine; Val=valine; Lys=Lysine; Gly=glycine; Arg=arginine; Ile=isoleucine; Ser=serine; Thr=threonine; Leu=leucine; Trp=tryptophan; Cys=cysteine; Tyr=tyrosine; Met=methionine; Gln=glutamine; Glu=glutamic acid; Phe=phenylalanine; His=histidine, for other amino acids: Nal=2-amino-3-(naphth-1-yl)-propanoic acid (or napthyl-alanine); Bta=2-amino-3-benzo[b]thiophen-3-yl-propanoic acid (or 3-benzothienylalanine); Aad=alpha-aminoadipic acid; Smp═Phe(4-CH₂—SO₃H); Pmp═Phe(4-CH₂—PO(OH)₂); F₂Pmp═Phe(4-CF₂—PO(OH)₂); Asu=alpha aminosuberic acid; Hyp=4-hydroxyproline; Nle=norleucine; Nva=norvaline; hLeu=homoleucine; Pip=pipecolinic acid; 3-MePro=3-methyl-proline; 2-MePro=2-methyl-proline; Isc =1-isoindolinecarboxylic acid; Oic=octahydroindolyl-2-carboxylic acid; Ac6=1-amino-1-cyclohexanecarboxylic acid; Ac5=1-amino-1-cyclopentanecarboxylic acid; Ac3=1-amino-1-cyclopropanecarboxylic acid; Thiopro=L-thiazolidine-4-carboxylic acid; Iva=2-amino-2-methylbutanoic acid; Tyr(mal)=Tyr(O—(CH(COOH)₂); Tyr(Fmal)=Tyr(O—(CF(COOH)₂); 2-Oxn=(8-hydroxy-quinolin-2-yl)methyl glycine; dehydrPro=3,4-dehydroproline; dehydro Val=dehydrovaline; Aib=alpha-aminoisobutyric acid; Pra=propargylic glycine; Phg=phenyl glycine ; SmPhg═Phg(4-CH₂—SO₃H).

Furthermore the following abbreviations have the meaning of Xaa=amino acid, hXaa=homo amino acid, (NMe)Xaa=amino acid methylated at the amino group, MeXaa=amino acid methylated at the alpha carbon or the position indicated, Xaa(R)=amino acid with a group R functionalized side chain, and D,L-Xaa=mixture of D- and L-isomer (50/50 or the ratio indicated below in parenthesis).

In the synthetic procedures the amino group of the amino acid is usually protected with the Fmoc-group, in a few cases with the Alloc- or Boc-group. Carboxylic acid moieties in the side chain of the amino acids are protected as tert. butyl esters, when the carboxylic acid moiety is desired in the final product. The tert. butyl esters are hydrolysed under the certain conditions used for the cleavage of the peptidyl derivatives from polymers or resin. All natural amino acids and their corresponding D-isomers can be purchased from Novabiochem or Bachem.

The other amino acids were purchased from:

Fmoc—Nal(1)-OH (Synthetech Inc.), Fmoc—Bta—OH (Peptech Corp.), Fmoc—Aad(tBu)—OH (Bachem), Fmoc—Smp—OH (RSP Amino Acid Analogues), Fmoc—(Pmp(Et)₂)—OH (Neosystem), Fmoc—Asu(tBu)—OH (Peninsula Labs), Fmoc—Hyp(tBu)—OH (Novabiochem), Fmoc—Nle—OH (Novabiochem), Fmoc—Nva—OH (Novabiochem), Fmoc—hLeu—OH (Neosystem), Fmoc—Pip—OH (Bachem), Fmoc-(2-Me)Pro—OH (Bachem), Fmoc—Isc—OH (Neosystem), Fmoc—Oic—OH (Bachem), Fmoc—Ac6-OH (Neosystem), Fmoc—Ac5-OH (Neosystem), Fmoc—Ac3-OH (Advanced Chem Tech), Fmoc—Thiapro—OH (Neosystem), Fmoc—Iva—OH (Acros), Fmoc(Tyr(mal(tBu)₂)—OH (Bachem), Fmoc—DehydrPro—OH (Bachem), Fmoc-Dehydro Val—OH (Advanced Chem Tech), Fmoc—Aib—OH (Senn Chemicals), Fmoc—Pra—OH (Advanced Chemtech), Fmoc—Phg—OH (Novabiochem), SmPhg (RSP Amino Acid Analogues), Fmoc-(3-Cl—Phe)—OH (Peptech), Fmoc-(2—Cl—Phe)—OH (Peptech), Fmoc-(4-Cl—Phe)—OH (Bachem), Fmoc-(4-CONH₂—Phe)—OH (RSP Amino Acid Analogues), Fmoc-(4-NH₂—Phe)—OH (Bachem), Fmoc-(3-NH₂—Phe)—OH (RSP Amino Acid Analogues), ), Fmoc-(4-NHAc—Phe)—OH (RSP Amino Acid Analogues), Fmoc-(4-COOtBu—Phe)—OH (Bachem), Fmoc-(4-CF₃—Phe)—OH (Apollo), Fmoc-(3-NO₂—Phe)—OH (Peptech), Fmoc-(4-Ph—Phe)—OH (Bachem), Fmoc-3-NH-pyridinon-1-yl—CH₂—COOH (Neosystem), Fmoc-3-NH-caprolactam-1-CH₂—COOH (Neosystem), Fmoc-6-NH-5-oxo-perhydropyrido[2,1-b]-(1,3)-thiazol-3-COOH (Neo-system), Fmoc-azetidine-carboxylic acid (Neosystem).

The following amino acids were prepared according to the following references:

Synthesis of Fmoc—(F₂Pmp(Et)₂)—OH, ref. T. R. Burke Jr. et al., J. Org. Chem. 1993, 58, 1336-1340; synthesis of Fmoc—(Tyr(Fmal(tBu)₂)—OH), ref. T. R. Burke Jr. et al., J. Med. Chem. 1996, 39, 1021-1027; synthesis of Fmoc—(O-carboxymethyl)-Tyr—OH, ref. T. R. Burke Jr. et al., Tetrahedron 1998, 54,9981-9994; synthesis of Fmoc-(2-Oxn)—OH, ref. G. K. Walkup et al., J. Org. Chem. 1998, 63, 6727-6731.

Furthermore, the following abbreviations are used herein:

EDCI=1-(3-dimethylaminopropyl)-3-ethylcarbodiimide hydrochloride

HATU=O-(7-azabenzotriazole-1-yl)-1,1,3,3-tetramethyluronium hexafluorophosphate

HBTU=2-(1H-benzotriazole-1-yl)-1,1,3,3-tetramethyluronium hexafluorophosphate

HOAt=1-hydroxy-7-azabenzotriazole

TBTU=2-(1H-benzotriazole-1-yl)-1,1,3,3-tetramethyluronium tetrafluoroborate

Fmoc=fluorenylmethoxycarbonyl

Rink amide AM=4-(2′,4′-dimethoxyphenyl-Fmoc-aminomethyl)-phenoxyacetamido-norleucyl aminomethyl.

The Rink amide AM is commercially available from Novabiochem.

The 2-Chlorotritylchloride resin was purchased from Novabiochem.

Analytical HPLC Conditions

Method A-E:

Column: Vydac 300 Angstrom, C18, flow rate: 0.8 mL/min (λ=214 nm)

Solvents: solvent A=0.1% trifluoroacetic acid/water, solvent B=0.1% trifluoroacetic acidlacetonitrile

Gradients:

Method A: 30 to 90% Solvent B in 30 min.

Method B: 5 to 45% Solvent B in 40 min.

Method C: 5 to 65% Solvent B in 60 min.

Method D: 15 to 60% Solvent B in 30 min.

Method E: 40 to 90% Solvent B in 25 min.

Methods F-Q

Column: C18 (4 mm×300 mm), flow rate: 0.5 mL/min with CH3CN/H2O(0.1% TFA)

Solvents: solvent A=0.1% trifluoroacetic acid/water, solvent B=0.1% trifluoroacetic acid/acetonitrile

Gradients:

Method F: 10% Solvent B for 5 min., from 10% to 90% in 20 min.

Method G: 50% Solvent B for 5 min., from 50% Solvent B to 90% in 20 min.

Method H: 10% Solvent B for 5 min., from 10% Solvent B to 100% in 12.5 min

Method I: 20% Solvent B for 5 min., from 20% Solvent B to 70% in 20 min.

Method J: 25% Solvent B for 5 min., from 25% Solvent B to 65% in 13 min, from 65% to 100% in 4 min.

Method K: 15% Solvent B for 5 min., from 15% Solvent B to 80% in 20 min

Method L: 40% Solvent B to 100% in 20 min.

Method M: 10% Solvent B for 5 min., from 10% Solvent B to 90% in 25 min.

Method N: 30% Solvent B for 5 min., from 30% Solvent to 70% in 12.5 min., from 70% to 100% in 5 min.

Method O: 50% Solvent B to 90% in 20 min

Method P: 25% Solvent B for 7 min., from 25% Solvent B to 100% in 20 min.

Method Q: 25% Solvent B for 5 min., from 25% Solvent B to 100% in 15 min.

Method R: Column: Vydac (2.1 mm×150 mm) 300 Angstrom C18, flow rate: 0.2 mL/min (λ=214 nm)

Solvent: solvent A=0.02% trifluoroacetic acid/0.08% formic acid/ water, solvent B=0.02% trifluoroacetic acid/0.08% formic acid/ acetonitrile

Method R: 5% Solvent B to 98% in 10 min.

### Synthesis of 2-CH₃O-Naphtyl-1-CO—Smp—Glu—Glu—Nal—Glu—NH₂, cdc1249

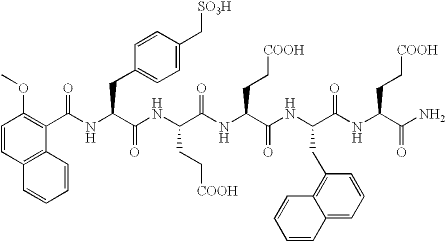

The peptide sequence is built up stepwise from the C-terminus by coupling the corresponding Fmoc-amino acid to the free amino group on the resin, then removal of the Fmoc-protecting group to liberate the amino group for the next coupling. The final capping is done with 2-methoxy-naphthyl-carboxylic acid.

Rink amide AM resin (223 mg resin, 0.1 mmol) was washed with dimethylformamide (2×25 mL), then removal of the Fmoc protection group was achieved with 20% piperidine in dimethylformamide (2×25 mL). All amino acids (0.15 mmol, 1.5 eq) were coupled using the coupling reagent TBTU (48 mg, 0.15 mmol, 1.5 eq) and the base N,N-diisopropylethylamine (38 mg, 0.3 mmol, 3 eq) in dimethylformamide except Fmoc—Phe(4-CH₂SO₃H)—OH and 2-methoxy-naphth-1-yl-carboxylic acid which were coupled with HATU (57 mg, 0.15 mmol, 1.5 eq and 76 mg, 0.2 mmol, 2.0 eq respectively) and N,N-diisopropylethylamine (39 mg, 0.3 mmol, 3.0 eq, and 65 mg, 0.5 mmol, 5.0 eq respectively) in dimethylformamide (25 mL) . Fmoc deprotections were done with 20% piperidine in dimethylformamide (2×25 mL). The completion of coupling and deprotection reactions was assessed by Kaiser test (ninhydrin). After each step, the resin was washed with dimethylformamide (2×25 mL), dichloromethane (3×25 mL) and methanol (2×25 mL) then dried in vacuo. Cleavage of the final peptide from the resin and simultaneous deprotection of the side chains was achieved with trifluoroacetic acid/water (95:5) at room temperature for two hours. Trifluoroacetic acid was removed in vacuo, then the remaining residue was triturated with diethylether (30 mL). The resulting solid was dried in vacuo.

HPLC purification was done on Waters Deltapack C₁₈ reverse phase silica gel using a 40 mm×200 mm 300 Angstrom column.

HPLC conditions: flow rate: 10 mL/min

Gradient: 10%-30% B in 57 min then 30%-90% B in 200 min

A=0.1% trifluoroacetic acid/water

B=0.1% trifluoroacetic acid/acetonitrile

Yield: 25 mg (0.024 mmol) 2-CH₃O-1-naphthyl-1-CO—Smp—Glu—Glu—Nal—Glu—NH₂

Analytical Data Rₜ 32.1 min (Method B) MS (ESI): MH⁺ 1027

H¹ NMR (d₆-DMSO, 400 MHz): δ4.83 (m, 1H), 4.69 (m, 1H), 4.43 (m, 1H), 4.26 (m, 1H), 4.19 (m, 1H) (characteristic alpha-protons).

### Synthesis of 2-EtO-1-naphthyl -1-CO—Smp—Glu—Glu—Bta—Glu—NH₂, cdc 1659

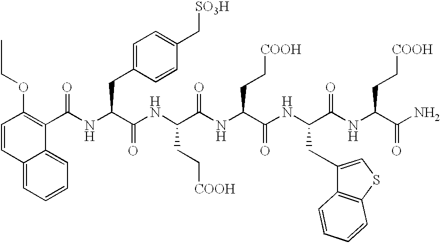

Rink amide AM resin (379 mg, 0.25 mmol) was washed with dimethylformamide (2×25 mL) then Fmoc deprotected with 20% piperidine in dimethylformamide (2×25 mL). The first four amino acids (1.0 mmol) from the C-terminus were coupled in 1-methyl-2-pyrrolidinone using 0.45 M HBTU in dimethylfornamide (2 g, 0.9 mmol) and 2 M N,N-diisopropylethylamine in 1-methyl-2-pyrrolidinone (1.0 mL). Fmoc—Phe(4-CH₂SO₃H)—OH (180 mg, 0.375 mmol) was then coupled with TBTU (120 mg, 0.375 mmol) and N,N-diisopropylethylamine (113 mg, 0.875 mmol) in dimethylformamide (25 mL). The resin was split at this point and 0.1 mmol was coupled with 2-ethoxy-1-naphthoic acid (43 mg, 0.2 mmol) using HATU (76 mg, 0.2 mmol) and N,N-diisopropylethylamine (58 mg, 0.45 mmol) in dimethylformamide (25 mL). Fmoc deprotections of each amino acid were done with 20% piperidine in dimethylformamide (2×25 mL). The completion of coupling and deprotection reactions was assessed by Kaiser test (ninhydrin). The resin was washed with dimethylformamide (2×25 mL), dichloromethane (3×25 mL) and methanol (2×25 mL) then dried in vacuo. The peptide was cleaved from the resin and the side chains were deprotected with trifluoroacetic acid/water (95:5) at room temperature for approx. 2 hours. The trifluoroacetic acid was removed in vacuo then the remaining residue was triturated with Et₂O (30 mL). The resulting solid was dried in vacuo. HPLC purification was done on Waters Deltapack C₁₈ reverse phase silica gel using a 40 mm×200 mm 300 Angstrom column.

Yield: 22 mg (0.021 mmol) 2-EtO-1-naphthyl-1-CO—Smp—Glu—Glu—Bta—Glu—NH₂ Analytical data Rₜ 34.3 min (Method B) MS (ESI): MH⁺ 1047

H¹ NMR (d₆-DMSO, 400 MHz): δ4.87 (m, 1H), 4.70 (m, 1H), 4.41 (m, 1H), 4.28 (m, 1H), 4.19 (m, 1H), (characteristic alpha-protons), 4.11 (q, 2H, CH₂), 1.19 (t, 3H, CH₃ ).

### Synthesis of 2-CH₃O-1-naphthyl-CO—Smp—Glu—Glu—Bta—Aad—NHtBu, cdc1671

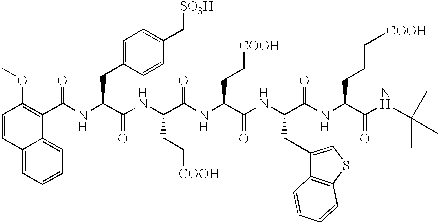

A. 2-chlorotritylchloride Resin Loading

Fmoc—-Aad(tBu)—OH (377 mg, 0.847 mmol) was dissolved in dichloromethane (15 mL) under a nitrogen atmosphere. 2-Chlorotritylchloride resin (1.5 g, 1.43 mmol) was added to this solution, followed by N,N-diisopropylethylamine (442 mg, 3.43 mmol). The suspension was stirred under nitrogen at room temperature for 6 hours. The reaction completion was checked by thin layer chromatography, monitoring the consumption of amino acid. The resin was filtered and washed with a mixture of dichloromethane/methanol/N,N-diisopropylethylamine (17:2:1, 3×25 mL), dichloromethane (3×25 mL), and dimethylformamide (2×25 mL). The Fmoc protecting group was removed with 20% piperidine in dimethylformamide (2×25 mL, monitored by Kaiser test). The resin was washed with dimethylformamide (2×25 mL), dichloromethane (3×25 mL) and methanol (2×25 mL), then dried in vacuo. Loading: approximately 0.6 mmol/g.

B. Amino Acid Couplings

The Aad(tBu) loaded trityl resin (417 mg, 0.25 mmol) was suspended in 1-methyl-2-pyrrolidinone (4 mL). The amino acids Fmoc—Bta—OH, Fmoc—Glu(tBu)—OH and, Fmoc—Glu(tBu)—OH (1.0 mmol each) were coupled using 0.45 M HBTU in dimethylformamide (2 g, 0.9 mmol) and 2 M N,N-diisopropylethylamine in 1-methyl-2-pyrrolidinone (1.0 mL). The resin was split at this point and 176 mg of the resin (0.083 mmol) was used for the coupling of Fmoc—Phe(4-CH₂SO₃H)—OH (60 mg, 0.125 mmol) with TBTU (40 mg, 0.125 mmol) and N,N-diisopropylethylamine (43 mg, 0.332 mmol) as coupling reagents. The final capping of the peptide was done with 2-methoxy-1-naphthoic acid (34 mg, 0.166 mmol) using HATU (63 mg, 0.166 mmol) and N,N-diisopropylethylamine (48 mg, 0.374 mmol). Fmoc deprotections after each coupling step were done with 20% piperidine in dimethylformamide (2×25 mL). The completion of coupling and deprotection reactions was assessed by Kaiser test (ninhydrin). The resin was washed with dimethylformamide (2×25 mL), dichloromethane (3×25 mL), and methanol (2×25 mL). Cleavage of the peptide from the resin was achieved with dichloromethane/trifluoroacetic acid/acetic acid (8:1:1, 10 mL) at room temperature for one hour. The side-chain protected pentapeptide acid was collected as a white solid (52 mg).

C. C-terminal Amide Synthesis

The side chain protected pentapeptide acid 2-EtO-1-naphthoyl-Phe(4-CH₂SO₃H)—Glu(tBu)—Glu(tBu)—Bta—Glu—OH (52 mg, 0.042 mmol) was dissolved in dichloromethane (3 mL), then tert-butylamine (6.2 mg, 0.084 mmol), HOAt (5.7 mg, 0.042 mmol), EDCI (16 mg, 0.084 mmol), and N,N-diisopropylethylamine (22 mg, 0.168 mmol) were added. The reaction mixture was stirred at room temperature for 18 hours. The reaction mixture was diluted with dichloromethane (150 mL) and washed with 1:1 water/brine (75 mL). The organic layer was dried over sodium sulfate, concentrated in vacuo and the remaining solid was dried in vacuo. The tert.-butyl esters were deprotected using trifluoroacetic acid/water (95:5) at room temperature for 1 hour. The reaction mixture was concentrated in vacuo and the remaining residue was triturated with diethylether (30 mL). The resulting solid was dried in vacuo. HPLC purification was done on Waters Deltapack C₁₈ reverse phase silica gel using a 40 mm×200 mm 300 Angstrom column.

HPLC conditions: flow rate: 10 mL/min

Gradient: 20%-40% B in 57 min then 40%-100% B in 257 min

A=0.1% trifluoroacetic acid/water

B=0.1% trifluoroacetic acid/acetonitrile

Yield: 13 mg (0.012 mmol) 2-CH₃O-1-naphthyl-CO—Smp—Glu—Glu—Bta—Aad—NHtBu Analytical data Rₜ 39 min (Method B) MS(ESI): MH⁺ 1103

H¹ NMR (d₆-DMSO, 400 MHz): δ4.82 (m, 1H), 4.71 (m, 1H), 4.45 (m, 1H), 4.30 (m, 1H), 4.17 (m, 1H) (characteristic alpha-protons), 1.24 (s, 9H, tBu).

### Synthesis of 2-EtO-1-naphthyl-CO—Smp—Nva-(3-Me)Pro—Bta—Aad—NHtBu, cdc 1747

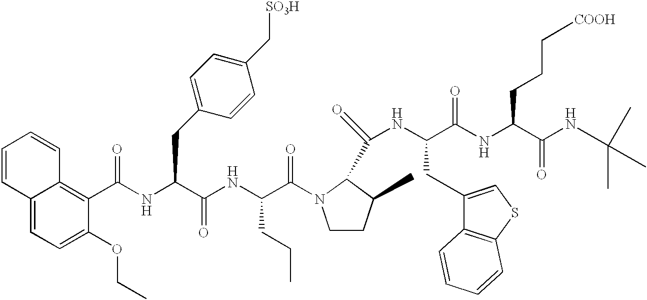

### a) Synthesis of 6-(tert-butylamino)-5-(((9H-9-fluorenylmethoxy)carbonyl)amino)-6-oxohexanoate

Fmoc-L-α-aminoadipic acid-δ-tert.-butylester (2 g, 4.55 mmol) was dissolved in dichloromethane (80 ml), then tert.-butylamine (0.665 g, 9.1 mmol), 1-hydroxy-7-azabenzotriazole (0.619 g, 4.55 mmol), 1-(3-dimethylaminopropyl)-3-ethylcarbodiimide hydrochloride (1.74 g, 9.1 mmol), and diisopropylethylamine (2.05 g, 15.92 mmol) were added. The yellow solution was stirred at room temperature for 21 hours. The reaction mixture was diluted with dichloromethane, then washed with saturated aqueous sodium bicarbonate, 5% aqueous citric acid and with a one to one mixture of water and brine. The organic layer was dried over sodium sulfate and concentrated under reduced pressure to give 2.07 g (4.19 mmol) tert-butyl 6-(tert-butylamino)-5-{[9H-9-fluorenylmethoxy)carbonyl]amino}-6-oxohexanoate.

MS(ESI): MH⁺=495 Rₜ=23.5 (Method E)

The crude tert-butyl 6-(tert-butylamino)-5-{[9H-9-fluorenylmethoxy)carbonyl]amino}-6-oxohexanoate (2.07 g, 4.19 mmol) was dissolved in minimal dichloromethane, then diluted with 95% trifluoroacetic acid/water (30 ml). The reaction mixture was stirred at room temperature for 1.5 hours then concentrated under reduced pressure. The remaining oil was triturated with diethylether and filtered. The diethylether filtrate was concentrated under reduced pressure to give 2.48 g of 6-(tert-butylamino)-5-(((9H-9-fluorenylmethoxy)carbonyl)amino)-6-oxohexanoate.

MS(ESI): MH⁺=439 Rₜ=14.6 (Method E)

### b) Loading of 5-amino-6-(tert-butylamino)-6-oxohexanoic acid on chlorotrityl chloride Resin

6-(tert-butylamino)-5-(((9H-9-fluorenylmethoxy)carbonyl)amino)-6-oxohexanoate (2.48 g, 5.66 mmol) was dissolved in dichloromethane (80 ml) under a nitrogen atmosphere then added chlortritylchloride resin (4.85 g, 7.23 mmol) and diisopropylethylamine (2.92 g, 22.64 mmol). The dark purple reaction mixture was stirred for 5 hours under a nitrogen atmosphere. The suspension was filtered, then washed the resin three times with a mixture of dichloromethane/methanol/diisopropylethylamine (17:2:1); three times with dichloromethane and twice with dimethylformamide. The resin was suspended in dimethylformamide (25 ml), then treated with 20% piperidine in dimethylformamide the first time for 5 min, a second time for 20 min, followed by washing it five times with dimethylformamide. Finally, the resin was washed with three times with dichloromethane, twice with methanol and dried in vacuo to give 5.21 g of loaded resin.

### c) 2-EtO-1-naphthyl-CO—Smp—Nva-(3-Me)Pro—Bta—Aad—NHtBu

The peptide sequence was built up stepwise from the C-terminus by coupling the corresponding Fmoc-amino acid to the free amino group on the resin, then removal of the Fmoc-protecting group to liberate the amino group for the next coupling. The final capping is done with 2-ethoxy-naphthyl-carboxylic acid.

The previously loaded trityl resin (187 mg resin, 0.15 mmol) was suspended in dimethylformamide (25 ml). The four amino acids (in the order Fmoc—Bta—OH, Fmoc-(3-Me)Pro—OH, Fmoc—Nva—OH, Fmoc—Smp—OH) (0.225 mmol, 1.5 eq) were coupled using the coupling reagent TBTU (72 mg, 0.225 mmol, 1.5 eq) and the base N,N-diisopropylethylamine (77 mg, 0.6 mmol, 4 eq) in dimethylformamide, whereas the final coupling with 2-ethoxy-naphth-1-yl-carboxylic acid was done with HATU (114 mg, 0.3 mmol, 2 eq) and N,N-diisopropylethylamine (87 mg, 0.675 mmol, 4.5 eq) in dimethylformamide (25 mL). Fmoc deprotections were done with 20% piperidine in dimethylformamide (2×25 mL). The completion of coupling and deprotection reactions were assessed by Kaiser test (ninhydrin). After each step, the resin was washed with dimethylformamide (2×25 mL), dichloromethane (3×25 mL) and methanol (2×25 mL) then dried in vacuo. Cleavage of the final peptide from the resin was achieved with dichloromethane/trifluoroethanol/acetic acid (8:1:1, 10 ml) at room temperature for 45 min. The suspension was filtered, washing with dichloromethane. The filtrate was concentrated under reduced pressure and the remaining residue was triturated with diethylether. The resulting solid was dried in vacuo to give 2-EtO-naphth-1-yl-CO—Smp—Nva-(3-Me)Pro—Bta—Aad—NHtBu as a pale yellow solid; (105 mg, 66%).

MS(ESI): MH⁺=1069 Rₜ=15.8 (Method A)

The compounds presented in Tables 1-6 were obtained in a similar fashion as the four pentapeptides described above. The column Cdc# gives the reference number for the compounds. The columns R₁, A1, A2, A3, A4, A5, U, R₂, these are the residues of the peptidyl compounds as defined for Formula I, above. The tables also provide mass spec data (MH⁺) and analytical data for the compounds (retention time and the HPLC method used).

TABLE 1

| | | | | | | | | | HPLC- | |
|---|---|---|---|---|---|---|---|---|---|---|
| | | | | | | | | | Ret. | |
| | | | | | | | | | Rₜ | |
| Cdc # | R₁ | A1 | A2 | A3 | A4 | A5 | U | MH+ | (min) | Meth. |
| 1788 | Ac | Smp | Nva | Pro | Bta | Glu | NHtBu | 885 | 9.3 | A |
| 1789 | 9-Anthracenyl-CO | Smp | Nva | Pro | Bta | Glu | NHtBu | 1047 | 15.9 | A |
| 1790 | Mesityl-SO2 | Smp | Nva | Pro | Bta | Glu | NHtBu | 1025 | 27.5 | D |
| 1797 | 2-Me-naphthyl-CO | Smp | Nva | Pro | Bta | Glu | NHtBu | 1011 | 14.6 | A |
| 1814 | 2,6-diMe-Bzl-CO | Smp | Nva | Pro | Bta | Glu | NHtBu | 975 | 13.4 | A |
| 1815 | 2,6-di(MeO)-Bzl-CO | Smp | Nva | Pro | Bta | Glu | NHtBu | 1007 | 12.3 | A |
| 1799 | Ac-Aib | Smp | Nva | Pro | Bta | Glu | NHtBu | 970 | 10.5 | A |
| 1823 | 9-Anthracenyl-CO | Smp | Glu(t | 2-MePro | Bta | Aad | NHtBu | 1161 | 18.6 | A |
| | | | Bu) | | | | | | | |
| 1949 | 2,4-Di-OMe-5-Me—Ph— | Smp | Nva | 2-MePro | Bta | Aad | NHtBu | 1049 | 13.8 | A |
| | CO | | | | | | | | | |
| 1948 | 2-Naphthyl-NH—CO | Smp | Nva | 2-MePro | Bta | Aad | NHtBu | 1040 | 16.6 | A |
| 1947 | 1-Naphthyl-NH—CO | Smp | Nva | 2-MePro | Bta | Aad | NHtBu | 1040 | 15.6 | A |
| 1946 | Ph—CH₂—NH—CO— | Smp | Nva | 2-MePro | Bta | Aad | NHtBu | 1004 | 14.1 | A |
| 1945 | Ph—NH—CO | Smp | Nva | 2-MePro | Bta | Aad | NHtBu | 990 | 14.4 | A |
| 1944 | Pivaloyl | Smp | Nva | 2-MePro | Bta | Aad | NHtBu | 955 | 13.6 | A |
| 1943 | Adamantyl-CO | Smp | Nva | 2-MePro | Bta | Aad | NHtBu | 1033 | 16.9 | A |
| 1939 | 1-Dibenzofuranyl-CO | Smp | Nva | 2-MePro | Bta | Aad | NHtBu | 1065 | 16.7 | A |
| 1938 | Di-Phenyl-1-CO | Smp | Nva | 2-MePro | Bta | Aad | NHtBu | 1051 | 15.7 | A |
| 1937 | Naphthyl-1-CO | Smp | Nva | 2-MePro | Bta | Aad | NHtBu | 1025 | 15 | A |
| 1936 | 4-Fluorenyl-CO | Smp | Nva | 2-MePro | Bta | Aad | NHtBu | 1063 | 16.1 | A |
| 1935 | 1-Fluorenyl-CO | Smp | Nva | 2-MePro | Bta | Aad | NHtBu | 1063 | 17.3 | A |
| 1934 | 3-Indolyl-CO | Smp | Nva | 2-MePro | Bta | Aad | NHtBu | 1014 | 13.7 | A |
| 1933 | 1-EtO-Naphthyl-2-CO | Smp | Nva | 2-MePro | Bta | Aad | NHtBu | 1055 | 16.3 | A |
| 1907 | 2,3-Di-OMe—Ph—CO | Smp | Nva | 2-MePro | Bta | Aad | NHtBu | 1035 | 14.1 | A |
| 1906 | 4-Phenanthrenyl-CO— | Smp | Nva | 2-MePro | Bta | Aad | NHtBu | 1075 | 16.4 | A |
| 2060 | 9-Fluorenyl-CO— | Smp | Nva | 2-MePro | Bta | Aad | NHtBu | 1063 | 16.5 | A |
| 1972 | 5-NMe₂-Naphthyl-1-SO₂ | Smp | Nva | 2-MePro | Bta | Aad | NHtBu | 1104 | 10.8 | A |
| 2284 | 3,5-diCl—Ph—CO | Smp | Nva | Pro | Nal | Aad | NHtBu | 1023 | 20 | Q |
| 2285 | 2,5-diCl—Ph—CO | Smp | Nva | Pro | Nal | Aad | NHtBu | 1023 | 18 | Q |
| 2286 | 2-OH-5-Cl—Ph—CO | Smp | Nva | Pro | Nal | Aad | NHtBu | 1005 | 18 | Q |
| 2287 | 2-OH-3,5-diCl—Ph—CO | Smp | Nva | Pro | Nal | Aad | NHtBu | 1039 | 21 | Q |
| 2288 | 2,3-diCl—Ph—CO | Smp | Nva | Pro | Nal | Aad | NHtBu | 1023 | 18 | Q |
| 2289 | 4-OH-3-Cl—Ph—CO | Smp | Nva | Pro | Nal | Aad | NHtBu | 1005 | 17 | Q |
| 2290 | 2,6-diOMe-5-Cl—Ph—CO | Smp | Nva | Pro | Nal | Aad | NHtBu | 1049 | 18 | Q |
| 2291 | 2,3,5-triCl—Ph—CO | Smp | Nva | Pro | Nal | Aad | NHtBu | 1059 | 18 | Q |
| 2292 | 2,3,6-triCl—Ph—CO | Smp | Nva | Pro | Nal | Aad | NHtBu | 1059 | 18 | Q |
| 2293 | 2-F-5-Cl—Ph—CO | Smp | Nva | Pro | Nal | Aad | NHtBu | 1007 | 18 | Q |
| 2294 | 3-F-5-Cl—Ph—CO | Smp | Nva | Pro | Nal | Aad | NHtBu | 1007 | 18 | Q |
| 2295 | 2-Br-5-Cl—Ph—CO | Smp | Nva | Pro | Nal | Aad | NHtBu | 1069 | 18 | Q |
| 2296 | Ac | Smp | Nva | Pro | Nal | Aad | NHtBu | 893 | 18 | Q |
| 2297 | 2-Furanyl-CO | Smp | Nva | Pro | Nal | Aad | NHtBu | 945 | 17 | Q |
| 2298 | 2-Thiophenyl-CO | Smp | Nva | Pro | Nal | Aad | NHtBu | 961 | 16 | Q |
| 2299 | N—Me-pyrrolyl-2-CO | Smp | Nva | Pro | Nal | Aad | NHtBu | 958 | 17 | Q |
| 2300 | 3-pyridyl-CO | Smp | Nva | Pro | Nal | Aad | NHtBu | 956 | 17 | Q |
| 2301 | Ph—C≡C—CO | Smp | Nva | Pro | Nal | Aad | NHtBu | 979 | 18 | Q |
| | | Smp | | | | | | | | |
| 2302 | 4-F-3-(E)- | Smp | Nva | Pro | Nal | Aad | NHtBu | 1099 | 20 | Q |
| | (CHCHCO₂tBu)—Ph—CO | Smp | | | | | | | | |
| 2303 | 2-OMe-3-(E)- | Smp | Nva | Pro | Nal | Aad | NHtBu | | | |
| | (CHCHCO₂tBu)—Ph—CO | | | | | | | | | |
| 2304 | 4-Me-3-(E)- | Smp | Nva | Pro | Nal | Aad | NHtBu | 1095 | 20 | Q |
| | (CHCHCO₂tBu)—Ph—CO | | | | | | | | | |
| 2305 | 5-OMe-3-(E)- | Smp | Nva | Pro | Nal | Aad | NHtBu | 1111 | 20 | Q |
| | (CHCHCO₂tBu)—Ph—CO | | | | | | | | | |
| 2306 | 2-Me-5-OMe-3-((E)- | Smp | Nva | Pro | Nal | Aad | NHtBu | 1125 | 20 | Q |
| | (CHCHCO₂tBu)—Ph—CO | | | | | | | | | |
| 2307 | 5-(E)-(CHCHCO₂tBu)- | Smp | Nva | Pro | Nal | Aad | NHtBu | 1123 | 20 | Q |
| | 2,3-dihydrobenzofuranyl- | | | | | | | | | |
| | 7-CO | | | | | | | | | |
| 2308 | 3-Cyano-Ph—CO | Smp | Nva | Pro | Nal | Aad | NHtBu | 980 | 17 | Q |
| 2309 | 3,5-di-tBu—Ph—CO | Smp | Nva | Pro | Nal | Aad | NHtBu | 1067 | 20 | Q |
| 2310 | Cyclopropy-CO | Smp | Nva | Pro | Nal | Aad | NHtBu | 919 | 17 | Q |
| 2311 | Cyclobutyl-CO | Smp | Nva | Pro | Nal | Aad | NHtBu | 933 | 17 | Q |
| 2312 | Benzocyclobutyl-CO | Smp | Nva | Pro | Nal | Aad | NHtBu | 981 | 18 | Q |
| 2313 | Cyclopentyl-CO | Smp | Nva | Pro | Nal | Aad | NHtBu | 947 | 17 | Q |
| 2314 | Cyclohexyl-CO | Smp | Nva | Pro | Nal | Aad | NHtBu | 961 | 18 | Q |
| 2315 | Cycloheptyl-CO | Smp | Nva | Pro | Nal | Aad | NHtBu | 975 | 18 | Q |
| 2316 | 2-Ethyl-butanoyl | Smp | Nva | Pro | Nal | Aad | NHtBu | 949 | 18 | Q |
| 2317 | Pivaloyl | Smp | Nva | Pro | Nal | Aad | NHtBu | 935 | 17 | Q |
| 2318 | 2-Tetrahydrofuranyl-CO | Smp | Nva | Pro | Nal | Aad | NHtBu | 949 | 16 | Q |
| 2319 | 4-N—Me-piperazin-1-yl- | Smp | Nva | Pro | Nal | Aad | NHtBu | 976 | 17 | Q |
| | CO | | | | | | | | | |
| 2320 | MeCOCH₂CH₂CO | Smp | Nva | Pro | Nal | Aad | NHtBu | 949 | 16 | Q |
| 2321 | HCONHCH₂CO | Smp | Nva | Pro | Nal | Aad | NHtBu | 936 | 16 | Q |
| 2322 | MeCONHCH₂CO | Smp | Nva | Pro | Nal | Aad | NHtBu | 950 | 16 | Q |
| 2323 | Me₂NCH₂CO | Smp | Nva | Pro | Nal | Aad | NHtBu | 936 | 17 | Q |
| 2324 | MeOCH₂CO | Smp | Nva | Pro | Nal | Aad | NHtBu | 923 | 17 | Q |
| 2325 | 2-Pyrrolidinone-5-CO | Smp | Nva | Pro | Nal | Aad | NHtBu | 962 | 16 | Q |
| 2326 | BzlOCH₂CO | Smp | Nva | Pro | Nal | Aad | NHtBu | 999 | 18 | Q |
| 2327 | (E)-Ph—CH═CH—CO | Smp | Nva | Pro | Nal | Aad | NHtBu | 981 | 18 | Q |
| 2328 | (CycloNHCONHCH₂CH)—CO | Smp | Nva | Pro | Nal | Aad | NHtBu | 962 | 16 | Q |
| 2329 | 3-Furanyl-CO | Smp | Nva | Pro | Nal | Aad | NHtBu | 945 | 17 | Q |
| 2330 | 3-Thiophenyl-CO | Smp | Nva | Pro | Nal | Aad | NHtBu | 961 | 17 | Q |
| 2342 | Ph—CH₂OCO-Gly | Smp | Nva | Pro | Nal | Aad | NHtBu | 1012 | 19 | Q |
| 2344 | Ph—CH₂OCO-Ala | Smp | Nva | Pro | Nal | Aad | NHtBu | 1026 | 17 | Q |
| 2346 | Ph—CH₂OCO-Val | Smp | Nva | Pro | Nal | Aad | NHtBu | 1054 | 18 | Q |
| 2348 | Ph—CH₂OCO-Leu | Smp | Nva | Pro | Nal | Aad | NHtBu | 1068 | 18 | Q |
| 2350 | Ph—CH₂OCO-Phg | Smp | Nva | Pro | Nal | Aad | NHtBu | 1088 | 18 | Q |
| 2351 | Ph—CH₂OCO-Tyr | Smp | Nva | Pro | Nal | Aad | NHtBu | 1118 | 18 | Q |
| 2353 | Ph—CH₂OCO-Pro | Smp | Nva | Pro | Nal | Aad | NHtBu | 1052 | 19 | Q |
| 2355 | Ph—CH₂OCO-D-Ala | Smp | Nva | Pro | Nal | Aad | NHtBu | 1026 | 17 | Q |
| 2036 | Piperonyloyl | Smp | Nva | Pro | Nal | Aad | NHtBu | 999 | 5.2 | R |
| 2037 | 4-(E)-(CH═CH—CO₂tBu)— | Smp | Nva | Pro | Nal | Aad | NHtBu | 1081 | 5.8 | R |
| | Ph—CO | | | | | | | | | |
| 2038 | (3-NO₂—Ph)—CO | Smp | Nva | Pro | Nal | Aad | NHtBu | 1000 | 5.3 | R |
| 2039 | 3-Cl—Ph—CO | Smp | Nva | Pro | Nal | Aad | NHtBu | 989 | 5.3 | R |
| 2040 | 3-MeSO₂—Ph—CO | Smp | Nva | Pro | Nal | Aad | NHtBu | 1033 | 5.1 | R |
| 2041 | 3-AcNH—Ph—CO | Smp | Nva | Pro | Nal | Aad | NHtBu | 1012 | 5 | R |
| 2042 | 3-MeO—Ph—CO | Smp | Nva | Pro | Nal | Aad | NHtBu | 985 | 5.2 | R |
| 2043 | (3-CF₃O—Ph)—CO | Smp | Nva | Pro | Nal | Aad | NHtBu | 1039 | 5.5 | R |
| 2044 | 3-NHCOEtPh | Smp | Nva | Pro | Nal | Aad | NHtBu | 1026 | 5.1 | R |
| 2045 | 3,4-diCl—Ph—CO | Smp | Nva | Pro | Nal | Aad | NHtBu | 1025 | 5.5 | R |
| 2046 | 3-(Ph—CO—)Ph—CO | Smp | Nva | Pro | Nal | Aad | NHtBu | 1059 | 5.4 | R |
| 2047 | 3,5-diBr-anisoyl | Smp | Nva | Pro | Nal | Aad | NHtBu | 1143 | 5.7 | R |
| 2048 | 3-(E)-(CH═CH—CO2tBu)— | Smp | Nva | Pro | Nal | Aad | NHtBu | 1081 | 5.7 | R |
| | Ph—CO | | | | | | | | | |
| 2178 | 5-Me-2-OH—Ph—CO | Smp | Nva | Pro | Nal | Aad | NHtBu | 985 | 23 | F |
| 2179 | 5-I-2-OH—Ph—CO | Smp | Nva | Pro | Nal | Aad | NHtBu | 1097 | 23 | F |
| 2200 | 4-(E)-(CH═CH—CO₂H)— | Smp | Nva | Pro | Nal | Aad | NHtBu | 1025 | 21 | F |
| | Ph—CO | | | | | | | | | |
| 2201 | 3-(E)-(CH═CH—CO₂H)— | Smp | Nva | Pro | Nal | Aad | NHtBu | 1025 | 22 | F |
| | Ph—CO | | | | | | | | | |
| 2388 | 4-F-3-(E)-(CH═CH— | Smp | Nva | Pro | Nal | Aad | NHtBu | 1043 | 5.5 | R |
| | CO₂H)—Ph—CO | | | | | | | | | |
| 2389 | 2-OMe-3-(E)-(CH═CH— | Smp | Nva | Pro | Nal | Aad | NHtBu | 1055 | 5.5 | R |
| | CO₂H)—Ph—CO | | | | | | | | | |
| 2390 | 4-Me-3-(E)-(CH═CH— | Smp | Nva | Pro | Nal | Aad | NHtBu | 1039 | 5.5 | R |
| | CO₂H)—Ph—CO | | | | | | | | | |
| 2391 | 5-OMe-3-(E)-(CH═CH— | Smp | Nva | Pro | Nal | Aad | NHtBu | 1055 | 5.5 | R |
| | CO₂H)—Ph—CO | | | | | | | | | |
| 2392 | 5-OMe-2-Me-3-(E)- | Smp | Nva | Pro | Nal | Aad | NHtBu | 1069 | 5.5 | R |
| | (CH═CH—CO₂H)—Ph—CO | | | | | | | | | |

TABLE 2

| | | | | | | | | | HPLC- | |
|---|---|---|---|---|---|---|---|---|---|---|
| | | | | | | | | | Ret. | |
| | | | | | | | | | Rₜ | |
| Cdc # | R1 | A1 | A2 | A3 | A4 | A5 | U | MH⁺ | (min.) | Meth. |
| 1731 | 2-EtO- | Smp | Nva | Pip | Bta | Aad | NHtBu | 1069 | 16.6 | A |
| | naphthyl-1- | | | | | | | | | |
| | CO | | | | | | | | | |
| 1747 | 2-EtO- | Smp | Nva | 3-MePro | Bta | Aad | NHtBu | 1069 | 15.8 | A |
| | naphthyl-1- | | | | | | | | | |
| | CO | | | | | | | | | |
| 1749 | 2-EtO- | Smp | Nva | MePro | Bta | Aad | NHtBu | 1069 | 15.7 | A |
| | naphthyl-1- | | | | | | | | | |
| | CO | | | | | | | | | |
| 1761 | 2-EtO- | Smp | Nva | Pro | (NMe)- | Aad | NHtBu | 1069 | 15.7 | A |
| | naphthyl-1- | | | | Bta | | | | | |
| | CO | | | | | | | | | |
| 1765 | 2-EtO- | Smp | Nva | D,L-Isc | Bta | Aad | NHtBu | 1103 | 16.8 | A |
| | naphthyl-1- | | | | | | | | | |
| | CO | | | | | | | | | |
| 1772 | 2-EtO- | Smp | Nva | Hyp(tBu) | Bta | Aad | NHtBu | 1127 | 17.7 | A |
| | naphthyl-1- | | | | | | | | | |
| | CO | | | | | | | | | |
| 1787 | 2-EtO- | Smp | Nva | Hyp | Bta | Aad | NHtBu | 1071 | 13.4 | A |
| | naphthyl-1- | | | | | | | | | |
| | CO | | | | | | | | | |
| 1794 | 2-EtO- | Smp | Nva | Oic | Bta | Aad | NHtBu | 1109 | 18.2 | A |
| | naphthyl-1- | | | | | | | | | |
| | CO | | | | | | | | | |
| 1808 | 2-EtO- | Smp | Nva | 5-phenyl- | Bta | Aad | NHtBu | 1131 | 19.1 | A |
| | naphthyl-1- | | | Pro | | | | | | |
| | CO | | | | | | | | | |
| 1816 | 2-EtO- | Smp | Nva | Ac6 | Bta | Aad | NHtBu | 1083 | 19.5 | A |
| | naphthyl-1- | | | | | | | | | |
| | CO | | | | | | | | | |
| 1817 | 2-EtO- | Smp | Nva | (N— | Bta | Aad | NHtBu | 1043 | 15.2 | A |
| | naphthyl-1- | | | Me)Ala | | | | | | |
| | CO | | | | | | | | | |
| 1771 | 2-EtO- | Smp | Nva | Pro | Bta | Aad | NHtBu | 1069 | 15.6 | A |
| | naphthyl-1- | | | | | | | | | |
| | CO | | | | | | | | | |
| 1746 | 2-EtO- | Smp | Nva | Thiopro | Bta | Aad | NHtBu | 1073 | 15.6 | A |
| | naphthyl-1- | | | | | | | | | |
| | CO | | | | | | | | | |
| 1796 | 2-EtO- | Smp | Nva | Ac5 | Bta | Glu | NHtBu | 1055 | 18.1 | A |
| | naphthyl-1- | | | | | | | | | |
| | CO | | | | | | | | | |
| 1807 | 2-EtO- | Smp | Nva | Aib | Bta | Glu | NHtBu | 1029 | 16.1 | A |
| | naphthyl-1- | | | | | | | | | |
| | CO | | | | | | | | | |
| 1810 | 2-EtO- | Smp | Nva | Thiopro | Bta | Glu | NHtBu | 1059 | 15.6 | A |
| | naphthyl-1- | | | | | | | | | |
| | CO | | | | | | | | | |
| 2059 | 2-EtO- | Smp | Nva | Pro | Bta | MeGlu | OH | 999 | 12.6 | A |
| | naphthyl-1- | | | | | | | | | |
| | CO | | | | | | | | | |
| 1970 | 2-EtO- | Smp | Nva | Pro | Bta | Aad | NH- | 1095 | 16.4 | A |
| | naphthyl-1- | | | | | | Cyclohexyl | | | |
| | CO | | | | | | | | | |
| 1941 | 2-EtO- | Smp | Nva | Pro | Bta | Aad(tBu) | NH- | 1151 | 20.7 | A |
| | naphthyl-1- | | | | | | Cyclohexyl | | | |
| | CO | | | | | | | | | |
| 1721 | 2-EtO- | Smp | Nva | Pro | Bta | Glu | NHtBu | 1041 | 15.1 | A |
| | naphthyl-1- | | | | | | | | | |
| | CO | | | | | | | | | |
| 1904 | 2-EtO- | Smp | Nva | 2-MePro | Bta | Aad | NH-(2,4- | 1111 | 18 | A |
| | naphthyl-1- | | | | | | diMe-pent-3- | | | |
| | CO | | | | | | yl) | | | |
| 1903 | 1-EtO- | Smp | Nva | 2-MePro | Bta | Aad | NH-3a- | 1121 | 17.9 | A |
| | naphthyl-2- | | | | | | (bicyclooct- | | | |
| | CO | | | | | | [3.3.0]-yl) | | | |
| 1901 | 2-EtO- | Smp | Nva | 2-MePro | Bta | Aad | NH-(2-Me- | 1083 | 16.6 | A |
| | naphthyl-1- | | | | | | but-2-yl) | | | |
| | CO | | | | | | | | | |
| 1898 | 2-EtO- | Smp | Nva | 2-MePro | Bta | Aad | N- | 1067 | 14.3 | A |
| | naphthyl-1- | | | | | | Pyrrolidinyl | | | |
| | CO | | | | | | | | | |
| 1894 | 2-EtO- | Smp | Nva | 2-MePro | Bta | Aad(tBu) | OH | 1070 | 17.9 | A |
| | naphthyl-1- | | | | | | | | | |
| | CO | | | | | | | | | |
| 1812 | 2-EtO- | Smp | Nva | 2-MePro | Bta | Aad(N- | NHtBu | 1122 | 16.9 | A |
| | naphthyl-1- | | | | | pyrrolidinyl) | | | | |
| | CO | | | | | | | | | |
| 1905 | 2-EtO- | Smp | Nva | Ac5 | Bta | Aad(NH— | NHtBu | 1126 | 19.2 | A |
| | naphthyl-1- | | | | | OtBu) | | | | |
| | CO | | | | | | | | | |
| 1940 | 2-EtO- | Smp | Nva | Ac5 | Bta | Aad(NHOH) | NHtBu | 1070 | 17.2 | A |
| | naphthyl-1- | | | | | | | | | |
| | CO | | | | | | | | | |
| 2275 | Phthalimid | Smp | Nva | 2-MePro | Bta | Aad | NHtBu | 1001 | 13.7 | A |
| 1243 | Ac | Smp | Glu | Glu | Nal | Glu | NH₂ | 885 | 23.7 | B |
| 1517 | Indolyl-2- | Smp | Glu | Glu | Bta | Glu | NH₂ | 992 | 32.6 | B |
| | CO— | | | | | | | | | |
| 1660 | Benzothio- | Smp | Glu | Glu | Bta | Glu | NH₂ | 1009 | 35.5 | B |
| | phenyl-2-CO | | | | | | | | | |
| 1661 | 3,5-Di-tBu- | Smp | Glu | Glu | Bta | Glu | NH₂ | 1065 | 46 | C |
| | Benzoyl | | | | | | | | | |
| 1670 | 2-Me- | Smp | Glu | Glu | Bta | Glu | NH₂ | 1017 | 35.1 | B |
| | naphthyl-1- | | | | | | | | | |
| | CO | | | | | | | | | |
| 1763 | 2-EtO- | Smp | Glu | Pro | Bta | Aad | NHtBu | 1085 | 12.2 | A |
| | naphthyl-1- | | | | | | | | | |
| | CO | | | | | | | | | |
| 1303 | 2-MeO- | Smp | Glu | Asp | Nal | Glu | NH₂ | 1013 | 31.8 | B |
| | naphthyl-1- | | | | | | | | | |
| | CO | | | | | | | | | |
| 1373 | 2-MeO- | Smp | Glu | Gln | Nal | Glu | NH₂ | 1026 | 30.6 | B |
| | naphthyl-1- | | | | | | | | | |
| | CO | | | | | | | | | |
| 1376 | 2-MeO- | Smp | Glu | Glu | Nal | Glu | NH₂ | 1026 | 31.6 | B |
| | naphthyl-1- | | | | | | | | | |
| | CO | | | | | | | | | |
| 1416 | 2-MeO- | Smp | Glu | Glu | Nal | Gln | NH₂ | 1026 | 31 | B |
| | naphthyl-1- | | | | | | | | | |
| | CO | | | | | | | | | |
| 1419 | 2-MeO- | Smp | Glu | Glu | Bta | Glu | NH₂ | 1033 | 31.7 | B |
| | naphthyl-1- | | | | | | | | | |
| | CO | | | | | | | | | |
| 1516 | 2-MeO- | Smp | Glu | Glu | Bta | Glu | OH | 1034 | 32.3 | B |
| | naphthyl-1- | | | | | | | | | |
| | CO | | | | | | | | | |
| 1521 | 2-MeO- | Smp | Glu | Glu | Bta | Glu | NHtBu | 1089 | 37.5 | B |
| | naphthyl-1- | | | | | | | | | |
| | CO | | | | | | | | | |
| 1522 | 2-MeO- | Smp | Glu | Glu | Bta | Glu | NH-5- | 1149 | 43.1 | C |
| | naphthyl-1- | | | | | | Indanyl | | | |
| | CO | | | | | | | | | |
| 1565 | 2-MeO- | Smp | Glu | Glu | Bta | Glu | NH- | 1167 | 44 | C |
| | naphthyl-1- | | | | | | Adamantyl | | | |
| | CO | | | | | | | | | |
| 1659 | 2-EtO- | Smp | Glu | Glu | Bta | Glu | NH2 | 1047 | 34.3 | B |
| | naphthyl-1- | | | | | | | | | |
| | CO | | | | | | | | | |
| 1720 | 2-EtO- | Smp | Glu | D-Glu | Bta | Glu | NHtBu | 1103 | 37.7 | B |
| | naphthyl-1- | | | | | | | | | |
| | CO | | | | | | | | | |
| 1899 | 2-EtO- | Smp | Glu | 2-MePro | Bta | Asu | NH-tBu | 1183 | 18.2 | A |
| | naphthyl-1- | | | | | | | | | |
| | CO | | | | | | | | | |
| 1679 | 2-EtO- | Smp | Glu(tBu) | Pro | Bta | Glu | NHtBu | 1127 | 18.7 | A |
| | naphthyl-1- | | | | | | | | | |
| | CO | | | | | | | | | |
| 1719 | 2-EtO- | Smp | Glu(tBu) | Pro | Bta | Aad | NHtBu | 1141 | 17.2 | A |
| | naphthyl-1- | | | | | | | | | |
| | CO | | | | | | | | | |
| 1820 | 2-EtO- | Smp | Glu(tBu) | Ac5 | Bta | Aad | NHtBu | 1155 | 20 | A |
| | naphthyl-1- | | | | | | | | | |
| | CO | | | | | | | | | |
| 1822 | 2-EtO- | Smp | Glu(tBu) | 2-MePro | Bta | Aad | NHtBu | 1155 | 17.6 | A |
| | naphthyl-1- | | | | | | | | | |
| | CO | | | | | | | | | |
| 1902 | 2-EtO- | Smp | Glu(tBu) | 4,4- | Bta | Aad | NHtBu | 1169 | 18.7 | A |
| | naphthyl-1- | | | diMePro | | | | | | |
| | CO | | | | | | | | | |
| 1893 | 2-EtO- | Smp | Glu(tBu) | Iva | Bta | Aad | NHtBu | 1143 | 19.1 | A |
| | naphthyl-1- | | | | | | | | | |
| | CO | | | | | | | | | |
| 1892 | 2-EtO- | Smp | Glu(tBu) | Iva | Bta | Aad | NHtBu | 1143 | 18.9 | A |
| | naphthyl-1- | | | | | | | | | |
| | CO | | | | | | | | | |
| 1891 | 2-EtO- | Smp | Glu(tBu) | Aib | Bta | Aad | NHtBu | 1129 | 18 | A |
| | naphthyl-1- | | | | | | | | | |
| | CO | | | | | | | | | |
| 1837 | 2-EtO- | Smp | Glu(tBu) | Ac3 | Bta | Aad | NHtBu | 1127 | 16.5 | A |
| | naphthyl-1- | | | | | | | | | |
| | CO | | | | | | | | | |
| 1836 | 2-EtO- | Smp | Glu(tBu) | Dehydro- | Bta | Aad | NHtBu | 1141 | 17.8 | A |
| | naphthyl-1- | | | val | | | | | | |
| | CO | | | | | | | | | |
| 2237 | 2-EtO- | Smp | Glu(tBu) | 2-MePro | Ala | Aad | NH-3a- | 1075 | 15 | A |
| | naphthyl-1- | | | | | | (bicyclooct- | | | |
| | CO | | | | | | [3.3.0]-yl) | | | |
| 2206 | 2-EtO- | Smp | Glu(tBu) | 2-MePro | Nal | Aad | NH-3a- | 1201 | 20.1 | A |
| | naphthyl-1- | | | | | | (bicyclooct- | | | |
| | CO | | | | | | [3.3.0]-yl) | | | |
| 2167 | 2-EtO- | Smp | Glu(tBu) | 2-MePro | Bta | Aad | NH-3a- | 1207 | 19.8 | A |
| | naphthyl-1- | | | | | | (bicyclooct- | | | |
| | CO | | | | | | [3.3.0]-yl) | | | |
| 1819 | 2-EtO- | Smp | MeGlu | Pro | Bta | Glu | NHtBu | 1141 | 17.3 | A |
| | naphthyl-1- | | (tBu) | | | | | | | |
| | CO | | | | | | | | | |
| 1897 | Ac | (N—Me) | Glu(tBu) | Aib | Bta | Aad | NHtBu | 987 | 13.2 | A |
| | | Smp | | | | | | | | |
| 1896 | 2-EtO- | MeSmp | Glu(tBu) | Aib | Bta | Aad | NHtBu | 1143 | 17.4, | A |
| | naphthyl-1- | | | | | | | | 17.7 | |
| | CO | | | | | | | | | |
| 1895 | 2-EtO- | MeSmp | Glu(tBu) | MeAib | Bta | Aad | NHtBu | 1143 | 17.3 | A |
| | naphthyl-1- | | | | | | | | | |
| | CO | | | | | | | | | |
| 2168 | 1-Fluorenyl- | Smp | Glu(tBu) | 2-MePro | Bta | Aad | NH-3a- | 1201 | 20.2 | A |
| | CO | | | | | | (bicyclooct- | | | |
| | | | | | | | [3.3.0]-yl) | | | |
| 2219 | 1-Fluorenyl- | Smp | Glu(tBu) | 2-MePro | Bta | Aad(Me) | NH-3a- | 1215 | 21.9 | A |
| | CO | | | | | | (bicyclooct- | | | |
| | | | | | | | [3.3.0]-yl) | | | |
| 1564 | 2-MeO- | Smp | Glu(tBu) | Glu(tBu) | Bta | Glu(tBu) | OH | 1202 | 14.8 | E |
| | naphthyl-1- | | | | | | | | | |
| | CO | | | | | | | | | |
| 1672 | 2-EtO- | Smp | Glu(tBu) | Glu(tBu) | Bta | Glu | OtBu | 1216 | 17.6 | E |
| | naphthyl-1- | | | | | | | | | |
| | CO | | | | | | | | | |
| 1673 | 2-EtO- | Smp | Glu(tBu) | Glu(tBu) | Bta | Glu | NHtBu | 1215 | 16.8 | E |
| | naphthyl-1- | | | | | | | | | |
| | CO | | | | | | | | | |
| 1730 | 2-EtO- | Smp | Val | Pro | Bta | Aad | NHtBu | 1055 | 15 | A |
| | naphthyl-1- | | | | | | | | | |
| | CO | | | | | | | | | |
| 1743 | 2-EtO- | Smp | Leu | Pro | Bta | Aad | NHtBu | 1069 | 16.1 | A |
| | naphthyl-1- | | | | | | | | | |
| | CO | | | | | | | | | |
| 1748 | 2-EtO- | Smp | Nle | Pro | Bta | Aad | NHtBu | 1069 | 16 | A |
| | naphthyl-1- | | | | | | | | | |
| | CO | | | | | | | | | |
| 1759 | 2-EtO- | Smp | Pro | Pro | Bta | Aad | NHtBu | 1053 | 13.3 | A |
| | naphthyl-1- | | | | | | | | | |
| | CO | | | | | | | | | |
| 1764 | 2-EtO- | Smp | D-Pro | Pro | Bta | Aad | NHtBu | 1053 | 14.7 | A |
| | naphthyl-1- | | | | | | | | | |
| | CO | | | | | | | | | |
| 1762 | 2-EtO- | Smp | Pro | D-Pro | Bta | Aad | NHtBu | 1053 | 19.9 | A |
| | naphthyl-1- | | | | | | | | | |
| | CO | | | | | | | | | |
| 1723 | 2-EtO- | Smp | Phe | Pro | Bta | Glu | NHtBu | 1089 | 16.3 | A |
| | naphthyl-1- | | | | | | | | | |
| | CO | | | | | | | | | |
| 1792 | 2-EtO- | Smp | Ac6 | Pro | Bta | Glu | NHtBu | 1067 | 14.4 | A |
| | naphthyl-1- | | | | | | | | | |
| | CO | | | | | | | | | |
| 2218 | 2-EtO- | Smp | Lys | 2-MePro | Nal | Aad | NH-3a- | 1144 | 16.6 | A |
| | naphthyl-1- | | | | | | (bicyclooct- | | | |
| | CO | | | | | | [3.3.0]-yl) | | | |
| 2208 | 2-EtO- | Smp | Lys(Boc) | 2-MePro | Nal | Aad | NH-3a- | 1245 | 19.4 | A |
| | naphthyl-1- | | | | | | (bicyclooct- | | | |
| | CO | | | | | | [3.3.0]-yl) | | | |
| 1813 | 2-EtO- | Smp | Aib | Aib | Bta | Aad | NHtBu | 1029 | 14.2 | A |
| | naphthyl-1- | | | | | | | | | |
| | CO | | | | | | | | | |
| 1811 | 2-EtO- | Smp | Aib | Nva | Bta | Aad | NHtBu | 1043 | 15.7 | A |
| | naphthyl-1- | | | | | | | | | |
| | CO | | | | | | | | | |
| 1678 | 2-EtO- | Smp | Ala | Ala | Bta | Glu | NHtBu | 987 | 26.4 | D |
| | naphthyl-1- | | | | | | | | | |
| | CO | | | | | | | | | |
| 1760 | 2-EtO- | Smp | 2- | Nva | Bta | Aad | NHtBu | 1069 | 14.8 | A |
| | naphthyl-1- | | MePro | | | | | | | |
| | CO | | | | | | | | | |
| 2362 | 2-EtO- | Smp | Nva | Pro | Bta | Aad | NH-(2,4- | 1097 | 19 | L |
| | naphthyl-1- | | | | | | diMe-pent-3- | | | |
| | CO | | | | | | yl) | | | |
| 1843 | 2-MeO- | Smp | Pro | Gly | Nal | Glu | NH₂ | 923 | | A |
| | naphthyl-1- | | | | | | | | | |
| | CO | | | | | | | | | |
| 1844 | 2-MeO- | Smp | Pro | Ala | Nal | Glu | NH₂ | 937 | | A |
| | naphthyl-1- | | | | | | | | | |
| | CO | | | | | | | | | |
| 1845 | 2-MeO- | Smp | Ala | Pro | Nal | Glu | NH₂ | 937 | | A |
| | naphthyl-1- | | | | | | | | | |
| | CO | | | | | | | | | |
| 1846 | 2-EtO- | Smp | Asp(tBu) | Pro | Nal | Aad | NHtBu | 1121 | 17 | A |
| | naphthyl-1- | | | | | | | | | |
| | CO | | | | | | | | | |
| 1847 | 2-EtO- | Smp | Aad(tBu) | Pro | Nal | Aad | NHtBu | 1149 | 16 | A |
| | naphthyl-1- | | | | | | | | | |
| | CO | | | | | | | | | |
| 1848 | 2-EtO- | Smp | Glu | Pro | Nal | Aad | NHtBu | 1161 | 19 | A |
| | naphthyl-1- | | (cyclo- | | | | | | | |
| | CO | | hexyl) | | | | | | | |
| 1849 | 2-EtO- | Smp | Cys(Me) | Pro | Nal | Aad | NHtBu | 1067 | 15 | A |
| | naphthyl-1- | | | | | | | | | |
| | CO | | | | | | | | | |
| 1850 | 2-EtO- | Smp | Met | Pro | Nal | Aad | NHtBu | 1081 | 15 | A |
| | naphthyl-1- | | | | | | | | | |
| | CO | | | | | | | | | |
| 1851 | 2-EtO- | Smp | Pra | Pro | Nal | Aad | NHtBu | 1045 | 14 | A |
| | naphthyl-1- | | | | | | | | | |
| | CO | | | | | | | | | |
| 1852 | 2-EtO- | Smp | 3- | Pro | Nal | Aad | NHtBu | 1035 | 14 | A |
| | naphthyl-1- | | MeAla | | | | | | | |
| | CO | | | | | | | | | |
| 1853 | 2-EtO- | Smp | Ser(Me) | Pro | Nal | Aad | NHtBu | 1051 | 14 | A |
| | naphthyl-1- | | | | | | | | | |
| | CO | | | | | | | | | |
| 1854 | 2-EtO- | Smp | hLeu | Pro | Nal | Aad | NHtBu | 1077 | 17 | A |
| | naphthyl-1- | | | | | | | | | |
| | CO | | | | | | | | | |
| 1855 | 2-EtO- | Smp | Nva | Pro | Nal | Aad | NHtBu | 1049 | 15 | A |
| | naphthyl-1- | | | | | | | | | |
| | CO | | | | | | | | | |
| 1856 | 2-EtO- | Smp | Nva | azetane- | Nal | Aad | NHtBu | 1035 | 15 | A |
| | naphthyl-1- | | | CO | | | | | | |
| | CO | | | | | | | | | |
| 1857 | 2-EtO- | Smp | Glu(tBu) | azetane- | Nal | Aad | NHtBu | 1121 | 17 | A |
| | naphthyl-1- | | | CO | | | | | | |
| | CO | | | | | | | | | |
| 1858 | 2-EtO- | Smp | Aad(tBu) | azetane- | Nal | Aad | NHtBu | 1135 | 18 | A |
| | naphthyl-1- | | | CO | | | | | | |
| | CO | | | | | | | | | |
| 1859 | 2-EtO- | Smp | Nva | 3,4- | Nal | Aad | NHtBu | 1047 | | A |
| | naphthyl-1- | | | dehydro- | | | | | | |
| | CO | | | Pro | | | | | | |
| 1860 | 2-EtO- | Smp | Glu(tBu) | 3,4- | Nal | Aad | NHtBu | 1133 | 17 | A |
| | naphthyl-1- | | | dehydro- | | | | | | |
| | CO | | | Pro | | | | | | |
| 1861 | 2-EtO- | Smp | Aad(tBu) | 3,4- | Nal | Aad | NHtBu | 1147 | 17 | A |
| | naphthyl-1- | | | dehydro- | | | | | | |
| | CO | | | Pro | | | | | | |
| 1733 | 2-MeO- | Smp | Gly | Pro | Nal | Glu | NH₂ | 923 | 10.6 | R |
| | naphthyl-1- | | | | | | | | | |
| | CO | | | | | | | | | |
| 1734 | 2-MeO- | Smp | Glu | Pro | Nal | Glu | NH₂ | 995 | 10.1 | R |
| | naphthyl-1- | | | | | | | | | |
| | CO | | | | | | | | | |
| 1735 | 2-MeO- | Smp | D-Glu | Pro | Nal | Glu | NH₂ | 995 | 10.6 | R |
| | naphthyl-1- | | | | | | | | | |
| | CO | | | | | | | | | |
| 1885 | 2-EtO- | Smp | Glu | Glu | 1-Nal | Glu | OH | 1042 | 23 | F |
| | naphthyl-1- | | | | | | | | | |
| | CO | | | | | | | | | |
| 1888 | 2-EtO- | Smp | Nva | Pro | Bta | Aad | NH₂ | 999 | | |
| | naphthyl-1- | | | | | | | | | |
| | CO | | | | | | | | | |
| 1890 | Ac | Smp | Nva | Pro | Bta | Aad | NH₂ | 843 | | |

TABLE 3

| | | | | | | | | | HPLC- | |
|---|---|---|---|---|---|---|---|---|---|---|
| Cdc | | | | | | | | | Ret. | |
| # | R₁ | A1 | A2 | A3 | A4 | A5 | U | MH⁺ | Rₜ (min.) | Meth. |
| 2172 | 2-EtO- | Pmp(Et)₂ | Nva | 2-MePro | Bta | Aad | NHtBu | 1125 | 22.5 | A |
| | naphthyl- | | | | | | | | | |
| | 1-CO | | | | | | | | | |
| 1900 | 2-MeO- | D,L-Smphg | Glu | Glu | Bta | Glu | NH₂ | 1033 | 31.9, | B |
| | naphthyl- | | | | | | | | 31.2 | |
| | 1-CO | | | | | | | | | |
| 1035 | 2-MeO- | Tyr(mal) | Glu | Glu | Phe | Glu | NH₂ | 1001 | 31.1 | B |
| | naphthyl- | | | | | | | | | |
| | 1-CO | | | | | | | | | |
| 1040 | Ac | Tyr(mal) | Glu | Glu | Nal | Glu | NH₂ | 909 | 26.5 | B |
| 1041 | Ac | Tyr(Fmal) | Glu | Glu | Nal | Glu | NH₂ | 927 | 25.7 | B |
| 1315 | 2-MeO- | Tyr(O— | Glu | Glu | Nal | Glu | NH₂ | 1043 | 33.3 | B |
| | naphthyl- | CH₂PO₃H₂) | | | | | | | | |
| | 1-CO | | | | | | | | | |
| 1374 | 2-MeO- | F₂Pmp | Glu | Glu | Nal | Glu | NH₂ | 1063 | 33.1 | B |
| | naphthyl- | | | | | | | | | |
| | 1-CO | | | | | | | | | |
| 1375 | 2-MeO- | D-F₂Pmp | Glu | Glu | Nal | Glu | NH₂ | 1063 | 34 | B |
| | naphthyl- | | | | | | | | | |
| | 1-CO | | | | | | | | | |
| 1420 | 2-MeO- | 4- | Glu | Glu | Nal | Glu | NH₂ | 1026 | 38 | B |
| | naphthyl- | (NHSO₂Me)- | | | | | | | | |
| | 1-CO | Phe | | | | | | | | |
| 1025 | Ac | Tyr(mal) | Glu | Glu | Phe | Glu | NH₂ | 859 | 20.7 | B |
| 1244 | Ac | 4-SO₃H-Phe | Glu | Glu | Nal | Glu | NH₂ | 871 | 22.7 | B |
| 1245 | 2-MeO- | 4-SO₃H-Phe | Glu | Glu | Phe | Glu | NH₂ | 1013 | 30.5 | B |
| | naphthyl- | | | | | | | | | |
| | 1-CO | | | | | | | | | |
| 1798 | 2-EtO- | D,L-Smphg | Nva | Pro | Nal | Glu | NHtBu | 1027 | 14.7, | A |
| | naphthyl- | | | | | | | | 15.0 | |
| | 1-CO | | | | | | | | | |
| 1821 | 2-EtO- | D,L-Smphg | Nva | Ac5 | Bta | Aad | NHtBu | 1055 | 17.7. | A |
| | naphthyl- | | | | | | | | 18.5 | |
| | 1-CO | | | | | | | | | |
| 2394 | 2-EtO- | Pmp | Nva | Pro | Bta | Aad | 3-NH-2,4- | 1095 | 14 | L |
| | Naphthyl- | | | | | | diMe- | | | |
| | 1-CO | | | | | | pentyl | | | |
| 2395 | 2-EtO- | 4-(PO₃H₂)-Phe | Nva | Pro | Bta | Aad | 3-NH-2,4- | 1083 | | |
| | Naphthyl- | | | | | | diMe- | | | |
| | 1-CO | | | | | | pentyl | | | |
| 2396 | 2-EtO- | Tyr(Fmal) | Nva | Pro | Bta | Aad | 3-NH-2,4- | 1140 | 16 | L |
| | Naphthyl- | | | | | | diMe- | | | |
| | 1-CO | | | | | | pentyl | | | |
| 2397 | 2-EtO- | 4-SO₃H-Phe | Nva | Pro | Bta | Aad | 3-NH-2,4- | 1081 | 12 | L |
| | Naphthyl- | | | | | | diMe- | | | |
| | 1-CO | | | | | | pentyl | | | |
| 2416 | 2-EtO- | Tyr(O— | Nva | Pro | Bta | Aad | 3-NH-2,4- | 1113 | | |
| | Naphthyl- | CH₂PO₃H₂) | | | | | diMe- | | | |
| | 1-CO | | | | | | pentyl | | | |
| 2420 | 2-EtO- | 4- | Nva | Pro | Bta | Aad | 3-NH-2,4- | 1075 | | |
| | Naphthyl- | (CH₂CO₂Me)- | | | | | diMe- | | | |
| | 1-CO | Phe | | | | | pentyl | | | |
| 2439 | 2-EtO- | 4-(CH₂CO₂H)- | Nva | Pro | Bta | Aad | 3-NH-2,4- | 1061 | | |
| | Naphthyl- | Phe | | | | | diMe- | | | |
| | 1-CO | | | | | | pentyl | | | |
| 2358 | 2-EtO- | Pmp(Et)₂ | Nva | Pro | Bta | Aad | 3-NH-2,4- | 1153 | 19 | L |
| | Naphthyl- | | | | | | diMe- | | | |
| | 1-CO | | | | | | pentyl | | | |
| 2359 | 2-EtO- | Tyr(mal) | Nva | Pro | Bta | Aad | 3-NH-2,4- | 1121 | 16 | L |
| | Naphthyl- | | | | | | diMe- | | | |
| | 1-CO | | | | | | pentyl | | | |
| 2360 | 2-EtO- | 4-COOH-Phe | Nva | Pro | Bta | Aad | 3-NH-2,4- | 1047 | 17 | L |
| | Naphthyl- | | | | | | diMe- | | | |
| | 1-CO | | | | | | pentyl | | | |
| 2361 | 2-EtO- | 4- | Nva | Pro | Bta | Aad | 3-NH-2,4- | 1118 | 19 | L |
| | Naphthyl- | (NHCOCO₂Et)- | | | | | diMe- | | | |
| | 1-CO | Phe | | | | | pentyl | | | |
| 2363 | 2-EtO- | 4-(PO(OEt)₂)- | Nva | Pro | Bta | Aad | 3-NH-2,4- | 1139 | 19 | L |
| | Naphthyl- | Phe | | | | | diMe- | | | |
| | 1-CO | | | | | | pentyl | | | |

TABLE 4

| | | | | | | | | | HPLC- | |
|---|---|---|---|---|---|---|---|---|---|---|
| | | | | | | | | | Ret. | |
| Cdc # | R₁ | A1 | A2 | A3 | A4 | A5 | U | MH⁺ | (min.) | Meth. |
| 2435 | 2-EtO- | 3-(CH₂—NH—CH₂-2- | Nva | Pro | Bta | Aad | 3-NH-2,4- | 1210 | 7.3 | R |
| | Naphthyl- | tetrahydrofuranyl)- | | | | | diMe-pentyl | | | |
| | 1-CO | Smp | | | | | | | | |
| 2436 | 2-EtO- | 3-(CH₂—N(Ac)—CH₂- | Nva | Pro | Bta | Aad | 3-NH-2,4- | 1252 | 6.9 | R |
| | Naphthyl- | 2-tetrahydrofuranyl)- | | | | | diMe-pentyl | | | |
| | 1-CO | Smp | | | | | | | | |
| 2437 | 2-EtO- | 3-(CH₂—NH—CH₂— | Nva | Pro | Bta | Aad | 3-NH-2,4- | 1197 | 6.8 | R |
| | Naphthyl- | CH₂—NMe₂)-Smp | | | | | diMe-pentyl | | | |
| | 1-CO | | | | | | | | | |
| 2438 | 2-EtO- | 3-(CH₂—N(Ac)—CH₂— | Nva | Pro | Bta | Aad | 3-NH-2,4- | 1239 | 6.8 | R |
| | Naphthyl- | CH₂—NMe₂)-Smp | | | | | diMe-pentyl | | | |
| | 1-CO | | | | | | | | | |
| 2452 | 2-EtO- | 2-(CH₂—N(Ac)—CH₂— | Nva | Pro | Bta | Aad | 3-NH-2,4- | 1272 | | |
| | Naphthyl- | CH₂—Ph)-Smp | | | | | diMe-pentyl | | | |
| | 1-CO | | | | | | | | | |
| 2453 | 2-EtO- | 3-(CH₂—NH— | Nva | Pro | Bta | Aad | 3-NH-2,4- | 1184 | 7 | R |
| | Naphthyl- | (CH₂)₃OH)-Smp | | | | | diMe-pentyl | | | |
| | 1-CO | | | | | | | | | |
| 2454 | 2-EtO- | 3-(CH₂—N(Ac)— | Nva | Pro | Bta | Aad | 3-NH-2,4- | 1226 | 6.7 | R |
| | Naphthyl- | (CH₂)₃OH)-Smp | | | | | diMe-pentyl | | | |
| | 1-CO | | | | | | | | | |
| 2455 | 2-EtO- | 3-(CH₂—NH—(CH₂)₃— | Nva | Pro | Bta | Aad | 3-NH-2,4- | 1251 | 7.1 | R |
| | Naphthyl- | N-pyrrolidinonyl)- | | | | | diMe-pentyl | | | |
| | 1-CO | Smp | | | | | | | | |
| 2456 | 2-EtO- | 3-(CH₂—N(Ac)— | Nva | Pro | Bta | Aad | 3-NH-2,4- | 1293 | 6.7 | R |
| | Naphthyl- | (CH₂)₃—N- | | | | | diMe-pentyl | | | |
| | 1-CO | pyrrolidinonyl)-Smp | | | | | | | | |
| 2479 | 2-EtO- | 2-(E)-(CH═CH— | Nva | Pro | Bta | Aad | 3-NH-2,4- | 1167 | 20 | N |
| | Naphthyl- | COOH)-Smp | | | | | diMe-pentyl | | | |
| | 1-CO | | | | | | | | | |
| 2480 | 2-EtO- | 2-Br-Smp | Nva | Pro | Bta | Aad | 3-NH-2,4- | 1177 | 24 | N |
| | Naphthyl- | | | | | | diMe-pentyl | | | |
| | 1-CO | | | | | | | | | |
| 2408 | 2-EtO- | 3-CHO-Smp | Nva | Pro | Bta | Aad | 3-NH-2,4- | 1125 | | |
| | Naphthyl- | | | | | | diMe-pentyl | | | |
| | 1-CO | | | | | | | | | |

TABLE 5

| | | | | | | | | | HPLC- | |
|---|---|---|---|---|---|---|---|---|---|---|
| Cdc | | | | | | | | | Ret. | |
| # | R₁ | A1 | A2 | A3 | A4 | A5 | U | MH+ | Rₜ (min.) | Meth. |
| 2280 | 2-EtO-Naphthyl- | Smp | Glu | Glu | Phe | Glu | OH | 992 | 16 | H |
| | 1-CO | | | | | | | | | |
| 2243 | 2-EtO-Naphthyl- | Smp | Glu | Glu | 3-Cl-Phe | Glu | OH | 1026 | 17 | H |
| | 1-CO | | | | | | | | | |
| 2244 | 2-EtO-Naphthyl- | Smp | Glu | Glu | 2-Cl-Phe | Glu | OH | 1026 | 17 | H |
| | 1-CO | | | | | | | | | |
| 2245 | 2-EtO-Naphthyl- | Smp | Glu | Glu | 4-Cl-Phe | Glu | OH | 1026 | 17 | H |
| | 1-CO | | | | | | | | | |
| 2246 | 2-EtO-Naphthyl- | Smp | Glu | Glu | 4-COOH-Phe | Glu | OH | 1036 | 16 | H |
| | 1-CO | | | | | | | | | |
| 2247 | 2-EtO-Naphthyl- | Smp | Glu | Glu | 4-CONH₂-Phe | Glu | OH | 1035 | 16 | H |
| | 1-CO | | | | | | | | | |
| 2248 | 2-EtO-Naphthyl- | Smp | Glu | Glu | 4-NHCOMe- | Glu | OH | 1049 | 16 | H |
| | 1-CO | | | | Phe | | | | | |
| 2249 | 2-EtO-Naphthyl- | Smp | Glu | Glu | 4-NH₂-Phe | Glu | OH | 1007 | 15 | H |
| | 1-CO | | | | | | | | | |
| 2434 | 2-EtO-Naphthyl- | Smp | Glu | Glu | 3-NH₂-Phe | Glu | OH | 1007 | 5.4 | R |
| | 1-CO | | | | | | | | | |
| 2263 | 2-EtO-Naphthyl- | Smp | Glu | Glu | 4-NHCO₂Et- | Glu | OH | 1080 | 16 | H |
| | 1-CO | | | | Phe | | | | | |
| 2264 | 2-EtO-Naphthyl- | Smp | Glu | Glu | 4-NHCOPh- - | Glu | OH | 1111 | 17 | H |
| | 1-CO | | | | Phe | | | | | |
| 2270 | 2-EtO-Naphthyl- | Smp | Glu | Glu | 3-NHAc-Phe | Glu | OH | No | 16 | H |
| | 1-CO | | | | | | | MS | | |
| 2271 | 2-EtO-Naphthyl- | Smp | Glu | Glu | 3-NHCOPh- - | Glu | OH | 1110 | 17 | H |
| | 1-CO | | | | Phe | | | | | |
| 2281 | 2-EtO-Naphthyl- | Smp | Glu | Glu | 4-CF₃-Phe | Glu | OH | 1060 | 17 | H |
| | 1-CO | | | | | | | | | |
| 2282 | 2-EtO-Naphthyl- | Smp | Glu | Glu | 4-Ph-Phe | Glu | OH | 1068 | 17 | H |
| | 1-CO | | | | | | | | | |
| 2283 | 2-EtO-Naphthyl- | Smp | Glu | Glu | 3-NO₂-Phe | Glu | OH | 1037 | 16 | H |
| | 1-CO | | | | | | | | | |
| 2335 | 2-EtO-Naphthyl- | Smp | Nva | Pro | 4-CF₃-Phe | Aad | 3-NH-2,4- | 1109 | 23 | O |
| | 1-CO | | | | | | diMepentyl | | | |
| 2336 | 2-EtO-Naphthyl- | Smp | Nva | Pro | 4-NHAc-Phe | Aad | 3-NH-2,4- | 1098 | 30 | P |
| | 1-CO | | | | | | diMepentyl | | | |
| 2337 | 2-EtO-Naphthyl- | Smp | Nva | Pro | 4-COOH-Phe | Aad | 3-NH-2,4- | 1085 | 29 | P |
| | 1-CO | | | | | | diMepentyl | | | |
| 2338 | 2-EtO-Naphthyl- | Smp | Nva | Pro | 4-Ph-Phe | Aad | 3-NH-2,4- | 1117 | 14 | O |
| | 1-CO | | | | | | diMepentyl | | | |
| 2364 | 2-EtO-Naphthyl- | Smp | Glu | Glu | 3-NHCO₂Et- | Glu | OH | 1079 | 5.1 | R |
| | 1-CO | | | | Phe | | | | | |
| 2368 | 2-EtO-Naphthyl- | Smp | Nva | Pro | 4-NHCOPh- | Aad | 3-NH-2,4- | 1160 | 27 | P |
| | 1-CO | | | | Phe | | diMepentyl | | | |
| 2369 | 2-EtO-Naphthyl- | Smp | Nva | Pro | 2-Cl-Phe | Aad | 3-NH-2,4- | 1075 | 28 | P |
| | 1-CO | | | | | | diMepentyl | | | |
| 2370 | 2-EtO-Naphthyl- | Smp | Nva | Pro | 3-Cl-Phe | Aad | 3-NH-2,4- | 1075 | 28 | P |
| | 1-CO | | | | | | diMepentyl | | | |
| 2371 | 2-EtO-Naphthyl- | Smp | Nva | Pro | 4-CONH₂-Phe | Aad | 3-NH-2,4- | 1084 | 26 | P |
| | 1-CO | | | | | | diMepentyl | | | |
| 2372 | 2-EtO-Naphthyl- | Smp | Nva | Pro | Phe | Aad | 3-NH-2,4- | 1041 | 20 | L |
| | 1-CO | | | | | | diMepentyl | | | |
| 2373 | 2-EtO-Naphthyl- | Smp | Nva | Pro | 4-NHCO₂Et- | Aad | 3-NH-2,4- | 1128 | 16 | L |
| | 1-CO | | | | Phe | | diMepentyl | | | |

TABLE 6

| | | | | | | | | | HPLC- | |
|---|---|---|---|---|---|---|---|---|---|---|
| Cdc | | | | | | | | | Ret. | |
| # | R₁ | A1 | A2 | A3 | A4 | A5 | U | MH⁺ | Rₜ (min.) | Meth. |
| 1950 | 2-EtO- | Smp | Nva | Pro | (7-O)-Nal | Aad | NHtBu | 1065 | 13.7 | A |
| | naphthyl-1-CO | | | | | | | | | |
| 1875 | 2-EtO- | Smp | Glu | Glu | 7-(OiBu)-1-Nal | Glu | NH₂ | 1114 | 19 | J |
| | Naphthyl-1-CO | | | | | | | | | |
| 1876 | 2-EtO- | Smp | Glu | Glu | 7-(OCH₂CH₂—N- | Glu | NH₂ | 1169 | 6.6 | R |
| | Naphthyl-1-CO | | | | piperazinyl)-1-Nal | | | | | |
| 1878 | 2-EtO- | Smp | Glu | Glu | 7-(OBzl)-1-Nal | Glu | NH₂ | 1147 | | |
| | Naphthyl-1-CO | | | | | | | | | |
| 1923 | 2-EtO- | Smp | Glu | Glu | 7-MeO-1-Nal | Glu | OH | 1072 | 24.5 | F |
| | Naphthyl-1-CO | | | | | | | | | |
| 1924 | 2-EtO- | Smp | Glu | Glu | 7-(CH₂—C₆H₁₁)-1- | Glu | OH | 1154 | 27 | F |
| | Naphthyl-1-CO | | | | Nal | | | | | |
| 1925 | 2-EtO- | Smp | Glu | Glu | 7-(OCH₂CH(OH)— | Glu | OH | 1132 | 23 | F |
| | Naphthyl-1-CO | | | | CH₂OH)-1-Nal | | | | | |
| 1926 | 2-EtO- | Smp | Glu | Glu | 7-OPr-1-Nal | Glu | OH | 1100 | 29 | F |
| | Naphthyl-1-CO | | | | | | | | | |
| 1927 | 2-EtO- | Smp | Glu | Glu | 7-(OCH₂CH₂-1- | Glu | OH | 1169 | 24 | I |
| | Naphthyl-1-CO | | | | piperazinyl)-1-Nal | | | | | |
| 1958 | 2-EtO-Naphthyl- | Smp | Glu | Glu | 7-(O(CH₂)₂—NH₂)-1-Nal | Glu | OH | 1101 | 22 | F |
| | 1-CO | | | | | | | | | |
| 1959 | 2-EtO-Naphthyl- | Smp | Glu | Glu | 7-(O(CH₂)₃—NH₂)-1-Nal | Glu | OH | 1115 | 23 | F |
| | 1-CO | | | | | | | | | |
| 1960 | 2-EtO-Naphthyl- | Smp | Glu | Glu | 7-(O(CH₂)₂—C₆H₁₁)-1-Nal | Glu | OH | 1168 | 27 | F |
| | 1-CO | | | | | | | | | |
| 1961 | 2-EtO-Naphthyl- | Smp | Glu | Glu | 7-(O(CH₂)₃—C₆H₁₁)-1-Nal | Glu | OH | 1182 | 27 | F |
| | 1-CO | | | | | | | | | |
| 1962 | 2-EtO-Naphthyl- | Smp | Glu | Glu | 7-(O(CH₂)₄—C₆H₁₁)-1-Nal | Glu | OH | 1196 | 28 | F |
| | 1-CO | | | | | | | | | |
| 1963 | 2-EtO-Naphthyl- | Smp | Glu | Glu | 7-(O(CH₂)₂OH)-1-Nal | Glu | OH | 1102 | 23 | K |
| | 1-CO | | | | | | | | | |
| 1999 | 2-EtO-Naphthyl- | Smp | Glu | Glu | 7-(O(CH₂)₂—CH(OH)— | Glu | OH | 1147 | 24 | F |
| | 1-CO | | | | CH₂OH)-1-Nal | | | | | |
| 2000 | 2-EtO-Naphthyl- | Smp | Glu | Glu | 7-(O(CH₂)₂N- | Glu | OH | 1171 | 24 | F |
| | 1-CO | | | | morpholinyl)-1-Nal | | | | | |
| 2001 | 2-EtO-Naphthyl- | Smp | Glu | Glu | 7-(O—C₅H₉)-1-Nal | Glu | OH | 1126 | 26 | F |
| | 1-CO | | | | | | | | | |
| 2002 | 2-EtO-Naphthyl- | Smp | Glu | Glu | 7-OiPr-1-Nal | Glu | OH | 1100 | 26 | F |
| | 1-CO | | | | | | | | | |
| 2009 | 2-EtO-Naphthyl- | Smp | Glu | Glu | 7-(O(CH₂)₂-4-pyridyl)-1- | Glu | OH | 1163 | 23 | F |
| | 1-CO | | | | Nal | | | | | |
| 2010 | 2-EtO-Naphthyl- | Smp | Glu | Glu | 7-(OCH₂—COOH)-1-Nal | Glu | OH | 1117 | 23 | F |
| | 1-CO | | | | | | | | | |
| 2011 | 2-EtO-Naphthyl- | Smp | Glu | Glu | 7-(O(CH₂)₃COOH)-1-Nal | Glu | OH | 1145 | 24 | F |
| | 1-CO | | | | | | | | | |
| 2012 | 2-EtO-Naphthyl- | Smp | Glu | Glu | 7-(O(CH₂)₂-(4-HO—)Ph)- | Glu | OH | 1178 | 24 | F |
| | 1-CO | | | | 1-Nal | | | | | |
| 2013 | 2-EtO-Naphthyl- | Smp | Glu | Glu | 7-O(CH₂)₂-(4-NH₂)Ph)-1- | Glu | OH | 1178 | 25 | F |
| | 1-CO | | | | Nal | | | | | |
| 2014 | 2-EtO-Naphthyl- | Smp | Glu | Glu | 7-OH-1-Nal | Glu | OH | 1159 | 19 | F |
| | 1-CO | | | | | | | | | |
| 2026 | 2-EtO-Naphthyl- | Smp | Glu | Glu | 6-OH-1-Nal | Glu | OH | 1056 | 16 | H |
| | 1-CO | | | | | | | | | |
| 2027 | 2-EtO-Naphthyl- | Smp | Glu | Glu | 7-(O(CH₂)₂-2-thienyl)- | Glu | OH | 1166 | 27 | F |
| | 1-CO | | | | 1-Nal | | | | | |
| 2028 | 2-EtO-Naphthyl- | Smp | Glu | Glu | 7-(O(CH₂)₃COMe)-1- | Glu | OH | 1140 | 24 | F |
| | 1-CO | | | | Nal | | | | | |
| 2049 | 2-EtO-Naphthyl- | Smp | Glu | Glu | 7-(O(CH₂)₃OH)-1-Nal | Glu | OH | 1114 | 20 | F |
| | 1-CO | | | | | | | | | |
| 2159 | 2-EtO-Naphthyl- | Smp | Glu | Glu | 7-(O(CH₂)₂-(2-Me- | Glu | OH | 1181 | 26 | F |
| | 1-CO | | | | thiazolyl))-1-Nal | | | | | |
| 2160 | 2-EtO-Naphthyl- | Smp | Glu | Glu | 7-(O(CH₂)₂-2-pyridyl)- | Glu | OH | 1163 | 20 | M |
| | 1-CO | | | | 1-Nal | | | | | |
| 2182 | 2-EtO-Naphthyl- | Smp | Glu | Glu | 6-(O(CH₂)₃—C₆H₁₁)-1- | Glu | OH | 1182 | 20 | H |
| | 1-CO | | | | Nal | | | | | |
| 2186 | 2-EtO-Naphthyl- | Smp | Glu | Glu | 7-O(CH₂)₃— | Glu | OH | 1157 | 21 | M |
| | 1-CO | | | | NHC(NH)NH₂)-1-Nal | | | | | |
| 2187 | 2-EtO-Naphthyl- | Smp | Glu | Glu | 7-(O(CH₂)₂— | Glu | OH | 1143 | 21 | M |
| | 1-CO | | | | NHC(NH)NH₂)-1-Nal | | | | | |
| 2188 | 2-EtO-Naphthyl- | Smp | Glu | Glu | 7-(O(CH₂)₂-4- | Glu | OH | 1170 | 23 | M |
| | 1-CO | | | | tetrahydro- | | | | | |
| | | | | | pyranyl)-1-Nal | | | | | |
| 2189 | 2-EtO-Naphthyl- | Smp | Glu | Glu | 7-(O(CH₂)₂-2- | Glu | OH | 1156 | 23 | M |
| | 1-CO | | | | tetrahydofuranyl)-1- | | | | | |
| | | | | | Nal | | | | | |
| 2190 | 2-EtO-Naphthyl- | Smp | Glu | Glu | 7-(O(CH₂)₂-(4- | Glu | OH | 1206 | 23 | M |
| | 1-CO | | | | COOH)Ph)-1-Nal | | | | | |
| 2191 | 2-EtO-Naphthyl- | Smp | Glu | Glu | 7-(O(CH₂)₂-4- | Glu | OH | 1169 | 21 | M |
| | 1-CO | | | | piperazinyl)-1-Nal | | | | | |
| 2192 | 2-EtO-Naphthyl- | Smp | Glu | Glu | 7-(OCH(Me)COOH)- | Glu | OH | 1130 | 21 | M |
| | 1-CO | | | | 1-Nal | | | | | |
| 2193 | 2-EtO-Naphthyl- | Smp | Glu | Glu | 7-(O(CH₂)₂—N- | Glu | OH | 1169 | 21 | M |
| | 1-CO | | | | pyrrolidinyl)-1-Nal | | | | | |
| 2194 | 2-EtO- | Smp | Glu | Glu | 6-(O(CH₂)₂—N- | Glu | OH | 1169 | 17 | H |
| | Naphthyl-1-CO | | | | pyrrolidinonyl)- | | | | | |
| | | | | | 1-Nal | | | | | |
| 2195 | 2-EtO- | Smp | Glu | Glu | 6- | Glu | OH | 1115 | 16 | H |
| | Naphthyl-1-CO | | | | (O(CH₂)₃NH₂)- | | | | | |
| | | | | | 1-Nal | | | | | |
| 2197 | 2-EtO- | Smp | Glu | Glu | 6-(O(CH₂)₂-4- | Glu | OH | 1163 | 16 | H |
| | Naphthyl-1-CO | | | | pyridyl)-1-Nal | | | | | |
| 2198 | 2-EtO- | Smp | Glu | Glu | 6-(O(CH₂)₂-4- | Glu | OH | 1170 | 18 | H |
| | Naphthyl-1-CO | | | | tetrahydro- | | | | | |
| | | | | | pyranyl)-1-Nal | | | | | |
| 2199 | 2-EtO- | Smp | Glu | Glu | 6- | Glu | OH | 1146 | 16 | H |
| | Naphthyl-1-CO | | | | (O(CH₂)₂CH(OH) | | | | | |
| | | | | | CH₂OH)-1- | | | | | |
| | | | | | Nal | | | | | |
| 2225 | 2-EtO- | Smp | Glu | Glu | 7-(CHCH—Ph)-1- | Glu | OH | 1144 | | |
| | Naphthyl-1-CO | | | | Nal | | | | | |
| 2339 | 2-EtO- | Smp | Glu | Glu | 7-(CH₂)₂- | Glu | OH | 1114 | 3.6 | R |
| | Naphthyl-1-CO | | | | COOH)-1-Nal | | | | | |
| 2340 | 2-EtO- | Smp | Nva | Pro | 2-Oxn | Aad | 3-NH-2,4-diMe- | 1108 | 6.4 | R |
| | Naphthyl-1-CO | | | | | | pentyl | | | |
| 2204 | 2-EtO- | Smp | Glu(tBu) | 2- | (alpha- | Aad | NH-3a- | 1205 | 21.8 | A |
| | naphthyl-1-CO | | | MePro | Me(tetrahydro- | | (bicyclooct[3.3.0]- | | | |
| | | | | | Nal)) | | yl) | | | |
| 2174 | 2-EtO- | Smp | Glu(tBu) | 2- | (beta-Me)Nal | Aad | NH-3a- | 1215 | 21.2 | A |
| | naphthyl-1-CO | | | MePro | | | (bicyclooct[3.3.0]- | | | |
| | | | | | | | yl) | | | |
| 2169 | 2-EtO- | Smp | Glu(tBu) | 2- | (tetrahydro-Nal) | Aad | NH-3a- | 1205 | 21.7 | A |
| | naphthyl-1-CO | | | MePro | | | (bicyclooct[3.3.0]- | | | |
| | | | | | | | yl) | | | |

TABLE 7

| | | | | | | | | HPLC-Ret. | |
|---|---|---|---|---|---|---|---|---|---|
| cdc # | R₁ | A1 | A2 | A4 | A5 | U | M⁻ = | Rₜ (min.) | Meth. |
| 1793 | 2-EtO-naphthyl- | Smp | 3-NH-pyridinon-1-yl- | Bta | Glu | NHtBu | 995 | 13.75 | A |
| | 1-CO | | CH₂-CO | | | | | | |
| 1785 | 2-EtO-naphthyl- | Smp | 3-NH-caprolactam-1- | Bta | Glu | NHtBu | 1013 | 13.4 | A |
| | 1-CO | | CH₂-CO | | | | | | |
| 1786 | 2-EtO-naphthyl- | Smp | 6-NH-5-oxo- | Bta | Glu | NHtBu | 1043 | 15.23 | A |
| | 1-CO | | perhydropyrido[2,1-b]- | | | | | | |
| | | | (1,3)-thiazolyl-3-CO | | | | | | |
| 1795 | 2-EtO-naphthyl- | Smp | 3-NH-Ph-CO | Bta | Aad | NHtBu | 978 | 14.92 | A |
| | 1-CO | | | | | | | | |
| 1680 | 2-EtO-naphthyl- | Smp | NH-(CH₂)₄-CO | Bta | Glu | NHtBu | 944 | 15.92 | A |
| | 1-CO | | | | | | | | |

### Synthesis of 2-EtO-1-naphthyl-CO—Smp—Nva—Pro—Bta—NH—CH₂-(3-HOOC—CH₂)—Ph, cdc2276

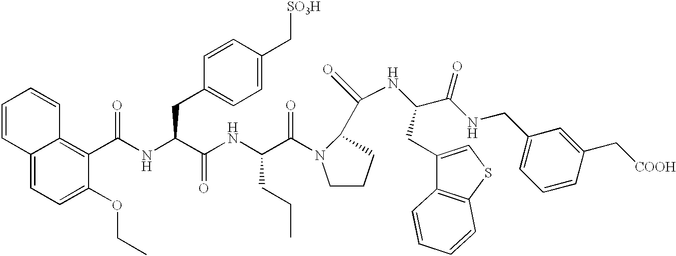

The tetrapeptide acid sequence is built up stepwise from the C-terminus as described previously (see example 3) by coupling the corresponding Fmoc-amino acid (Fmoc—Bta—OH) to the chlortrityl chloride resin, then removal of the Fmoc-protecting group to liberate the amino group for the next coupling. The final capping is done with 2-ethoxy-naphthyl-carboxylic acid. The tetrapeptide is cleaved from the resin in a similar fashion as described in example 3.

### a) 2-EtO-1-naphthyl-CO—Smp—Nva—Pro—Bta—NH—CH₂-(3-MeOOC—CH₂)—Ph

The tetrapeptide acid (50 mg, 0.058 mmol) was dissolved in dichloromethane (5 ml) then methyl 2-[3-(aminomethyl)phenyl]acetate hydrochloride (23 mg, 0.116 mmol), 1-hydroxy-7-azabenzotriazole (8 mg, 0.058 mmol), 1-(3-dimethylaminopropyl)-3-ethylcarbodiimide hydrochloride (22 mg, 0.116 mmol), and diisopropylethylamine (49 mg, 0.377 mmol) were added. The reaction mixture was stirred at room temperature for 18 hours. The reaction mixture was diluted with dichloromethane and washed with 5% aqueous citric acid (1x). The organic layer was dried over sodium sulfate and concentrated under reduced pressure. The remaining residue was redissolved in dichloromethane. Heptane was then added until a precipitate formed. The mixture was concentrated in vacuo and the remaining solid dried in vacuo to give the product as a white solid 60 mg of 2-EtO-1-naphthyl-CO—Smp—Nva—Pro—Bta—NH—CH₂-(3-MeOOC—CH₂)—Ph).

MS (ESI): MH⁺=1018.0 Rₜ=16.49 (Method A)

### a) 2-EtO-1-naphthyl-CO—Smp—Nva—Pro—Bta—NH—CH₂-(3-HOOC—CH₂)—Ph

The crude tetrapeptide ester (60 mg, 0.06 mmol) was dissolved in tetrahydrofuran (5 ml) and water (1 ml), then 1N aqueous lithium hydroxide (150 μl, 0.15 mmol) was added. The reaction mixture was stirred at room temperature for 2 days. The reaction was incomplete based on HPLC, therefore more 1N aqueous lithium hydroxide (60 μl, 0.06 mmol) was added at room temperature and the reaction mixture was then heated to 40° C. for 3 hours. The reaction mixture was cooled to 0° C. and quenched with aqueous 1N hydrochloric acid (240 μl, 4 eq). The mixture was concentrated under reduced pressure. The remaining residue was redissolved in minimal acetonitrile/water and then lypholized to give 50 mg 2-EtO-1-naphthyl-CO—Smp—Nva—Pro—Bta—NH—CH₂-(3-HOOC—CH₂)—Ph as a mixture of diastereomers (ratio 85:6)

MS(ESI): MH⁺=1004 Rₜ=14.51, 14.98 (Method A)

The following tetrapeptides were prepared to the method described above:

TABLE 8

| | | | | | | | | HPLC- | |
|---|---|---|---|---|---|---|---|---|---|
| | | | | | A4 | | | Ret.ₜ | |
| cdc # | R₁ | A1 | A2 | A3 | (ratio) | R₂ | M⁻ = | Rₜ (min) | Meth. |
| 2378 | 2-EtO- | Smp | Nva | Pro | L,D-Bta | NH—CH₂—(3-NO₂—Ph) | 991 | 15.2, | A |
| | naphthyl-1-CO | | | | (75:15) | | | 15.6 | |
| 2377 | 2-EtO- | Smp | Nva | Pro | L,D-Bta | NH—CH₂—(2-(COOH— | 1018 | 16.6, | A |
| | naphthyl-1-CO | | | | (76:13) | (CH₂)₂)—Ph) | | 17 | |
| 2277 | 2-EtO- | Smp | Nva | Pro | L,D-Bta | NH—CH₂—(4-(HOOC— | 1004 | 14.1, | A |
| | naphthyl-1-CO | | | | (76:13) | CH₂)—Ph) | | 14.5 | |
| 2276 | 2-EtO- | Smp | Nva | Pro | L,D-Bta | NH—CH₂—(3-(HOOC— | 1004 | 14.5, | A |
| | naphthyl-1-CO | | | | (85:6) | CH₂)—Ph) | | 15 | |
| 2274 | 2-EtO- | Smp | Nva | Pro | Bta | NH—CH₂—CH₂—CH₂— | 942 | 12.7 | A |
| | naphthyl-1-CO | | | | | COOH | | | |
| 2273 | 2-EtO- | Smp | Nva | Pro | L,D-Bta | NH—CH₂—(4-COOH—Ph) | 990 | 13.7, | A |
| | naphthyl-1-CO | | | | (65:13) | | | 14.2 | |
| 2272 | 2-EtO- | Smp | Nva | Pro | L,D-Bta | NH—CH₂—CH₂—(4- | 1004 | 14.1, | A |
| | naphthyl-1-CO | | | | (78:18) | COOH—Ph) | | 14.5 | |
| 2254 | 2-EtO- | Smp | Nva | Pro | L,D-Bta | NH—CH₂—(4-(HOOC— | 1018 | 14.6, | A |
| | naphthyl-1-CO | | | | (71:12) | (CH₂)₂)—Ph) | | 15 | |
| 2253 | 2-EtO- | Smp | Nva | Pro | L,D-Bta | NH—CH₂—(3-(HOOC— | 1018 | 15.1, | A |
| | naphthyl-1-CO | | | | (67:15) | (CH₂)₂)—Ph) | | 15.5 | |
| 2252 | 2-EtO- | Smp | Nva | Pro | L,D-Bta | NH—(3-(HOOC—CH₂— | 1006 | 15, | A |
| | naphthyl-1-CO | | | | (63:13) | O)—Ph) | | 15.6 | |
| 2251 | 2-EtO- | Smp | Nva | Pro | L,D-Bta | NH—CH₂—CH₂—(3- | 1004 | 17.1, | A |
| | naphthyl-1-CO | | | | (95:5) | COOH—Ph) | | 17.8 | |
| 2236 | 2-EtO- | Smp | Nva | 2- | Bta | NH—(4-(HOOC—CH₂)— | 1004 | 15.5 | A |
| | naphthyl-1-CO | | | MePro | | Ph | | | |
| 2235 | 2-EtO- | Smp | Nva | 2- | Bta | NH—(3-HOOC—CH₂)— | 1004 | 16.2 | A |
| | naphthyl-1-CO | | | MePro | | Ph | | | |
| 2207 | 2-EtO- | Smp | Nva | 2- | Bta | NH—(CH₂)₄—COOH | 970 | 13.7 | A |
| | naphthyl-1-CO | | | MePro | | | | | |
| 2205 | 2-EtO- | Smp | Nva | 2- | Bta | NH—CH₂-t-(4-HOOC)- | 1010 | 14.2 | A |
| | naphthyl-1-CO | | | MePro | | cycloC₆H₁₀ | | | |
| 2171 | 2-EtO- | Smp | Nva | 2- | Bta | NH—(3-COOH)—Ph | 990 | 16.4 | A |
| | naphthyl-1-CO | | | MePro | | | | | |
| 2170 | 2-EtO- | Smp | Nva | 2- | Bta | NH—CH₂—(3-COOH— | 1004 | 14.9 | A |
| | naphthyl-1-CO | | | MePro | | Ph) | | | |
| 2058 | 2-EtO- | Smp | Nva | Pro | Bta | NH—CH₂—CH₂—Ph | 960 | 17.2 | A |
| | naphthyl-1-CO | | | | | | | | |
| 2056 | 2-EtO- | Smp | Nva | Pro | Bta | NH—(CH₂)₄—CH₃ | 940 | 18.7 | A |
| | naphthyl-1-CO | | | | | | | | |
| 2209 | 2-EtO- | Smp | Glu | Glu | Bta | NH—CH₂—(3-COOH— | 1052 | 10.1 | A |
| | naphthyl-1-CO | | | | | Ph) | | | |
| 2217 | 2-EtO- | Smp | Glu(tBu) | Glu(tBu) | Bta | NH—CH₂—(3-COOH— | 1164 | 18.7 | A |
| | naphthyl-1-CO | | | | | Ph) | | | |
| 1428 | 2-MeO- | Smp | Glu | Glu | Nal | NH₂ | 898 | 32.5 | B |
| | naphthyl-1-CO | | | | | | | | |
| 1518 | 2-MeO- | Smp | Glu | Glu | Bta | NH—(CH₂)₅—COOH | 1018 | 34.4 | B |
| | naphthyl-1-CO | | | | | | | | |
| 1519 | 2-MeO- | Smp | Glu | Glu | Bta | NH—(CH₂)₄—COOH | 1004 | 33.4 | B |
| | naphthyl-1-CO | | | | | | | | |
| 1520 | 2-MeO- | Smp | Glu | Glu | Bta | NH—(CH₂)₃—COOH | 990 | 33.1 | B |
| | naphthyl-1-CO | | | | | | | | |
| 1561 | 2-MeO- | Smp | Glu(tBu) | Glu(tBu) | Bta | NH—(CH₂)₃—COOH | 1102 | 50.8 | C |
| | naphthyl-1-CO | | | | | | | | |
| 1566 | 2-MeO- | Smp | Glu(Me) | Glu | Bta | NH—(CH₂)₃—COOH | 1004 | 21.4 | D |
| | naphthyl-1-CO | | | | | | | | |
| 1567 | 2-MeO- | Smp | Glu | Glu(Me) | Bta | NH—(CH₂)₃—COOH | 1004 | 21.7 | D |
| | naphthyl-1-CO | | | | | | | | |
| 1570 | 2-MeO- | Smp | Glu | Glu | Bta | NH—(CH₂)₃—COOCH₃ | 1004 | 22.1 | D |
| | naphthyl-1-CO | | | | | | | | |
| 1571 | 2-MeO- | Smp | Glu(Me) | Glu(Me) | Bta | NH—(CH₂)₃—COOH | 1018 | 23.5 | D |
| | naphthyl-1-CO | | | | | | | | |
| 1572 | 2-MeO- | Smp | Glu(Me) | Glu | Bta | NH—(CH₂)₃—COOCH₃ | 1018 | 23.7 | D |
| | naphthyl-1-CO | | | | | | | | |
| 1573 | 2-MeO- | Smp | Glu(Me | Glu(Me) | Bta | NH—(CH₂)₃—COOCH₃ | 1032 | 25.3 | D |
| | naphthyl-1-CO | | | | | | | | |

### Example 2

### Protein Purification

GST-tagged Proteins (ΔN1A, ΔN1B, ΔN1C)

Expression cloning of the open reading frames for the GST-tagged expression of the catalytic domains of Cdc25A, Cdc25B, and Cdc25C was performed by cloning the appropriate fragment into the PGEX-2T or PGEX-KT (Pharmacia) vector utilizing the EcoRI and HinDIII sites (see Table 9). The resulting plasmids were transformed into BL-21 (DE3) and the proteins were overproduced by induction of mid-log cells 0.5 mM IPTG for 3 h at 25° C.

Purification of Cdc25 Catalytic Domain from GST-Cdc25

Cell pellets from an approximately 4 liter E. coli high density fermentation were thawed. The cells were resuspended in 600 ml phosphate-buffered saline (PBS), 10 mM dithiothreitol w/a protease inhibitor cocktail (complete tablets Boehringer Mannheim cat#1697498) supplemented w/1/1000 pepstatin A (10 mg/ml; 10 ug/ml final). The cells were lysed via 2 passes through a microfluidizer at 15,000 psi. The resulting suspension was centrifuged in a GSA rotor (Beckman) at 12,000 rpm for 1 hour.

The supernatant was batch bound to 250 ml GSH sepharose 4-B (Amersham Pharmacia Biotech) for 1 hour at 4 degrees with gentle rocking. The supernatant was decanted and resuspended with GSH sepharose 4-B and packed into xk50 column at 20 ml/min. The column was washed with 5-10 column volumes of 50 mM Tris pH 8.0, 0.500 mM NaCl, 1 mM EDTA, 1 mM DTT. The column was then washed at a rate of 5 ml/min. with 5 column volumes of 50 mM tris, pH 8.0, 1 mM EDTA, 1 mM DTT. The column was then eluted at a rate of 1.5 ml/min. with 50 mM tris pH 8.0, 25 mM reduced GSH, 1 mM EDTA, 1 mM DTT. The eluate was collected in 4 mL fractions.

The fractions were then subjected to size exclusion chromatography using a S300 Sephacryl xk 50/100 column eluted at 4 ml/min, equilibrated with 50 mM tris pH 8.0, 150 mM NaCl, 1 mM EDTA, 5 mM DTT. The eluate was collected as 10 mL fractions. GST-CDC25 eluted both as an aggregate in the column void volume and as a dimeric peak. Fractions corresponding to the dimer peak were pooled.

The dimeric GST-CDC25 was bound to fresh GSH sepharose beads at 4 mg fusion protein per 1 ml GSH beads. The beads were washed with 10 column volumes of 25 mM tris pH 8.0, 150 mM NaCl, 2.5 mM CaCl2, 1 mM DTT, 100 uM EDTA. The beads were resuspended in two volumes of buffer and then digested with 5 units thrombin (Calbiochem cat#604980 sp. activity 1900 units/mg) per mg fusion protein for 90 minutes at room temperature with gentle rocking.

The beads were filtered using a 0.45 μm cellulose acetate bottle top filtration system (Corning) to remove supernatant, and the beads were washed with 1 volume buffer. The wash was added to the pool. The thrombin was removed using ATIII agarose beads (Sigma cat#A-8293) at a ratio of 1 mL beads per 100 ug thrombin added. The solution was incubated for 1 hour at 4° C. with gentle rocking and then filtered using a 0.45 μm cellulose acetate bottle top filtration system (Corning) to remove the beads. 10 mM EDTA and 0.5 mM AEBSF (Calbiochem cat#101500) were then added to inactivate any remaining thrombin. The solution was then concentrated to 6 mL using a centriprep 10,000 dalton molecular weight cutoff device (Millipore) and filtered through 0.22 μm filter.

The concentrated, filtered solution was injected onto a Superdex 75 xk26/100 column equilibrated in 50 mM NaPi pH 6.75, 100 mM NaCl, 1 mM DTT, 1 mM EDTA at 2 ml/min, and the elute was collected as 2.5 mL fractions. Fractions containing the Cdc25 catalytic domain were pooled and concentrated to 20 mg/ml vs BSA. EDTA was added to 5 mM , DTT to 10 mM, AEBSF to 0.5 mM, and Na azide to 0.02% final concentrations.

Native Proteins (ΔN5A, ΔN8A, ΔN8A-c17, ΔN5B, ΔN8B, ΔN8B-c17, ΔN8B-c18, ΔN9C)

Expression cloning of the open reading frames for the native expression of the catalytic domains of Cdc25A, Cdc25B, and Cdc25C was performed by cloning the appropriate fragment into the pET-3d vector (Novagen) by incorporation of a NcoI site at the start codon and a HinDIII site following the stop codon (see Table 9). The resulting plasmids were transformed into BL-21 (DE3) and the proteins were overproduced by induction of mid-log cells with 0.5 mM IPTG for 3 h at 25° C. All steps in the purification were performed at 4° C. and phosphatase activity was followed by assays using pNPP as a substrate. In a typical preparation, 33 g of frozen cell pellets were thawed in 150 mL of buffer A (3 mM potassium phosphate (pH 7.4), 75 mM NaCl, 1 mM EDTA, 1 mM DTT, and a cocktail of protease inhibitors (0.001 mg/ml Aprotinin, 0.001 mg/ml Leupeptin, 0.01 mg/ml Soybean Trypsin Inhibitor, 0.01 mg/ml L-1-Chloro-3-(4-tosylamido)-7-amino-2-heptanone hydrochloride and 0.01 mg/ml L-1-Chloro-3-(4-tosylamido)-4-phenyl-2-butanone). Following centrifuigation at 18K for 30 min the cleared lysate was bound to 15 mL of SP-Sepharose equilibrated in buffer A. The Cdc25 protein was eluted with buffer A containing 150 mM NaCl, or by a gradient in buffer A up to 250 mM NaCl, and further purified by S-200 chromatography in phosphatase reaction buffer (50 mM Tris—HCl (pH 8.0), 50 mM NaCl, 1 mM EDTA, 1 mM DTT). Protein yields varied from 1-25 mg per liter of cell culture.

TABLE 9
Polypeptides comprising Cdc25 catalytic domain

| Construct | | | | |
|---|---|---|---|---|
| name | derived from | Source | N-terminus | C-terminus |
| ΔN1A | Cdc25A | GST-tagged (2T) | (GS)-Leu336 | Leu523 |
| ΔN1B | Cdc25B | GST-tagged (2T) | (GS)-Leu378 | Gln566 |
| ΔN1C | Cdc25C | GST-tagged (KT) | (GS)-Leu282 | Pro473 |
| ΔN5A | Cdc25A | Native | Gly323 | Leu523 |
| ΔN8A | Cdc25A | Native | Glu326 | Arg519 |
| ΔN8A-c17 | Cdc25A | Native | Glu326 | Thr506 |
| ΔN5B | Cdc25B | Native | (M)-Asp365 | Gln566 |
| ΔN8B | Cdc25B | Native | (M)-Glu368 | Arg562 |
| ΔN8B-c17 | Cdc25B | Native | (M)-Glu368 | Ser549 |
| ΔN8B-c18 | Cdc25B | Native | (M)-Glu368 | Arg548 |
| ΔN9C | Cdc25C | Native | (M)-Gly280 | Val453 |

### Example 3

### Crystallization of Polypeptides and Crystal Structure Determination

Crystallization of Cdc25A(ΔN1A)

Frozen Cdc25A (ΔN1A construct; 25 mg/ml in 25 mM Tris.HCl, pH 7.5, 100 mM NaCl, 10 mM DTT, 5 mM EDTA, 0.5 mM AEBSF; 25 μL) was thawed and mixed with 1 μL DTT (100 mM), 1 μL Na₂WO₄ (100 mM), and 23 μL H₂O. This protein solution (1 μl) was mixed with 1 μL of a reservoir solution consisting of 15% (w/v) polyethylene glycol (PEG) 4000, 100 mM sodium citrate, pH 5.6, and suspended over the reservoir on the underside of a siliconized glass cover slip at 4° C. Long, pyramidal crystals appeared in one day. Crystals also grew under these conditions in the presence of varying amounts of PEG 4000, in the presence of 0-400 mM ammonium acetate, and at pH values from 4.8 to 5.6.

Cryoprotection of a Cdc25A(ΔN1A) Crystal

A Cdc25A(ΔN1A) crystal (crystal 1) grown as described above was transferred into a series of cryoprotective buffers containing 21-24% (w/v) PEG 4000, 100 mM sodium citrate, pH 5.6, 2 mM Na₂WO₄, and 0, 5, 10, 15, and 20% (v/v) glycerol. The crystal was first soaked in the 0% glycerol buffer for two min, and then allowed to soak sequentially in the 5, 10, 15, and 20% glycerol buffers for 5 min each. The crystal was picked up with a fiber loop and flash-cooled by plunging into liquid nitrogen. The crystal was stored in a liquid nitrogen refrigerator.

X-ray Diffraction Data Collection from a Cdc25A(ΔN1A) Crystal Grown from PEG

X-ray diffraction data were collected from crystal 1 on a Siemens SRA rotating anode generator (50 kV, 108 mA, 40% bias, graphite-monochromated Cu K₆₀ radiation) equipped with a MAR Research image plate detector using the rotation method. The Cdc25A(ΔN1A) crystal was maintained at a temperature of 100 K with an Oxford Cryosystems Cryostream cooler during data collection. For each frame of data (225 total) the crystal was rotated by 0.4°. The crystal was then re-oriented (approximately 60° rotation around the x-ray beam) and 225 additional data frames were collected. The data were processed with the CCP4 Suite of programs (Collaborative Computational Project, Number 4, 1994). After determining the crystal orientations with REFIX (Kabsch, 1993) and IDXREF (Collaborative Computational Project, Number 4, 1994), the data were integrated (in space group P4₁ or P4₃, a=43.79 Å, c=117.37 Å) with MOSFLM (Leslie, 1992), scaled and merged with SCALA (Evans, 1997), and placed on an absolute scale and reduced to structure factor amplitudes with TRUNCATE (French & Wilson, 1978). Five percent of the unique reflections were assigned, in a random fashion, to the “free” set, for calculation of the free R-factor (R_{free}); the remaining 95% of the reflections constituted the “working” set, for calculation of the R-factor (R). These data are summarized in Table 10.

Comparison of the Cdc25A(ΔN1A) Structure in Crystals Grown from Ammonium Sulfate or PEG

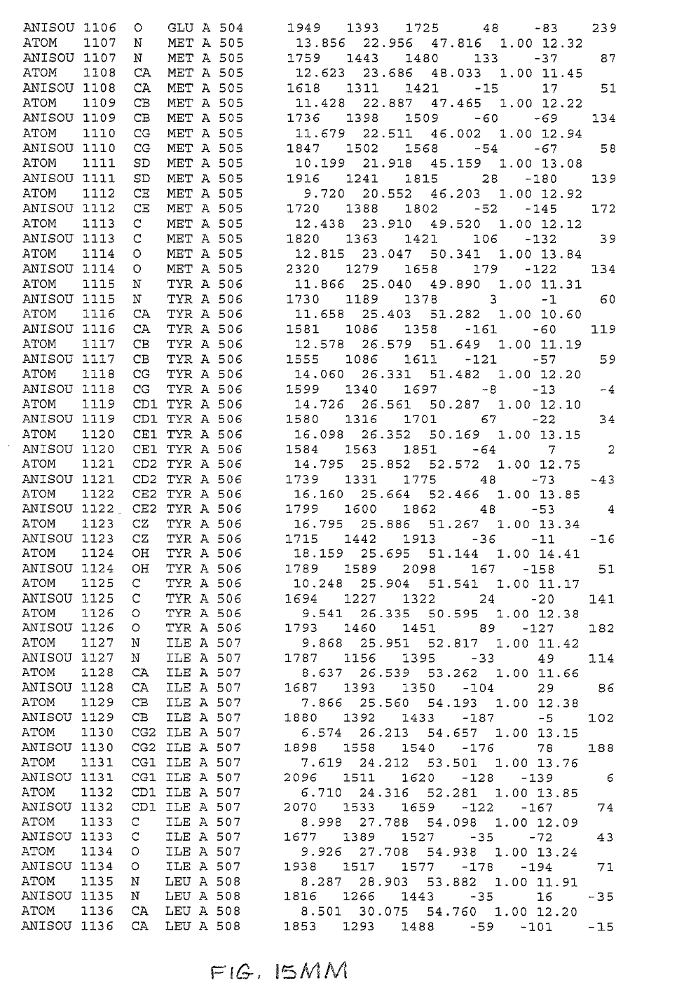

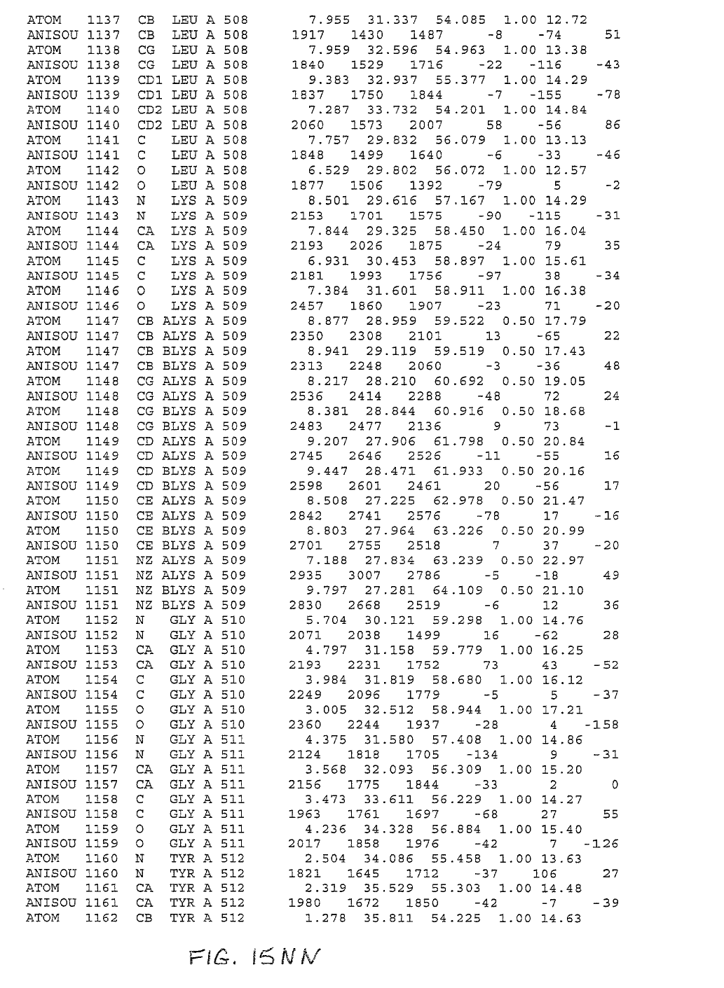

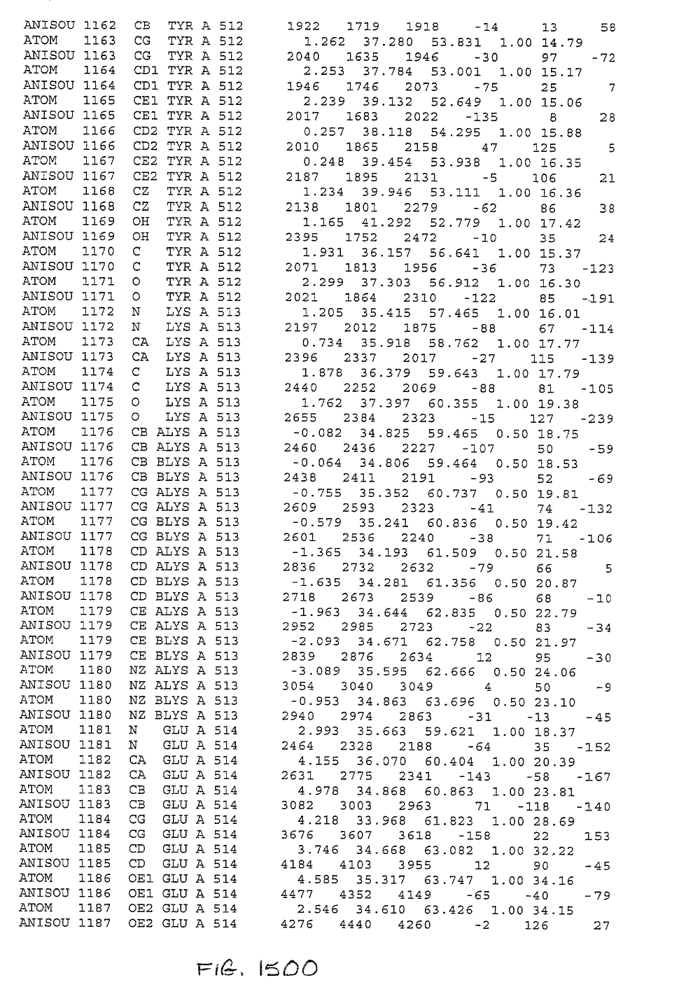

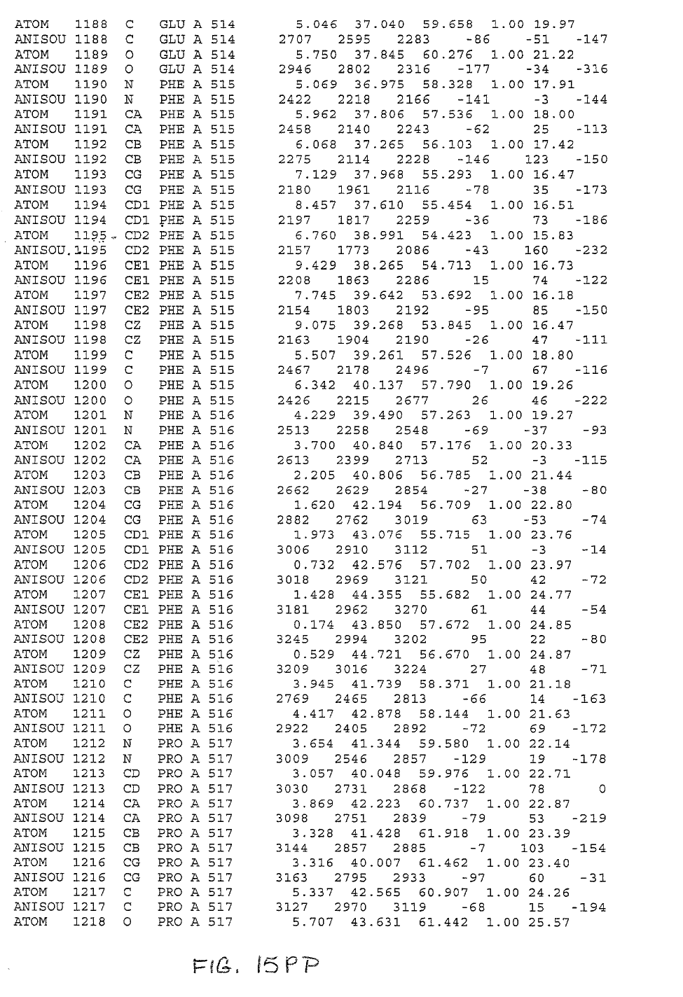

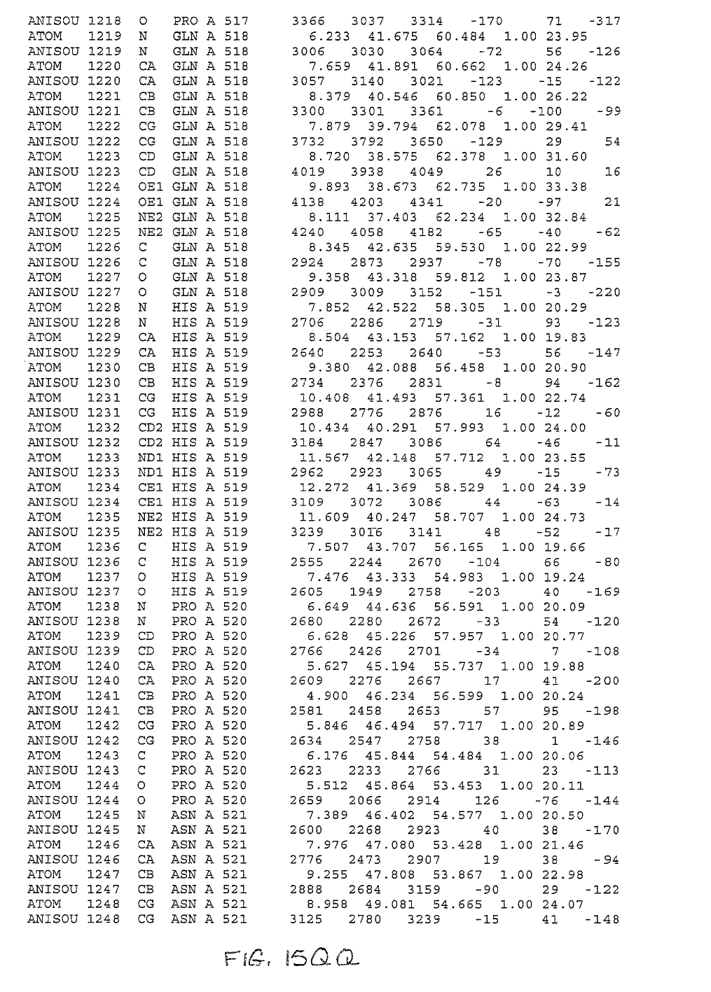

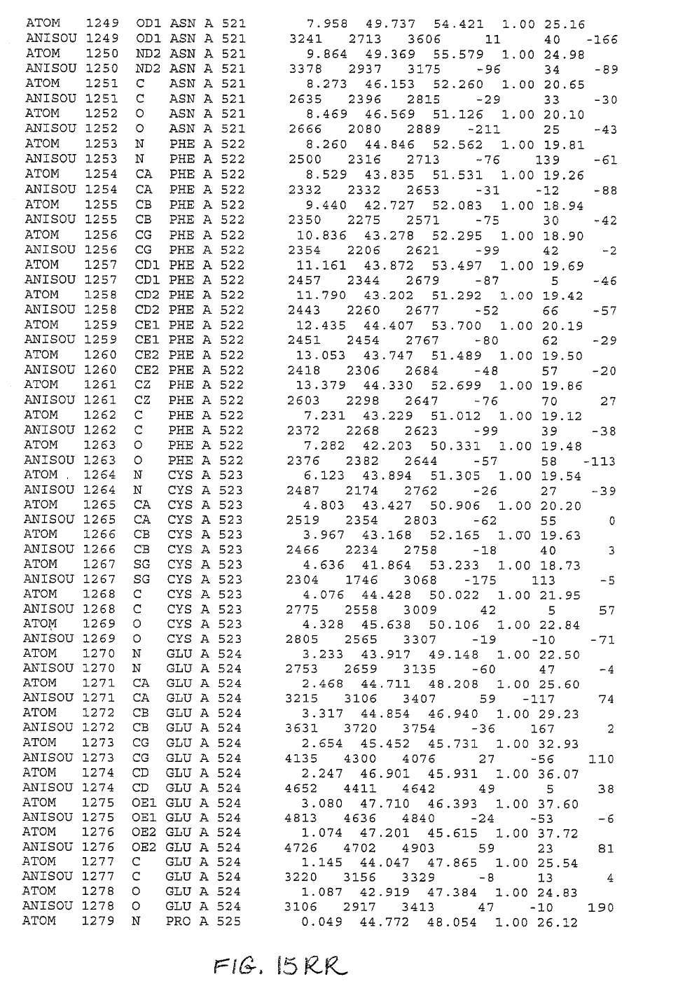

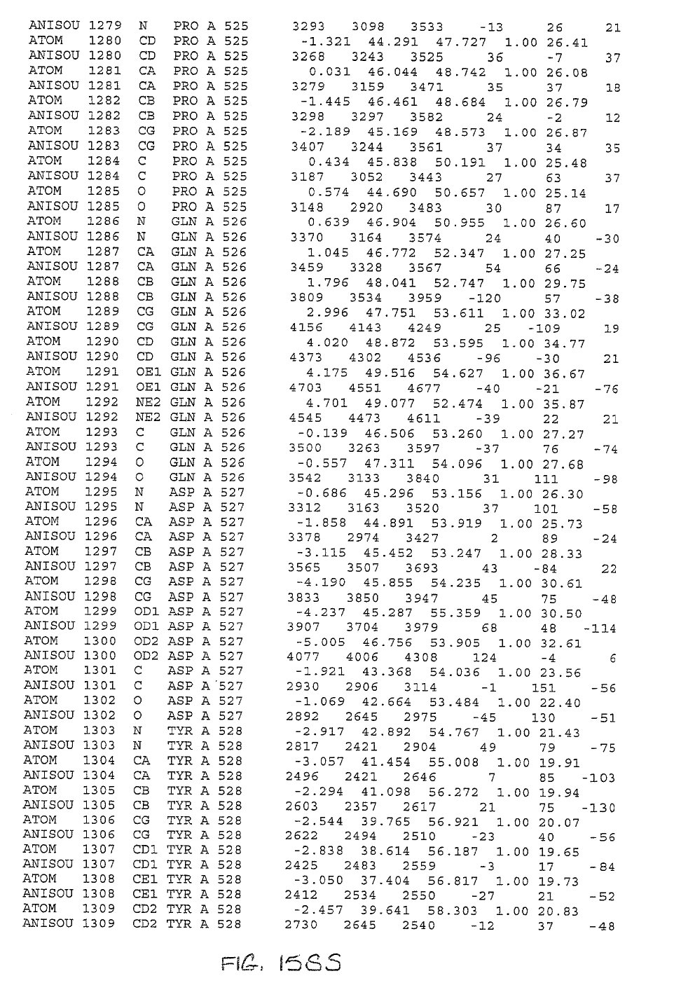

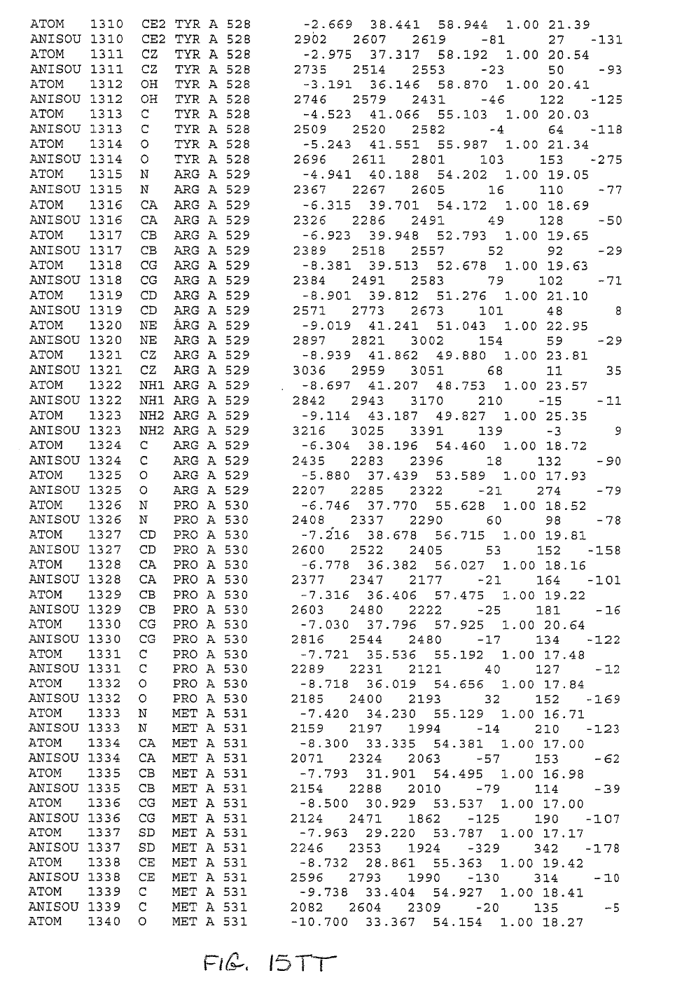

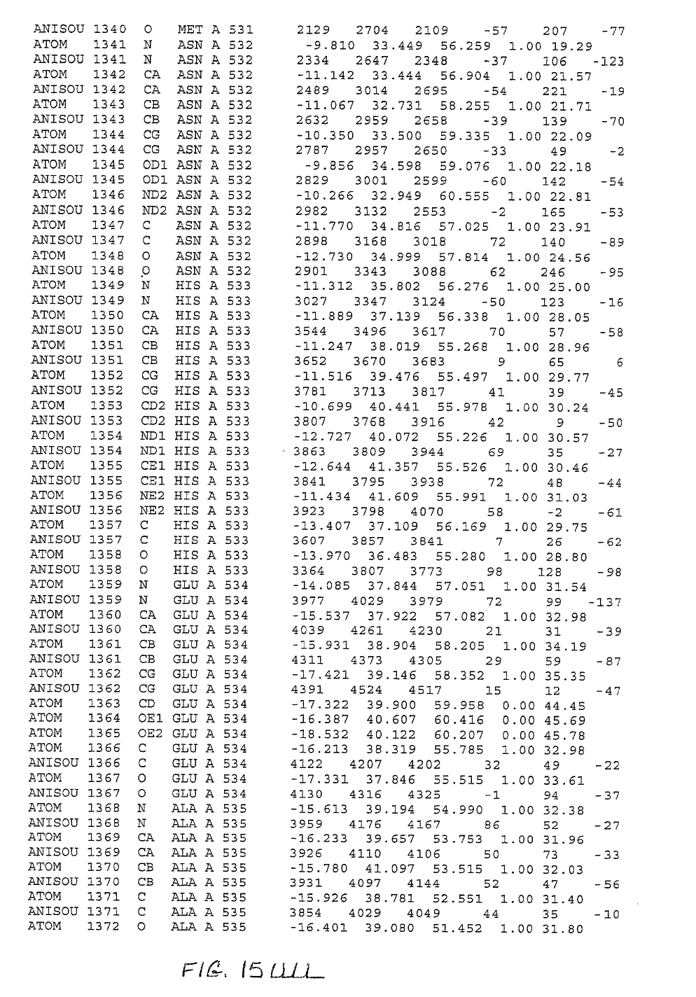

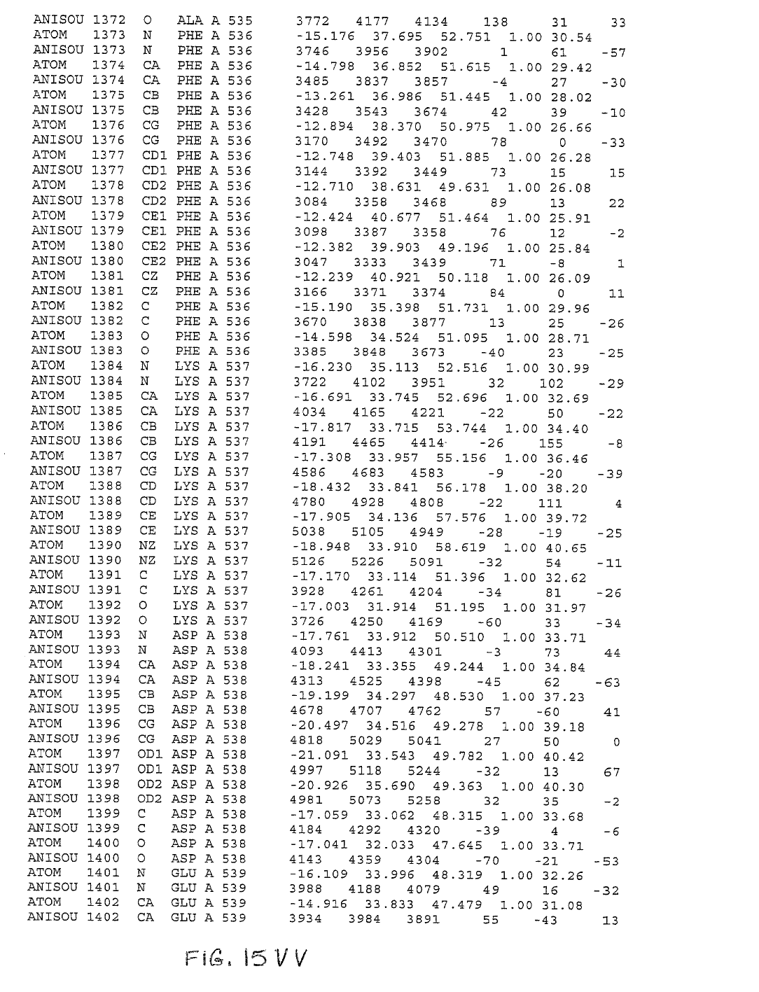

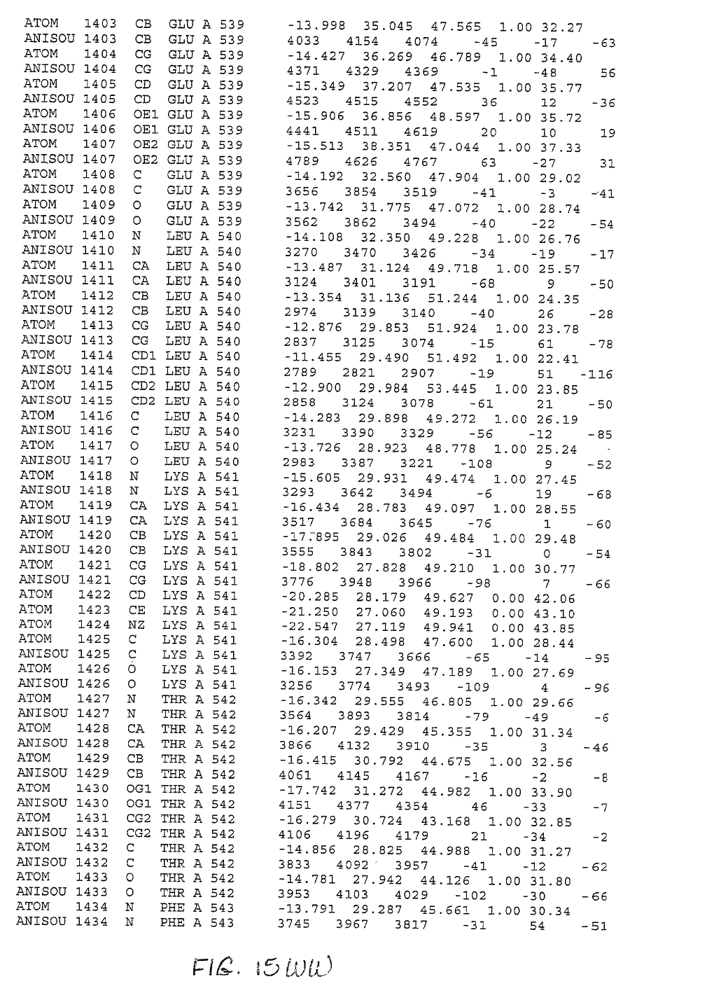

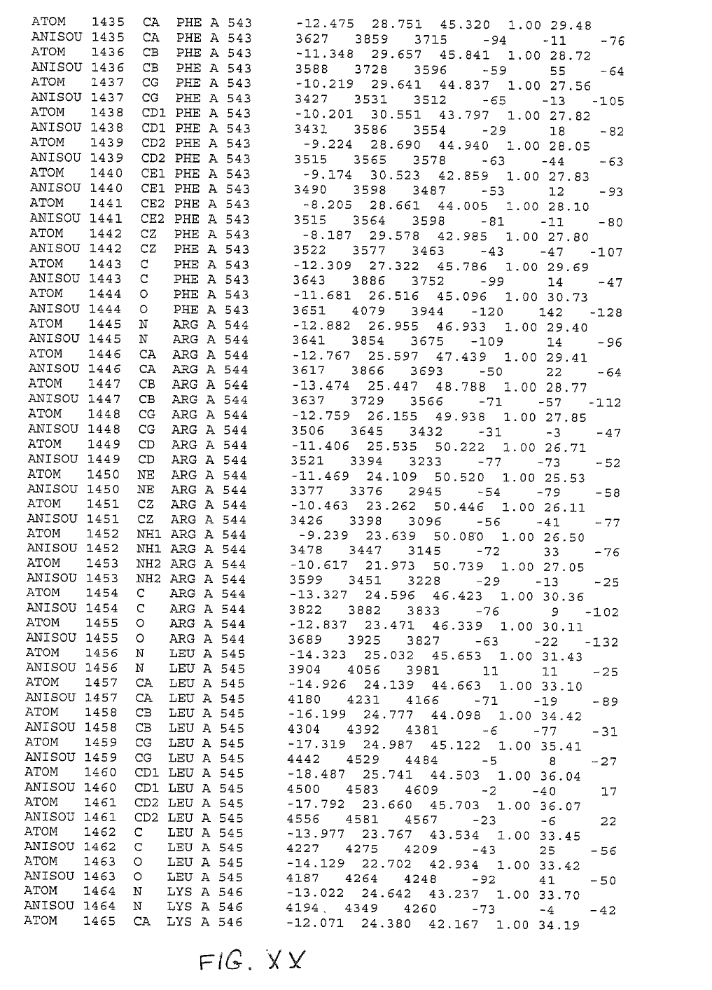

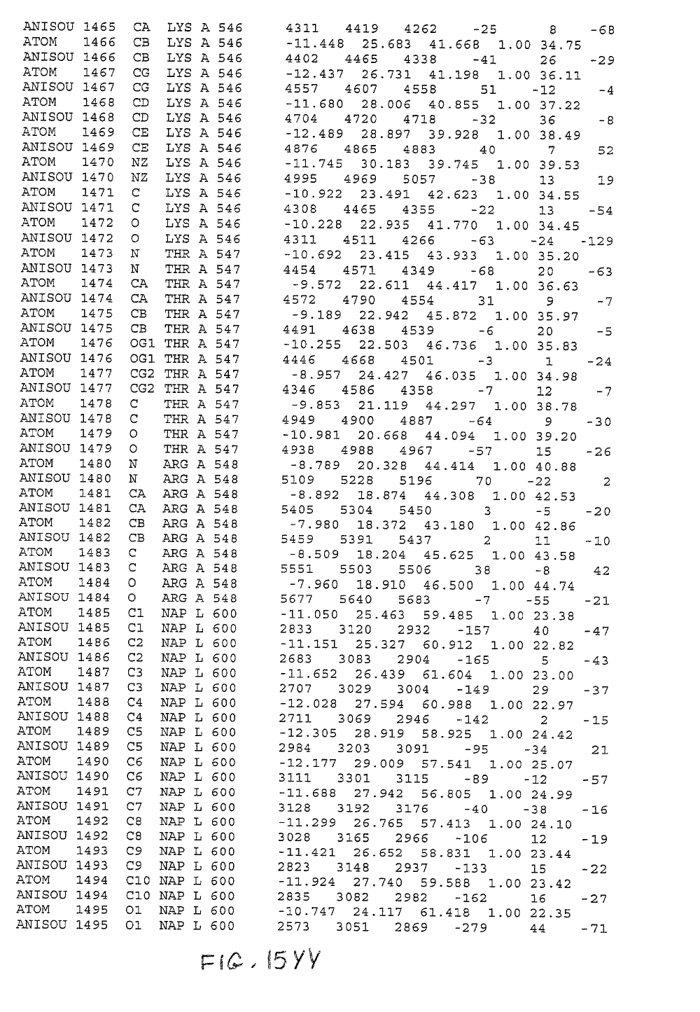

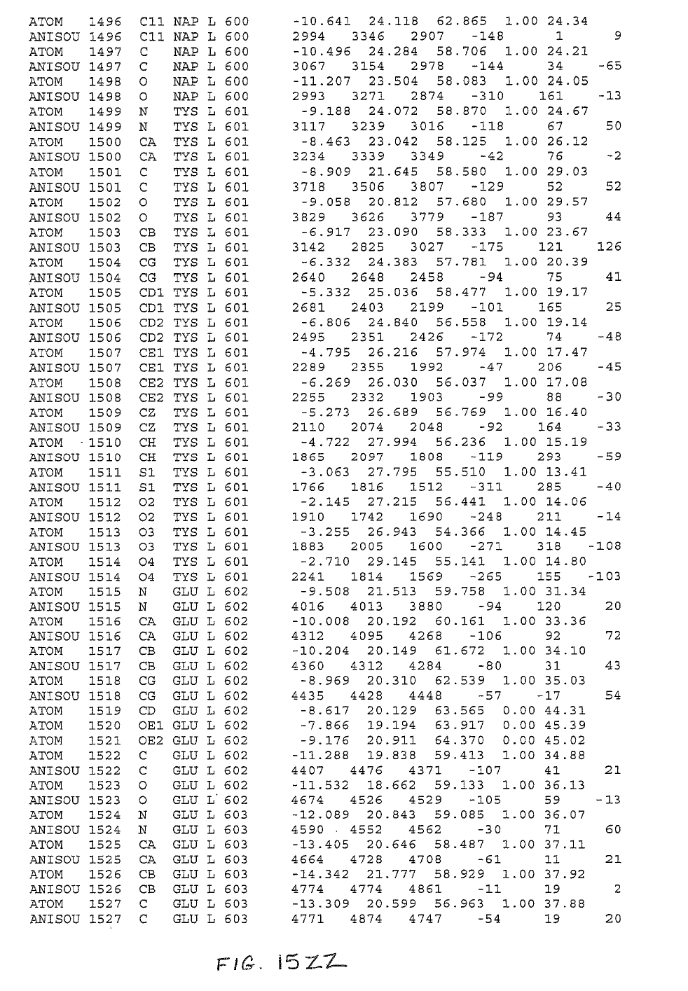

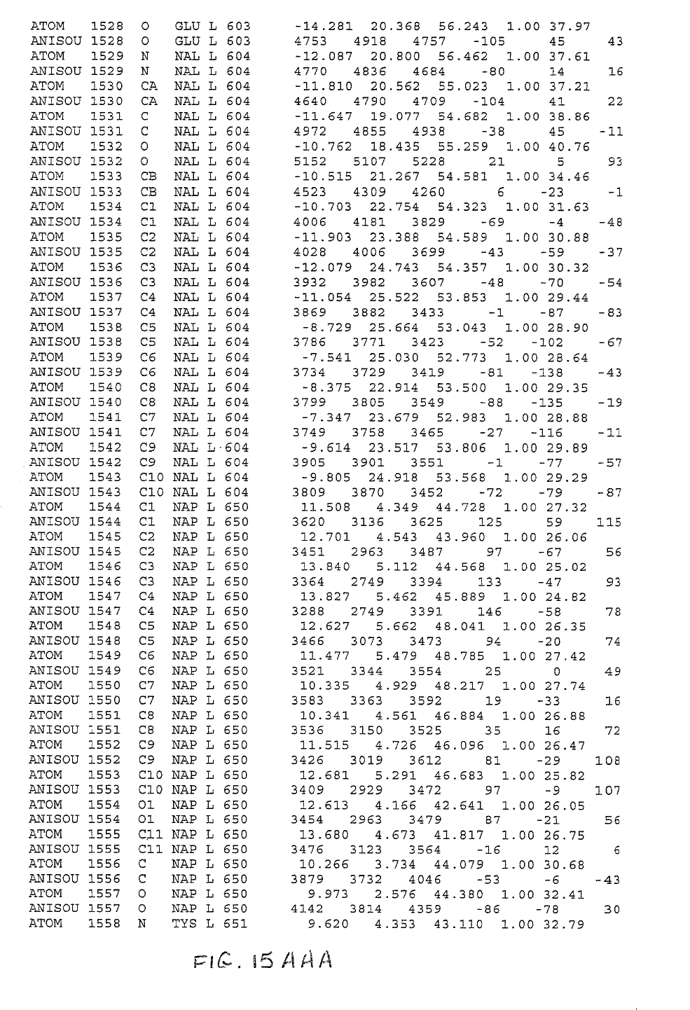

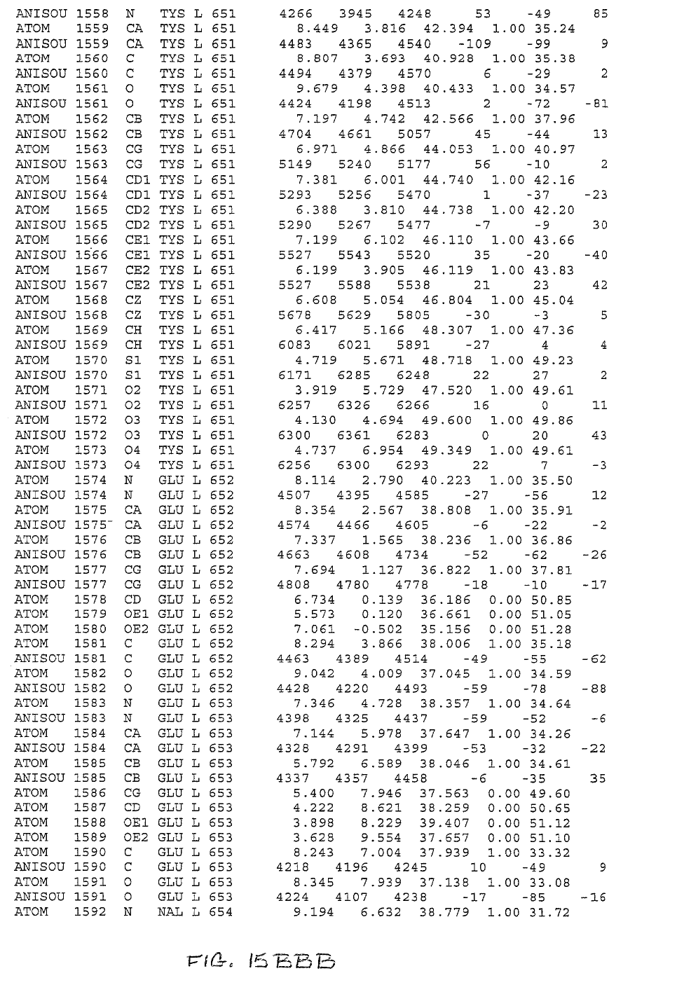

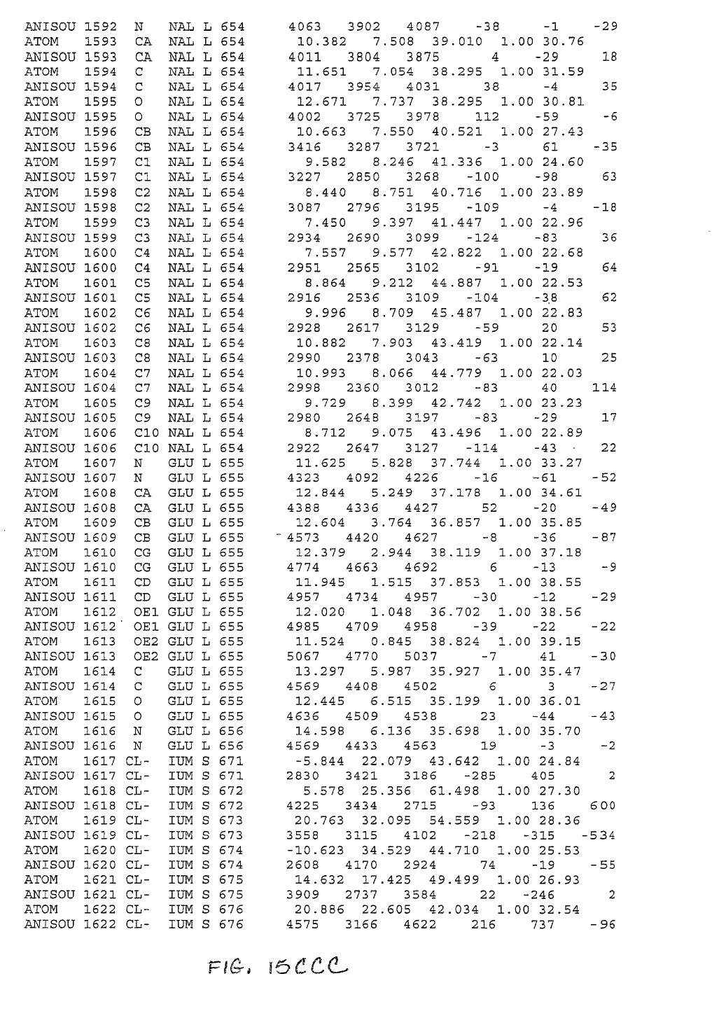

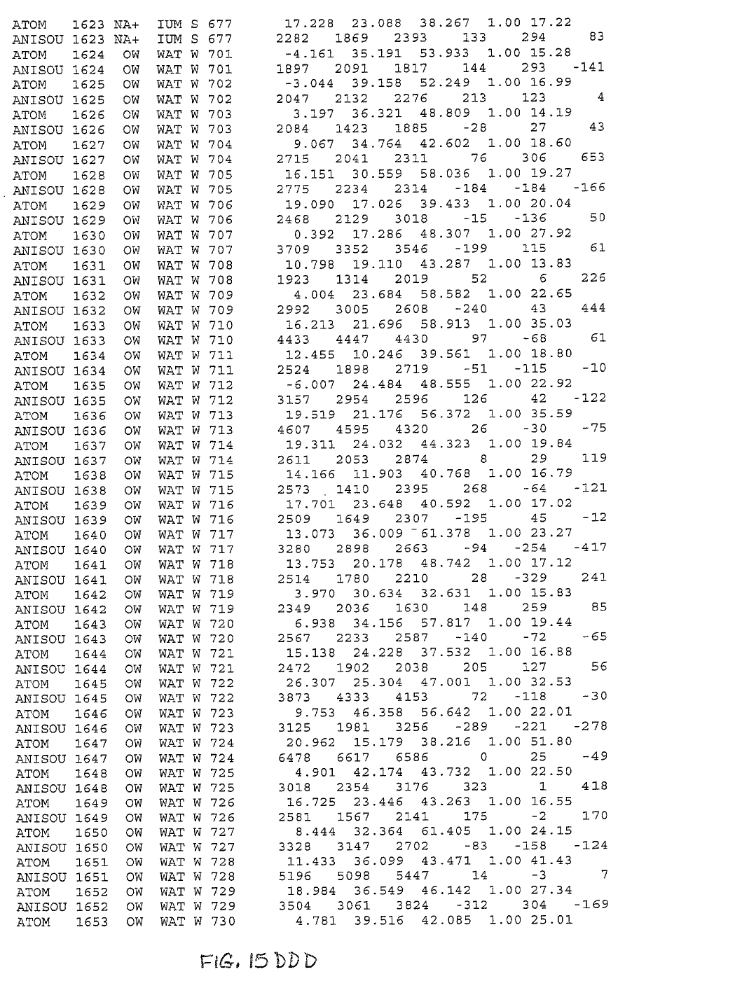

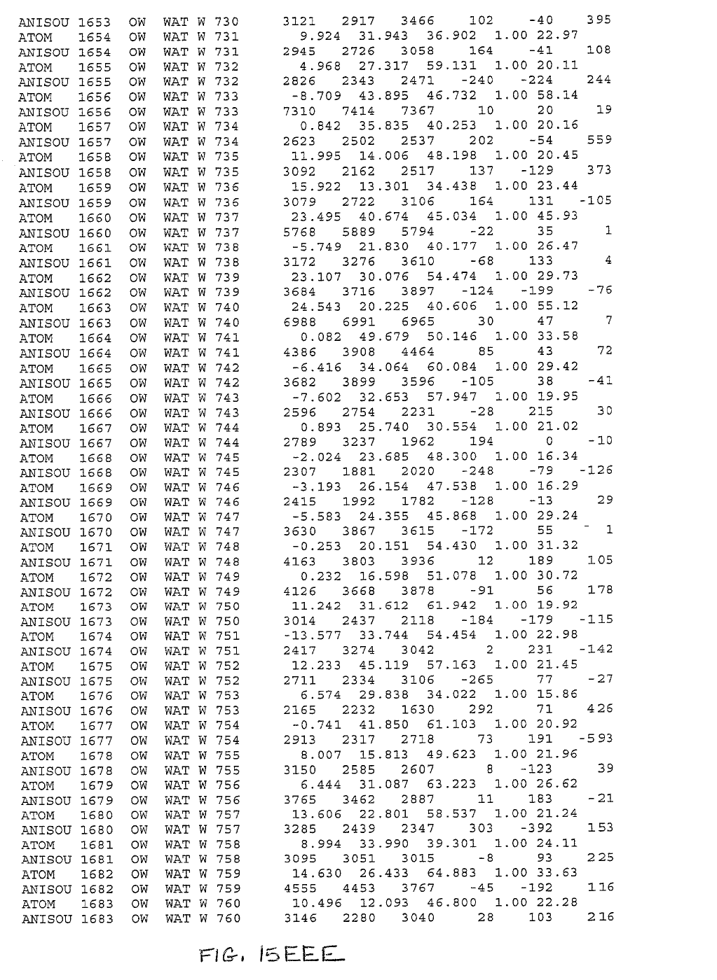

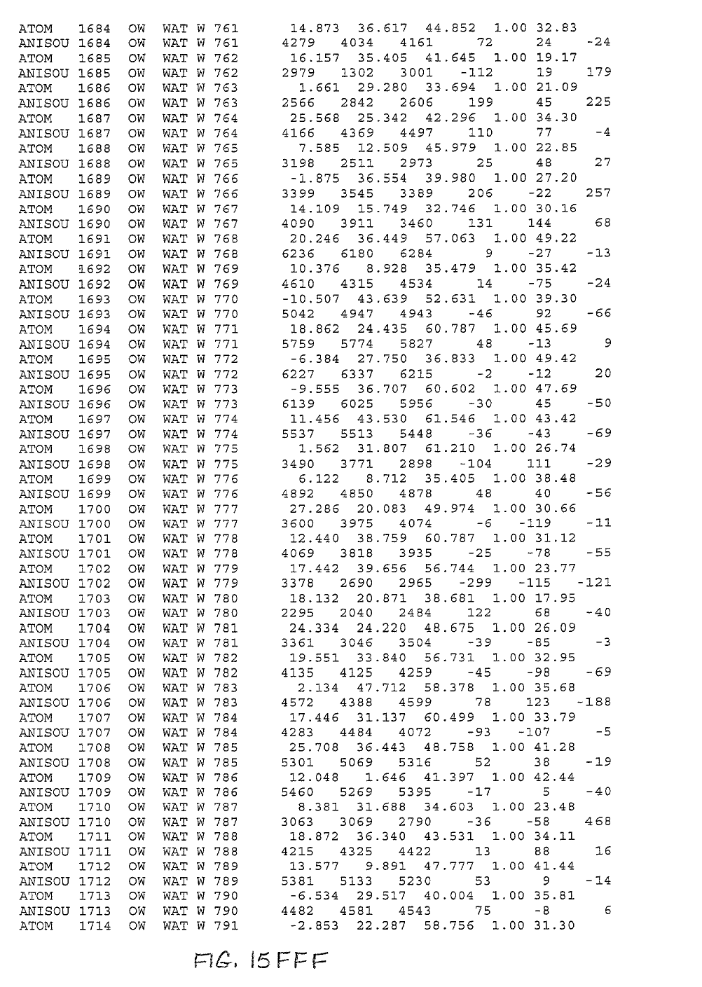

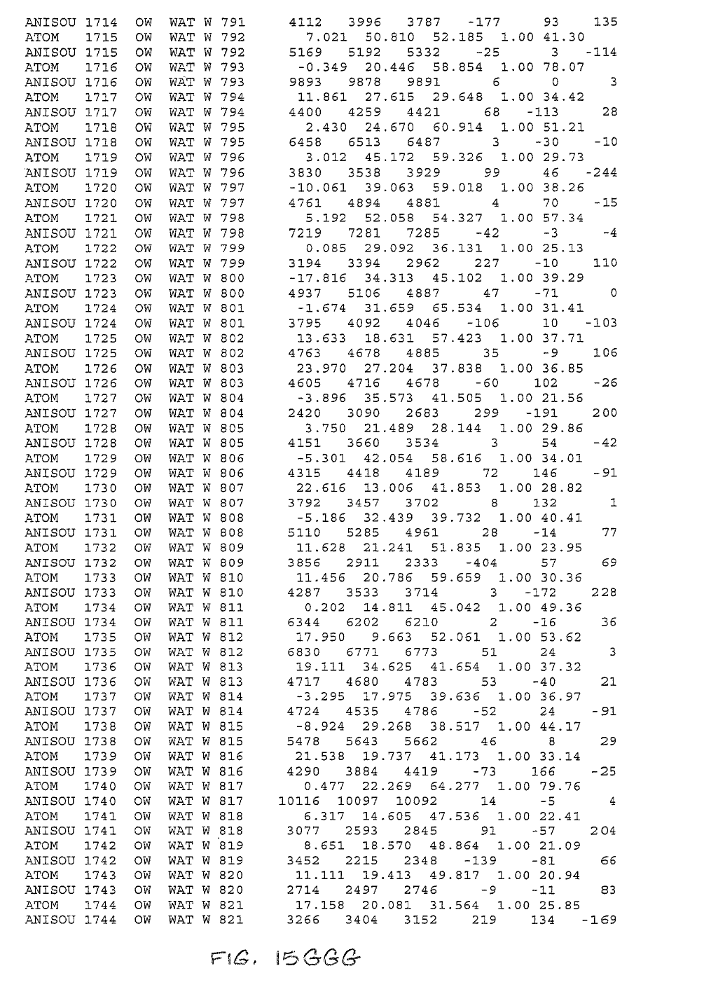

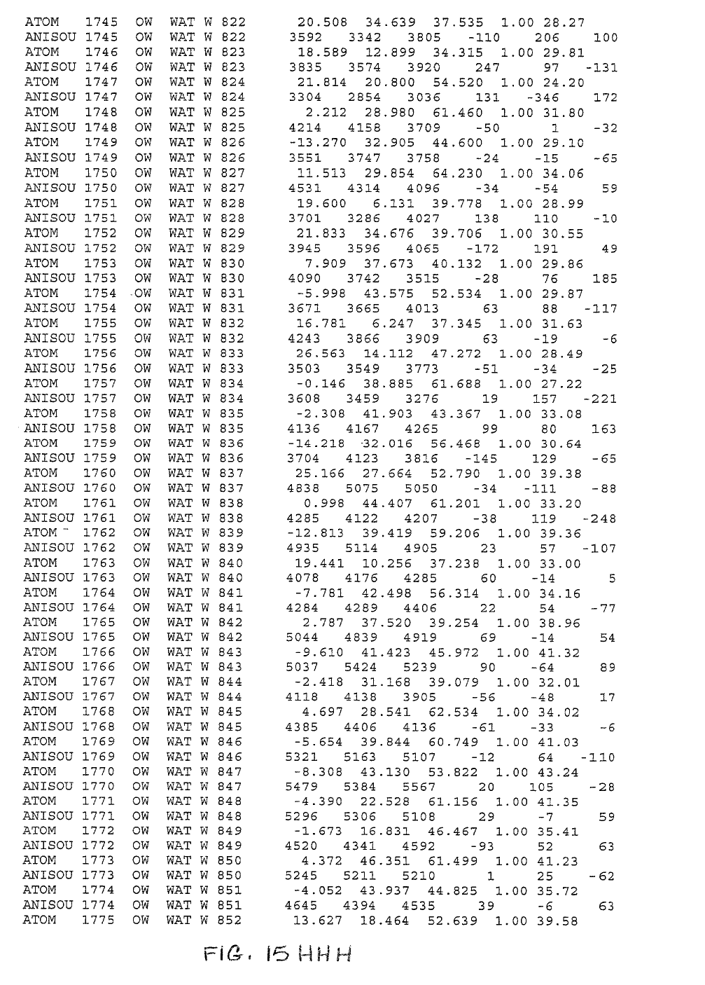

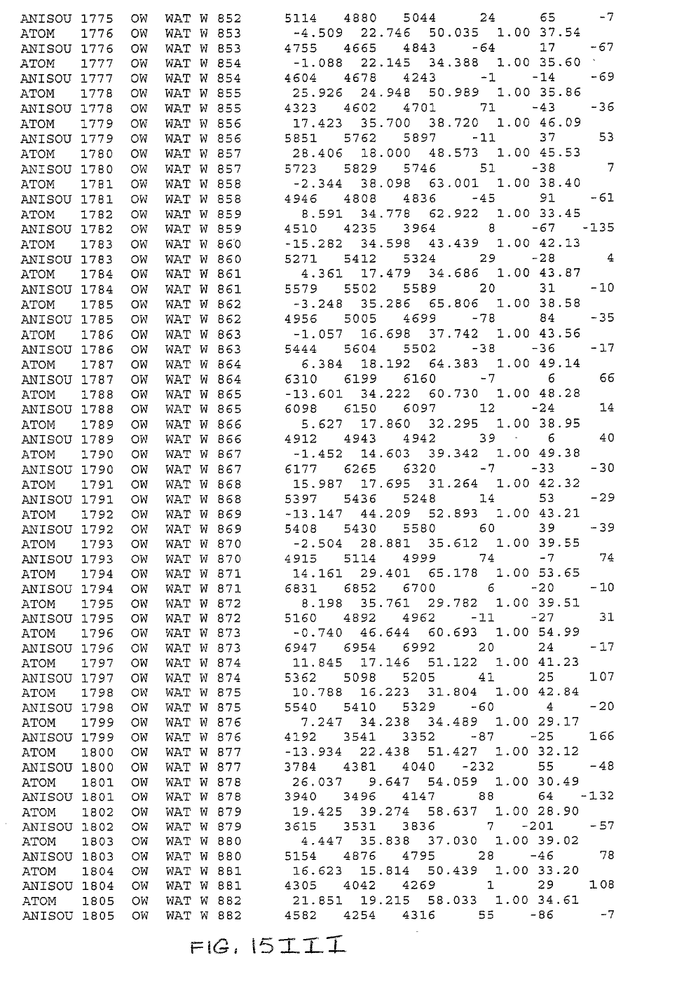

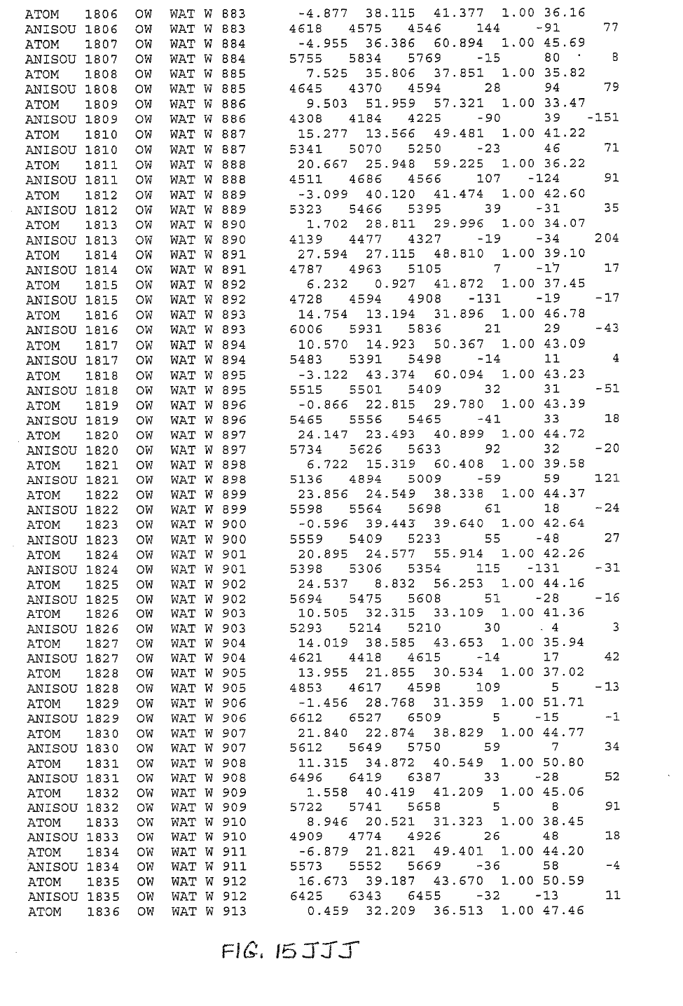

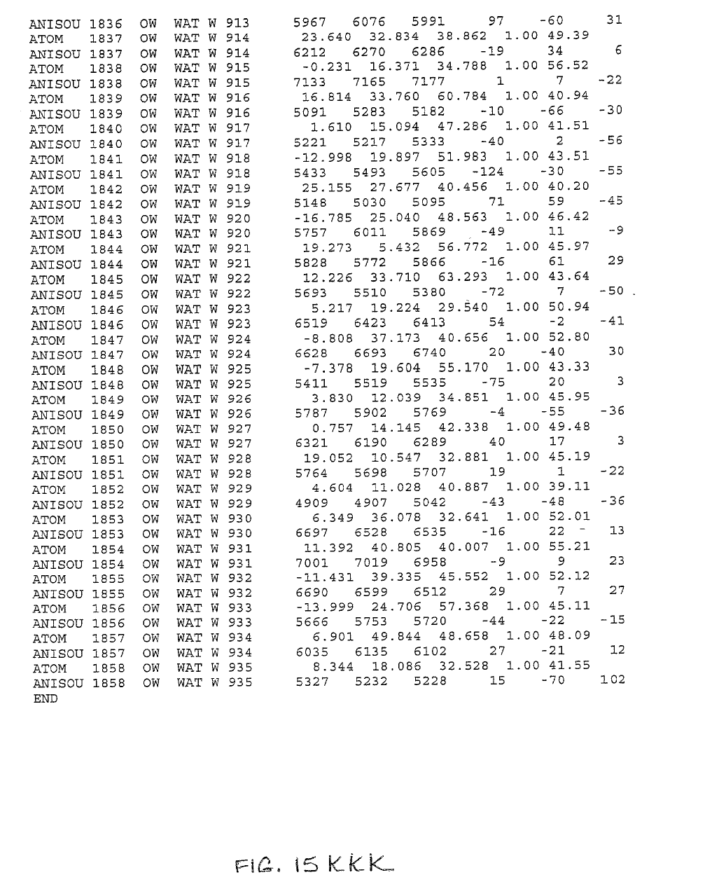

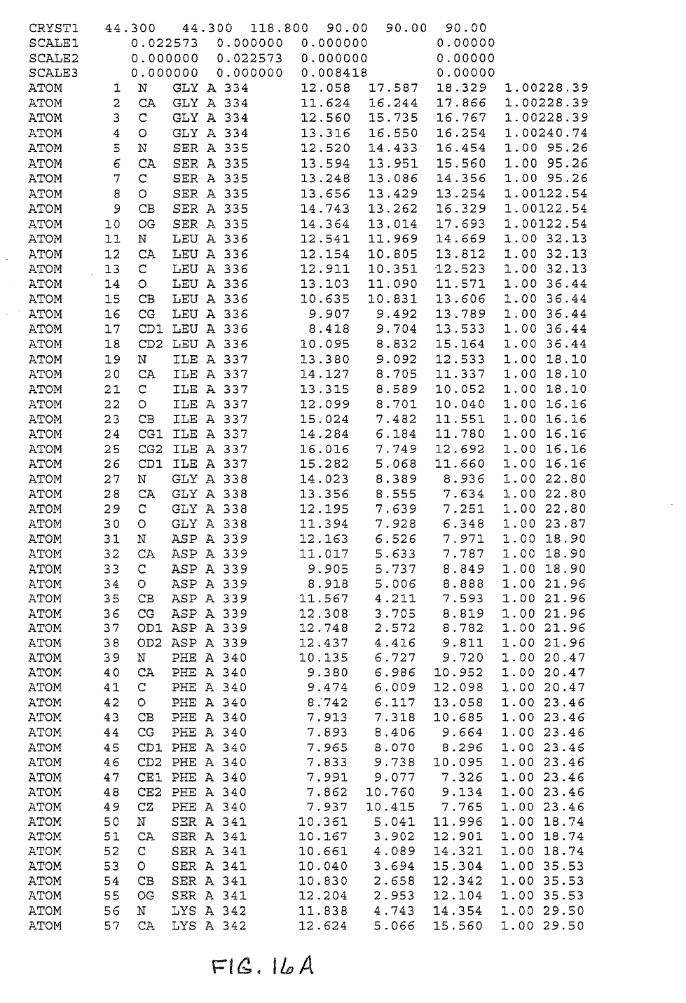

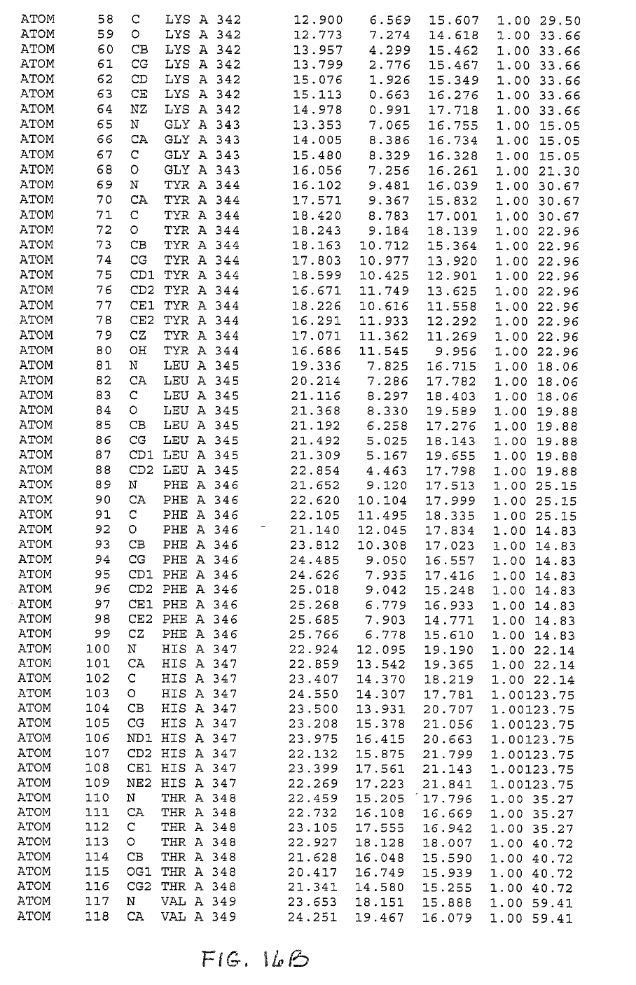

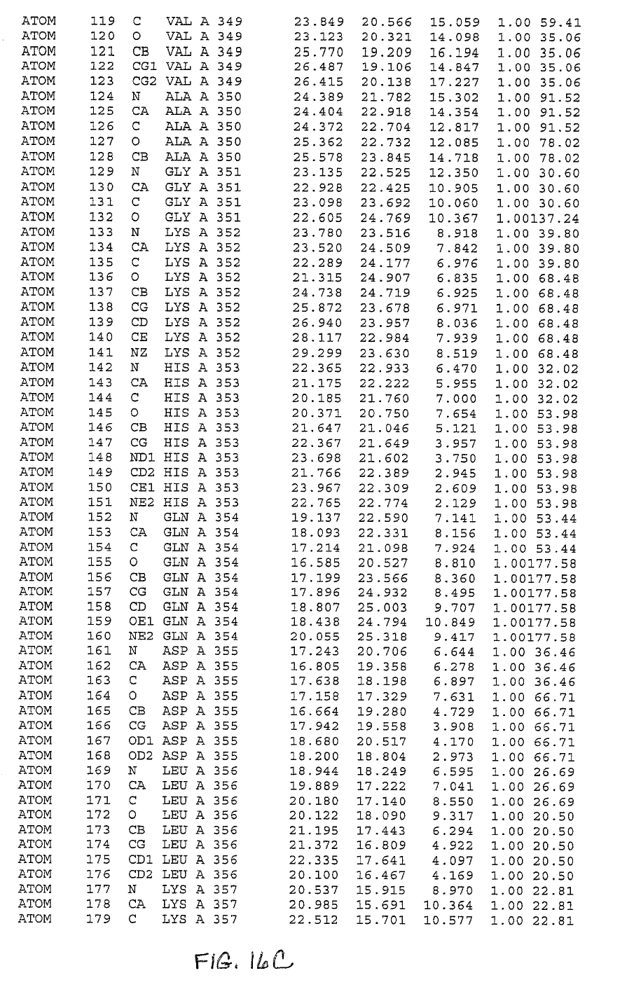

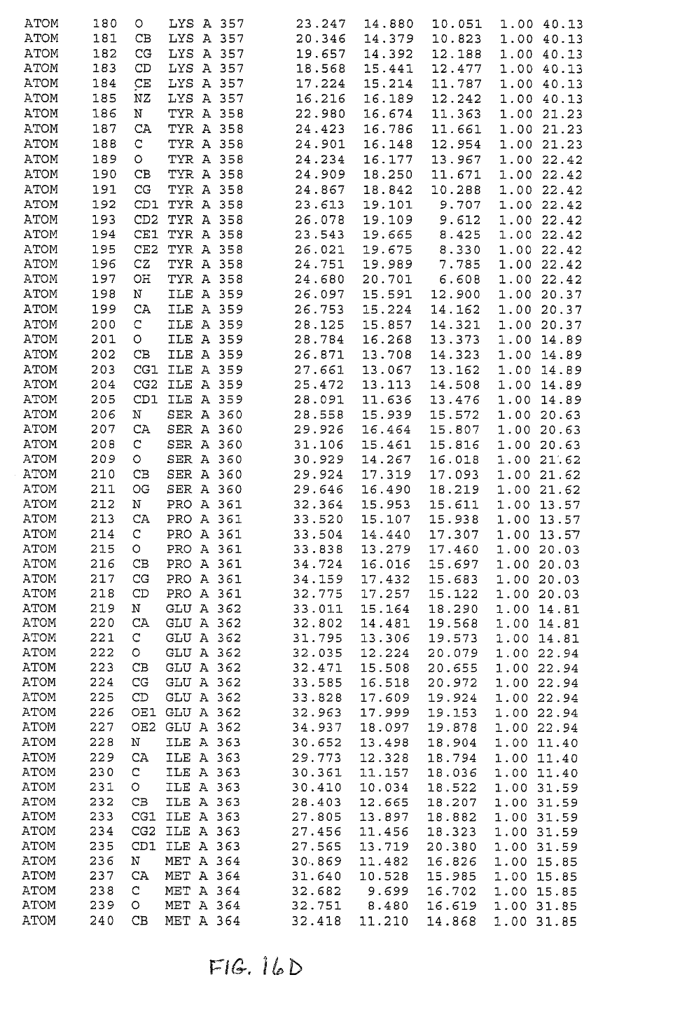

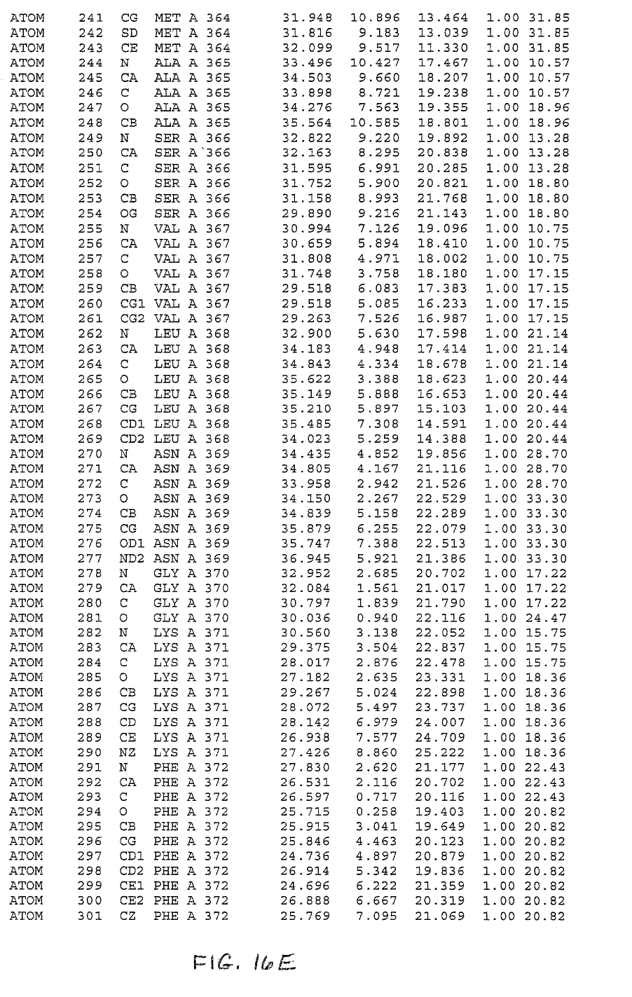

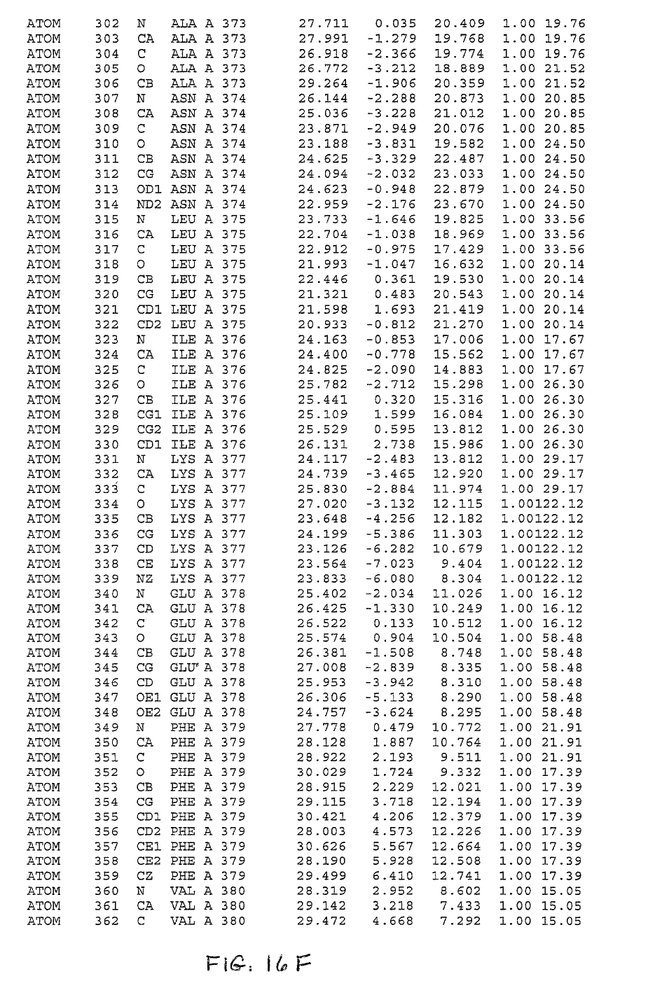

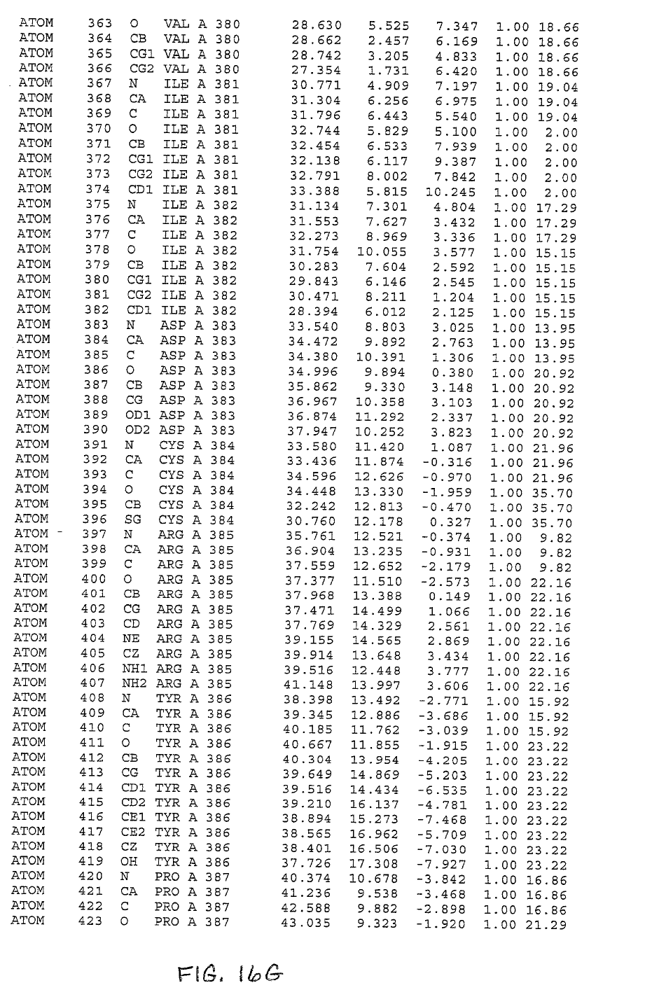

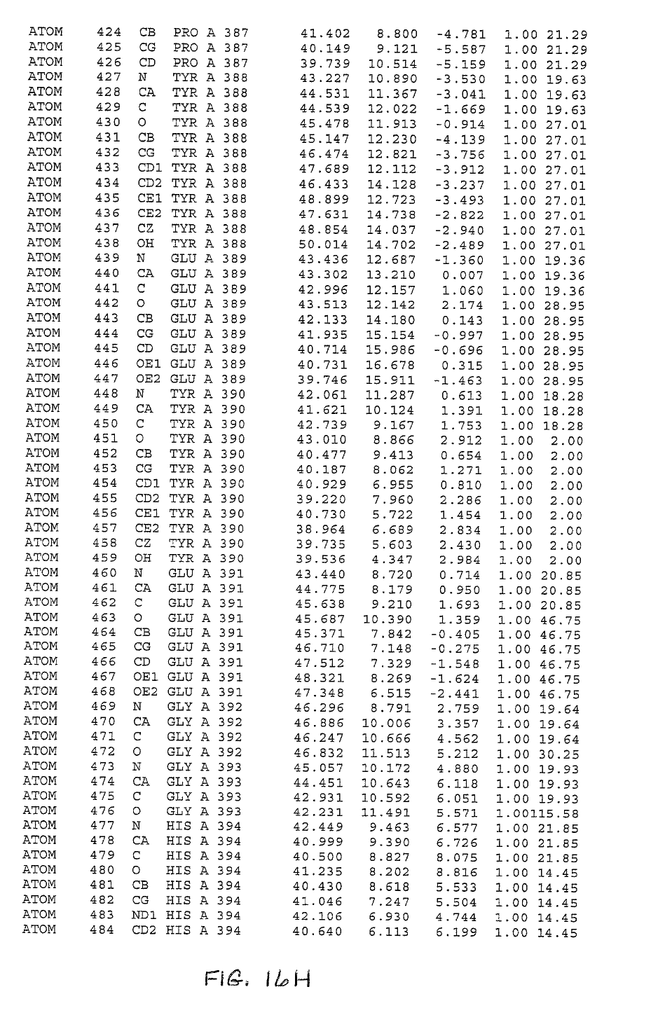

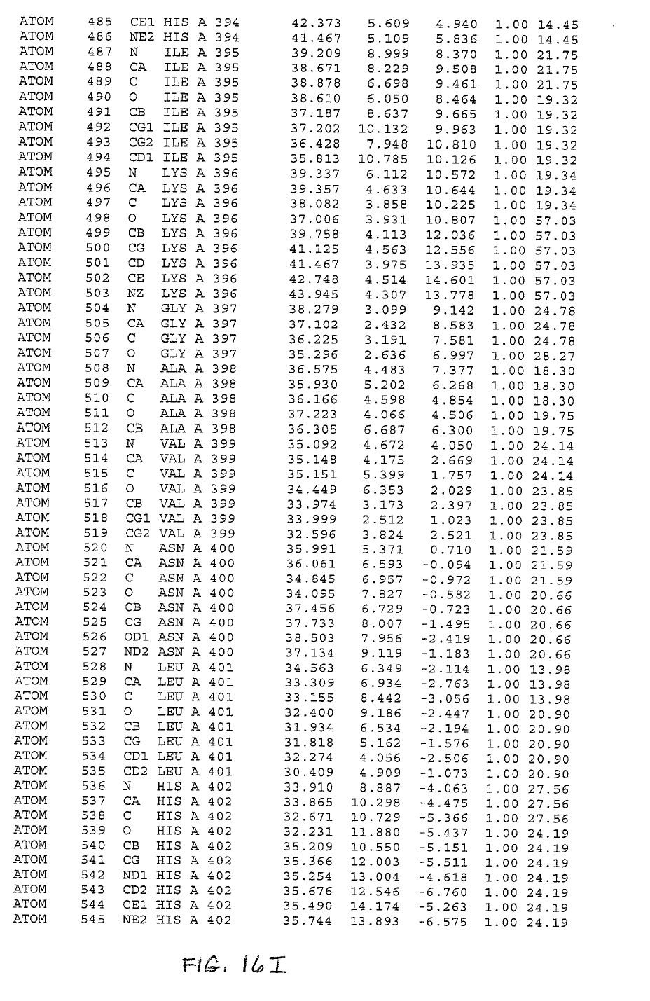

The diffraction data from crystal 1 described above were indexed in a tetragonal unit cell, space group P4₁ (or P4₃). In space groups P4₁ and P4₃, the direction of the polar c axis cannot be determined without reference to a molecular model for (part of) the unit cell contents. Accordingly, the data as initially indexed, “UP”, were reindexed, using the transformation h′=−k, k′=−h, l′=−l, to provide the “DOWN” indexing. Structure factors were calculated (Collaborative Computational Project, Number 4, 1994) in space group P4₁ using the partially-refined structural coordinates derived from crystals of Cdc25A(ΔN1A) (FIG. 16A to 16I) grown from ammonium sulfate as described below, which form in a similarly-sized tetragonal unit cell. These calculated structure factors were scaled against the “UP”-indexed data and the “DOWN”-indexed data. The “DOWN”-indexed data scaled substantially better than the “UP”-indexed data (R_{free} 39.1 vs. 52.4%, respectively), confirming that the correct indexing of the data was “DOWN”. Examination of SigmaA-weighted (Collaborative Computational Project, Number 4, 1994) 2Fₒ-F_{c} and Fₒ-F_{c} electron-density maps showed that the partially-refined structural coordinates for the Cdc25A(ΔN1A) molecule in the crystals grown from ammonium sulfate accounted for most of the structure of Cdc25A(ΔN1A) in the crystals grown from polyethylene glycol (‘PEG”).

Growth of Cdc25A(N1A) Crystals from Ammonium Sulfate

Cdc25A(N1A) was crystallized from 1.9-2.3 M (NH₄)₂SO₄, 50 mM sodium phosphate pH 6.5-7.0, 2 mM sodium tungstate at 4° C. Long square-based pyramidal crystals, with a slight tapering along their length, and often coming to a sharp point at the apex, grew over two weeks time. The crystals had unit cell dimensions a=b=44.17 Angstrom, c=118.65 Angstrom, and belonged to the tetragonal space group P4(1). There was one molecule of the protein in the crystallographic asymmetric unit. The phases required to obtain an interpretable electron density map were derived with 3 heavy atom derivatives of these crystals prepared by contacting the crystals with (1) KAu(CN)₂; (2) K₂PtCl₄; and (3) Thiomersal. Heavy atom phases were improved by solvent flattening. The atomic structure was refined using diffraction data to 2.1 Angstrom resolution (X-Plor). The R-factor was 26%, with an R(free) of 31%. The resulting atomic coordinates are presented in FIGS. 16A to 16I.

Preliminary Refinement of the PEG Cdc25A(ΔN1A) Crystal Structure (Crystal 1)

The partially-refined structural coordinates derived from crystals of Cdc25A(ΔN1A) grown from ammonium sulfate were refined against the “DOWN”-indexed data of the crystal described above using the program X-PLOR (Brünger, 1992). Rigid-body, Powell minimization, slowcool simulated annealing molecular dynamics, and temperature factor refinement resulted in an R of 31.7% (R_{free} 36.3%) for all reflections with /F/>2.0σ_{F} between 20 and 1.80 Å resolution. Examination of SigmaA-weighted 2Fₒ-F_{c} and Fₒ-F_{c} electron-density maps revealed that the active site loop (residues 431-434) was substantially disordered, and that no ligand (i.e. tungstate) was bound in the active site. Also, residues 493-523, at the C-terminus of the protein, were not located in the electron-density maps and were not included in the structural coordinates. These data are summarized in Table 10.

Soaking of a Cdc25A(ΔN1A) Crystal with cdc1316

A Cdc25A(ΔN1A) crystal grown as described above was transferred to 100 μL of a solution of 24% PEG 4000, 5 mM sodium-citrate, pH 5.6 at 4° C. After soaking for 2 min, the crystal was transferred to a fresh solution containing in addition 2 mM cdc1316. After 23 hrs, the crystal was transferred through a series of cryoprotective buffers containing 24% (w/v) PEG 4000, 10 mM sodium citrate, pH 5.6, 2 mM cdc1316, and 5, 10, and 20% (v/v) glycerol (5 min each). After an additional 5 min soak in the 20% glycerol buffer, the crystal was picked up with a fiber loop and flash-cooled by plunging into liquid nitrogen.

X-ray Diffraction Data Collection from a Cdc25A(ΔN1A) Crystal Soaked with cdc1316

A total of 235 data frames (0.4° each) were collected from a Cdc25A(ΔN1A) crystal soaked with cdc1316 as described above (crystal 2). The crystal was maintained at a temperature of 100 K during data collection. The diffraction data were processed and reduced to structure factor amplitudes, as described for crystal 1, in space group P4₁, a=43.69 Å, c=117.27 Å. The unique reflections were assigned to the same “free” and “working” sets as used for crystal 1. These data are summarized in Table 10. Additional data were collected from this crystal at the National Synchrotron Light Source (NSLS; beamline X25, λ=1.100 Å, Brandeis B4 CCD detector). The crystal was maintained at a temperature of 100 K with an Oxford Cryosystems Cryostream cooler during data collection. The diffraction data were processed and reduced to structure factor amplitudes, as described above. Only a small fraction of the unique data were collected due to instrumental limitations. These data show, however, that the crystals diffract x-rays to 1.35 Å resolution. These additional data are also summarized in Table 10.

Further Refinement of the PEG Cdc25A(ΔN1A) Crystal Structure

The partially-refined structural coordinates for crystal 1 were further refined against the diffraction data collected from the Cdc25A(ΔN1A) crystal 2 using X-PLOR. Refinement alternated with manual rebuilding of the structural coordinates (the “model”) using the molecular graphics program O (Jones et al., 1991). Rigid-body, Powell minimization, slowcool simulated annealing molecular dynamics, and individual temperature factor refinement resulted in an R of 29.9% (R_{free} 32.6%) for all reflections with /F/>1.5σ_{F} between 20 and 1.80 Å resolution. The model was rebuilt aided by inspection of simulated annealing omit maps. Several more rounds of rebuilding and refinement brought the R to 27.0% (R_{free} 28.9%; /F/>1.5σ_{F}, 20-1.80 Å). Additional refinement with REFMAC (Murshudov et al., 1997) brought the R to 23.0% (R_{free} 24.3%; /F/>0.0σ_{F}, 20-1.80 Å). This model (“Refmac1”) includes Cdc25A residues 335-413, 419-431, and 435-492 and 74 water molecules. No interpretable electron-density was present for either the rest of the active site loop (residues 432-434) or the ligands tungstate or cdc1316. Also, residues 493-523, at the C-terminus of the protein, were not located in the electron-density maps and were not included in the structural coordinates. Weak, but not readily interpretable density was present for residues 414-418. These data are summarized in Table 10.

Crystallization of Cdc25A(ΔN8A)

Frozen Cdc25A (ΔN8A construct; 17 mg/ml in 50 mM Tris.HCl, pH 7.5, 50 mM NaCl, 1 mM DTT, 1 mM EDTA; 150 μL) was thawed and mixed with 1.5 μL DTT (1 M), 0.3 μL NaN₃ (1.5 M), and 1.5 μL Na₂WO₄ (100 mM). This protein solution (1 μL) was mixed with 1 μL of a reservoir solution consisting of 20% (w/v) PEG 3000, 600 mM Li₂SO₄, 100 mM sodium citrate, pH 5.6, and suspended over the reservoir on the underside of a siliconized glass cover slip at 4° C. Long, pyramidal crystals appeared in about 2 weeks. Crystals also grew under these conditions in the presence of varying amounts of PEG 3000 or Li₂SO₄, in the presence of other salts instead of Li₂SO₄ (e.g. ammonium acetate or ammonium sulfate), in the absence of PEG 3000 entirely, and at pH values from 5.6 to 5.8.

Cryoprotection of a Cdc25A(ΔN8A) Crystal

A Cdc25A(ΔN8A) crystal (crystal 3) grown as described above was transferred into a series of cryoprotective buffers containing 20% (w/v) PEG 3000, 100 mM sodium citrate, pH 5.6, and 0 and 5% (v/v) glycerol, and same containing 10% glycerol and 25% or 30% PEG 3000. The crystal was soaked sequentially in the 0% glycerol, 5% glycerol, 25% PEG 3000/10% glycerol, and 30% PEG 3000/10% glycerol buffers for five sec each. The crystal was picked up with a fiber loop and flash-cooled by plunging into liquid nitrogen. The crystal was stored in a liquid nitrogen refrigerator.

X-ray Diffraction Data Collection from a Cdc25A(ΔN8A) Crystal

A total of 225 data frames (0.4° each) were collected from crystal 3 as described above. The crystal was maintained at a temperature of 100 K during data collection. The diffraction data were processed and reduced to structure factor amplitudes, as described above in space group P4₁, a=43.94 Å, c=117.39 Å. The unique reflections were assigned to the same “free” and “working” sets as used for crystal 1.

Annealing of a Cdc25A(ΔN8A) Crystal

Crystal 3 was thawed after x-ray data collection in a cryoprotective buffer containing 30% (w/v) PEG 3000, 100 mM sodium citrate, pH 5.6, and 10% (v/v) glycerol at 4° C. After 15 min, the crystal was picked up with a fiber loop and flash-cooled again by plunging into liquid nitrogen.

X-ray Diffraction Data Collection from the Annealed Cdc25A(ΔN8A) Crystal

A total of 180 data frames (0.5° each) were collected from the annealed crystal 3 using the method described above. The crystal was maintained at a temperature of 100 K during data collection. The diffraction data were processed, merged with the data collected as described above, and reduced to structure factor amplitudes in space group P4₁, a=43.92 Å, c=117.38 Å. These annealed data extended to higher resolution than the pre-annealed data (2.15 vs. 2.40 Å). The annealed crystal also had a lower mosaic spread (0.3° vs. 0.6°). The unique reflections were assigned to the same “free” and “working” sets as used for crystal 1. These final crystal 3 data are summarized in Table 10.

Refinement of the Cdc25A(ΔN8A) Crystal Structure

The refinement of the Cdc25A(ΔN8A) crystal structure (crystal 3) began with the intermediate “Powell8” structural coordinates for Cdc25A(ΔN1A), described above, from which water molecules had been removed. Rigid-body, Powell minimization, and overall and individual temperature factor refinement resulted in an R of 27.1% (R_{free} 29.2%) for all reflections with /F/>2.0σ_{F} between 20 and 2.15 Å resolution. Examination of SigmaA-weighted 2Fₒ-F_{c} and Fₒ-F_{c} electron-density maps revealed that the active site loop (residues 431-434) was partially ordered, in a conformation different from that observed in other phosphatases. Further refinement with REFMAC (Murshudov et al., 1997) brought the R to 22.8% (R_{free} 27.1%; /F/>0.0σ_{F}, 20-2.15 Å). This model (“Refmac1”) includes Cdc25A residues 335-413 and 419-492, and 67 water molecules. No interpretable electron-density was present for the ligand tungstate. Weak, but not readily interpretable density was present for residues 414-418. Active site loop residues 431-433 were built as alanines. Tungstate, residues preceding 335, and residues 493-523 were not located in the electron-density maps and were not included in the structural coordinates. These data are summarized in Table 10.

Soaking of a Cdc25A(ΔN8A) with Na₂WO₄ and X-ray Diffraction Data Collection

Crystal 3 was annealed for three days at 4° C. in a buffer supplemented with 10 mM Na₂WO₄. The crystal was flash-cooled in liquid nitrogen. A total of 40 data frames (1.0° each) were collected from the crystal (now crystal 4) as described above. The crystal was maintained at a temperature of 100 K during data collection. The diffraction data were processed and reduced to structure factor amplitudes, as described above, in space group P4₁, a=43.98 Å, c=117.72 Å. These soaked data were much weaker than those of crystal 3 (3.00 vs. 2.15 Å). The unique reflections were assigned to the same “free” and “working sets” as used for crystal 1. These data are summarized in Table 10.

Refinement of the Na₂WO₄-Soaked Cdc25A(ΔN8A) Crystal Structure

Refinement of the Na₂WO₄-soaked Cdc25A(ΔN8A) crystal structure (crystal 4) began with the “Powell8” structural coordinates for crystal 1 in which water molecules had been removed. Rigid-body, Powell minimization, and temperature factor refinement resulted in an R of 22.7% (R_{free} 25.7%) for all reflections with /F/>2.0σ_{F} between 20 and 3.00 Å resolution. Examination of SigmaA-weighted 2Fₒ-F_{c} and Fₒ-F_{c} electron-density maps showed that tungstate was not present in the active site. The model was not further refined. These data are summarized in Table 10.

Crystallization of the Cdc25B(ΔN1B).cdc1249 Inhibitor Complex

Frozen Cdc25B (ΔN1B construct; 17.5 mg/ml in 10 mM sodium phosphate, pH 6.7, 50 mM NaCl, 10 mM DTT, 5 mM EDTA; 212 μL) was thawed and mixed with 0.4 μL NaN₃ (1.5 M) and 7.4 μL cdc1249 (30 mM in 25 mM sodium HEPES, pH 7.5). This protein solution was aged for 4 days at 1° C. A slight precipitate that formed was removed by centrifugation. The supernatant (1 μL) was mixed with 1 μL of a reservoir solution consisting of 4.4 M NaCl, 50 mM sodium HEPES, pH 7.0, and suspended over the reservoir on the underside of a siliconized glass cover slip at 4° C. Block-like crystals appeared within 2-7 days. Crystals also grew under these conditions in the presence of varying amounts of NaCl, and at pH values from 5.75 to 8.0.

Crystallization of the Cdc25B(ΔN8B).cdc1249 Inhibitor Complex

Frozen Cdc25B (ΔN8B construct; 27.5 mg/ml in 50 mM Tris.HCl, pH 7.5, 50 mM NaCl, 1 mM DTT, 1 mM EDTA; 25 μL) was thawed and mixed with 0.25 μL NaN₃ (0.3 M), 0.2 μL DTT (1 M), and 0.85 μL cdc1249 (30 mM in 25 mM sodium HEPES, pH 7.5). This protein solution (2 μL) was mixed with 2 μL of a reservoir solution consisting of 82.5% saturated NaCl (˜4.5 M), 50 mM sodium MES, pH 6.5, and suspended over the reservoir on the underside of a siliconized glass cover slip at 4° C. Block-like crystals appeared in one day.

The Cdc25B(ΔN8B-c17).cdc1249, Cdc25B(66 N8B-c18).cdc1249, Cdc25B(ΔN1B).cdc 1671, Cdc25B(ΔN1B).cdc1885, Cdc25B(ΔN8B).cdc1659, and Cdc25B(ΔN8B).cdc1671 complexes were also crystallized using this general procedure.

Cryoprotection of Cdc25B(ΔN1B), Cdc25B(ΔN8B), Cdc25B(ΔN8B-c17), and Cdc25B(ΔN8B-c18) Inhibitor Complex Crystals

A Cdc25B inhibitor complex crystal grown as described above was transferred into a series of cryoprotective buffers containing 4.5 M NaCl, 50 mM sodium MES, pH 6.5 or 50 mM sodium HEPES, pH 7.0, and 0, 5, 10, and 16.5% (v/v) glycerol. The crystal was soaked sequentially in the 0 and 5% glycerol buffers, and then the 10 and 16.5% glycerol buffers (each of which also contained 0.5-2.0 mM of the appropriate inhibitor), for 5 min each. The crystal was picked up with a fiber loop and flash-cooled by plunging into liquid nitrogen. The crystal was stored in a liquid nitrogen refrigerator.

X-ray Diffraction Data Collection from a Cdc25B(ΔN1B).cdc1249 Inhibitor Complex Crystal

A total of 359 data frames (0.25° each) were collected from a Cdc25B(ΔN1B).cdc1249 inhibitor complex crystal (crystal 5) using the equipment described above. The crystal was maintained at a temperature of 100 K during data collection. The diffraction data were processed and reduced to structure factor amplitudes, as described above, in space group P4₁2₁2 or P4₃2₁2, a=70.15 Å, c=130.35 Å. Five percent of the unique reflections were assigned, in a random fashion, to the “free” set, for calculation of the free R-factor (R_{free}); the remaining 95% of the reflections constituted the “working set”, for calculation of the R-factor (R). These data are summarized in Table 10.

Molecular Replacement Solution of the Cdc25B(ΔN1B).cdc1249 Inhibitor Complex Crystal Structure

A cross-rotation function was calculated with the Cdc25B(ΔN1B).cdc 1249 inhibitor complex crystal data described above, using the program AMORE (Navaza, 1994). The search model was the partially-refined structural coordinates of Cdc25A(ΔN1A) (crystal 1). The cross-rotation function had one obvious solution, at Eulerian angles [27.66, 63.02, 94.53], which was 11.3 standard deviations above the mean level of the cross-rotation function; the next highest peak was 6.9 standard deviations above the mean. The translation function was calculated (AMORE) in space groups P4₁22, P4₃22, P4₁2₁2, and P4₃2₁2. One solution was obvious, in space group P4₃2₁2, with an R-factor of 49.3%, and a correlation coefficient of 31.4% (15-3.0 Å resolution).

Synchrotron X-ray Diffraction Data Collection from the Cdc25B(ΔN1B).cdc1249 Inhibitor Complex Crystal

A total of 35 data frames (1.0° each) were collected from crystal 5 at the National Synchrotron Light Source (NSLS; beamline X25, λ=1.100 Å, Brandeis B4 CCD detector). The crystal was maintained at a temperature of 100 K with an Oxford Cryosystems Cryostream cooler during data collection. The diffraction data were processed and reduced to structure factor amplitudes, as described above, in space group P4₃2₁2, a=70.15 Å, c=130.35 Å. The unique reflections were assigned to the same “free” and “working” sets as used for the laboratory data for crystal 5. These data are summarized in Table 10.

Refinement of the Cdc25B(ΔN1B).cdc1249 Inhibitor Complex Crystal Structure

The partially-refined structural coordinates of Cdc25A(ΔN1A) (crystal 1) were refined against the Cdc25B(ΔN1B).cdc1249 inhibitor complex crystal data using X-PLOR. Refinement alternated with manual rebuilding of the model using the molecular graphics program O. The molecular replacement model was modified by changing many of the amino acid side chains that differed between Cdc25A and Cdc25B to the Cdc25B amino acids. These side chains were located in clear electron-density. Unclear amino acids were truncated as alanine. Bulk solvent correction (˜60% solvent by volume in these crystals), Powell minimization, overall and then group temperature factor refinement, and manual rebuilding reduced the R to 37.7% (R_{free} 43.9%) for all reflections with /F/>1.5σ_{F} between 20 and 2.40 Å resolution. Torsion angle molecular dynamics improved the electron-density maps such that two missing loops (residues 456-464 and 474-477, the active site loop) could be built into very clear density. Clear density was also present at this stage of the refinement for much of cdc1249 bound in the active site. Continued refinement (including individual temperature factors) and rebuilding allowed the addition of residues 533-548 (the C-terminal α-helix), the first 5 (of 6 total) residues for cdc1249, and several water molecules (R 27.0%, R_{free} 31.2%). Further refinement including slowcool simulated annealing molecular dynamics revealed the presence of another molecule of cdc1249 bound, not at the active site, but between two Cdc25B(ΔN1B) molecules related by crystallographic symmetry. Addition of this second molecule of cdc1249, several water molecules, a Na⁺ ion, and several Cl⁻ ions followed by more refinement reduced the R to 22.8% (R_{free} 26.1%). The model was rebuilt aided by inspection of simulated annealing omit maps. Several rounds of refinement brought the R to 20.3% (R_{free} 22.9%; /F/>0.5σ_{F}, 20-2.30 Å). These structural coordinates (“Powell14”) for the Cdc25B(ΔN1B).cdc1249 inhibitor complex consist of Cdc25B residues 377-548, two cdc1249 molecules, 129 water molecules, one Na⁺ ion, and five Cl⁻ ions. At this point the synchrotron data described above became available. Further refinement with REFMAC, which included the addition of several side chains in alternate conformations, resulted in an R of 21.8% (R_{free} 24.4%; /F/>0.0σ_{F}, 20-1.95 Å). These final structural coordinates (“Refmac1”) for the Cdc25B(ΔN1B).cdc1249 inhibitor complex consist of Cdc25B residues 377-548, two cdc1249 molecules, 158 water molecules, one Na⁺ ion, and five Cl⁻ions. These data are summarized in Table 10.

X-ray Diffraction Data Collection from a Cdc25B(ΔN1B).cdc1249 Inhibitor Complex Crystal Soaked with cdc1316

A Cdc25B(ΔN1B).cdc1249 inhibitor complex crystal prepared as described above was transferred to 50 μL of a buffer containing 4.5 M NaCl, 50 mM sodium HEPES, pH 7.0, and 2 mM cdc1316. After 70 min, the crystal was cryoprotected and flash-cooled (buffers contained 2 mM cdc1316). A total of 180 data frames (0.5° each) were collected from this soaked crystal (crystal 6). The crystal was maintained at a temperature of 100 K during data collection. The diffraction data were processed and reduced to structure factor amplitudes, as described previously, in space group P4₃2₁2, a=70.21 Å, c=130.09 Å. The unique reflections were assigned to the same “free” and “working” sets as used for crystal 5. These data are summarized in Table 10.

Refinement of the Structure of the Cdc25B(ΔN1B).cdc1249 Inhibitor Complex Crystal Soaked with cdc1316

Refinement against the data collected with crystal 6 began with an intermediate model (“Powell11”) for the Cdc25B(ΔN1B).cdc1249 inhibitor complex from which both molecules of cdc1249 had been deleted. Slowcool simulated annealing molecular dynamics, Powell minimization, and individual temperature factor refinement lowered R to 27.4% (R_{free} 32.9%; /F/>1.0σ_{F}, 20-2.50 Å). Examination of electron-density maps showed strong, clear electron-density for cdc1249 in the active site. There was no evidence for replacement of cdc1249 by cdc1316 at the active site. The positive Fₒ-F_{c} electron-density was not as strong at the crystallographic symmetry site, however, suggesting that partial replacement of cdc1249 by cdc1316 may have occurred at this site. These data are summarized in Table 11.

X-ray Diffraction Data Collection from Cdc25B(ΔN8B).cdc1249 Inhibitor Complex Crystals

A total of 201 data frames (0.5° each) were collected from two Cdc25B(ΔN8B).cdc1249 inhibitor complex crystals (crystals 7 and 8). The crystals were maintained at a temperature of 100 K during data collection. The diffraction data were processed and reduced to structure factor amplitudes, as described previously in space group P4₃2₁2, a=70.28 Å, c=130.97 Å. The unique reflections were assigned to the same “free” and “working” sets as used for crystal 5. These data are summarized in Table 10.

Refinement of the Cdc25B(ΔN8B).cdc1249 Inhibitor Complex Crystal Structure

Refinement of the Cdc25B(ΔN8B).cdc1249 inhibitor complex crystal data began wvith the “Powell14” structural coordinates for the Cdc25B(ΔN1B).cdc1249 inhibitor complex. Rigid body, Powell minimization, and individual temperature factor refinement lowered R to 23.7% (R_{free} 25.5%; /F/>2.0σ_{F}, 20-2.00 Å). Electron-density maps revealed the presence of additional N-terminal amino acid residues, as expected for the Cdc25B(ΔN8B) construct compared to the Cdc25B(ΔN1B) construct. Both molecules of cdc1249 were in their expected locations at the active site and at the crystallographic symmetry site. Rebuilding followed by additional refinement lowered R to 22.6% (R_{free} 24.3%; /F/>1.5σ_{F}, 20-2.00 Å). These structural coordinates (“Powell2”) for the Cdc25B(ΔN8B).cdc1249 inhibitor complex consist of Cdc25B residues 370-548, two cdc1249 molecules, 128 water molecules, one Na⁺ ion, and five Cl⁻ ions. These data are summarized in Table 11.

Soaking of Cdc25B(ΔN8B).cdc1249 Inhibitor Complex Crystals with Low Molecular Weight Organic Compounds

Eight crystals of the Cdc25B(ΔN8B).cdc1249 inhibitor complex were transferred sequentially through solutions containing 4.5 M NaCl, 50 mM sodium HEPES, pH 7.0, 0.5-1.5 mM cdc1249, and increasing concentrations (0, 50, 100, 250, and 1000 mM) of t-BuNH₂, imidazole, (R)-3-hydroxypyrrolidine, or 2-methyl-1-propanol (˜500 mM maximum, saturated solution) at 4° C. Crystals were soaked for 10-15 min in each solution, and were then left in the final solution for 3 or 23 hrs. Crystals were then cryoprotected by the addition of glycerol (7.5% (v/v), 5 min; 17.5% (v/v), 5 min) and then flash-cooled by plunging into liquid nitrogen. Test x-ray diffraction images of all eight crystals showed that the crystalline order of each had been substantially unaffected by the soaking procedure, as the crystals diffracted x-rays to a maximum resolution of 2.2-2.6 Å. The crystals were stored in a liquid nitrogen refrigerator. X-ray Diffraction Data Collection from Cdc25B(ΔN8B).cdc1249 Inhibitor Complex Crystals Soaked with t-BuNH₂, Imidazole, or 2-Methyl-1-propanol

A total of 100 data frames (0.5° each) were collected from the Cdc25B(ΔN8B).cdc1249 inhibitor complex crystal (crystal 9) that had been soaked for 3 hrs with ˜0.5 M 2-methyl-1-propanol using the equipment described above. The crystal was maintained at a temperature of 100 K during data collection. The diffraction data were processed and reduced to structure factor amplitudes in space group P4₃2₁2, a=70.01 Å, c=129.92 Å. Similarly, a total of 225 data frames (0.4° each) were collected from the Cdc25B(ΔN8B).cdc1249 inhibitor complex crystal (crystal 10) that had been soaked for 23 hrs with 1 M t-BuNH₂. The crystal was maintained at a temperature of 100 K during data collection. The diffraction data were processed and reduced to structure factor amplitudes in space group P4₃2₁2, a=70.38 Å, c=131.15 Å. Similarly, a total of 112 data frames (0.5° each) were collected from the Cdc25B(ΔN8B).cdc1249 inhibitor complex crystal (crystal 11) that had been soaked for 23 hrs with 1 M imidazole. The crystal was maintained at a temperature of 100 K during data collection. The diffraction data were processed and reduced to structure factor amplitudes in space group P4₃2₁2, a=70.46 Å, c=131.36 Å. The unique reflections for all three data sets were assigned to the same “free” and “working” sets as used for crystal 5. These three data sets are summarized in Table 10.

X-ray Diffraction Data Collection from Cdc25B(ΔN8B).cdc1249 Inhibitor Complex Crystals Soaked with t-BuNH₂ or (R)-3-hydroxypyrrolidine

X-ray diffraction data were collected at NSLS as described above at a temperature of 100 K. A total of 60 data frames (1.0° each) were collected from the Cdc25B(ΔN8B).cdc1249 inhibitor complex crystal (crystal 12) that had been soaked for 3 hrs with 1 M t-BuNH₂. The diffraction data were processed and reduced to structure factor amplitudes in space group P4₃2₁2, a=70.24 Å, c=131.57 Å. Similarly, a total of 67 data frames (1.0° each) were collected from the Cdc25B(ΔN8B).cdc1249 inhibitor complex crystal (crystal 13) that had been soaked for 3 hrs with 1 M (R)-3-hydroxypyrrolidine. The diffraction data were processed and reduced to structure factor amplitudes in space group P4₃2₁2, a=70.04 Å, c=130.34 Å. And, a total of 99 data frames (0.5° each) were collected from the Cdc25B(ΔN8B).cdc1249 inhibitor complex crystal (crystal 14) that had been soaked for 23 hrs with 1 M (R)-3-hydroxypyrrolidine. The diffraction data were processed and reduced to structure factor amplitudes in space group P4₃2₁2, a=70.46 Å, c=131.00 Å. The unique reflections for these three data sets were assigned to the same “free” and “working” sets as used for crystal 5. These three data sets are summarized in Table 10.

Structural Refinement of the Cdc25B(ΔN8B).cdc1249 Inhibitor Complex Crystals Soaked with Low Molecular Weight Organic Compounds

Refinement of the soaked Cdc25B(ΔN8B).cdc1249 inhibitor complex crystal data began with the “Powell2” structural coordinates for the Cdc25B(ΔN8B).cdc1249 inhibitor complex from which water molecules and a Cl⁻ ion in the “Swimming Pool” region of the active site, adjacent to the Nal residue of cdc1249, had been removed. For crystal 12, rigid body, Powell minimization, individual temperature factor refinement and slowcool simulated annealing molecular dynamics lowered R to 24.2% (R_{free} 27.7%; /F/>1.0σ_{F}, 20-1.60 Å). Electron-density maps showed that t-BuNH₂ was not present in the “Swimming Pool”. The model for crystal 13 was refined similarly, resulting in an R of 24.7% (R_{free} 27.8%; /F/>1.0σ_{F}, 20-1.60 Å). Electron-density maps showed that (R)-3-hydroxypyrrolidine was not present in the “Swimming Pool”. The model for crystal 9 was refined as above, but without slowcool simulated annealing molecular dynamics, resulting in an R of 21.3% (R_{free}25.1%; /F/>2.0σ_{F}, 30-2.45 Å). Electron-density maps showed that 2-methyl-1-propanol was not present in the “Swimming Pool”. The model for crystal 10 was refined similarly, resulting in an R of 22.3% (R_{free}, 25.6%; /F/>2.0σ_{F}, 20-2.15 Å). Electron-density maps showed that t-BuNH₂ was not present in the “Swimming Pool”. The model for crystal 14 was refined as for crystal 10 resulting an R of 23.2% (R_{free} 25.2%; /F/>2.0σ_{F}, 30-2.30 Å). Electron-density maps showed that imidazole was not present in the “Swimming Pool”. Finally, the model for crystal 14 was refined as for crystals 12 and 13 (with an additional cycle of Powell minimization and individual temperature factor refinement) resulting in an R of 24.6% (R_{free} 27.9%; /F/>0.0σ_{F}, 20-1.60 Å). Electron-density maps showed that (R)-3-hydroxypyrrolidine was not present in the “Swimming Pool”. These data are summarized in Table 11.

X-ray Diffraction Data Collection from Crystals of the Cdc25B(ΔN1B).cdc1671, Cdc25B(ΔN8B).cdc1659, and Cdc25B(ΔN8B).cdc1671 Inhibitor Complexes

A total of 195 data frames (0.4° each) were collected from the Cdc25B(ΔN1B).cdc1671 inhibitor complex crystal (crystal 15). The crystals were maintained at a temperature of 100 K during data collection. The diffraction data were processed and reduced to structure factor amplitudes in space group P4₃2₁2, a=70.03 Å, c=130.07 Å. A total of 120 data frames (0.5° each) were collected similarly from a Cdc25B(ΔN8B).cdc1659 inhibitor complex crystal (crystal 16). The data were processed and reduced to structure factor amplitudes in space group P4₃2₁2, a=70.10 Å, c=129.47 Å. Test data were also collected from a Cdc25B(ΔN8B).cdc1671 inhibitor complex crystal (crystal 17) space group P4₃2₁2, a=70.10 Å, c=130.13 Å), but were judged not worthy of full data collection due to weak diffraction (maximum resolution, 3.00 Å) compared to the Cdc25B(ΔN1B).cdc1671 inhibitor complex crystal. The unique reflections for crystals 15 and 16 were assigned to the same “free” and “working” sets as used for crystal 5. These two data sets are summarized in Table 10.

X-ray Diffraction Data Collection from a Crystal of the Cdc25B(ΔN1B).cdc1885 Inhibitor Complex

A total of 100 data frames (0.5° each) were collected from the Cdc25B(ΔN1B).cdc1885 inhibitor complex crystal (crystal 18). Additional data (32 frames, 1.0° each) were collected at NSLS as described previously. The crystals were maintained at a temperature of 100 K during data collection. The laboratory and NSLS diffraction data were processed, merged, and reduced to structure factor amplitudes in space group P4₃2₁2, a=70.10 Å, c=130.13 Å. The unique reflections were assigned to the same “free” and “working” sets as used for crystal 5. These data are summarized in Table 10.

Refinement of the Cdc25B(ΔN1B).cdc1671 Inhibitor Complex Crystal Structure

Refinement of the Cdc25B(ΔN1B).cdc1671 inhibitor complex crystal data (crystal 15) began with the “Powell15” structural coordinates for the Cdc25B(ΔN1B).cdc1249 inhibitor complex. The cdc1249 molecules, water molecules, and ions were removed from the model, which was then subjected to rigid body, Powell minimization, and individual temperature factor refinement. Electron-density maps showed that both molecules of cdc1671 were in their expected locations (comparable to cdc1249) at the active site and at the crystallographic symmetry site. These inhibitors were added to the model. Additional refinement alternating with rebuilding lowered R to 20.2% (R_{free} 22.8%; /F/>1.0σ_{F}, 20-3.00 Å). These structural coordinates (“Powell6”) for the Cdc25B(ΔN1B).cdc1671 inhibitor complex consist of Cdc25B residues 377-548, two cdc1671 molecules, 32 water molecules, one Na⁺ ion, and five Cl⁻ ions. These data are summarized in Table 11.

Refinement of the Cdc25B(ΔN8B).cdc1659 Inhibitor Complex Crystal Structure

Refinement of the Cdc25B (ΔN8B ).cdc1659 inhibitor complex crystal data (crystal 16) began with the “Powell2” structural coordinates for the Cdc25B(ΔN8B).cdc1249 inhibitor complex. The cdc1249 molecules were removed from the model, which was then subjected to rigid body, Powell minimization, and individual temperature factor refinement. Electron-density maps showed that both molecules of cdc1659 were in their expected locations (comparable to cdc1249) at the active site and at the crystallographic symmetry site. These inhibitors were added to the model. Additional refinement alternating with rebuilding lowered R to 22.2% (R_{free} 25.3%; /F/>1.0σ_{F}, 20-2.20 Å). These structural coordinates (“Powell3”) for the Cdc25B(ΔN8B).cdc1659 inhibitor complex consist of Cdc25B residues 370-548, two cdc1659 molecules, 128 water molecules, one Na⁺ ion, and five Cl⁻ ions. These data are summarized in Table 11.

Refinement of the Cdc25B(ΔN1B).cdc1885 Inhibitor Complex Crystal Structure

Refinement of the Cdc25B(ΔN1B).cdc1885 inhibitor complex crystal data (crystal 18) began with the “Powell14” structural coordinates for the Cdc25B(ΔN1B).cdc1249 inhibitor complex. The cdc1249 molecules were removed from the model, which was then subjected to rigid body, Powell minimization, and individual temperature factor refinement. Electron-density maps showed that both molecules of cdc1885 were in their expected locations (comparable to cdc1249) at the active site and at the crystallographic symmetry site. These inhibitors were added to the model. Additional refinement alternating with rebuilding lowered R to 22.8% (R_{free} 25.1%; /F/>1.0σ_{F}, 30-1.70 Å). These structural coordinates (“Powell2”) for the Cdc25B(ΔN1B).cdc1885 inhibitor complex consist of Cdc25B residues 377-548, two cdc1885 molecules, 128 water molecules, one Na⁺ ion, and five Cl⁻ ions. These data are summarized in Table 11.

Synchrotron X-ray Diffraction Data Collection from a Cdc25B(ΔN8B).cdc1249 Inhibitor Complex Crystal Grown in the Presence of cdc1900

A total of 121 data frames (0.5° each) were collected from a Cdc25B(ΔN8B).cdc1249 inhibitor complex crystal grown in the presence of cdc1900 (crystal 19) at NSLS as described previously. The crystals were maintained at a temperature of 100 K during data collection. The diffraction data were processed and reduced to structure factor amplitudes in space group P4₃2₁2, a=70.29 Å, c=130.59 Å. The unique reflections were assigned to the same “free” and “working” sets as used for crystal 5. These data are summarized in Table 10.

High-Resolution Refinement of the Cdc25B(ΔN8B).cdc1249 Inhibitor Complex Crystal Structure

Refinement of the Cdc25B(ΔN8B).cdc1249 inhibitor complex crystal structure against the synchrotron data collected from crystal 19 began with the “Powell2” structural coordinates for the Cdc25B(ΔN8B).cdc1249 inhibitor complex; Rigid body, Powell minimization, and individual temperature factor refinement with X-PLOR, followed by the addition of several side chains in alternate conformations and refinement with REFMAC resulted in an R of 22.4% (R_{free} 24.1%; /F/>0.0σ_{F}, 15-1.45 Å). Further refinement with individual anisotropic temperature factors resulted in an R of 21.1% (R_{free} 23.1%; /F/>0.0σ_{F}, 15-1.45 Å). These final structural coordinates (“Refmac3”) for the Cdc25B(ΔN8B).cdc1249 inhibitor complex consist of cdc25B residues 369-548, two cdc1249 molecules, 235 water molecules, one Na⁺ ion, and six Cl⁻ ions. The inhibitor cdc1900 was not located, even though it had been included in the crystallization mixture. These data are summarized in Table 11.

X-ray Diffraction Data Collection from a Crystal of the Cdc25B(ΔN8B-c17).cdc1249 Inhibitor Complex

A total of 113 data frames (0.5° each) were collected from a Cdc25B(ΔN8B-c17).cdc1249 inhibitor complex crystal (crystal 20) using the equipment described above. The crystals were maintained at a temperature of 100 K during data collection. The diffraction data were processed and reduced to structure factor amplitudes, as described in 3, in space group P4₃2₁2, a=70.05 Å, c=131.44 Å. The unique reflections were assigned to the same “free” and “working” sets as used for crystal 5. These data are summarized in Table 10.

Refinement of the Cdc25B(ΔN8B-c17).cdc1249 Inhibitor Complex Crystal Structure

Refinement of the Cdc25B(ΔN8B-c17).cdc1249 inhibitor complex structure against the data collected from crystal 20 began with the “Powell2” structural coordinates for the Cdc25B(ΔN8B).cdc1249 inhibitor complex. The cdc1249 molecules were removed from the model, which was then subjected to rigid body, Powell minimization, and individual temperature factor refinement with X-PLOR. Electron-density maps showed that both molecules of cdc1249 were in their expected locations at the active site and at the crystallographic symmetry site. These inhibitors were added to the model. Additional refinement alternating with rebuilding lowered R to 22.6% (R_{free}, 27.4%; /F/>1.0σ_{F}, 30-2.70 Å). These final structural coordinates (“Powell2”) for the Cdc25B(ΔN8B-c17).cdc1249 inhibitor complex consist of Cdc25B residues 370-548, two cdc1249 molecules, 128 water molecules, one Na⁺ ion, and five Cl⁻ ions. The structure was extremely similar to that of the Cdc25B(ΔN8B).cdc1249 complex. These data are summarized in Table 11.

X-ray Diffraction Data Collection from a Crystal of the Cdc25B(ΔN8B).cdc1249 Inhibitor Complex Grown in the Presence of cdc1973

A total of 140 data frames (0.5° each) were collected from a Cdc25B(ΔN8B).cdc1249 inhibitor complex crystal grown in the presence of cdc1973 (crystal 21). The crystals were maintained at a temperature of 100 K during data collection. The diffraction data were processed and reduced to structure factor amplitudes in space group P4₃2₁2, a=70.22 Å, c=131.29 Å. The unique reflections were assigned to the same “free” and “working” sets as used for crystal 5. These data are summarized in Table 10.

Refinement of the Cdc25B(ΔN8B).cdc1249 Inhibitor Complex Crystal Structure, Crystal Grown in the Presence of cdc1973

Refinement of the Cdc25B(ΔN8B).cdc1249 inhibitor complex structure against the data collected from crystal 21 began with the “Powell2” structural coordinates for the Cdc25B(ΔN8B).cdc1249 inhibitor complex. The cdc1249 molecules were removed from the model, which was then subjected to rigid body, Powell minimization, and individual temperature factor refinement with X-PLOR. Electron-density maps showed that both molecules of cdc1249 were in their expected locations at the active site and at the crystallographic symmetry site. These inhibitors were added to the model. Additional refinement alternating with rebuilding lowered R to 20.5% (R_{free} 24.4%; /F/>1.0σ_{F}, 30-2.52 Å). These final structural coordinates (“Powell2”) for the Cdc25B(ΔN8B).cdc1249 inhibitor complex consist of Cdc25B residues 369-548, two cdc1249 molecules, 128 water molecules, one Na⁺ ion, and five Cl⁻ ions. There was no evidence for the replacement of cdc1249 by cdc1973 at either site. These data are summarized in Table 11.

Soaking of Cdc25B(ΔN8B).cdc1249 Inhibitor Complex Crystals with Other Inhibitors

Four crystals of the Cdc25B(ΔN8B).cdc1249 inhibitor complex prepared as described above were soaked in a buffer containing 4.5 M NaCl, 50 mM sodium HEPES, pH 7.0, 4° C. The buffer also contained 2 mM cdc1659, cdc1671, cdc1748, or cdc1973. After one day, the crystal in the cdc1748 soak had dissolved; the other crystals were intact. Similar soaks were set up at room temperature, using cdc1659, cdc1671, or cdc1973. After four additional days, the crystals were cryoprotected as described previously. Additional crystals were soaked in a similar fashion (1 mM inhibitor) for one day, and then cryoprotected as described previously.

X-ray Diffraction Data Collection from Crystals of the Cdc25B(ΔN8B).cdc1249 Inhibitor Complex Soaked in the Presence of cdc1659, cdc1671, or cdc1973

The x-ray diffraction characteristics of the soaked crystals described above (five days soak time) were examined. The crystals were maintained at a temperature of 100 K during data collection. Only the crystal that had been soaked with 2 mM cdc1973 at 4° C. diffracted x-rays. A total of 88 data frames (0.5° each) were collected from this crystal, crystal 22. The diffraction data were processed and reduced to structure factor amplitudes in space group P4₃2₁2, a=70.03 Å, c=131.30 Å. Then unique reflections were assigned to the same “free” and “working” sets as used for crystal 5. These data are summarized in Table 10.

Refinement of the Cdc25B(ΔN8B).cdc1249 Inhibitor Complex Crystal Structure, Crystal Soaked in the Presence of cdc1973

Refinement of the Cdc25B(ΔN8B).cdc1249 inhibitor complex structure against the data collected from crystal 22 began with the “Powell2” structural coordinates for the Cdc25B(ΔN8B).cdc1249 inhibitor complex. The cdc1249 and water molecules were removed from the model, which was then subjected to rigid body, Powell minimization, and individual temperature factor refinement with X-PLOR. Electron-density maps showed that both molecules of cdc1249 were in their expected locations at the active site and at the crystallographic symmetry site. These inhibitors were added to the model. Additional refinement alternating with rebuilding lowered R to 21.6% (R_{free} 25.3%; /F/>2.0σ_{F}, 30-3.50 Å). These final structural coordinates (“Powell3”) for the Cdc25B(ΔN8B).cdc1249 inhibitor complex (soaked with cdc1973) consist of Cdc25B residues 370-548, two cdc1249 molecules, one Na⁺ ion, and five Cl⁻ ions. A SigmaA-weighted Fₒ-F_{c} electron-density map showed no interpretable difference density for the cdc1249 molecule in the active site. At the crystallographic symmetry lattice site, however, a strong negative peak (>3σ) was centered over the sulfonate moiety of cdc1249, suggesting that some substitution of cdc1973 for cdc1249 had occurred at this site only. These data are summarized in Table 11.

X-ray Diffraction Data Collection from Crystals of the Cdc25B(ΔN8B).cdc1249 Inhibitor Complex Soaked in the Presence of cdc1659 or cdc1671 at Room Temperature

The x-ray diffraction characteristics of the soaked crystals described above (one day soak time at room temperature) were examined using the equipment described previously. The crystals were maintained at a temperature of 100 K during data collection. Only the crystal that had been soaked with 2 mM cdc1659 diffracted x-rays. A total of 59 data frames (0.75° each) were collected from this crystal, crystal 23. The diffraction data were processed and reduced to structure factor amplitudes in space group P4₃2₁2, a=70.05 Å, c=129.95 Å. The unique reflections were assigned to the same “free” and “working” sets as used for crystal 5. These data are summarized in Table 10.

Refinement of the Cdc25B(ΔN8B).cdc1249 Inhibitor Complex Crystal Structure, Crystal Soaked in the Presence of cdc1659 at Room Temperature

Refinement of the Cdc25B(ΔN8B).cdc1249 inhibitor complex structure against the data collected from crystal 24 began with the “Powell2” structural coordinates for the Cdc25B(ΔN8B).cdc1249 inhibitor complex. The cdc1249 and water molecules were removed from the model, which was then subjected to rigid body and, Powell minimization, and overall temperature factor refinement with X-PLOR. Electron-density maps showed that both molecules of cdc1249 were in their expected locations at the active site and at the crystallographic symmetry site. These inhibitors were added to the model. Additional refinement alternating with rebuilding lowered R to 24.3% (R_{free} 26.3%; /F/>1.0σ_{F}, 30-2.70 Å). These final structural coordinates (“Powell2”) for the Cdc25B(ΔN8B).cdc1249 inhibitor complex (soaked with cdc1659) consist of Cdc25B residues 370-548, two cdc1249 molecules, one Na⁺ ion, and five Cl⁻ ions. A SigmaA-weighted Fₒ-F_{c} electron-density map showed no interpretable difference density for either cdc1249 molecule, suggesting that no substitution of cdc1659 for cdc1249 had occurred. These data are summarized in Table 10.

TABLE 10
Summary of x-ray diffraction data collection

| | Poly- | Ihibitor | Resolution | Unique | Coverage | Multipli- | <I/σ_{I}> | R_{sym} |
|---|---|---|---|---|---|---|---|---|
| Crystal | Peptide | Or Soak | Å | Reflections | % * | city * | * | % * |
| 1 | ΔN1A | Na₂WO₄ | 1.79 | 20,146 | 96.6 (65.2) | 5.8 (3.2) | 19.4 | 3.6 |
| | | | | | | | (3.0) | (37.4) |
| 2 | ΔN1A | 2 mM cdc1316, | 1.79 | 19,644 | 95.5 (54.5) | 3.5 (1.8) | 15.0 | 3.7 |
| | | 23 hrs | | | | | (2.3) | (44.4) |
| 2 | ΔN1A | 2 mM cdc1316, | 1.35 | 14,015 | 29.3 (34.1) | 1.1 (1.1) | 5.8 | 8.7 |
| (NSLS) | | 23 hrs | | | | | (1.0) | (43.9) |
| 3 | ΔN8A | Na₂WO₄ | 2.15 | 12,117 | 100 (100) | 4.8 (3.5) | 10.7 | 8.4 |
| | | | | | | | (2.7) | (46.5) |
| 4 | ΔN8A | BASF-32 thawed, | 3.00 | 3,968 | 88.5 (94.6) | 1.8 (1.7) | 5.5 | 13.0 |
| | | soaked in 10 mM | | | | | (2.2) | (30.7) |
| | | Na₂WO₄, re- | | | | | | |
| | | frozen | | | | | | |
| 5 | ΔN1B | cdc1249 | 2.30 | 15,069 | 99.5 (96.4) | 6.2 (5.6) | 13.2 | 9.1 |
| | | | | | | | (2.8) | (55.6) |
| 5 | ΔN1B | cdc1249 | 1.95 | 23,616 | 97.2 (97.0) | 2.6 (2.6) | 7.9 | 7.8 |
| (NSLS) | | | | | | | (1.9) | (46.7) |
| 6 | ΔN1B | Cdc1249; 70 min | 2.50 | 11,878 | 99.9 (100) | 6.6 (6.7) | 12.5 | 11.0 |
| | | soak, 2 mM | | | | | (3.4) | (51.5) |
| | | cdc1316 | | | | | | |
| 7 & 8 | ΔN8B | cdc1249 | 2.00 | 22,658 | 98.8 (95.4) | 6.6 (4.1) | 12.7 | 7.9 |
| | | | | | | | (2.7) | (44.0) |
| 12 | ΔN8B | Cdc1249; 3 hr | 1.53 | 48,288 | 96.0 (81.7) | 3.8 (1.8) | 9.9 | 5.9 |
| (NSLS) | | soak, 1 M | | | | | (1.3) | (49.0) |
| | | t-BuNH₂ | | | | | | |
| 13 | ΔN8B | Cdc1249; 3 hr | 1.56 | 45,977 | 98.2 (85.2) | 4.4 (2.6) | 10.4 | 6.0 |
| (NSLS) | | soak, 1 M (R)-3- | | | | | (1.6) | (51.6) |
| | | HO-pyrrolidine | | | | | | |
| 9 | ΔN8B | Cdc1249; 3 hr | 2.45 | 12,459 | 99.7 (98.9) | 3.4 (3.8) | 6.3 | 14.3 |
| | | soak, ˜0.5 M 2- | | | | | (2.3) | (44.6) |
| | | Methyl-1- | | | | | | |
| | | propanol | | | | | | |
| 16 | ΔN8B | cdc1249; 3 hr | 2.16 | 18,228 | 99.2 (95.8) | 6.5 (5.8) | 11.1 | 11.1 |
| | | soak, 1 M | | | | | (2.9) | (52.2) |
| | | t-BuNH₂ | | | | | | |
| 14 | ΔN8B | cdc1249; 23 hr | 1.60 | 38,903 | 88.2 (77.8) | 3.1 (1.9) | 8.2 | 7.9 |
| (NSLS) | | soak, 1 M (R)-3- | | | | | (2.0) | (37.8) |
| | | HO-pyrrolidine | | | | | | |
| 15 | ΔN1B | cdc1671 | 2.90 | 7,651 | 99.5 (98.5) | 5.6 (5.9) | 8.0 | 17.9 |
| | | | | | | | (3.3) | (45.6) |
| 11 | ΔN8B | cdc1249; 23 hr | 2.30 | 14,698 | 95.7 (89.6) | 3.7 (2.4) | 7.3 | 12.7 |
| | | soak, 1 M | | | | | (2.0) | (40.8) |
| | | Imidazole | | | | | | |
| 16 | ΔN8B | cdc1659 | 2.20 | 17,098 | 99.9(99.6) | 4.4(4.5) | 8.9 | 10.0 |
| | | | | | | | (2.4) | (52.5) |
| 18 | ΔN1B | cdc1885 | 1.70 | 21,718 | 60.0 (14.2) | 2.6 (1.3) | 8.1 | 8.6 |
| | | | | | | | (1.4) | (43.3) |
| 19 | ΔN8B | cdc1249 + | 1.45 | 48,439 | 82.8 (45.2) | 3.7 (1.4) | 11.6 | 6.2 |
| | | cdc1900 | | | | | (1.7) | (35.1) |
| 20 | ΔN8B- | cdc1249 | 2.70 | 9,521 | 99.7 (99.9) | 4.3 (4.4) | 10.7 | 9.9 |
| | c17 | | | | | | (3.7) | (36.9) |
| 21 | ΔN8B | cdc1249 + | 2.52 | 11,507 | 98.5 (98.6) | 3.6 (3.4) | 8.4 | 10.9 |
| | | cdc1973 | | | | | (3.1) | (33.9) |
| 22 | ΔN8B | cdc1249; soaked | 3.50 | 3,963 | 90.1 (93.1) | 3.5 (3.5) | 4.7 | 21.4 |
| | | 5 days at 4 C w/ | | | | | (3.6) | (28.2) |
| | | 2 mM cdc 1973 | | | | | | |
| 23 | ΔN8B | cdc1249; soaked | 2.70 | 8,650 | 93.3 (98.2) | 3.5 (3.4) | 8.4 | 11.5 |
| | | 1 day at 22 C w/ | | | | | (2.8) | (38.9) |
| | | 2 mM cdc1659 | | | | | | |

* Highest resolution shell in parentheses.

TABLE 11
Summary of Crystallographic Refinement Statistics

| Crystal | Protein | Inhibitor | Resolution | R | R_{free} |
|---|---|---|---|---|---|
| No. | Construct | or Soak | Å | % | % |
| 1 | ΔN1A | Na₂WO₄ | 1.8 | 31.7 | 36.3 |
| 2 | ΔN1A | 2 mM cdc1316, 23 | 1.80 | 23.0 | 24.3 |
| | | hrs | | | |
| 3 | ΔN8A | Na₂WO₄ | 2.15 | 22.8 | 27.1 |
| 4 | ΔN8A | Crystal 3 thawed, | 3.00 | 22.7 | 25.7 |
| | | soaked in 10 mM | | | |
| | | Na₂WO₄, re-frozen | | | |
| 5 | ΔN1B | Cdc1249 | 1.95 | 21.8 | 24.4 |
| 6 | ΔN1B | Cdc1249; 70 min | 2.50 | 27.4 | 32.9 |
| | | soak, 2 mM | | | |
| | | cdc1316 | | | |
| 7 + 8 | ΔN8B | cdc1249 | 2.00 | 22.6 | 24.3 |
| 12 | ΔN8B | Cdc1249; 3 hr soak, | 1.60 | 24.2 | 27.7 |
| | | 1 M t-BuNH₂ | | | |
| 13 | ΔN8B | cdc1249; 3 hr soak, | 1.56 | 24.7 | 27.8 |
| | | 1 M (R)-3-HO- | | | |
| | | pyrrolidine | | | |
| 9 | ΔN8B | cdc1249; 3 hr soak, | 2.45 | 21.3 | 25.1 |
| | | ˜0.5 M 2-Methyl-1- | | | |
| | | propanol | | | |
| 10 | ΔN8B | cdc1249; 23 hr | 2.16 | 22.3 | 25.6 |
| | | soak, 1 M t-BuNH₂ | | | |
| 14 | ΔN8B | cdc1249; 23 hr | 1.60 | 24.6 | 27.9 |
| | | soak, 1 M (R)-3- | | | |
| | | HO-pyrrolidine | | | |
| 15 | ΔN1B | cdc1671 | 3.00 | 20.2 | 22.8 |
| 11 | ΔN8B | cdc1249; 23 hr soak, | 2.30 | 23.2 | 25.2 |
| | | 1 M Imidazole | | | |
| 16 | ΔN8B | cdc1659 | 2.20 | 22.2 | 25.3 |
| 18 | ΔN1B | cdc1885 | 1.70 | 22.8 | 25.1 |
| 19 | ΔN8B | cdc1249 + cdc1900 | 1.45 | 21.1 | 23.1 |
| 20 | ΔN8B-c17 | cdc1249 | 2.70 | 22.6 | 27.4 |
| 21 | ΔN8B | cdc1249 + cdc1973 | 2.52 | 20.5 | 24.4 |
| 22 | ΔN8B | cdc1249; soaked 5 | 3.50 | 21.6 | 25.3 |
| | | days at 4° C. w/2 mM | | | |
| | | cdc1973 | | | |
| 23 | ΔN8B | cdc 1249; soaked 1 | 2.70 | 24.3 | 26.3 |
| | | day at 22° C. w/2 mM | | | |
| | | cdc1659 | | | |

References:

Brünger, A. T. (1992)X-PLOR Version 3.1, A System for Crystallography and NMR (Yale University Press, New Haven, Conn.).

Collaborative Computational Project, Number 4. (1994). Acta Cryst. D50, 760-763.

Evans, P. R. (1997). Joint CCP4 and ESF-EACBM Newsletter 33, 22-24.

French, S. & Wilson, K. (1978). Acta Cryst. A34, 517-525.

Jones, T. A., Zou, J. Y., Cowan, S. W. & Kjelgaard, M. (1991). Acta Cryst. A47, 110-119.

Kabsch, W. (1993). J. Appl. Cryst. 24,795-800.

Leslie, A. G. W. (1992). CCP4 and ESF-EACMB Newsletter on Protein Crystallography No. 26.

Murshudov, G. N., Vagin, A. A., and Dodson, E. J. (1997) Acta Crystallogr. D53,240 255.

Navaza, J. (1994). Acta Cryst. A50, 157-163.

While this invention has been particularly shown and described with references to preferred embodiments thereof, it will be understood by those skilled in the art that various changes in form and details may be made therein without departing from the scope of the invention encompassed by the appended claims.

What is claimed is:

1. A crystalline polypeptide, said polypeptide comprising the catalytic domain of a Cdc25B protein or Cdc25C protein.

2. The crystalline polypeptide of claim 1 wherein the polypeptide comprises the catalytic domain of human Cdc25B.

3. A crystalline polypeptide-ligand complex, said polypeptide comprising the catalytic domain of a Cdc25 protein.

4. The crystalline polypeptide/ligand complex of claim 3 wherein the polypeptide comprises the catalytic domain of a mammalian Cdc25.

5. The crystalline polypeptide/ligand complex of claim 4 wherein the mammalian Cdc25 protein is Cdc25A, Cdc25B or Cdc25C.

6. The crystalline polypeptide/ligand complex of claim 5 wherein the mammalian Cdc25 protein is human Cdc25A human Cdc25B or human Cdc25C.

7. The crystalline polypeptide/ligand complex of claim 6 wherein the polypeptide comprises amino acids 336-523 of SEQ ID NO: 1.

8. The crystalline polypeptide of claim 2 wherein the polypeptide comprises amino acids 351-540 of SEQ ID NO: 2.

9. The crystalline polypeptide/ligand complex of claim 4 wherein the polypeptide comprises amino acids 351-540 of SEQ ID NO: 2.

10. The crystalline polypeptide/ligand complex of claim 9 wherein the ligand is of the formula:

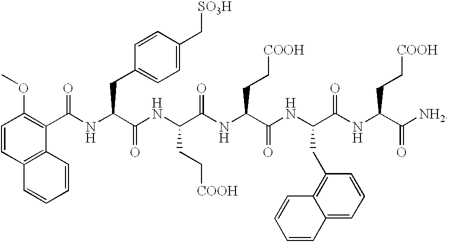

11. The crystalline polypeptide/ligand complex of claim 10 having unit cell parameters a and b are about 70 Å, c is about 130 Å and α=β=γ=90°.

12. A method of determining the three dimensional structure of a first polypeptide comprising the catalytic domain of a Cdc25 protein, said method comprising the steps of:
(a) obtaining a crystal of the first polypeptide comprising the catalytic domain of Cdc25;
(b) obtaining x-ray diffraction data for said crystal; and
(c) solving the crystal structure of said crystal using the atomic coordinates of a second polypeptide and said x-ray diffraction data, said second polypeptide comprising the catalytic domain of a Cdc25B protein.

13. The method of claim 12 wherein the crystal of the first polypeptide comprises the first polypeptide complexed with a ligand.

14. The method of claim 12 wherein the first polypeptide comprises the catalytic domain of a mammalian Cdc25 protein.

15. The method of claim 14 wherein the first polypeptide and the second polypeptide, independently, comprise the catalytic domain of a human Cdc25 protein.

16. The method of claim 15 wherein the first polypeptide comprises the catalytic domain of human Cdc25A, Cdc25B or Cdc25C and the second polypeptide comprises the catalytic domain of human Cdc25B.

17. The method of claim 16 wherein the first polypeptide comprises the catalytic domain of human Cdc25A.

18. The method of claim 16 wherein the first polypeptide comprises the catalytic domain of human Cdc25B.

19. The method of claim 16 wherein the first polypeptide comprises the catalytic domain of human Cdc25C.

20. A method of identifying a compound which is an inhibitor of a Cdc25 protein, said method comprising the steps of
(a) obtaining a crystal comprising a polypeptide comprising the catalytic domain of a Cdc25 protein;
(b) obtaining the atomic coordinates of the polypeptide;
(c) using said atomic coordinates to define the catalytic domain of Cdc25; and
(d) identifying a compound which fits the catalytic domain; wherein the compound which fits the catalytic domain is an inhibitor of a Cdc25 protein.

21. The method of claim 20 further comprising the step of assessing the ability of the compound identified in step (d) to inhibit Cdc25.

22. The method of claim 20 wherein the Cdc25 protein is a mammalian protein.

23. The method of claim 22 wherein the Cdc25 protein is a human protein.

24. The method of claim 23 wherein the Cdc25 protein is human Cdc25A, human Cdc25B or human Cdc25C.

25. The method of claim 20 wherein said crystal further comprises a ligand bound to said catalytic domain.

26. The method of claim 23 wherein the polypeptide comprises amino acids 351-540 of SEQ ID NO: 2.

27. The method of claim 24 wherein the ligand is of the formula:

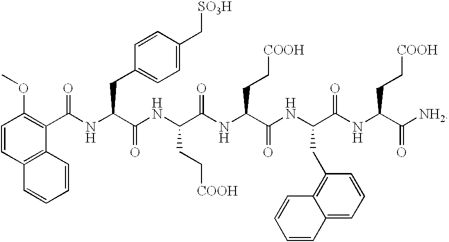

28. The method of claim 27 wherein said crystal has unit cell parameters a=b=70.15 Å, c=130.35 Å and α=β=γ=90°.

29. A method of identifying a compound which is a potential inhibitor of a Cdc25 protein, said method comprising the step of designing a compound that will interact with one or more subsites in the catalytic domain of the Cdc25 protein, based upon the crystal structure coordinates of a polypeptide comprising the catalytic domain; wherein said compound is identified as a potential inhibitor of the Cdc25 protein.

30. The method of claim 29 wherein the Cdc25 protein is a mammalian Cdc25 protein.

31. The method of claim 29 wherein the Cdc25 protein is a human Cdc25 protein.

32. The method of claim 31 wherein the Cdc25 protein is human Cdc25A, human Cdc25B or human Cdc25C.

33. The method of claim 32 wherein the polypeptide comprises amino acids 336-540 of SEQ ID NO: 2.

34. The method of claim 33 wherein the polypeptide has the amino acid sequence of SEQ ID NO. 5.

35. The method of claim 34 wherein the polypeptide has the amino acid sequence of SEQ ID NO. 11.

36. The method of claim 35 wherein the crystal structure coordinates are set forth in FIG. 15A-15PPP.

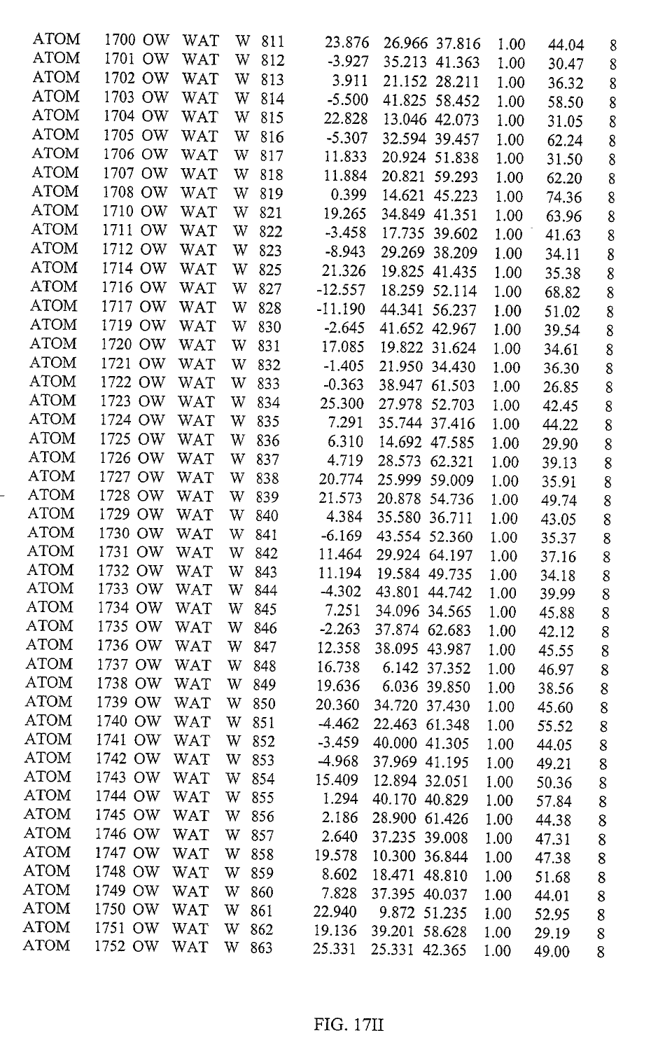

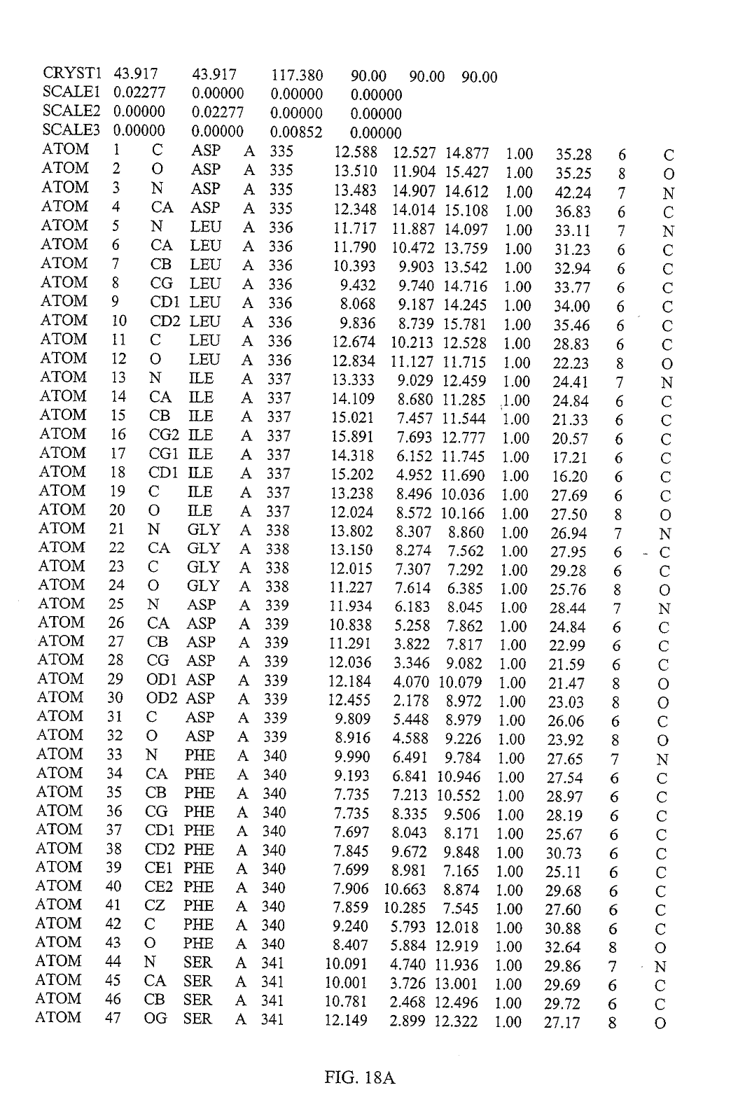

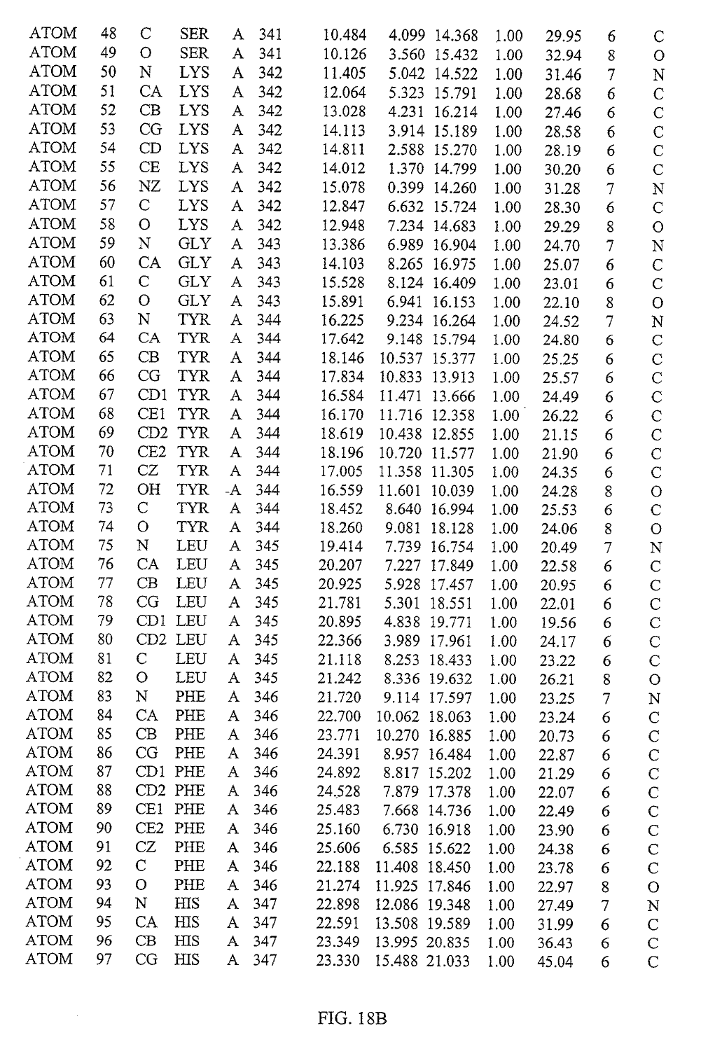

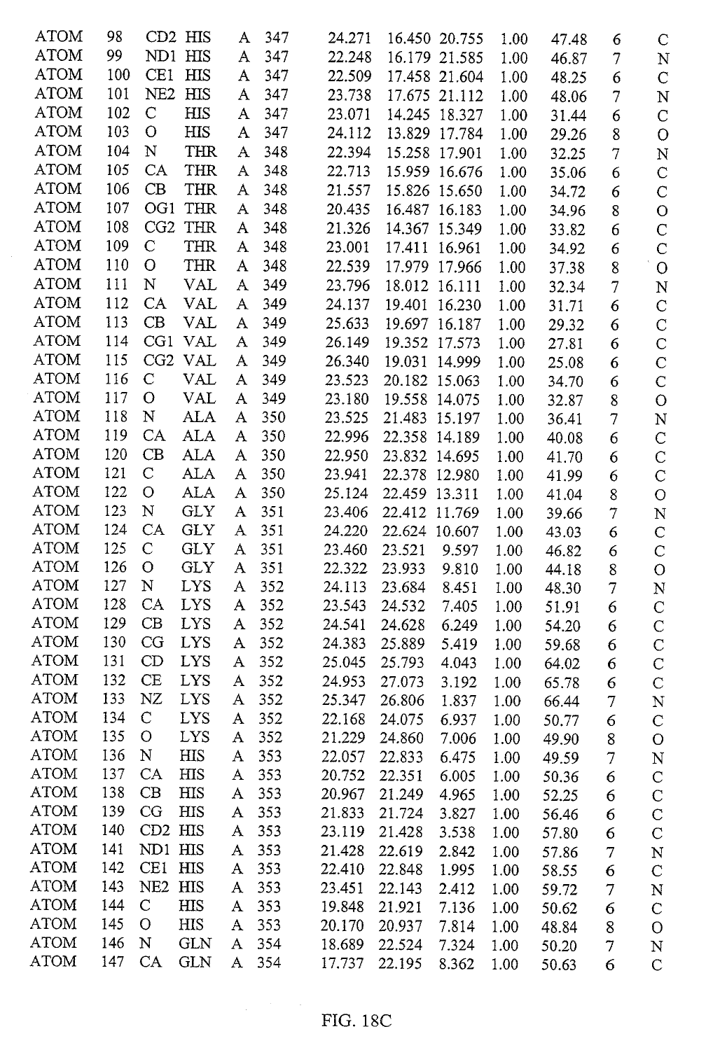

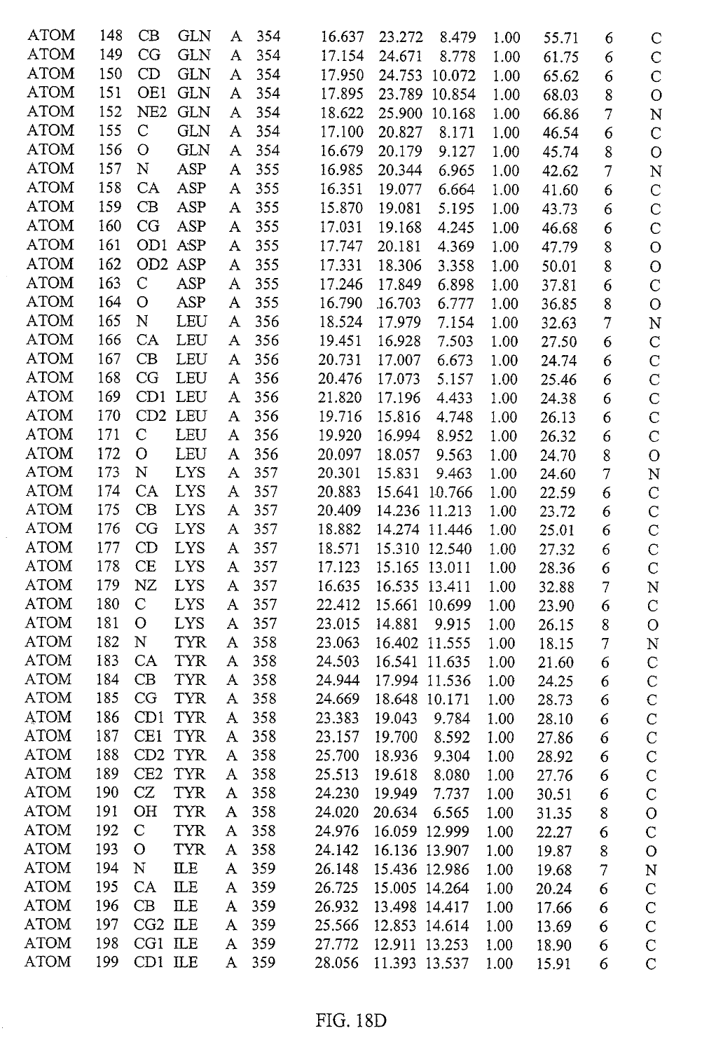

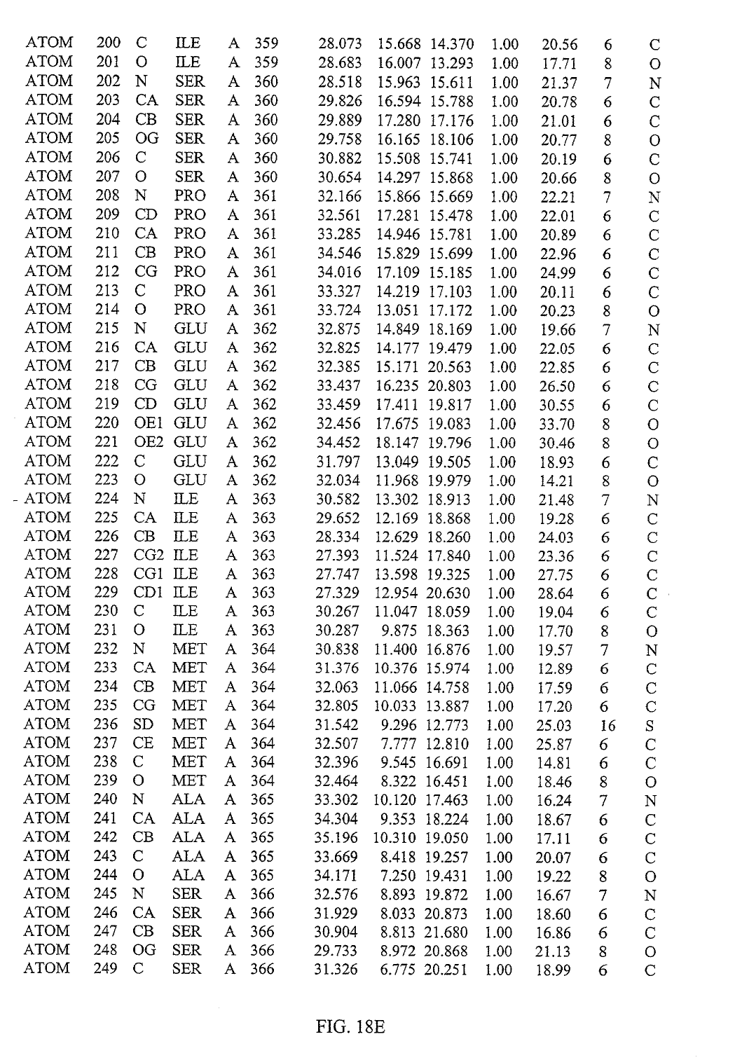

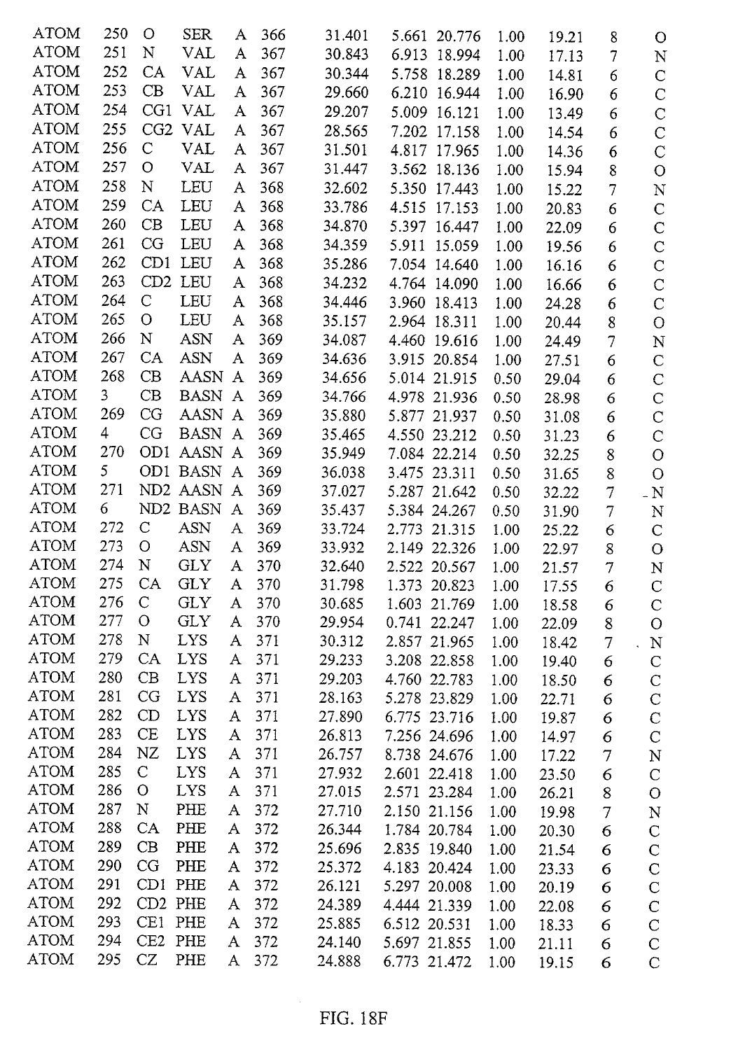

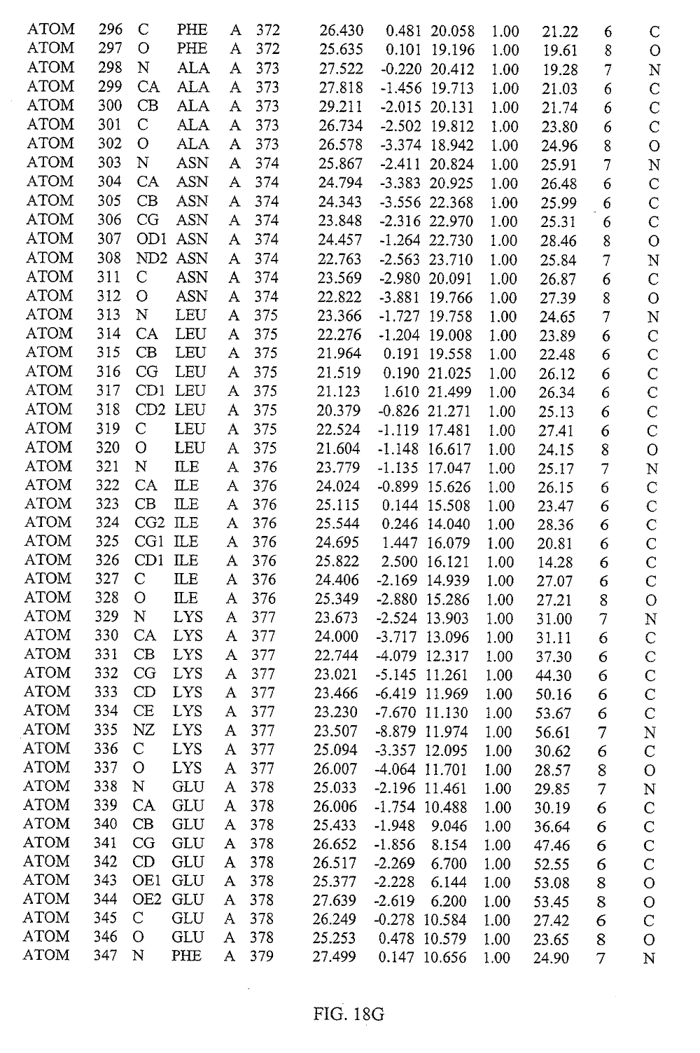

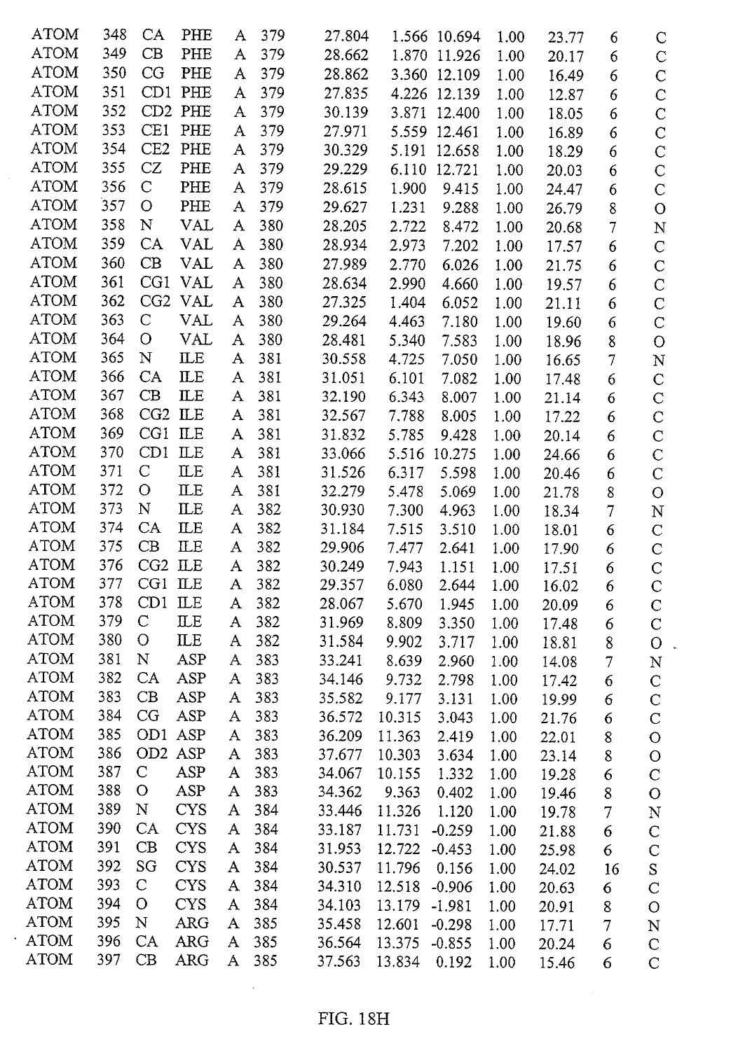

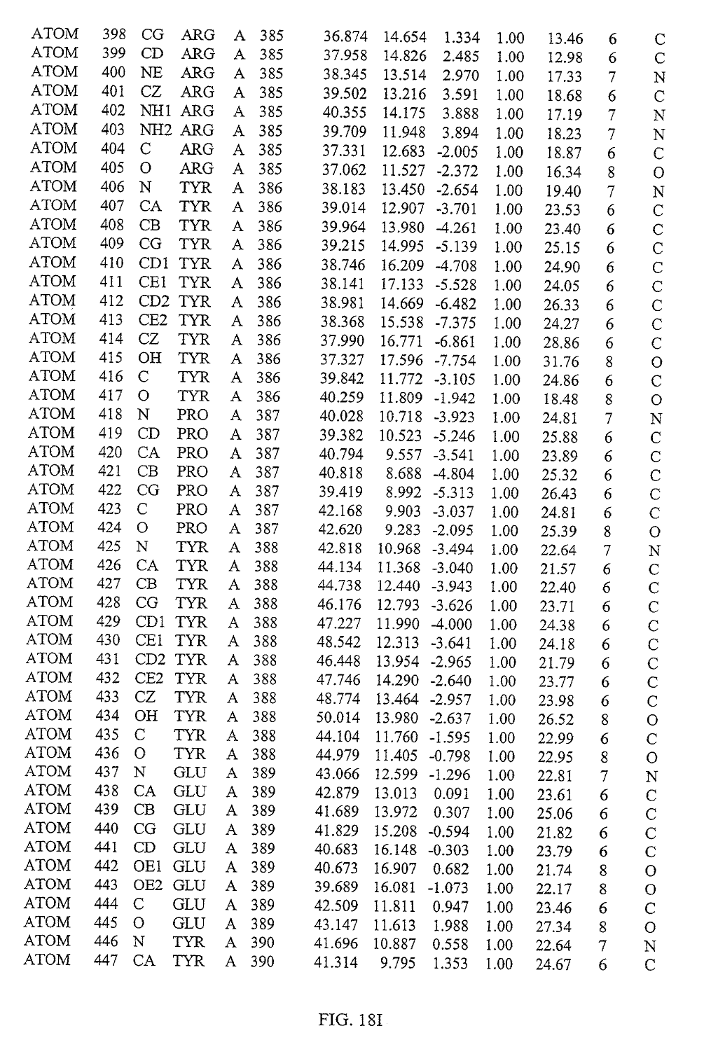

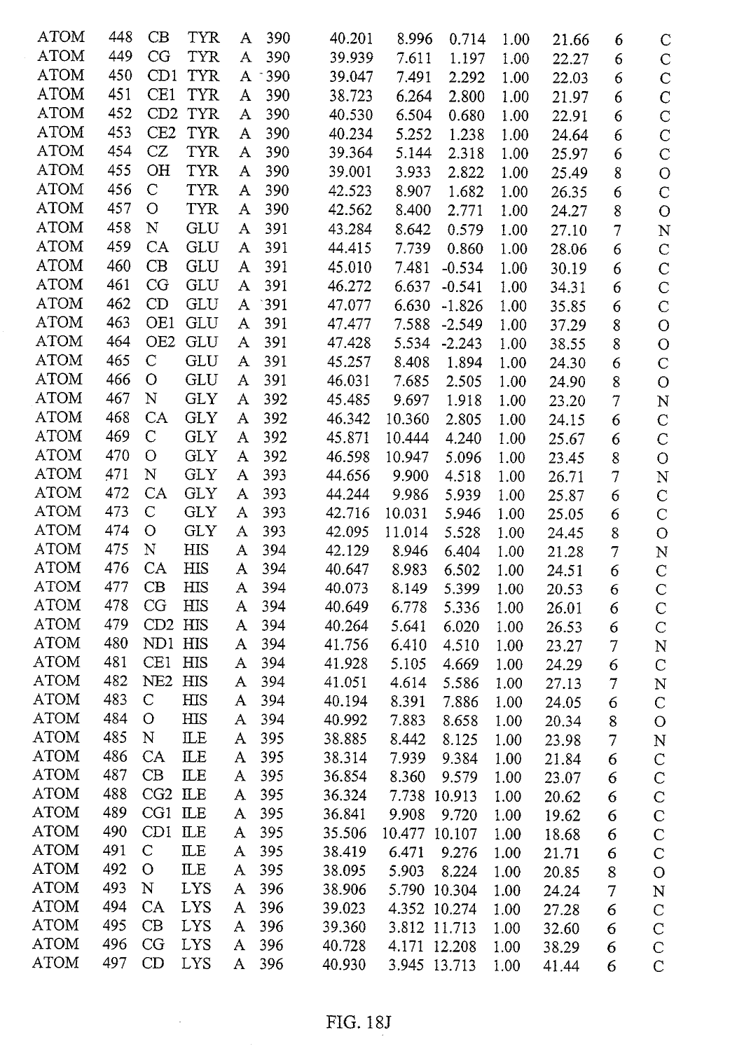

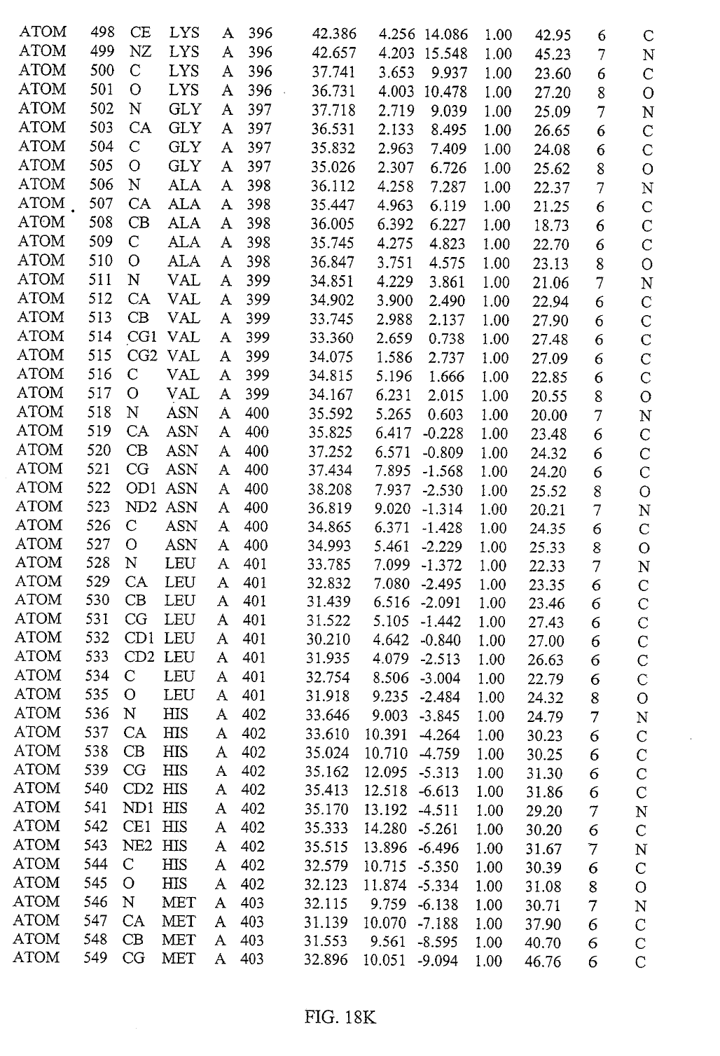

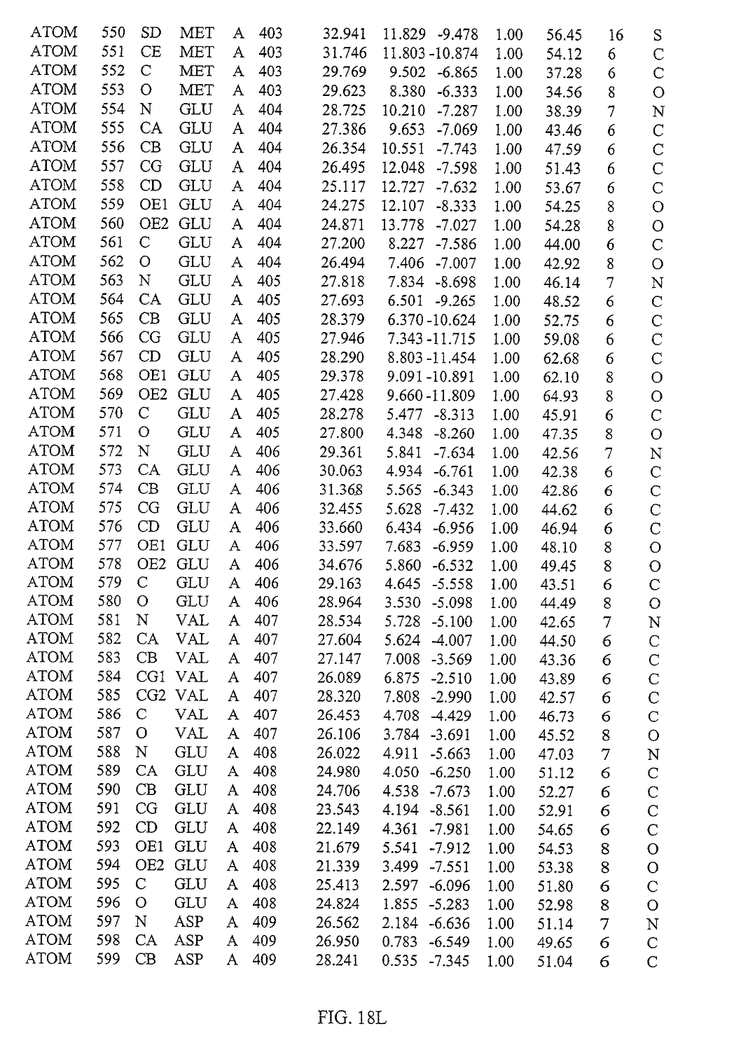

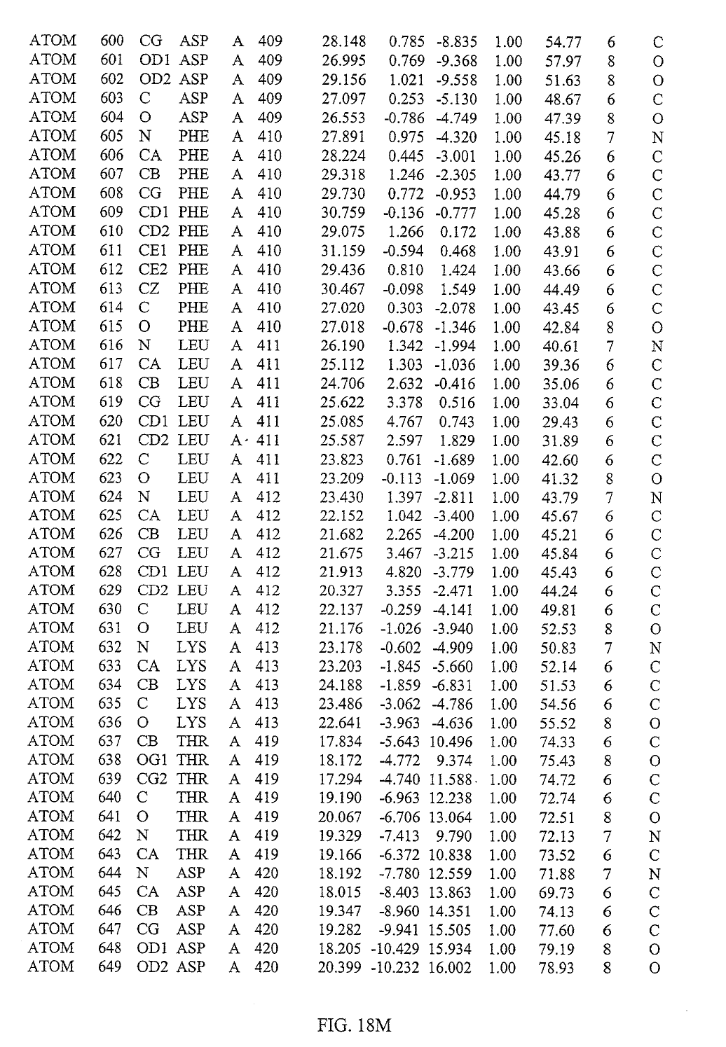

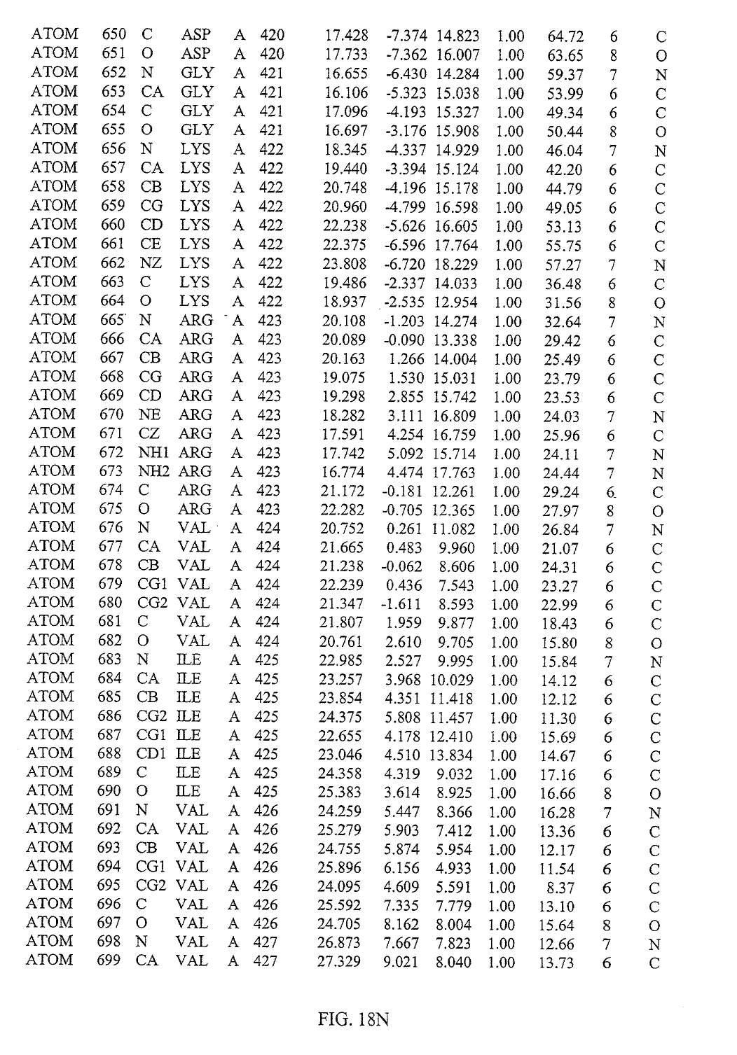

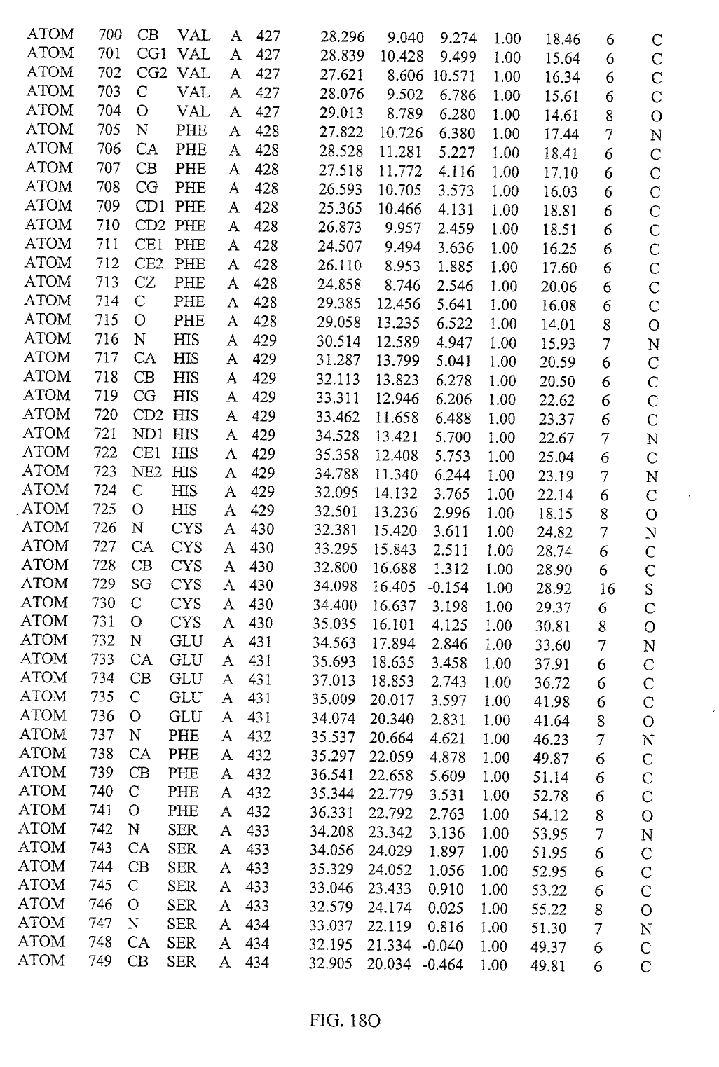

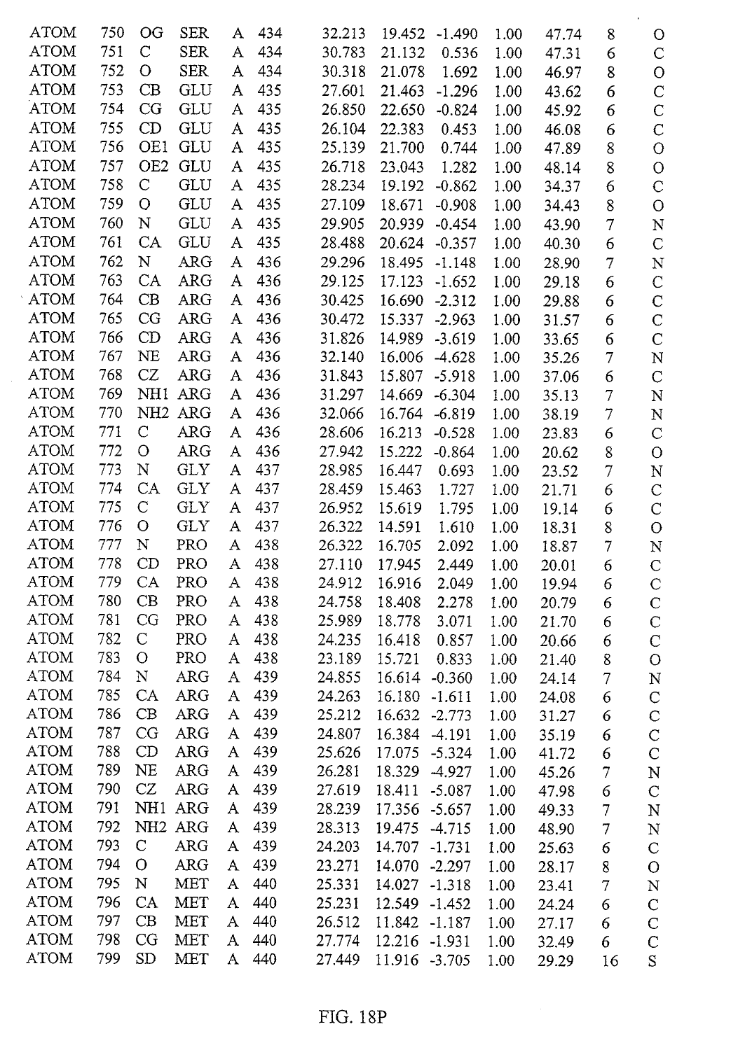

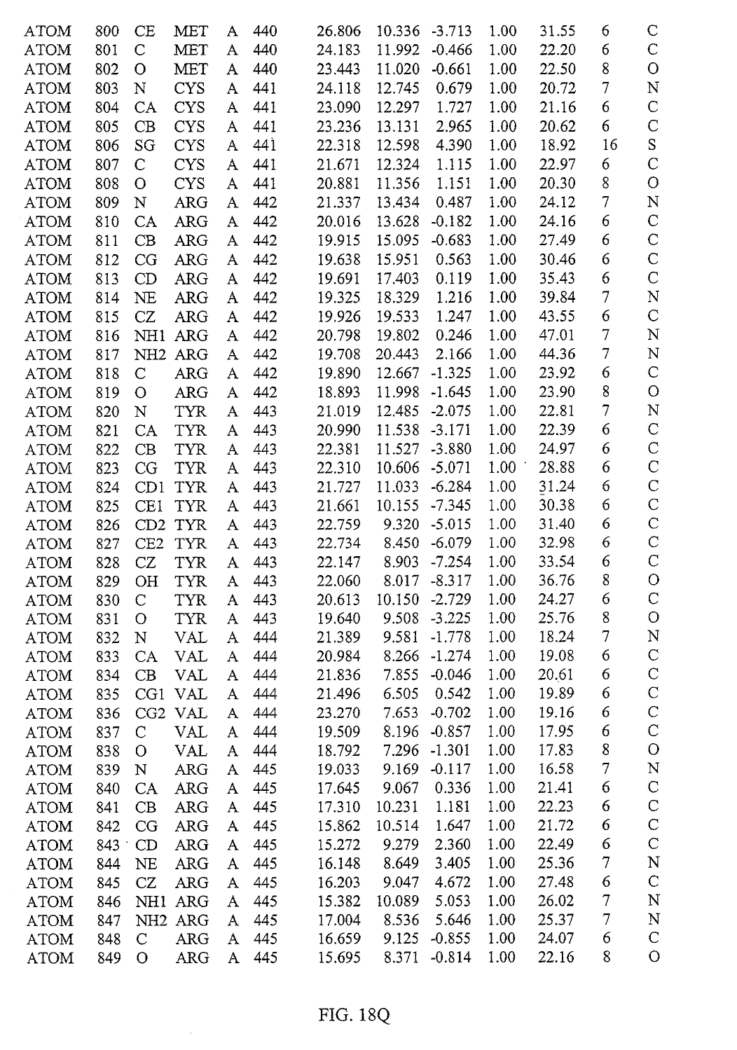

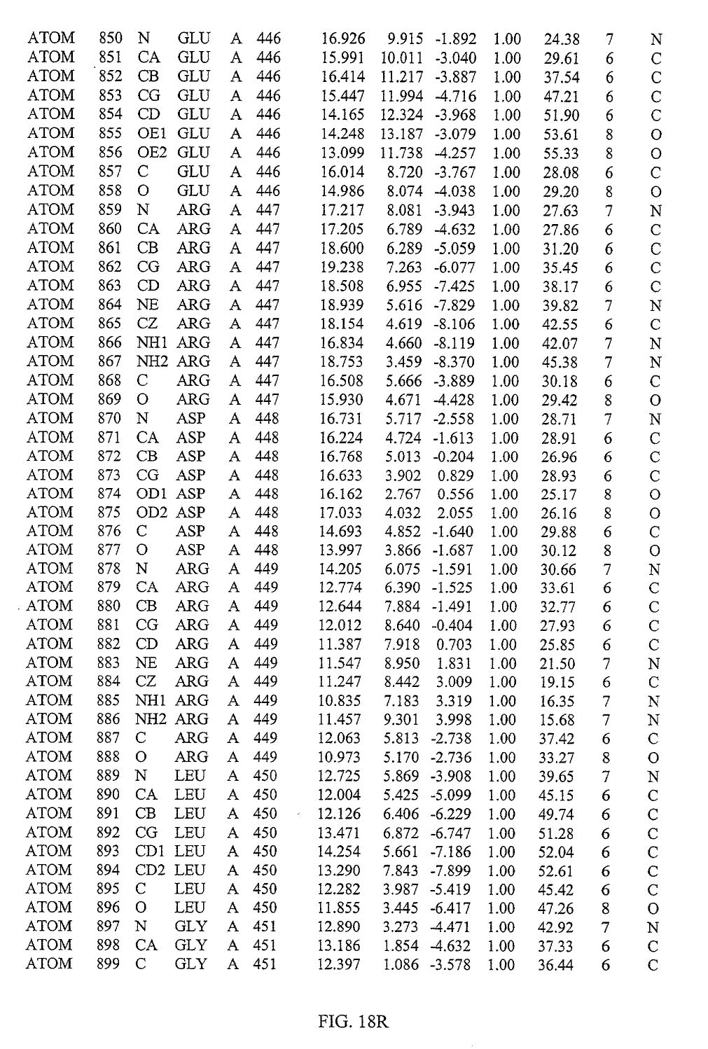

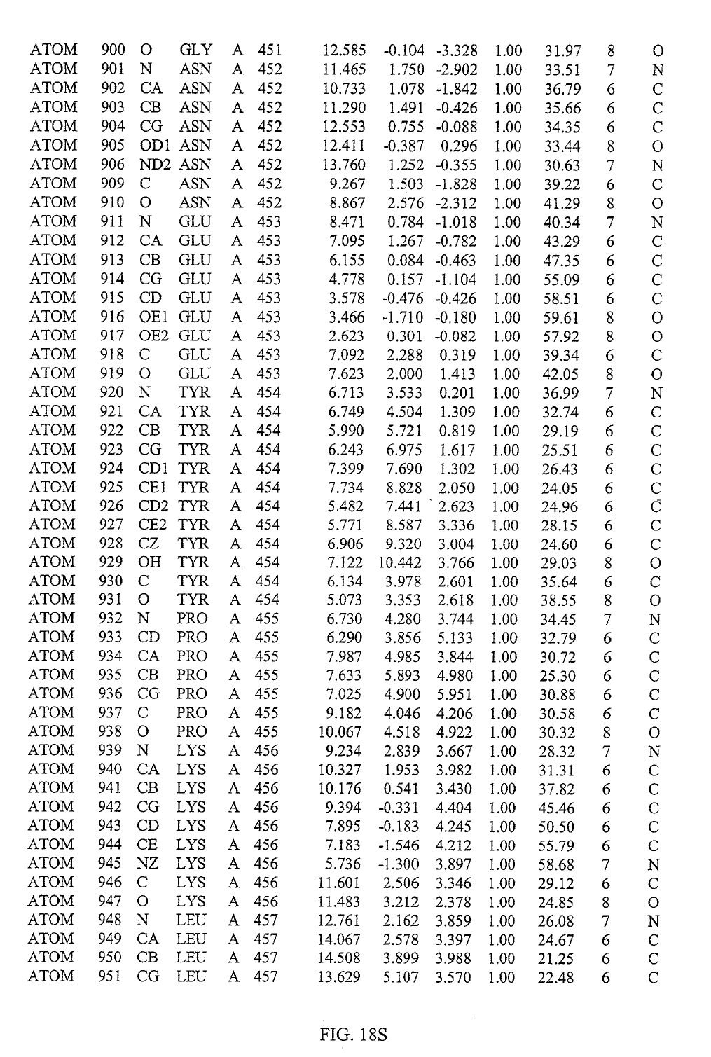

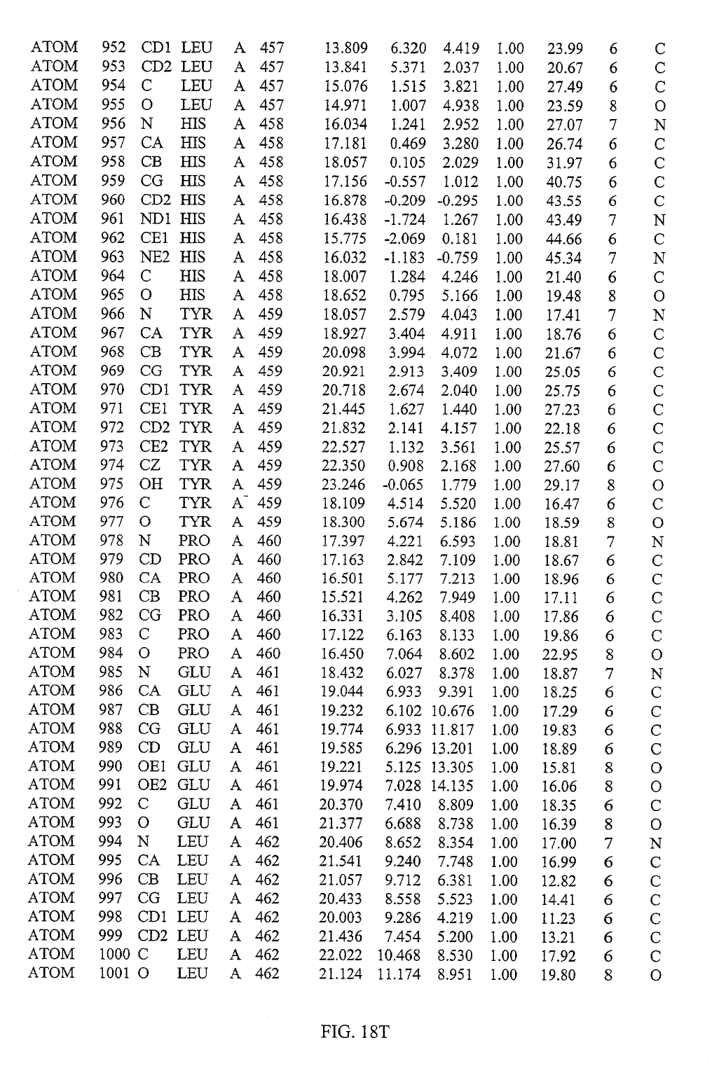

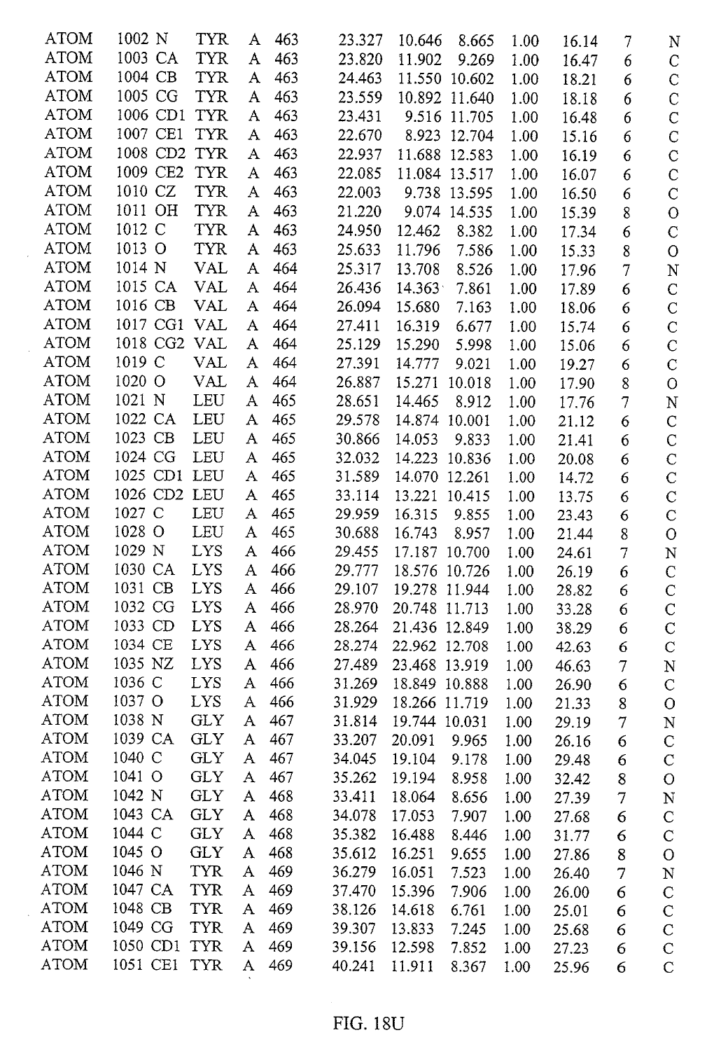

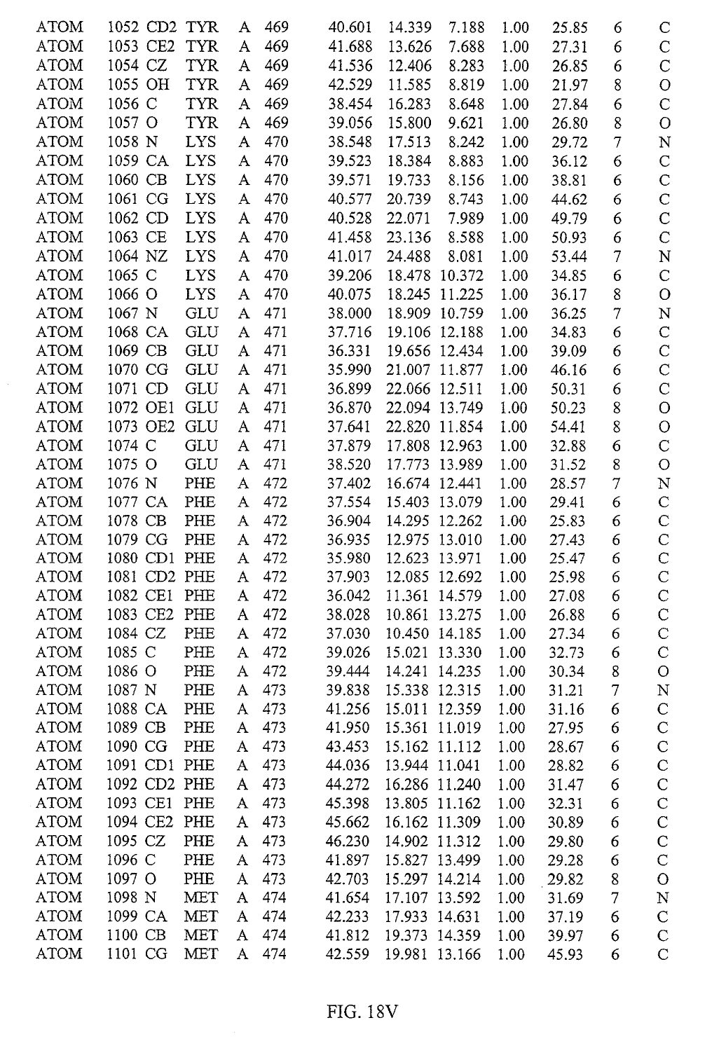

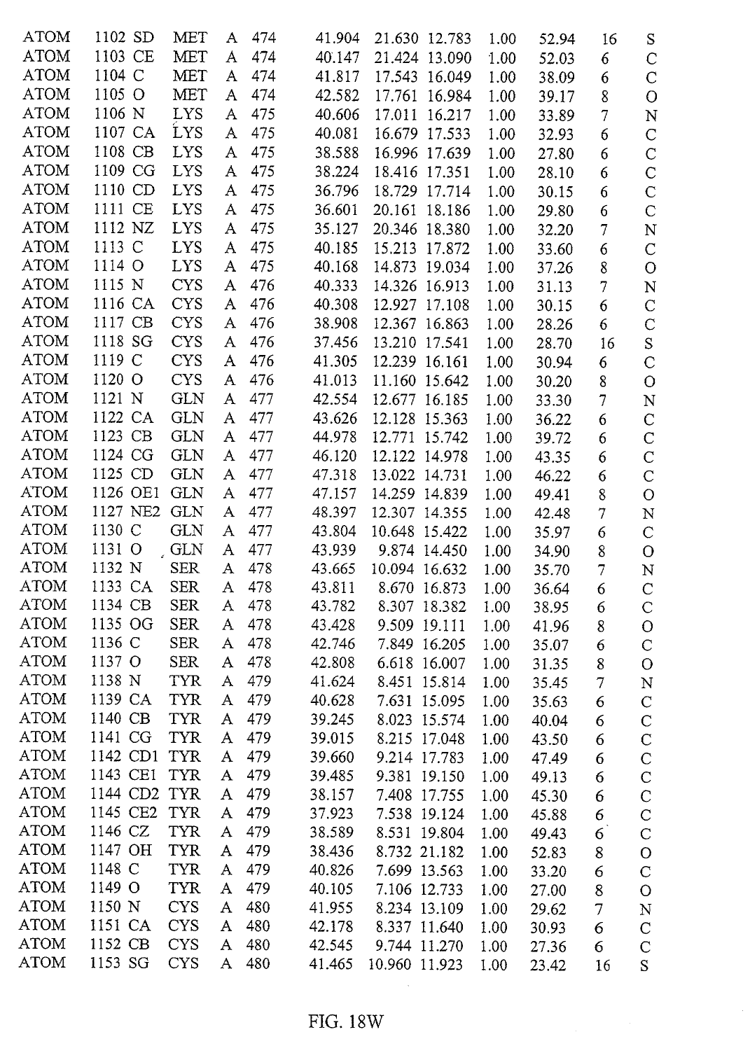

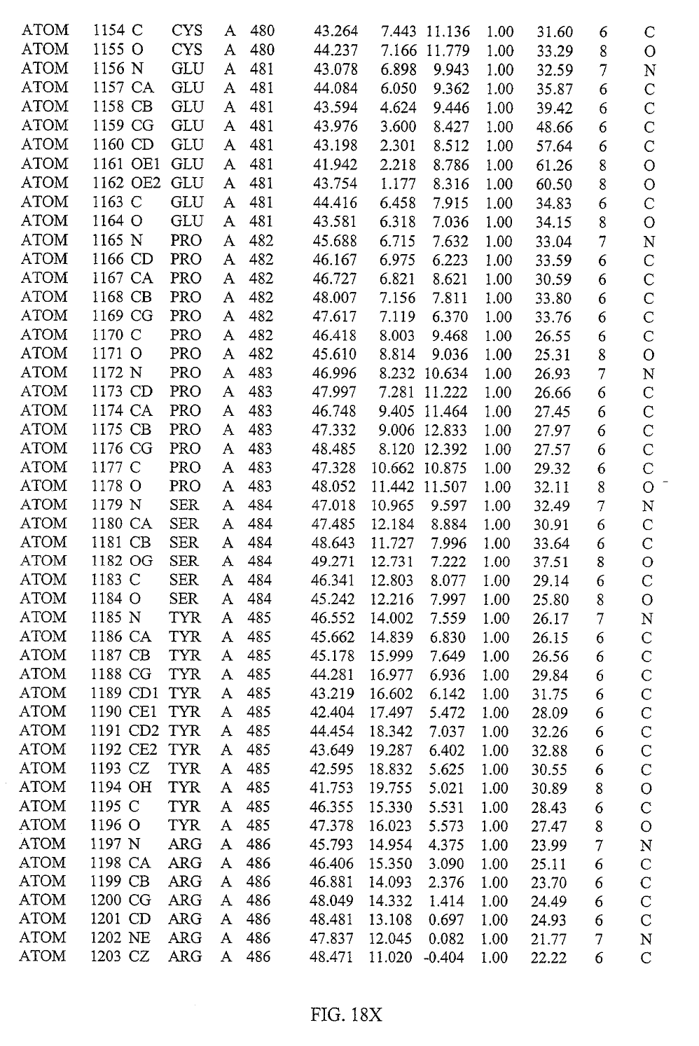

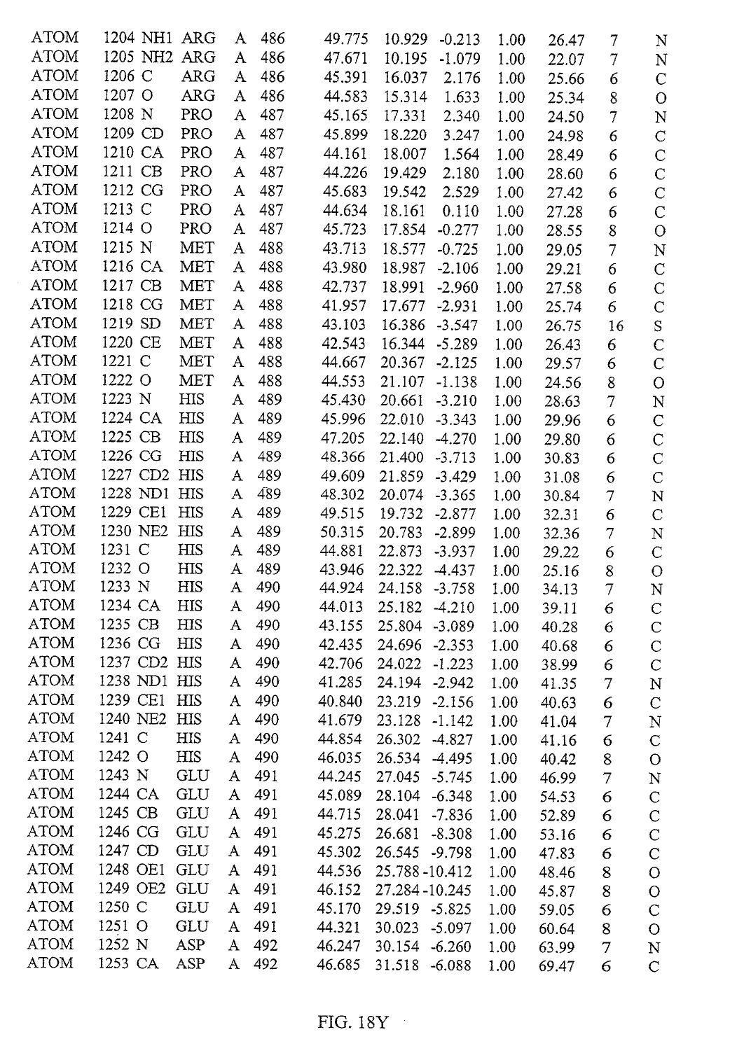

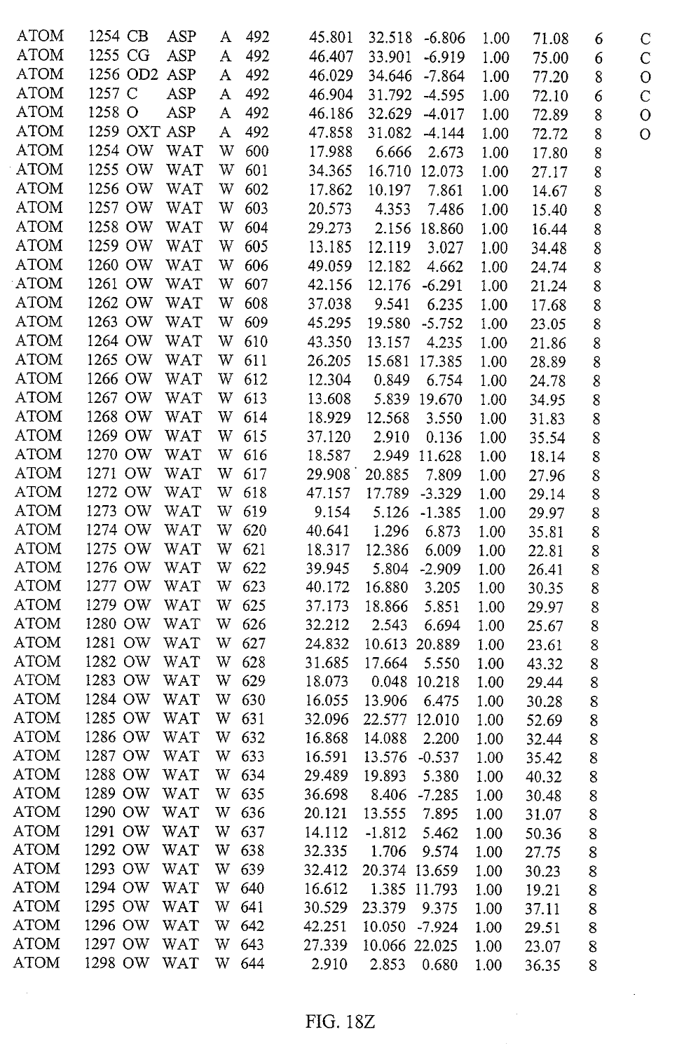

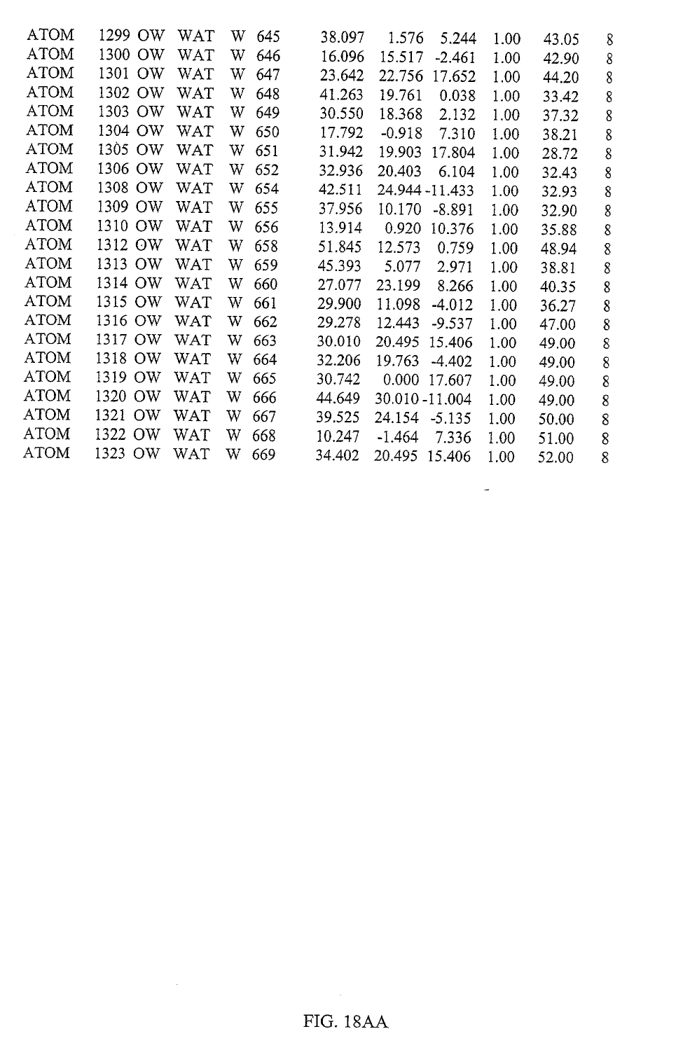

37. The method of claim 32 wherein the crystal structure coordinates are set forth in FIGS. 18A to 18X.

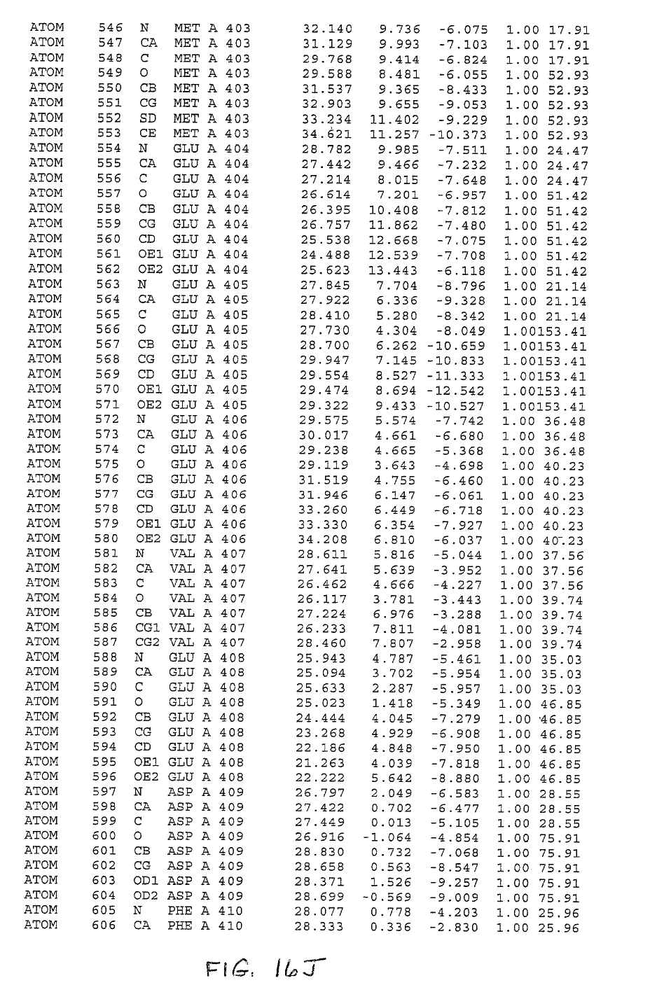

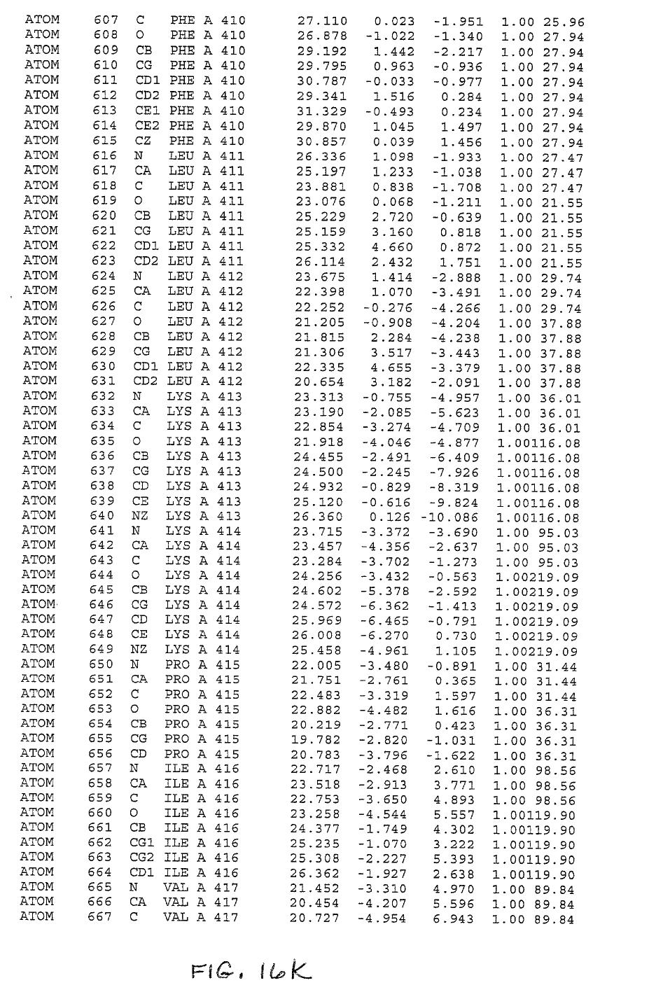

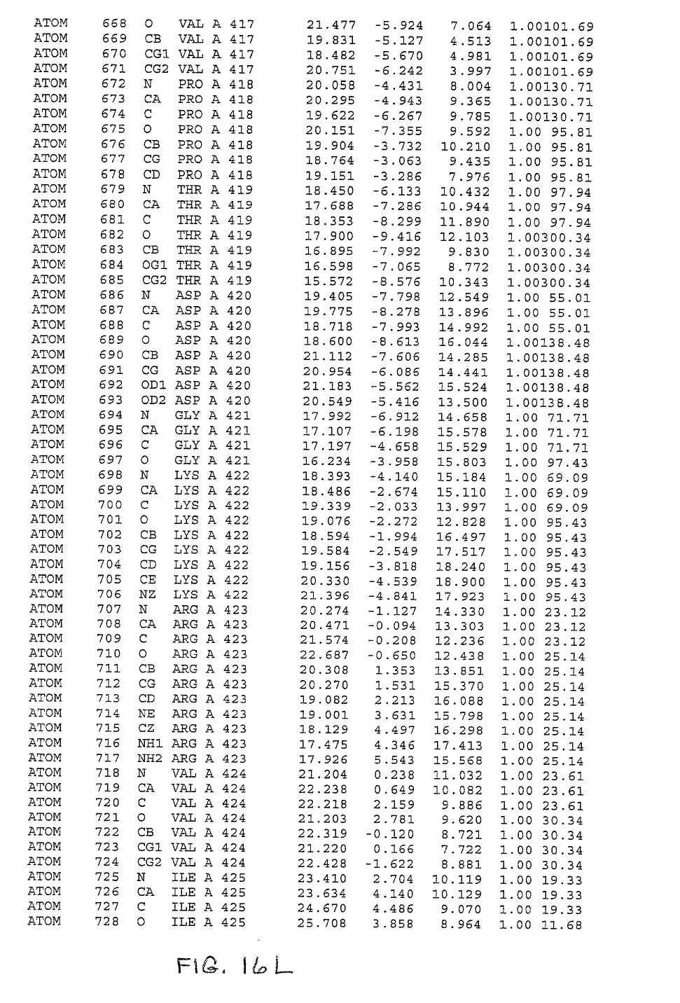

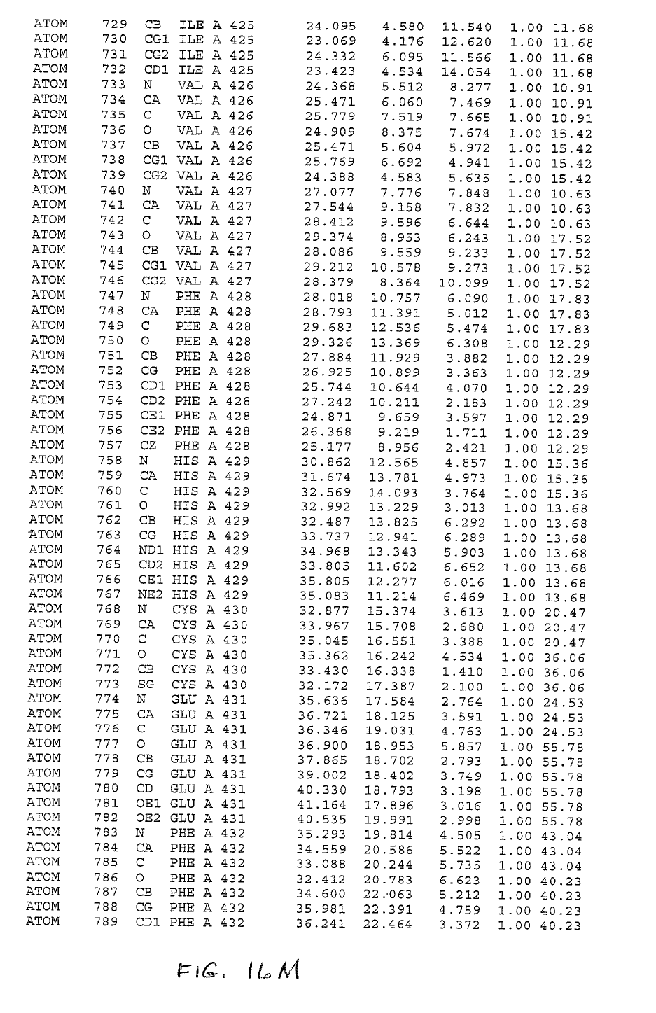

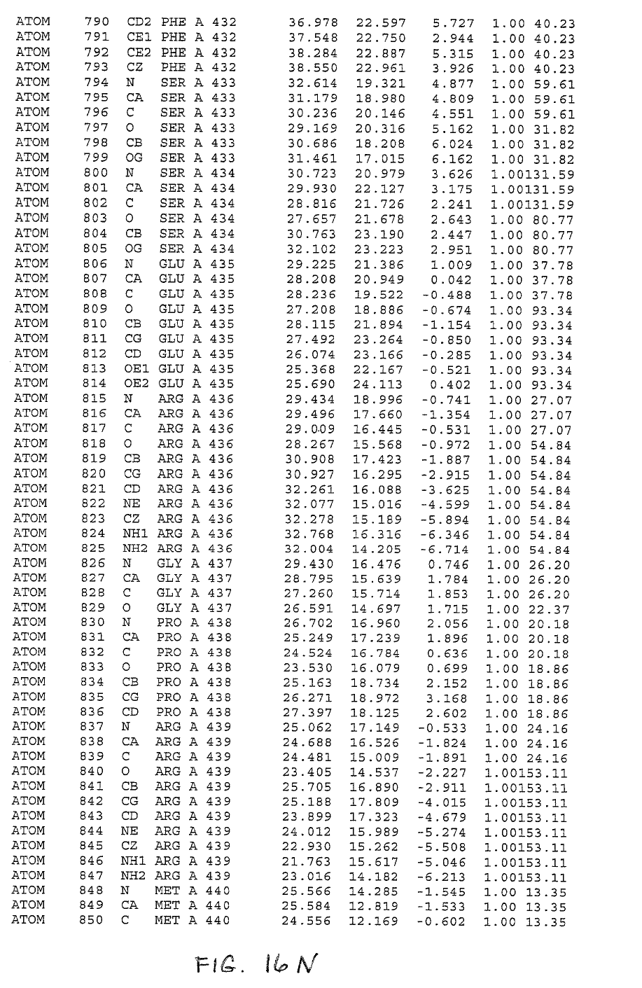

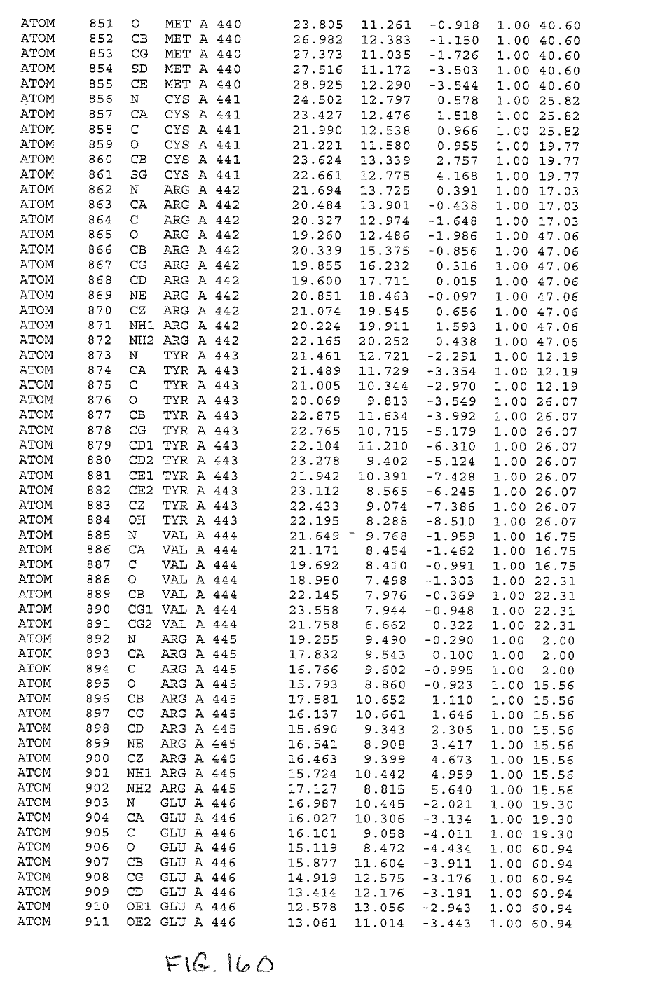

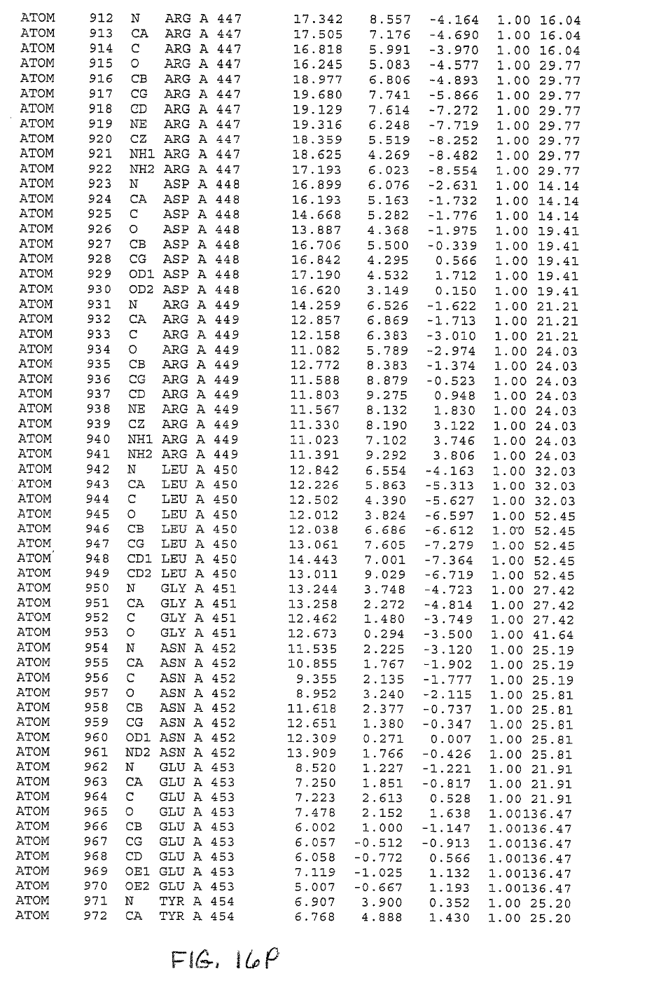

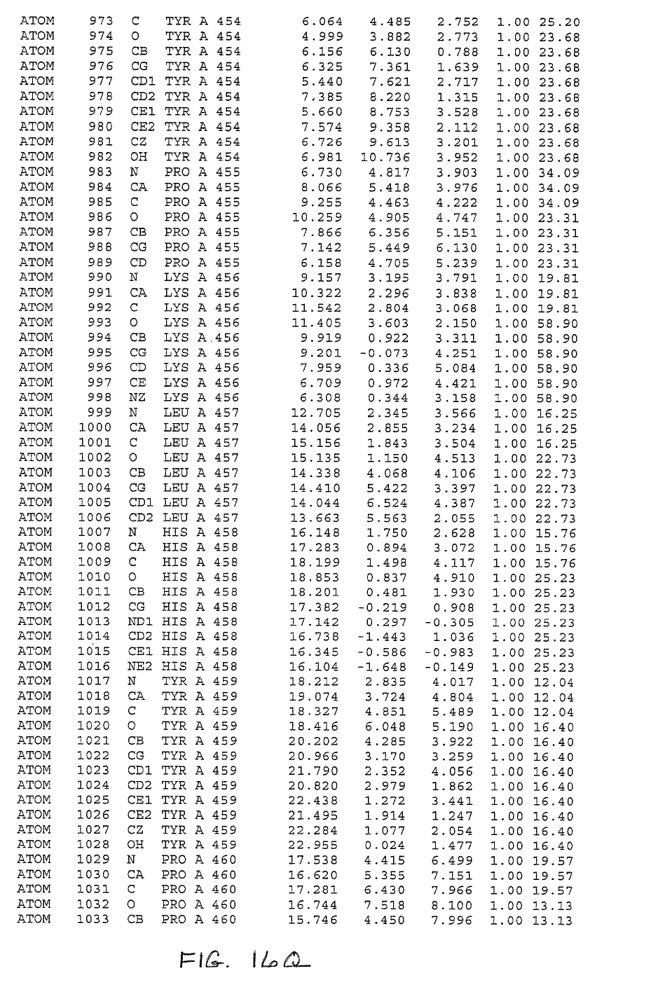

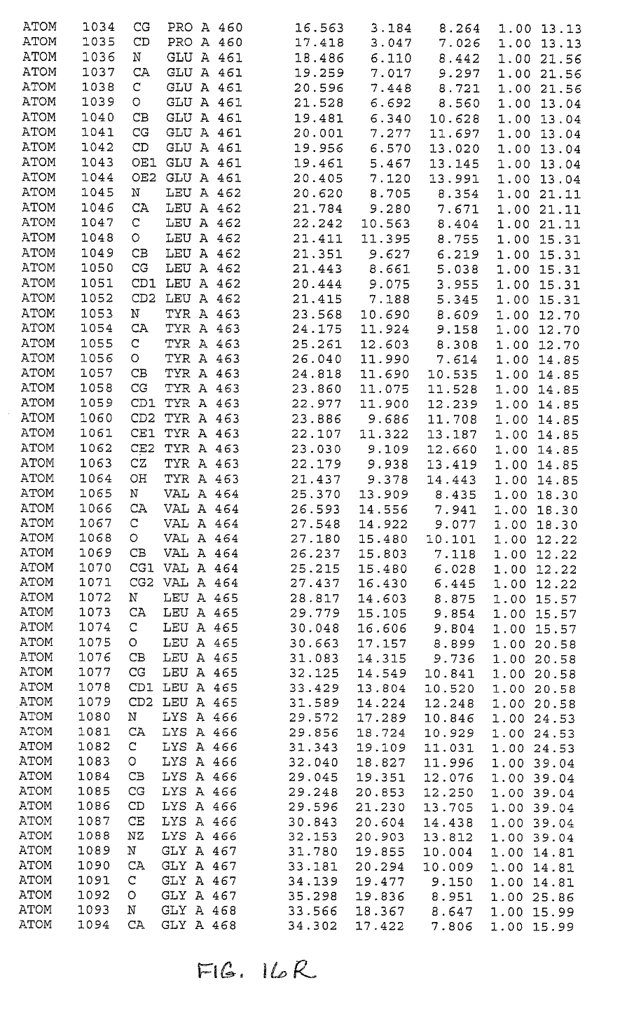

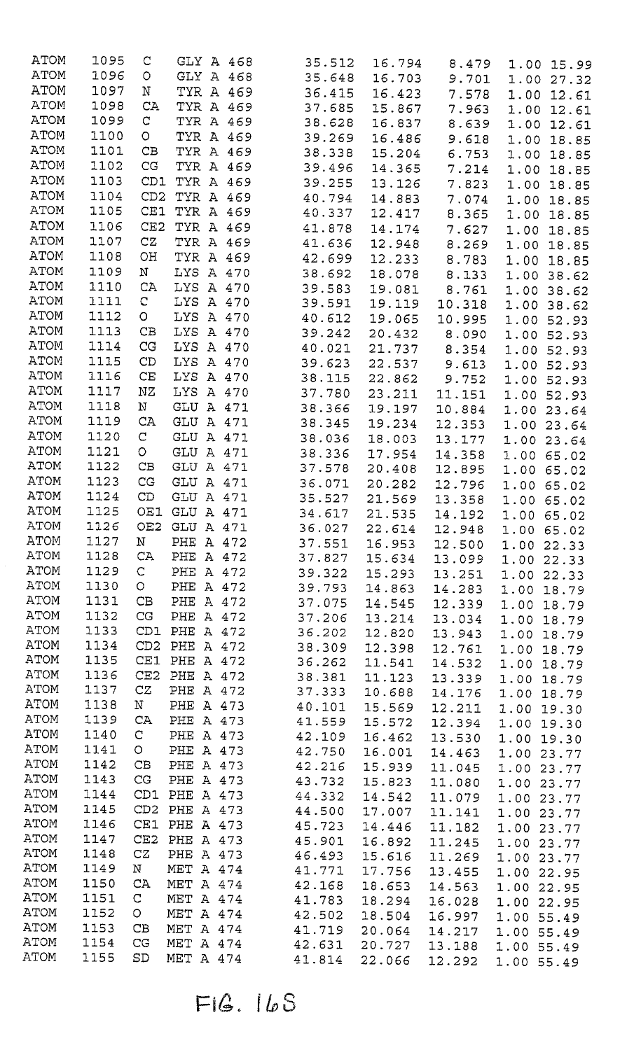

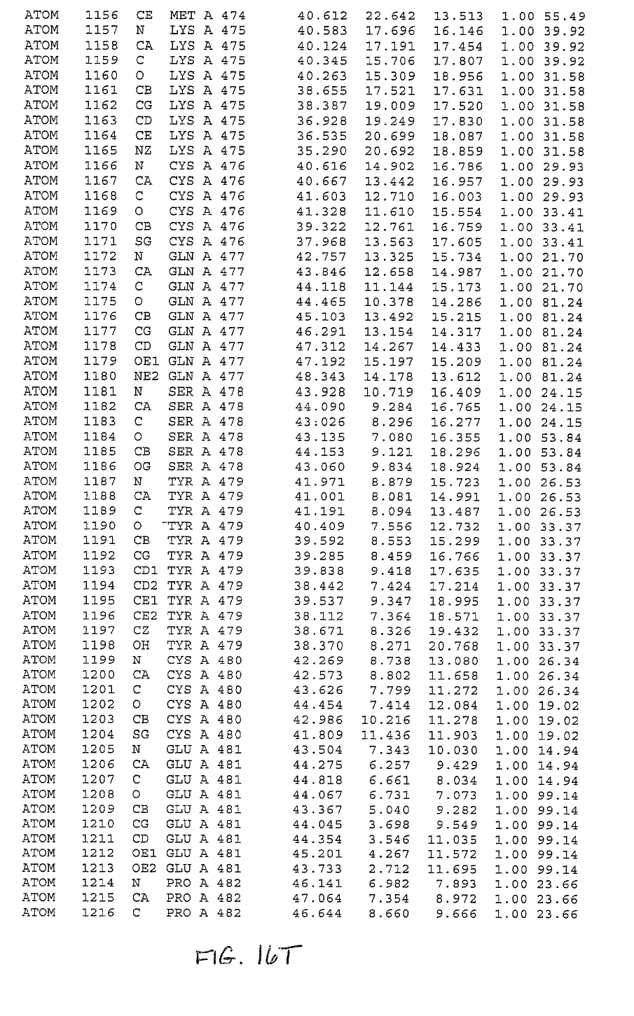

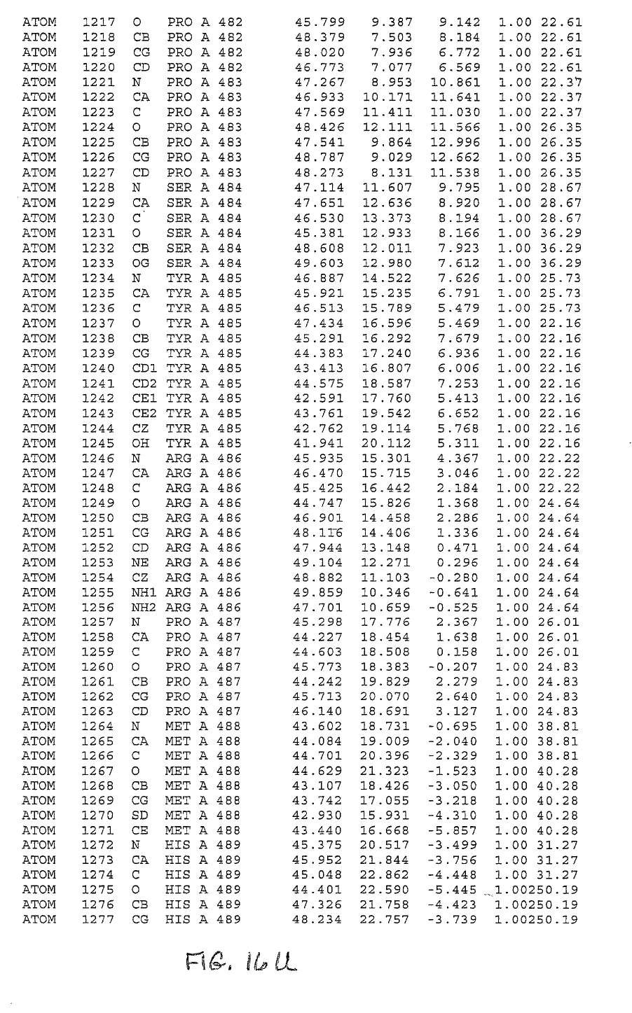

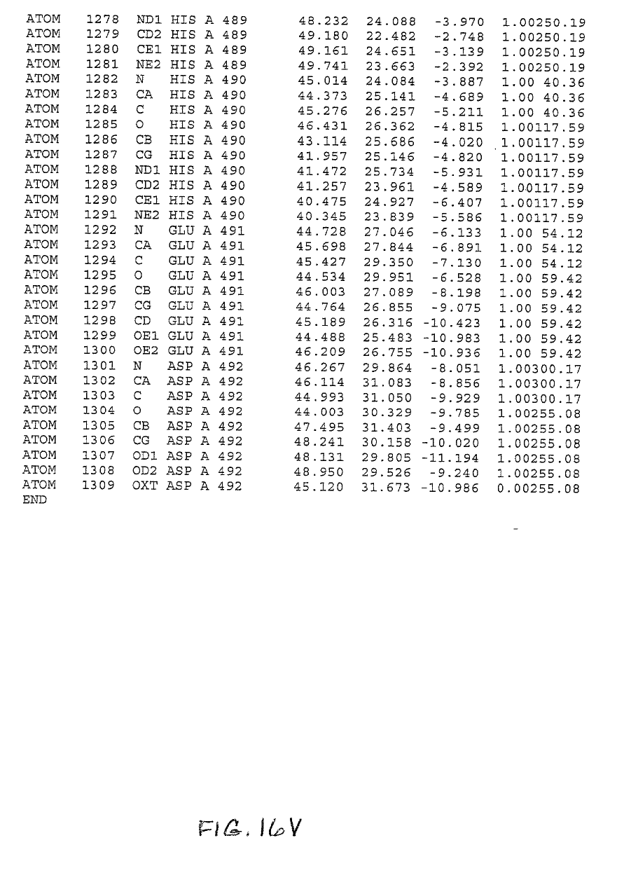

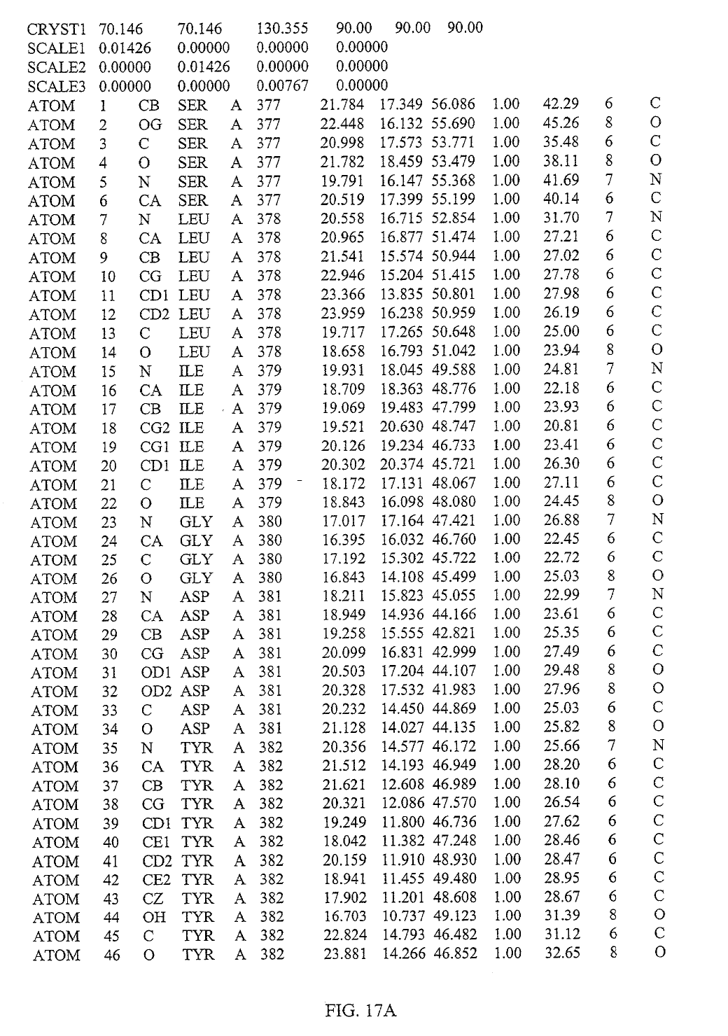

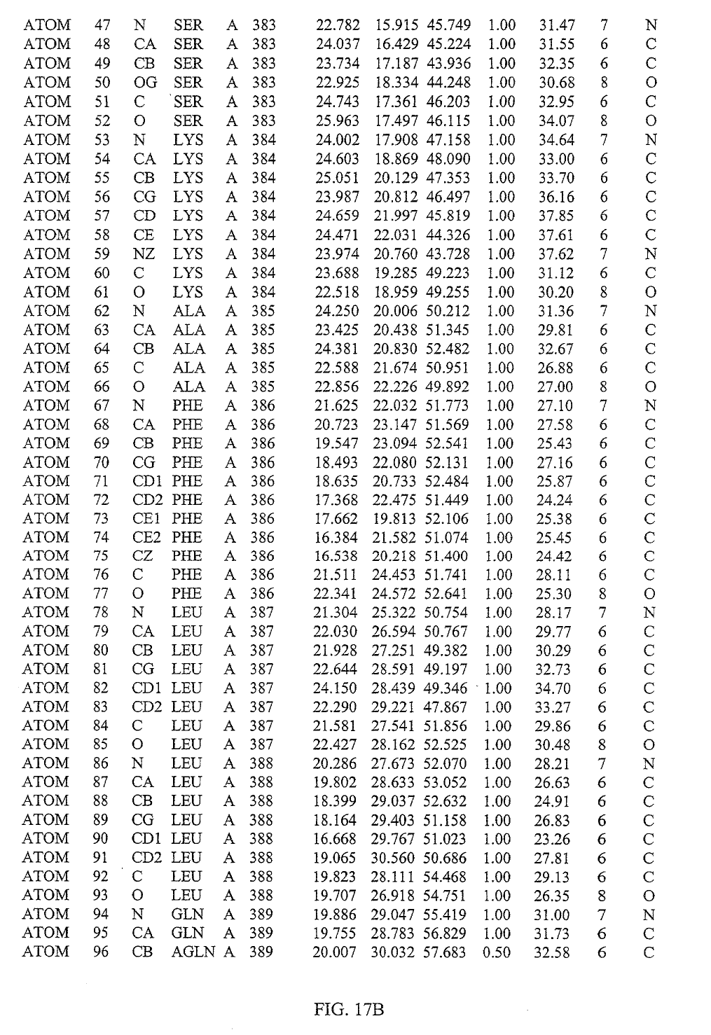

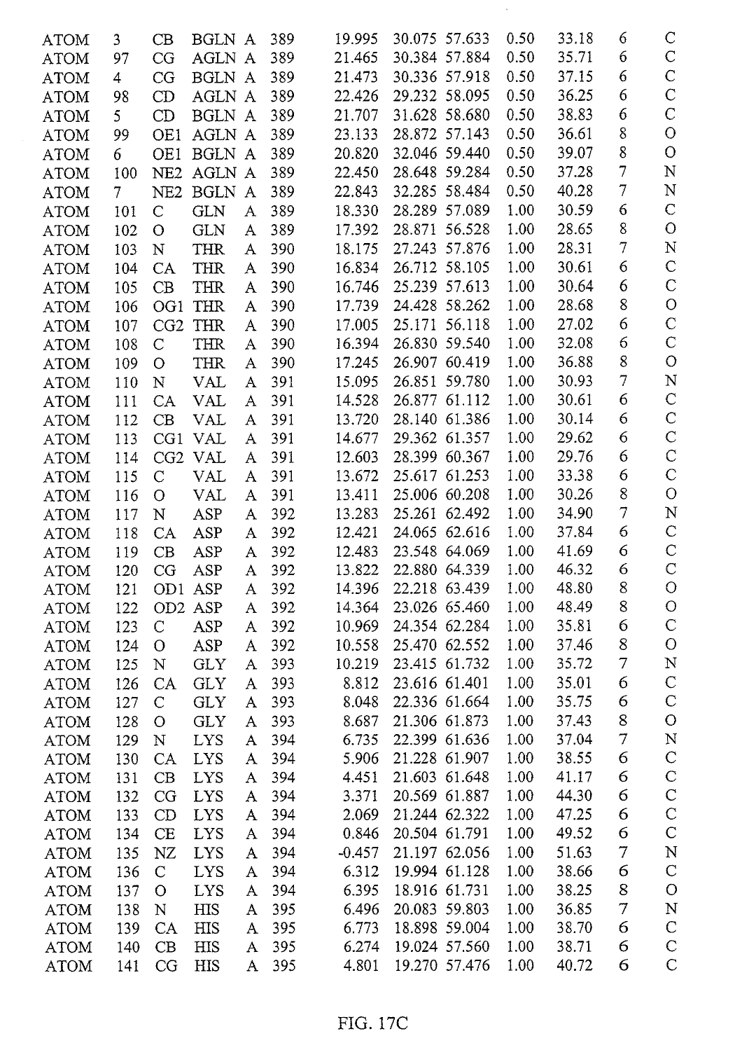

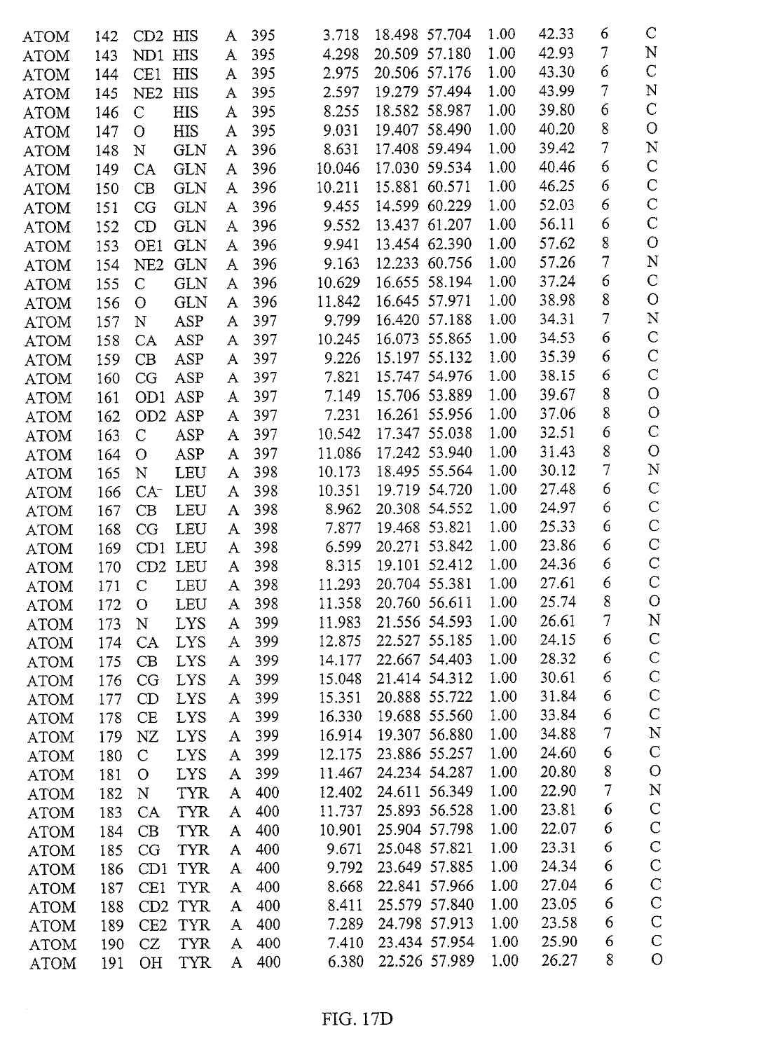

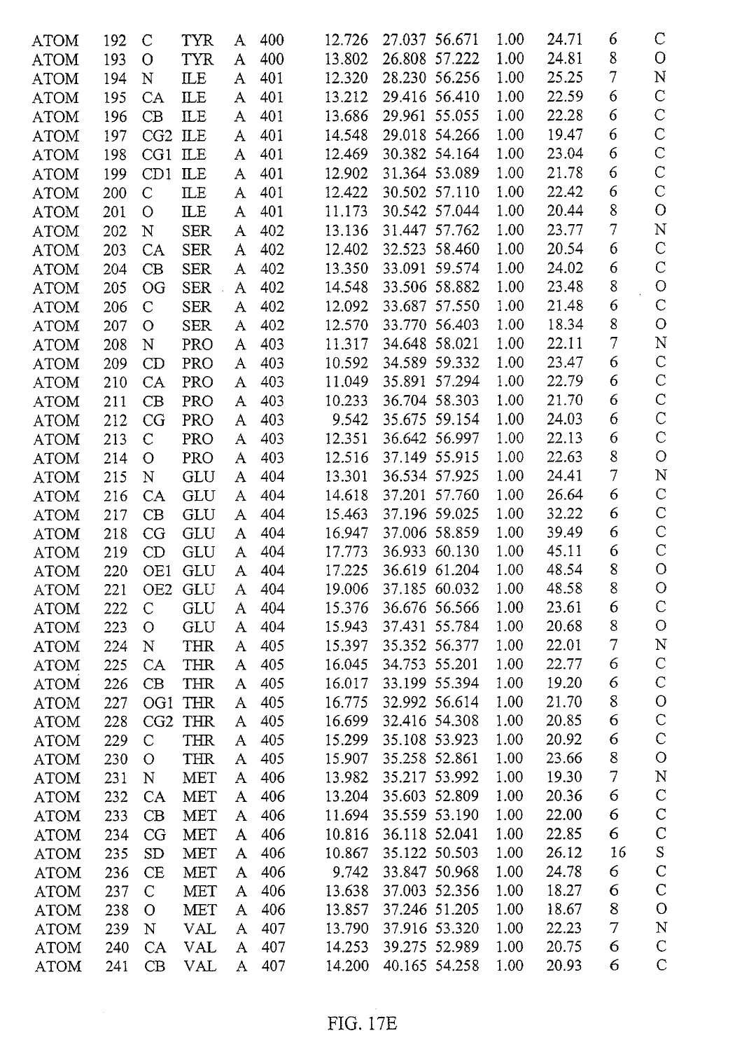

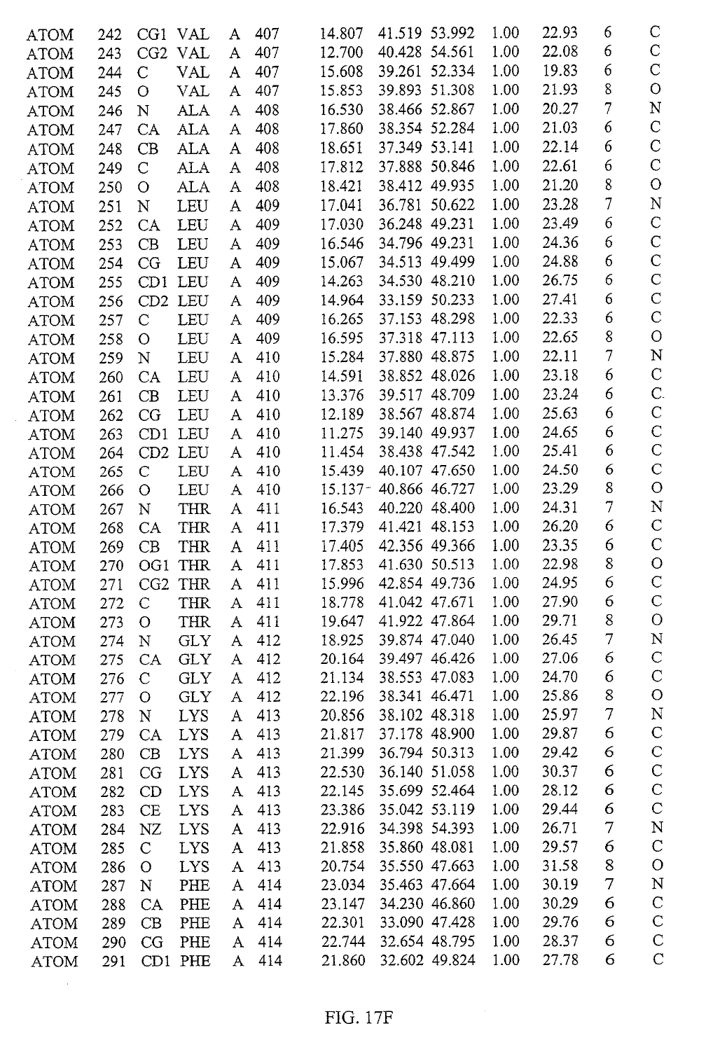

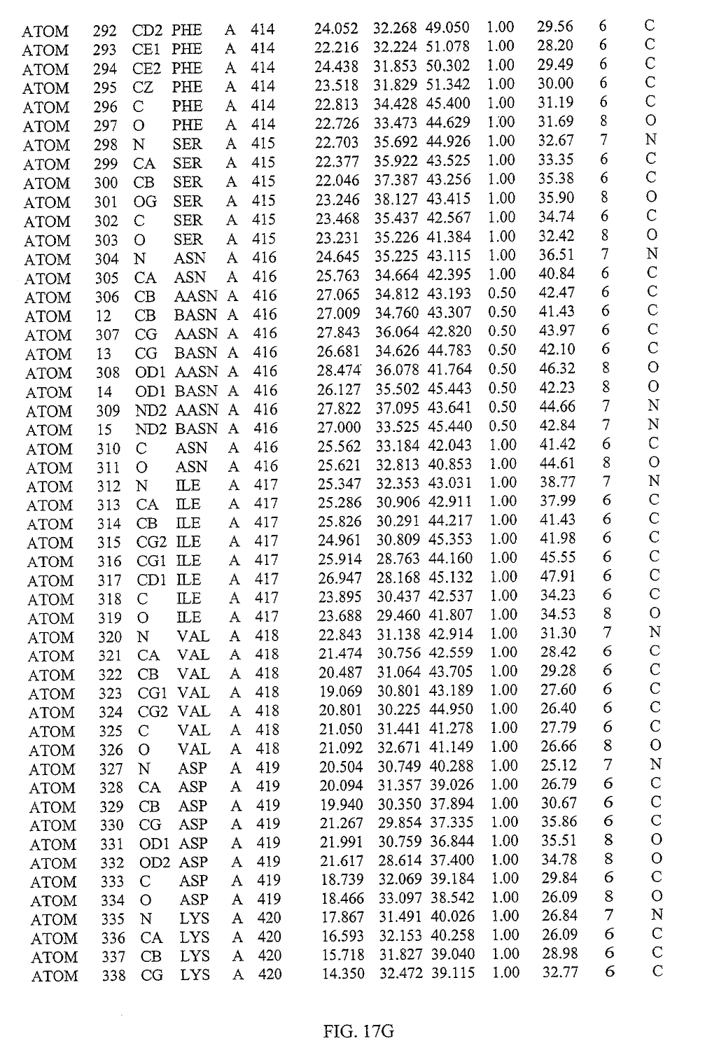

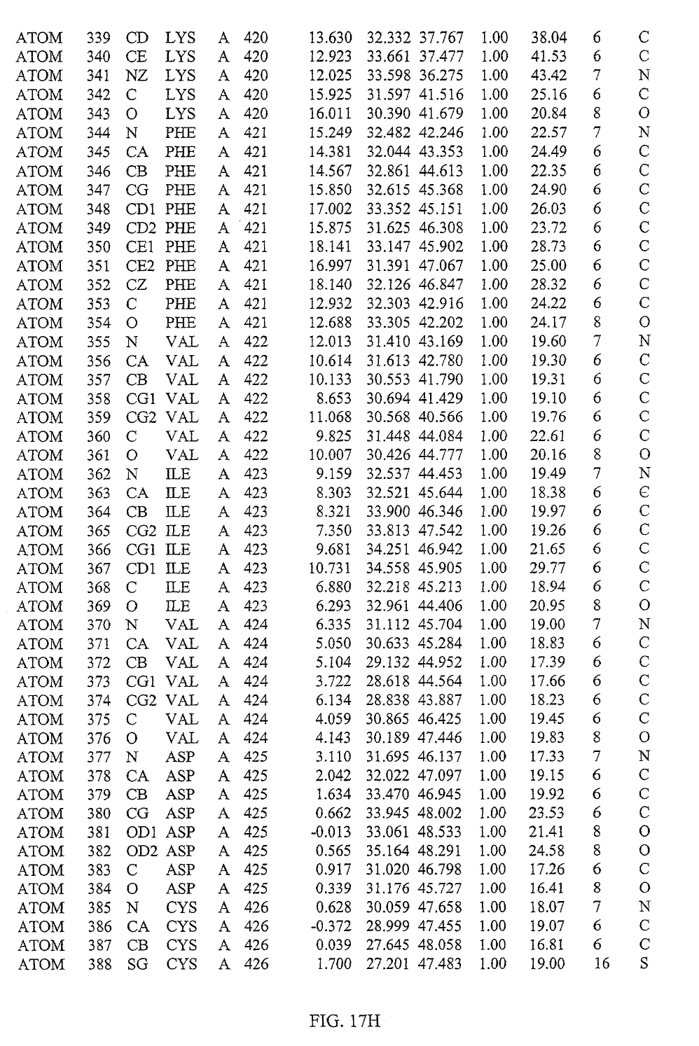

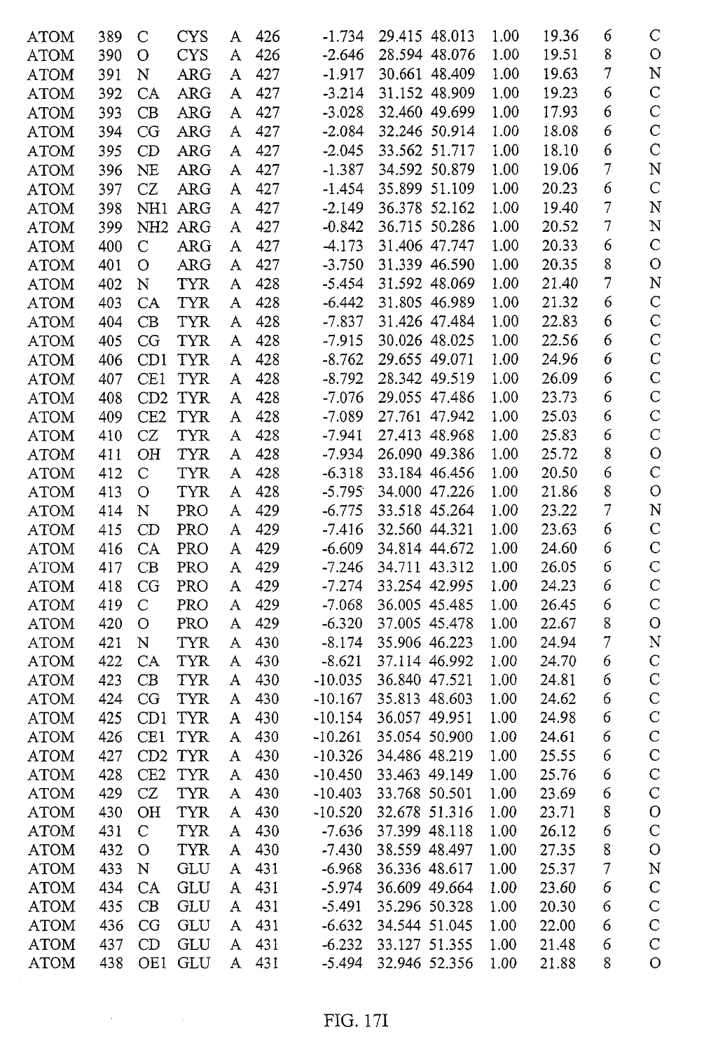

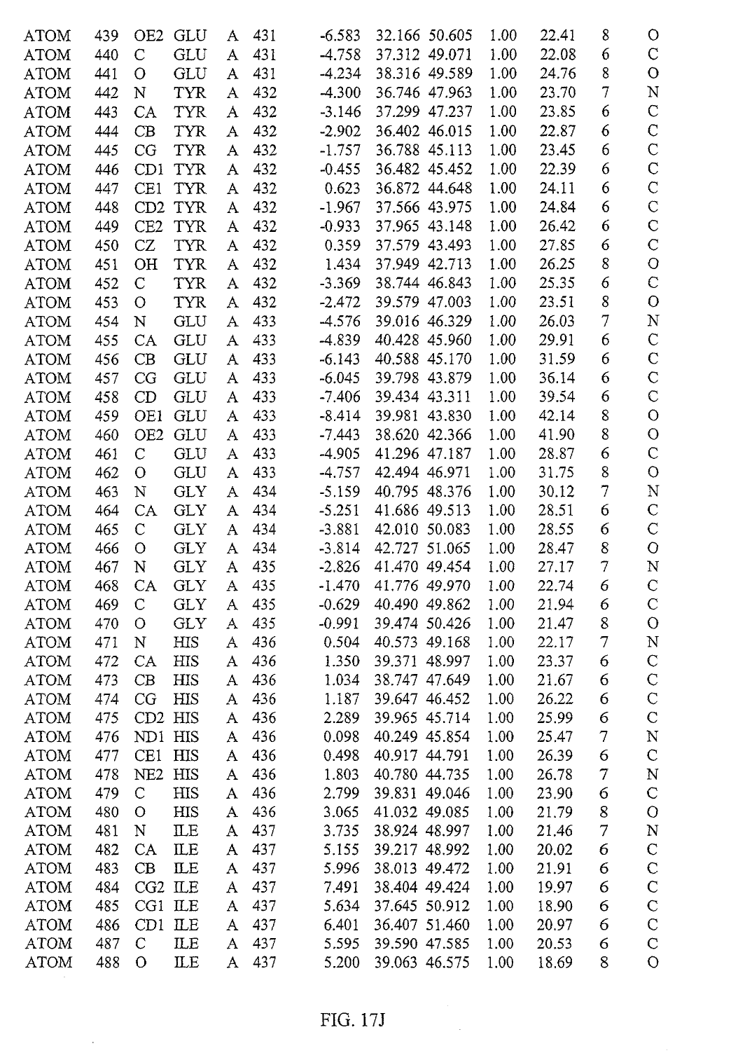

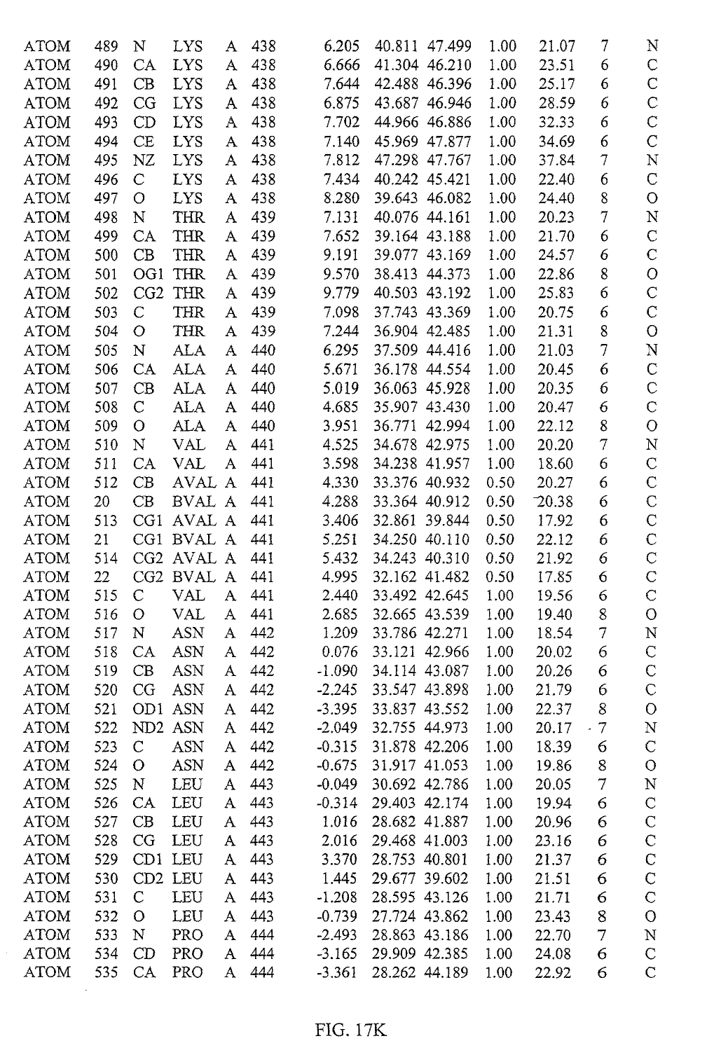

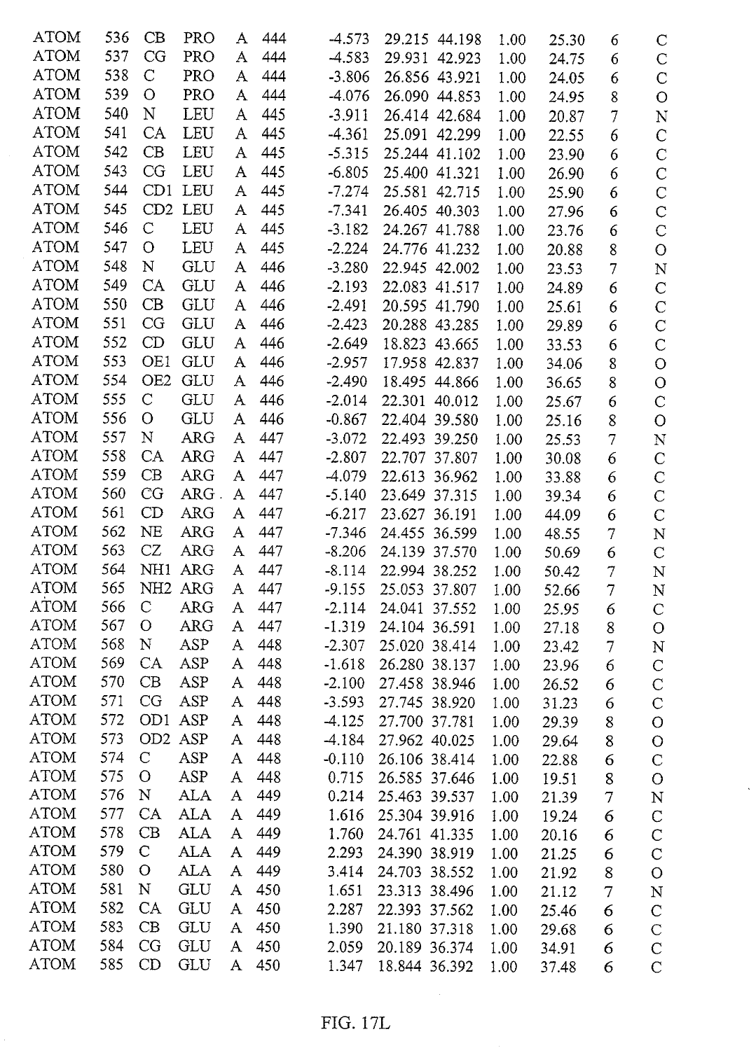

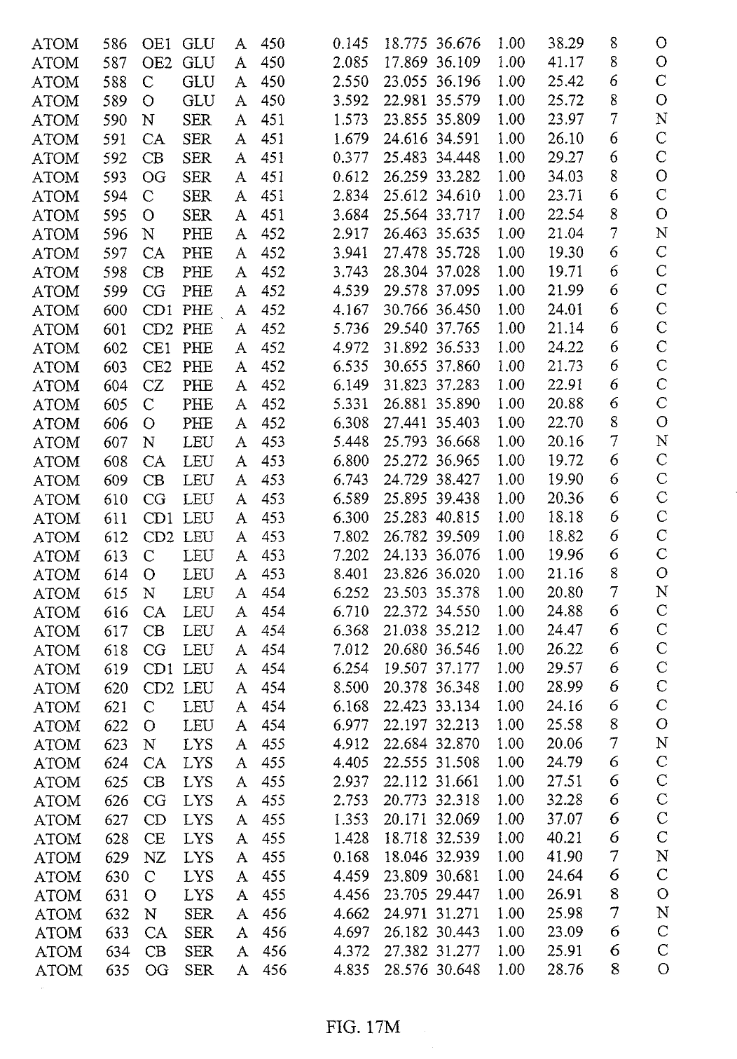

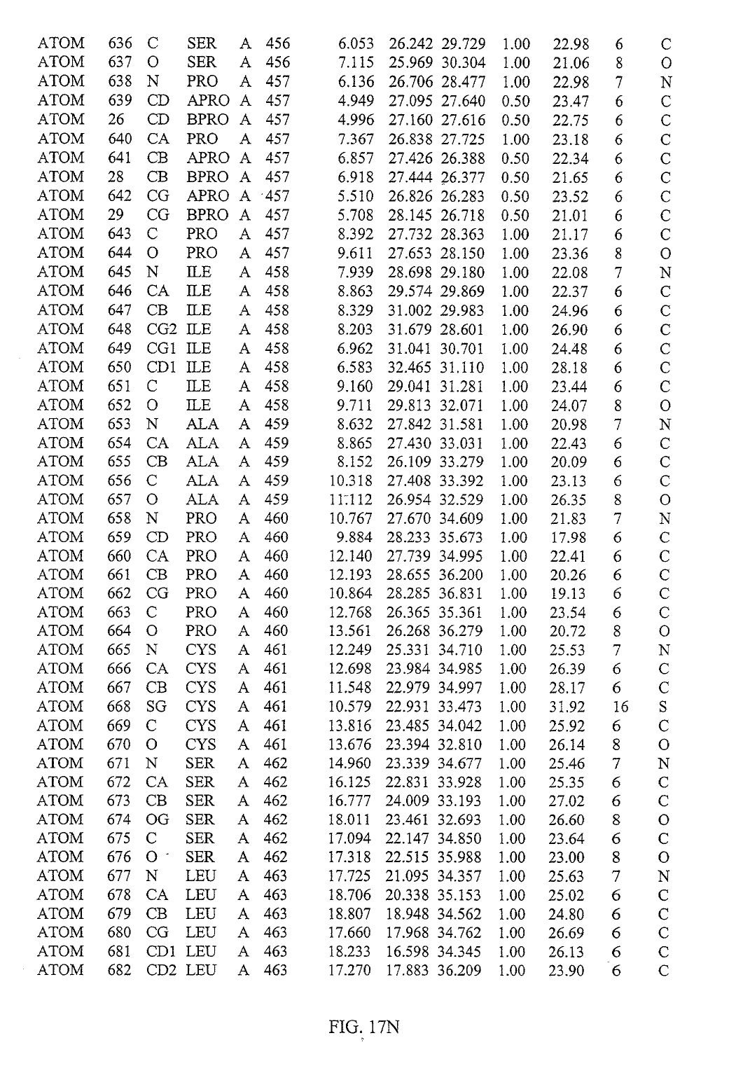

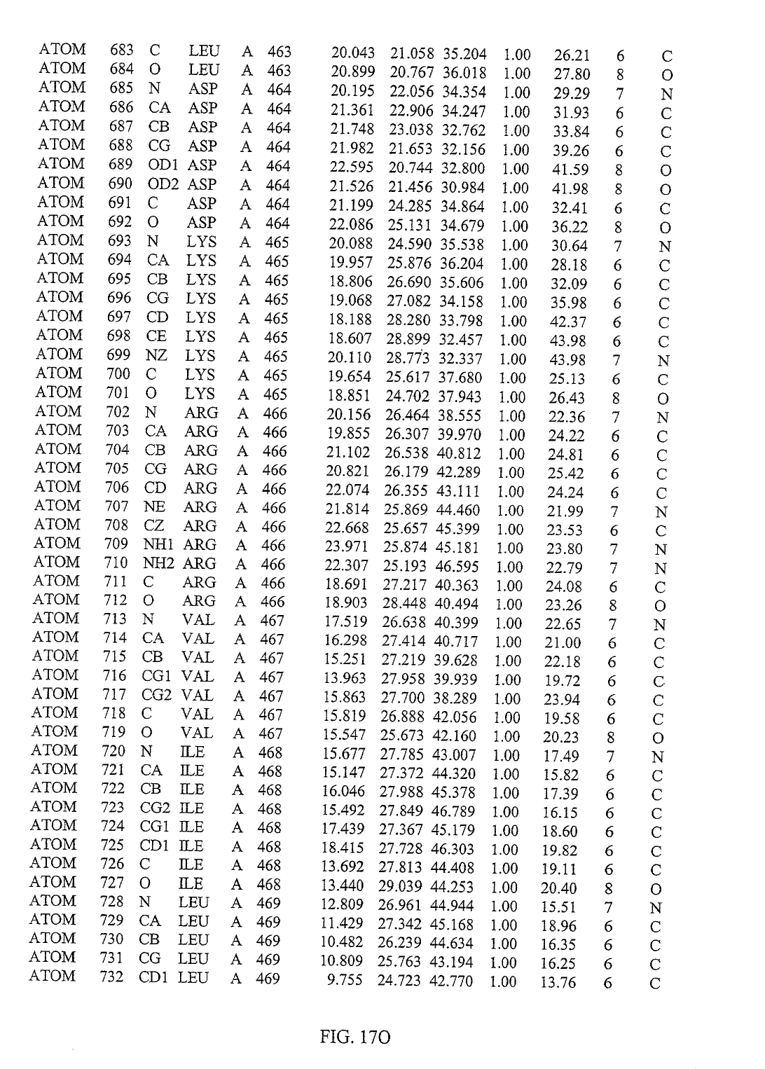

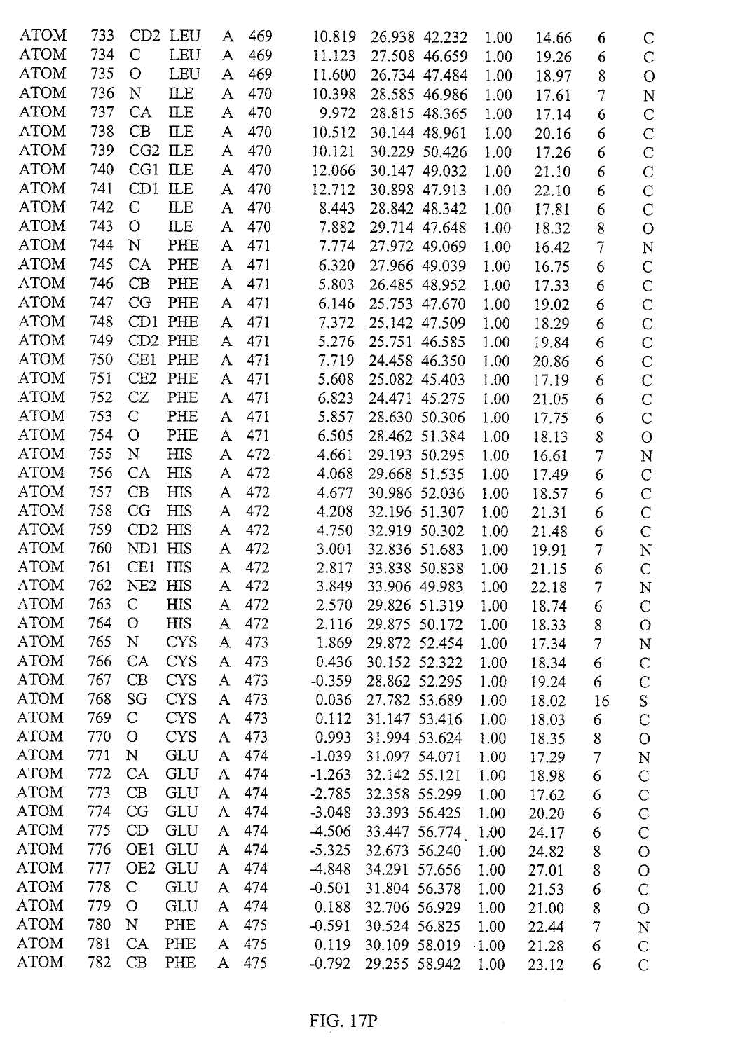

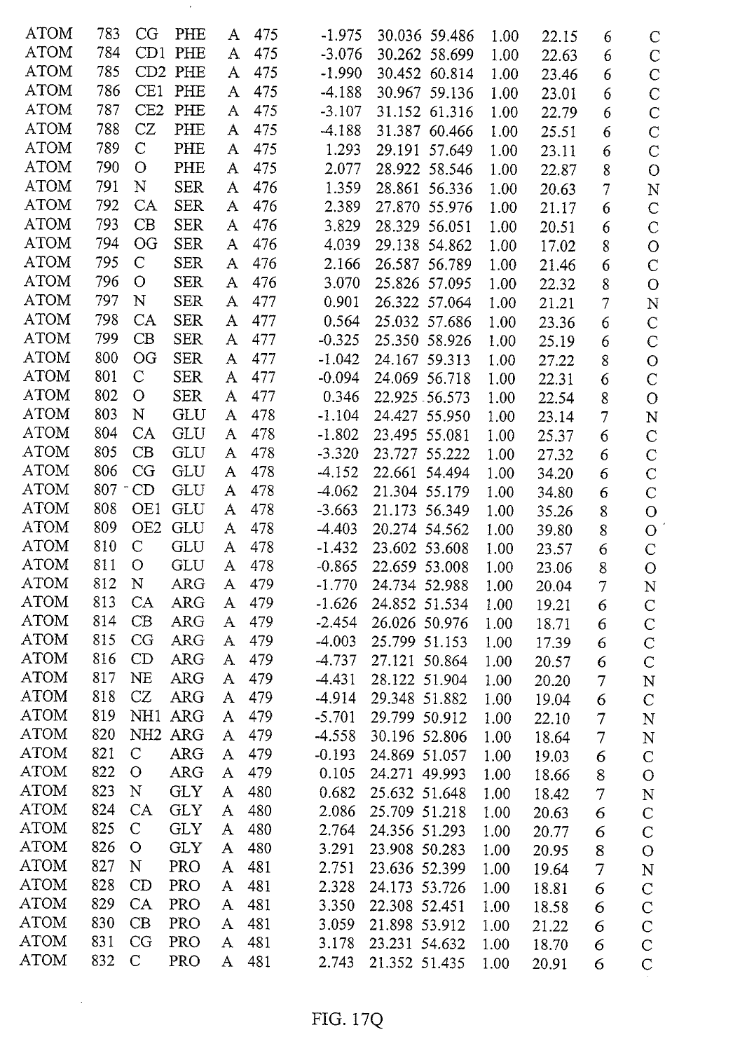

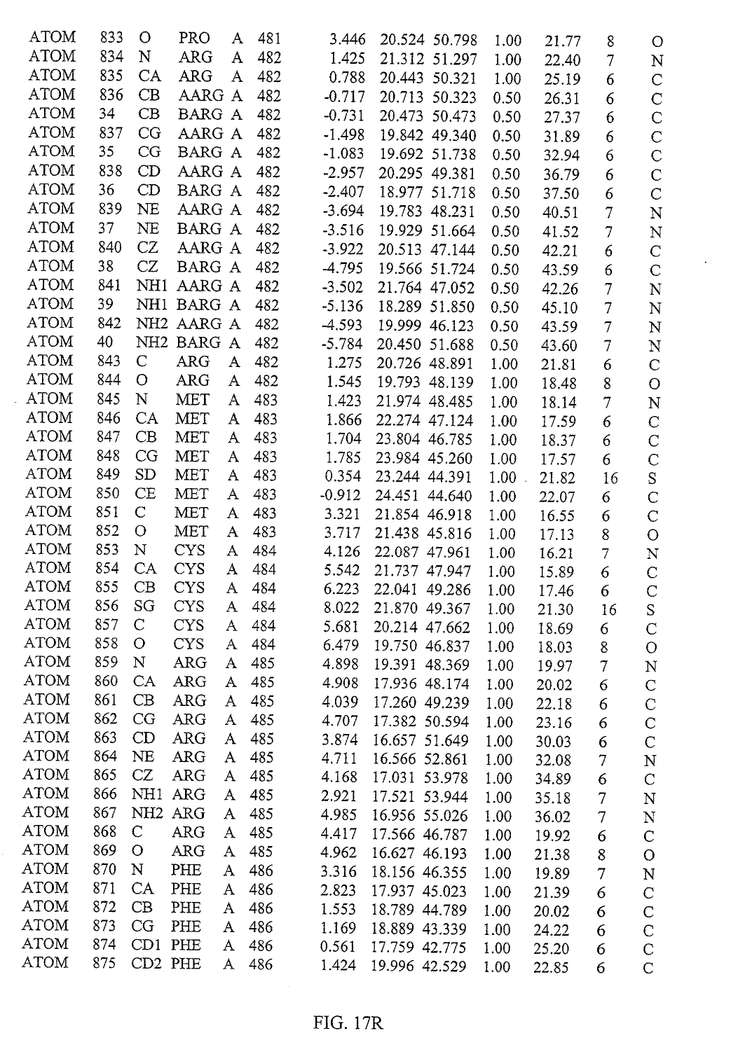

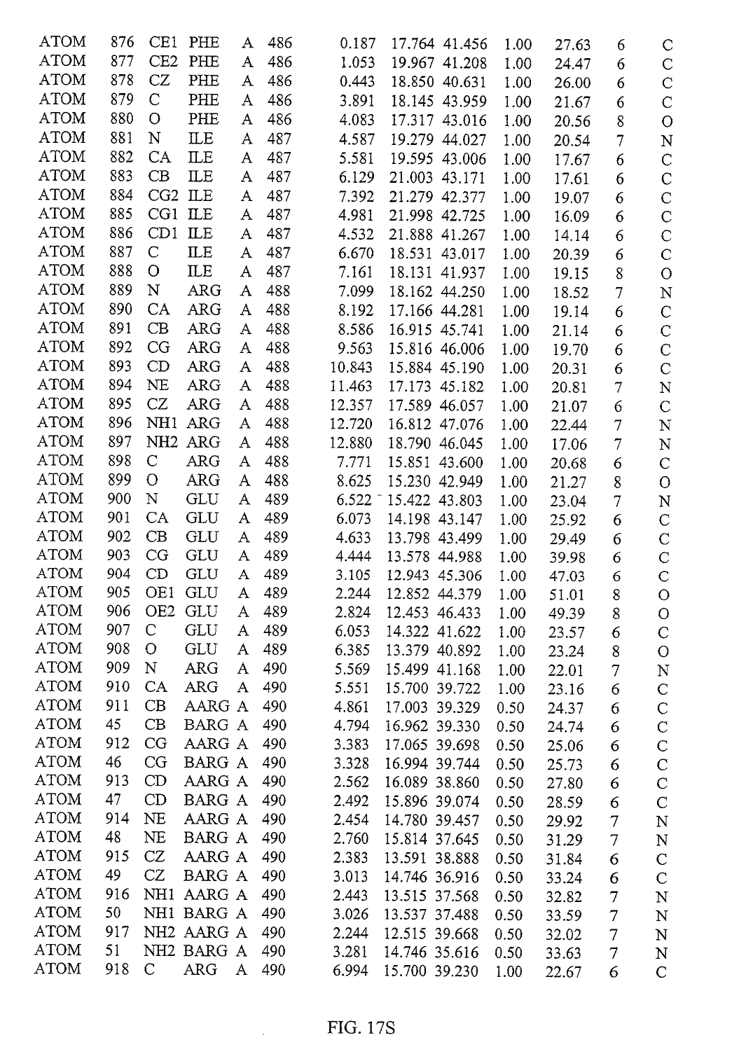

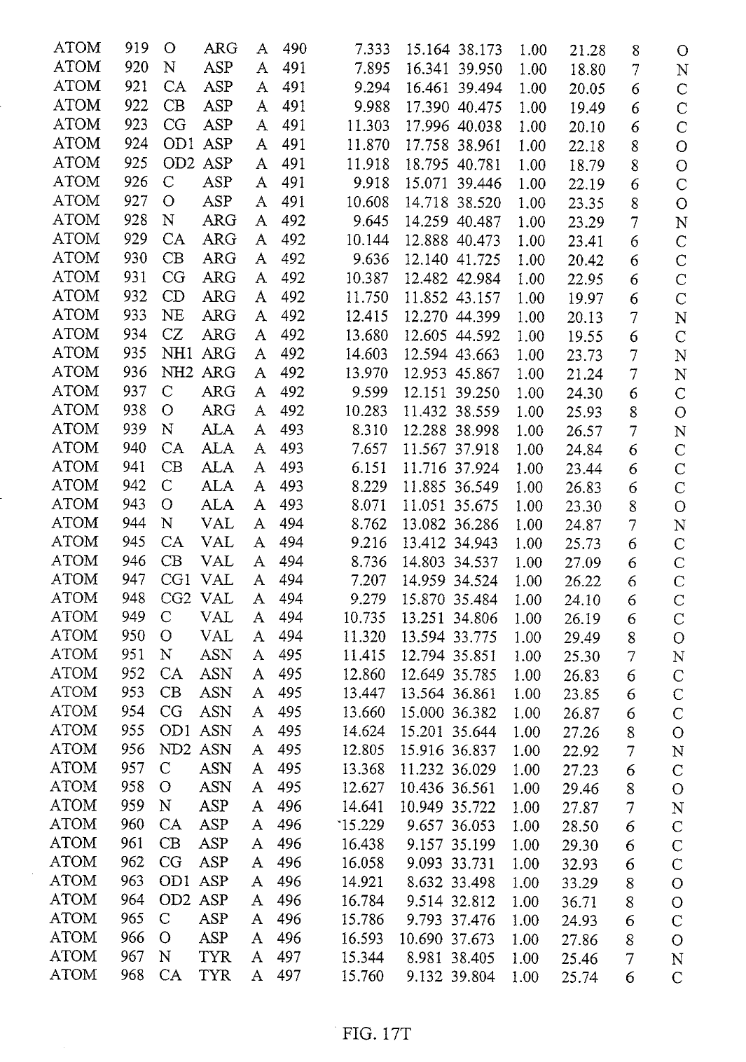

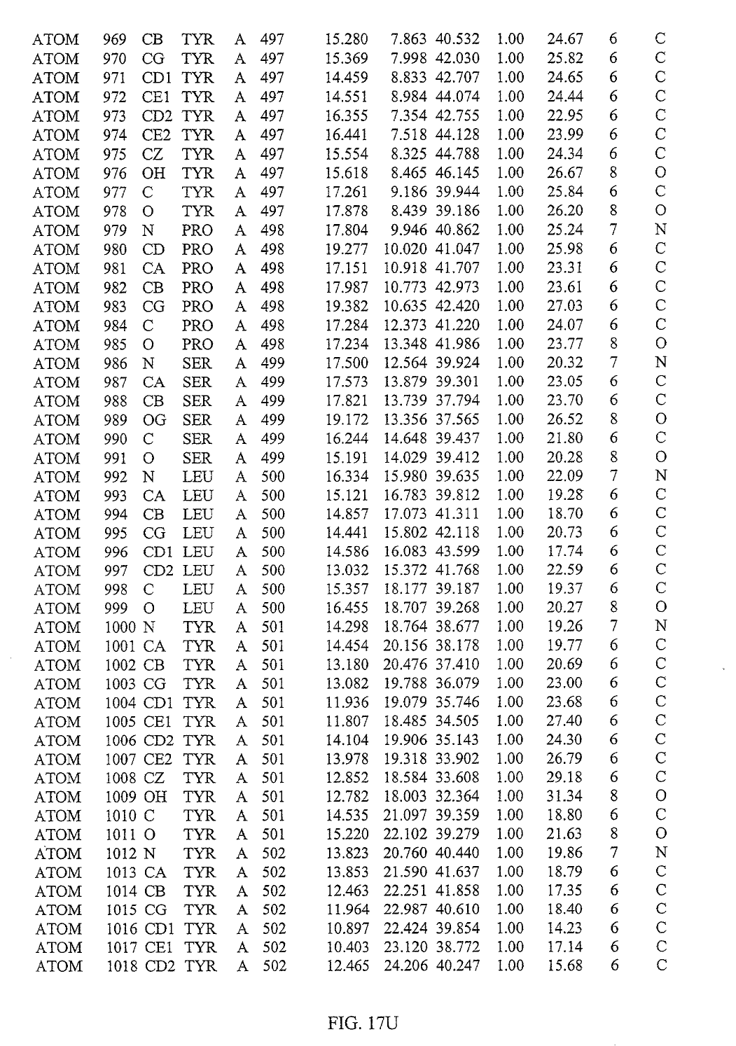

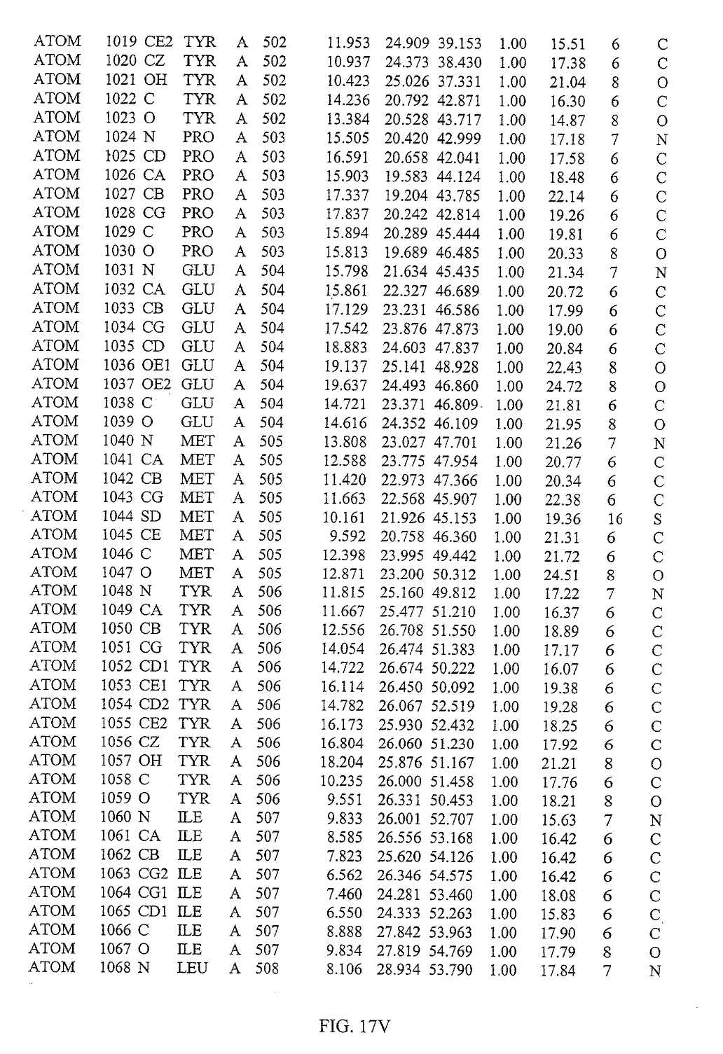

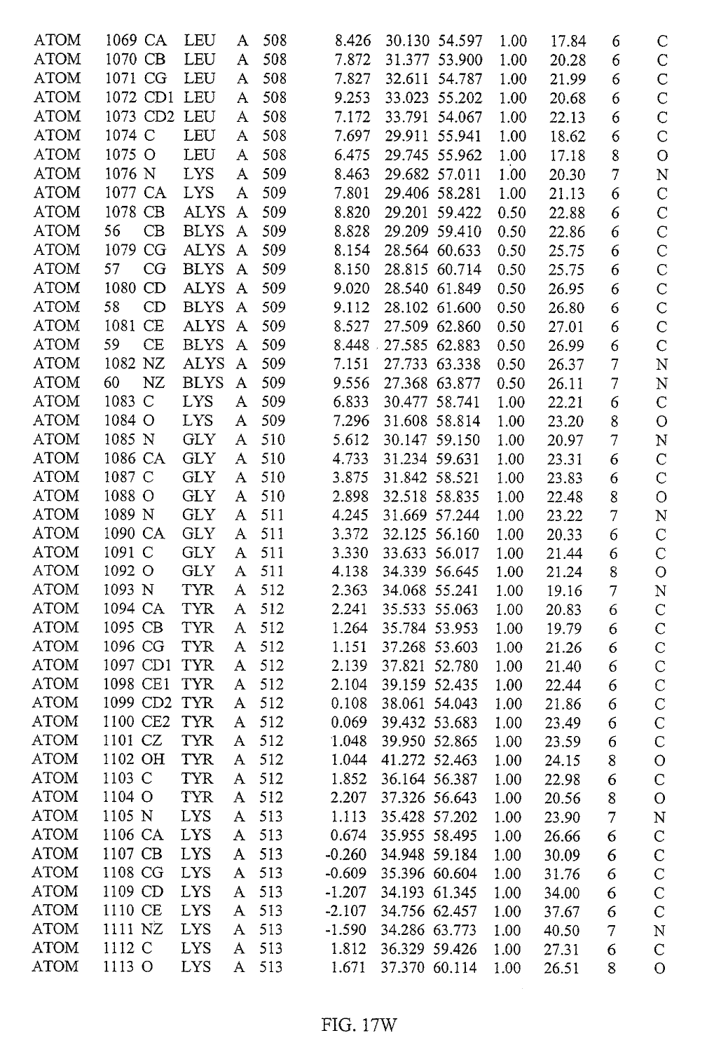

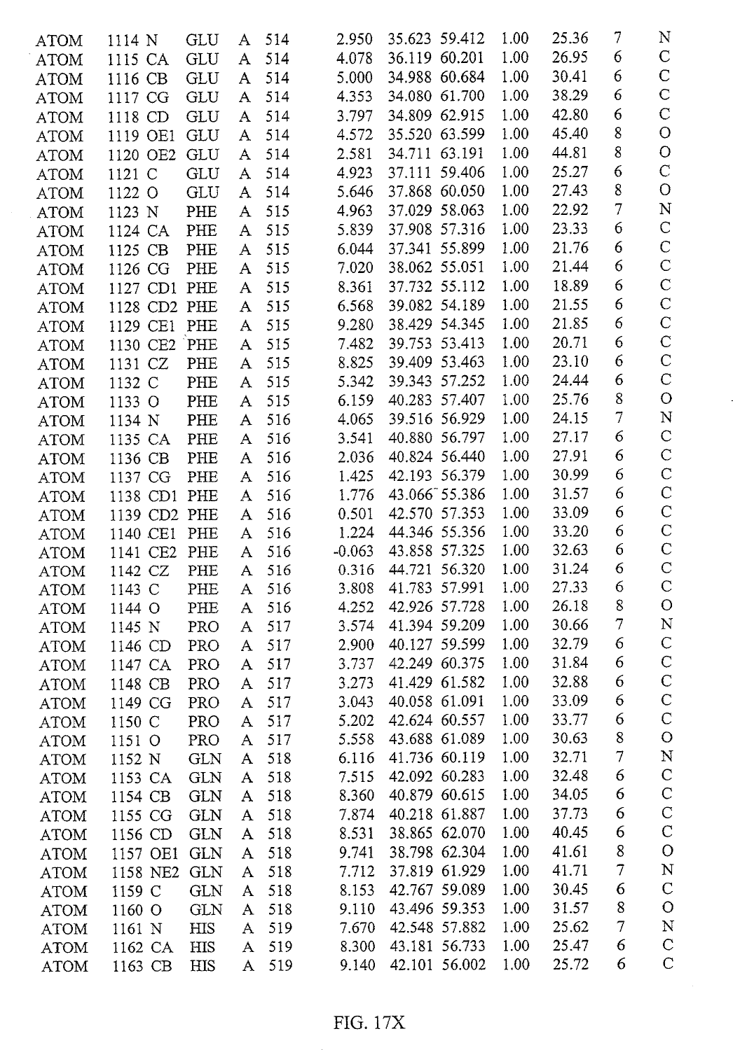

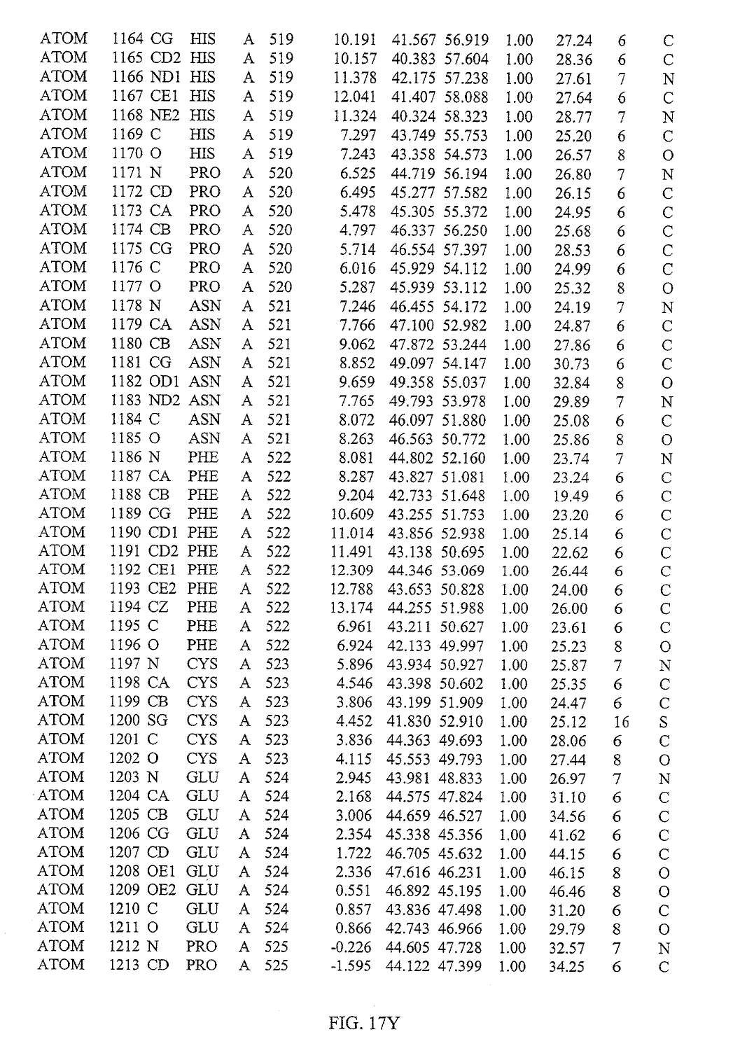

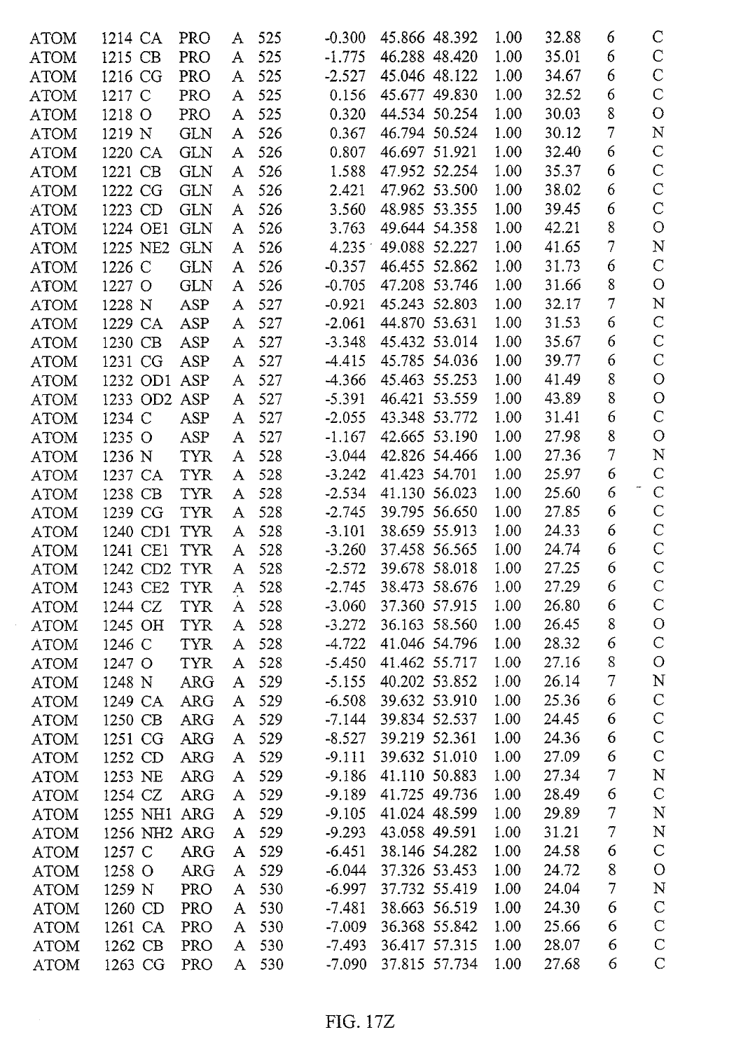

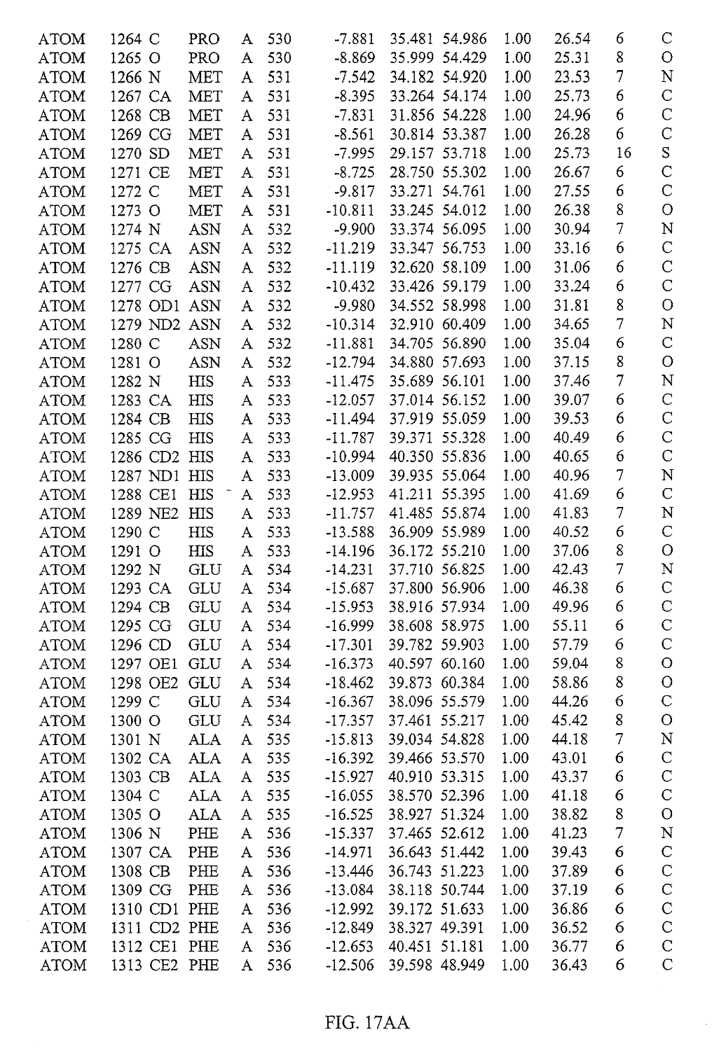

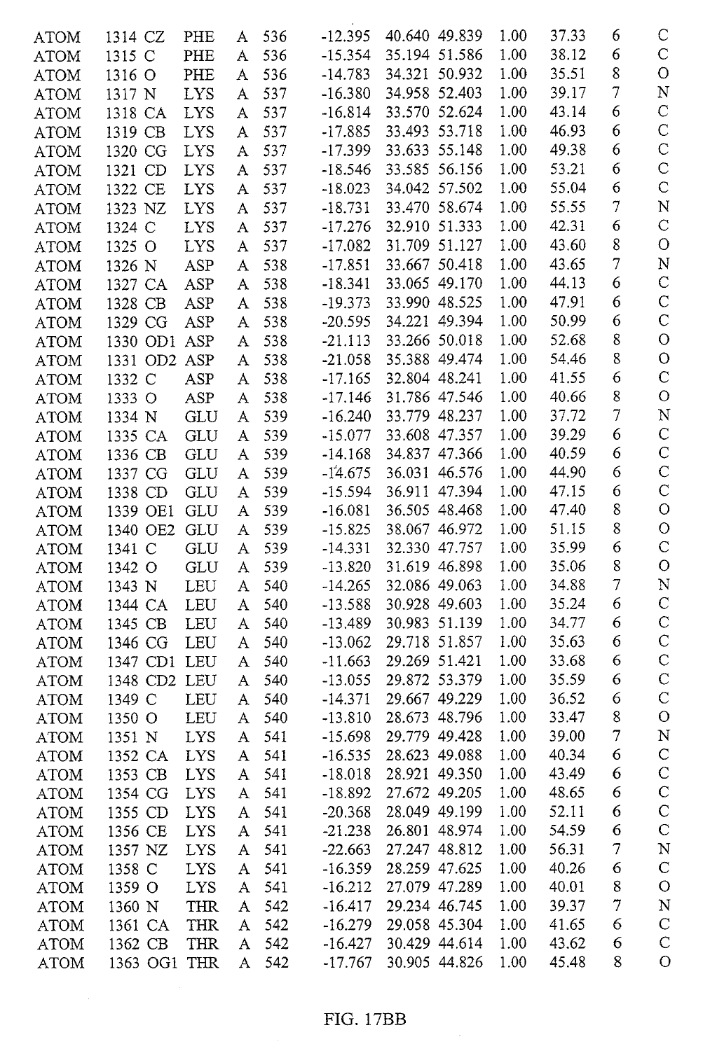

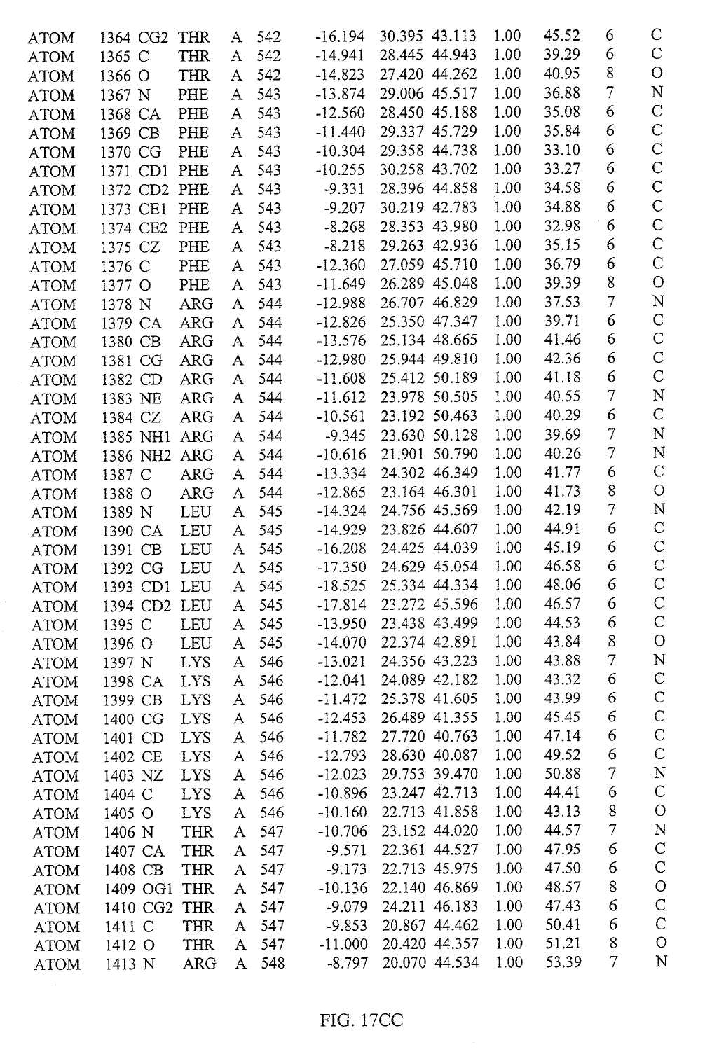

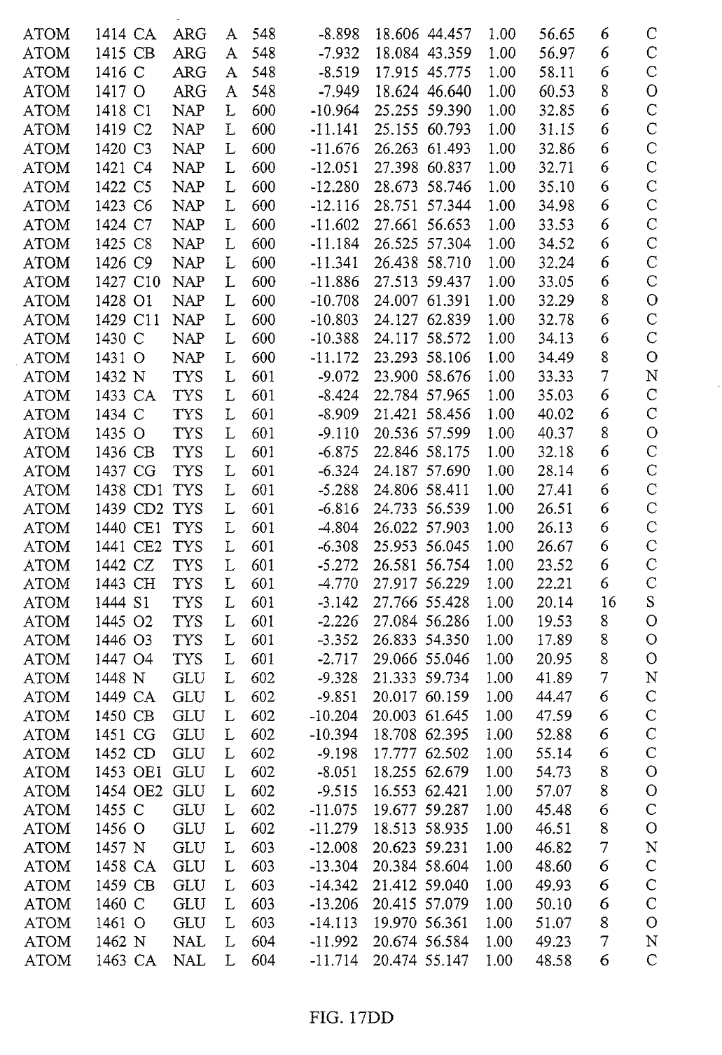

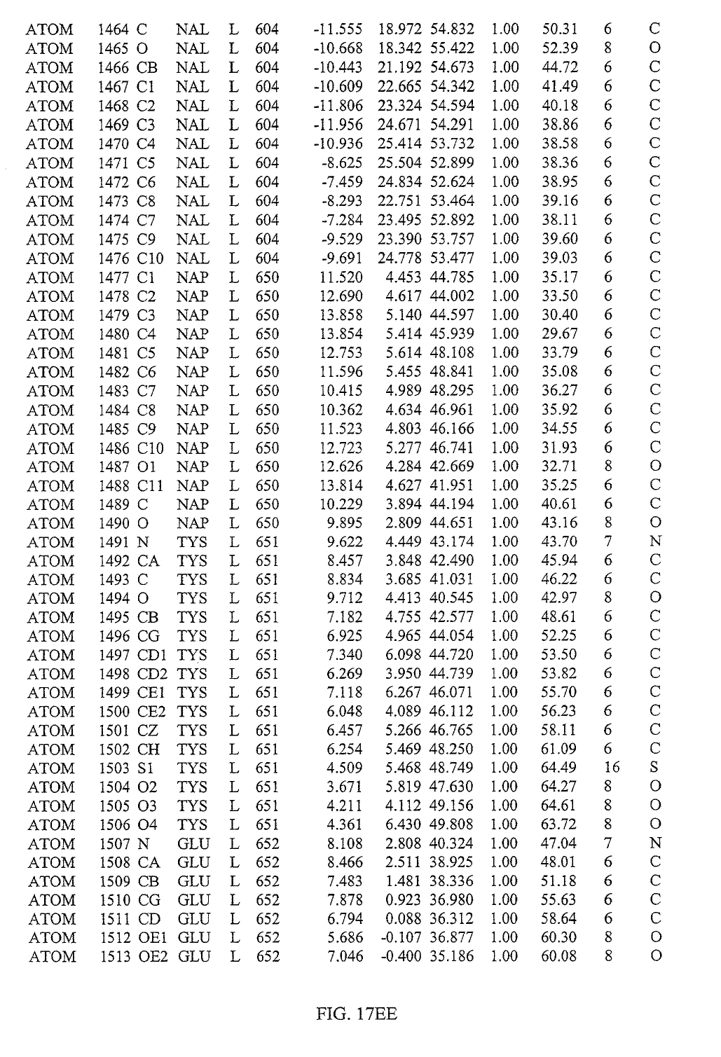

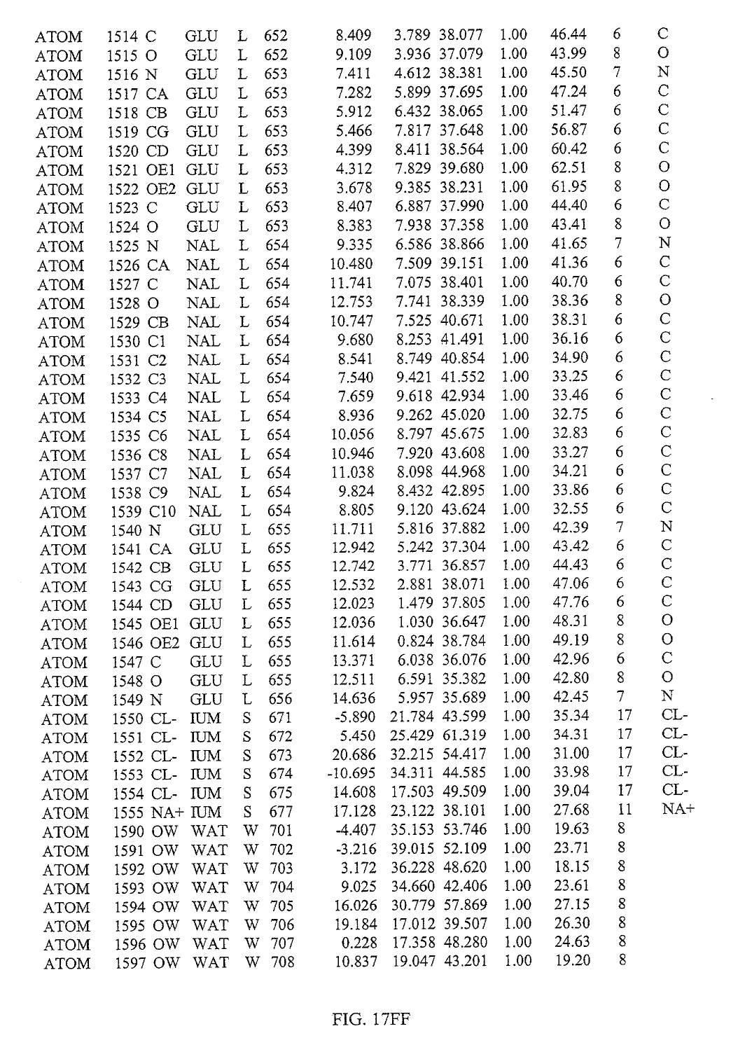

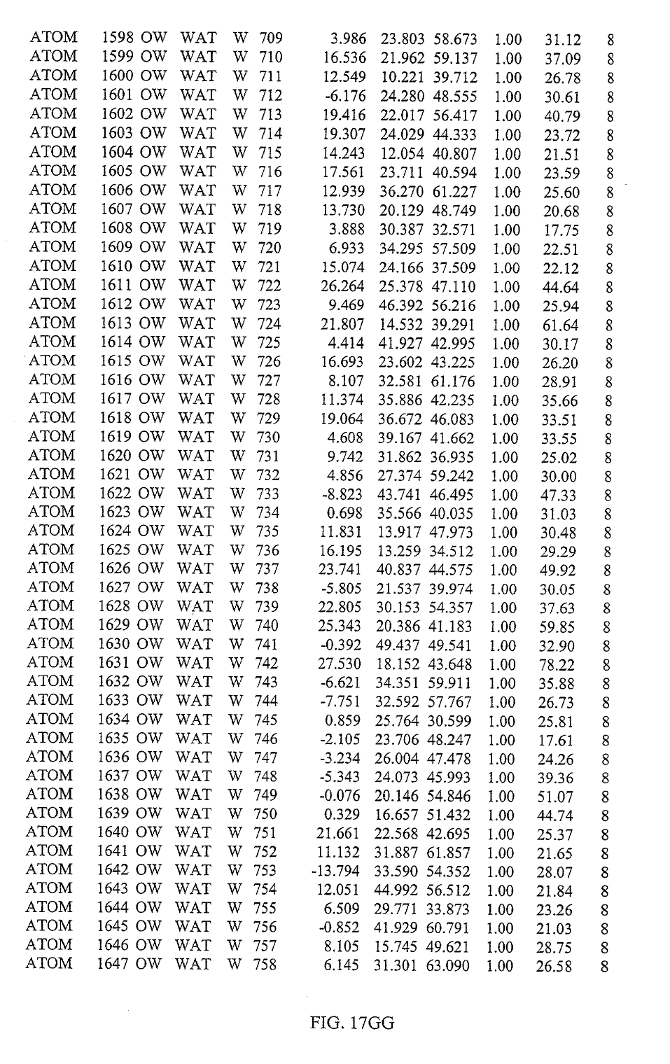

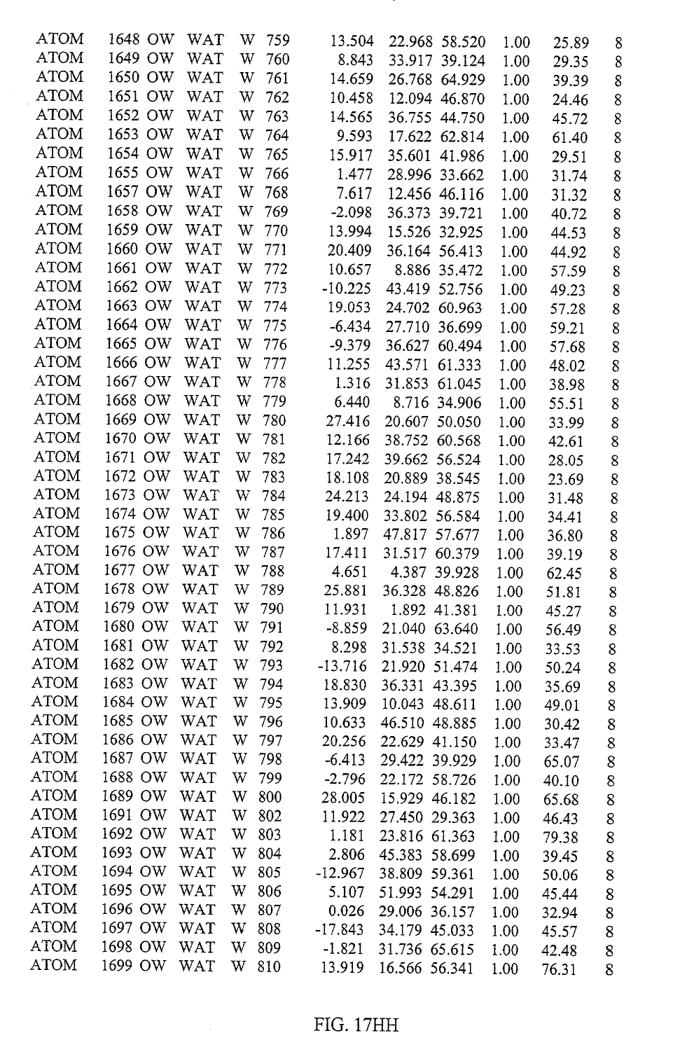

38. The method of claim 32 wherein the crystal structure coordinates are set forth in FIGS. 17A to 17EE.

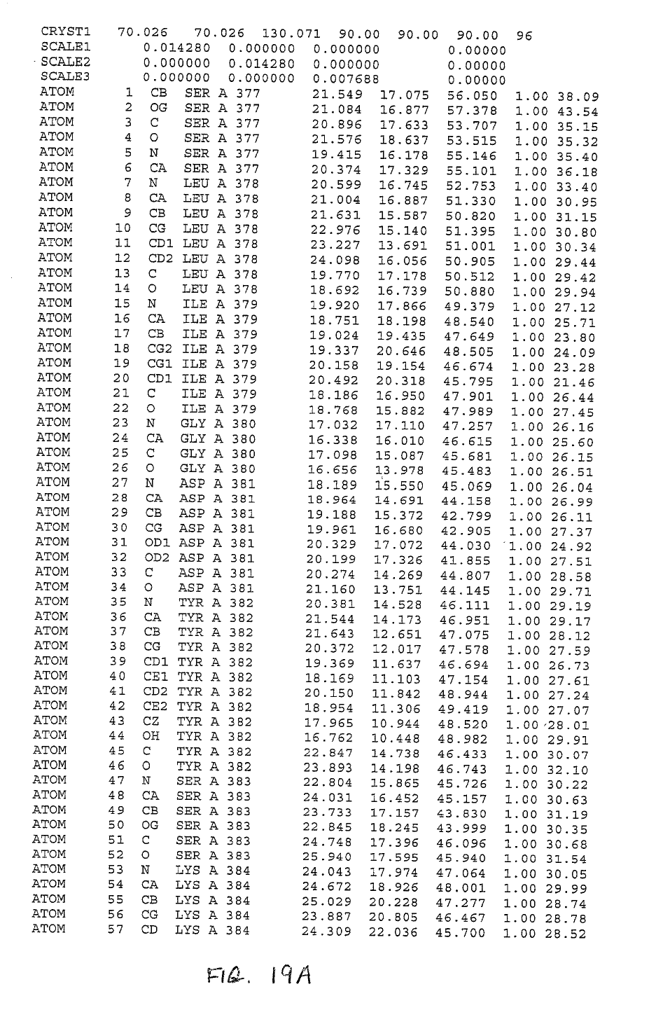

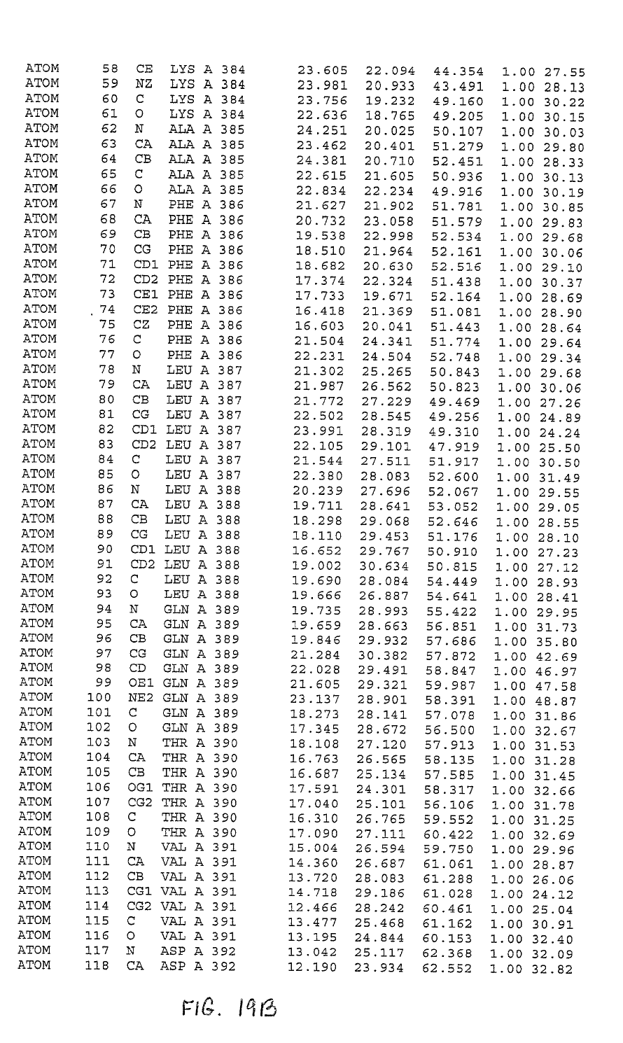

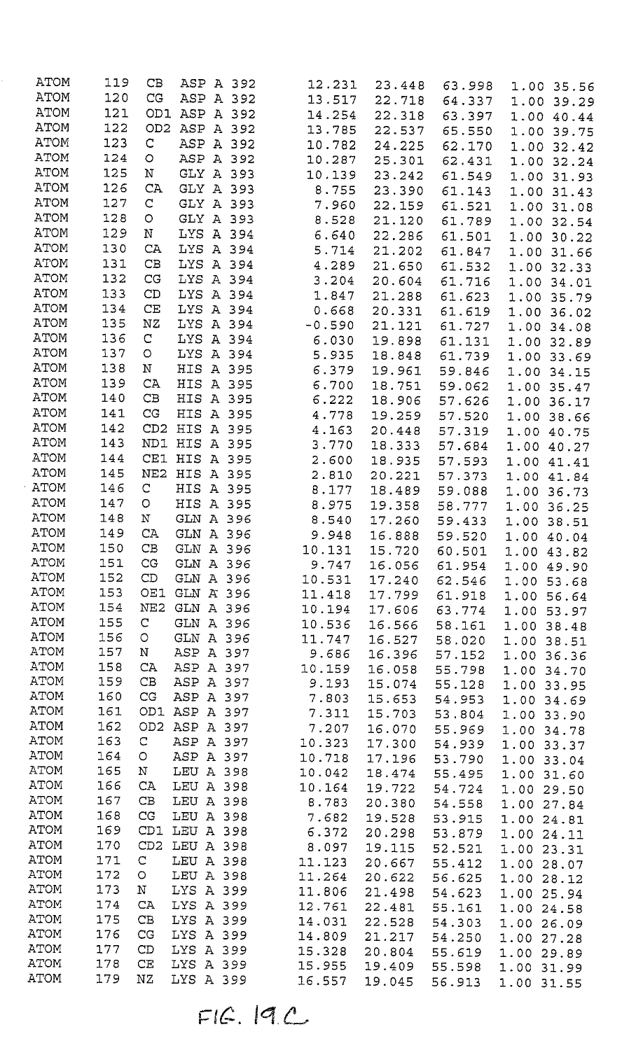

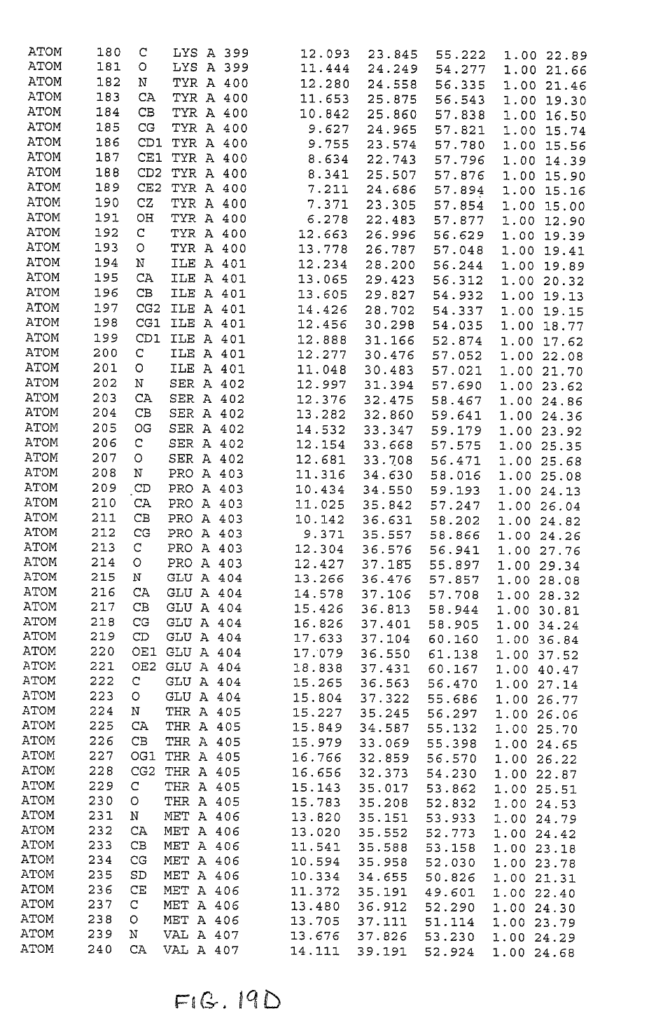

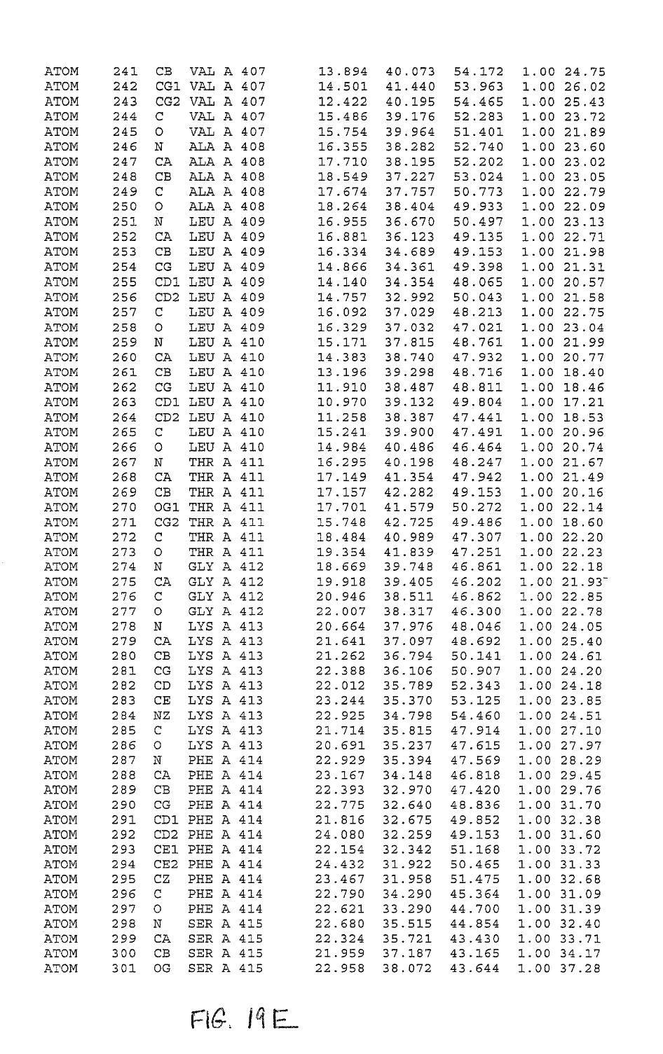

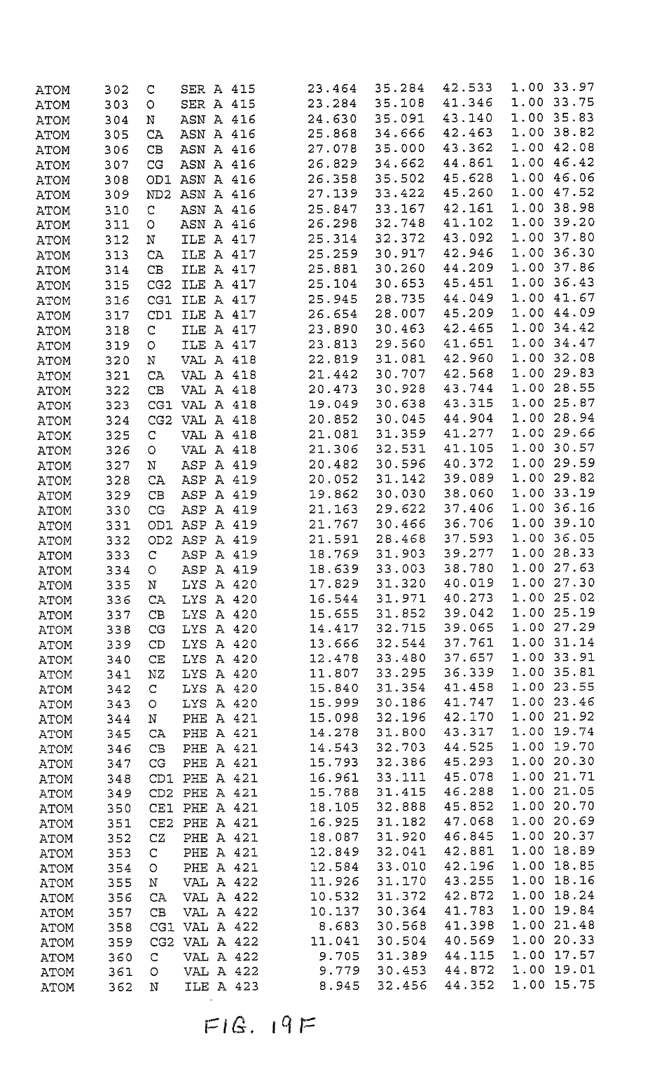

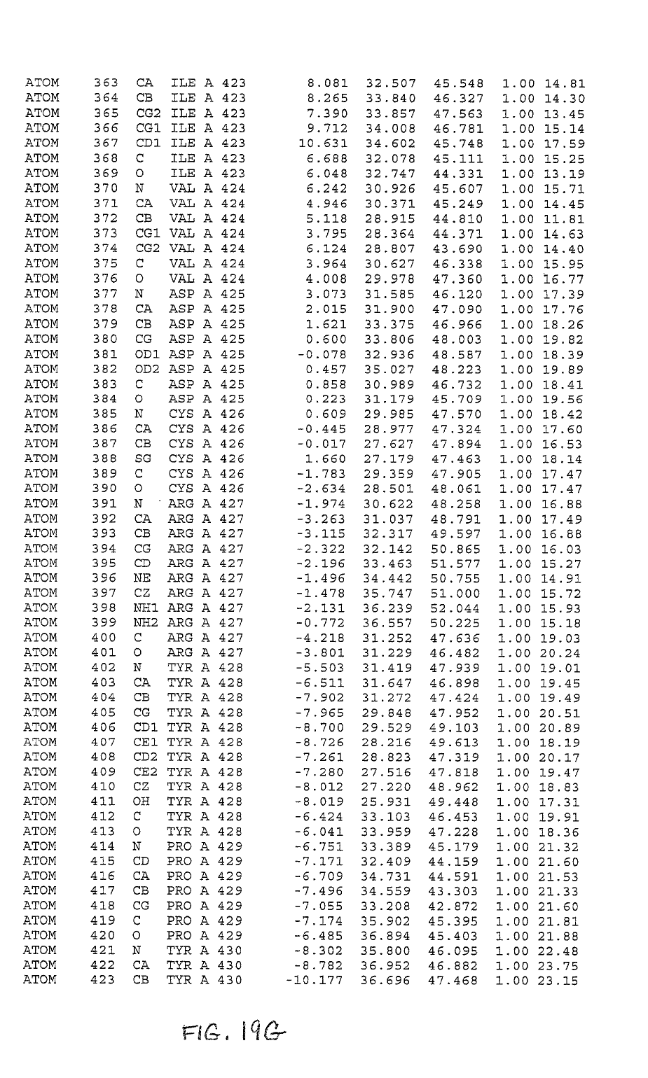

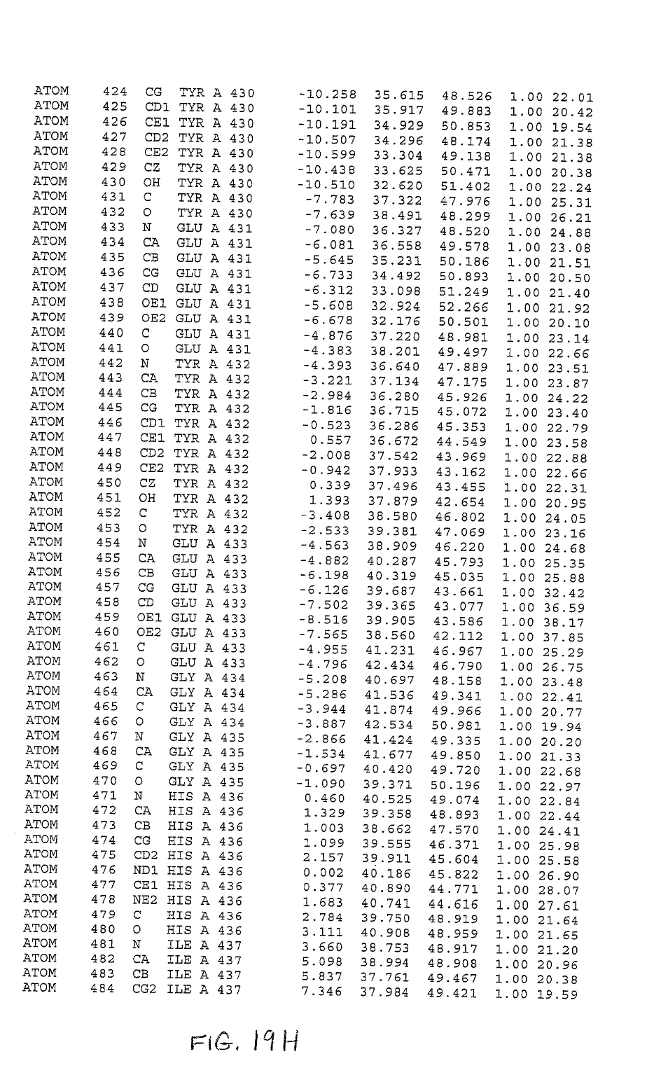

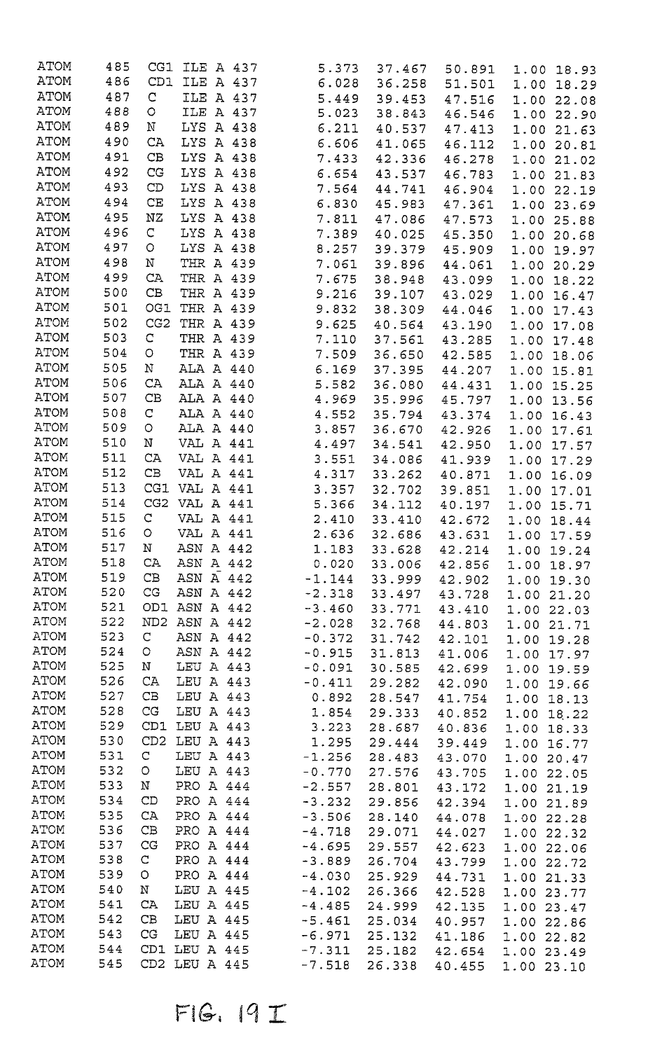

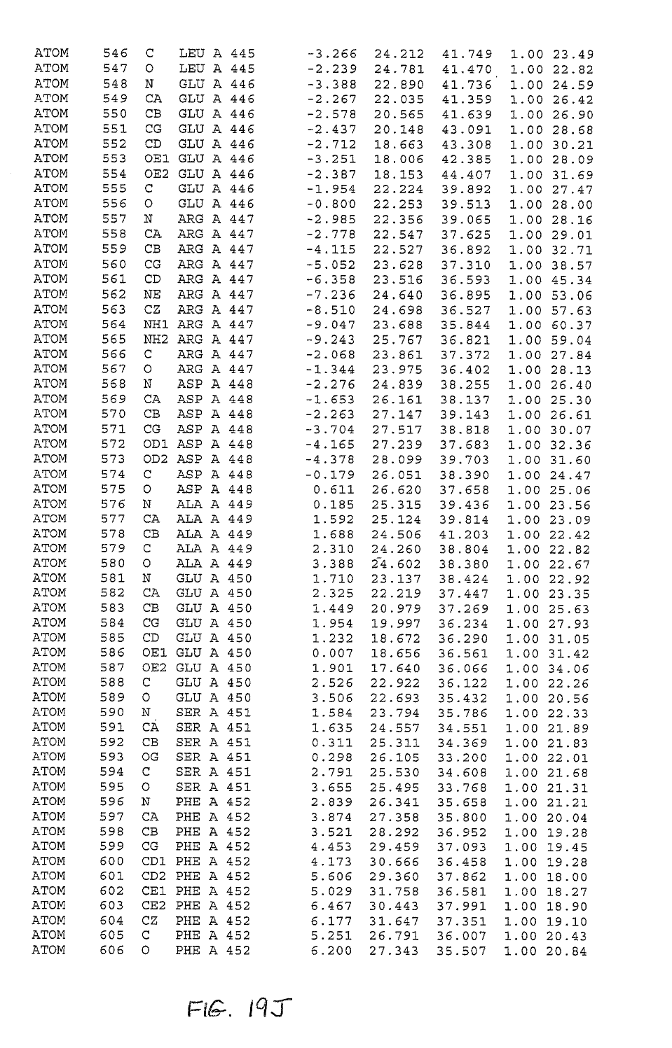

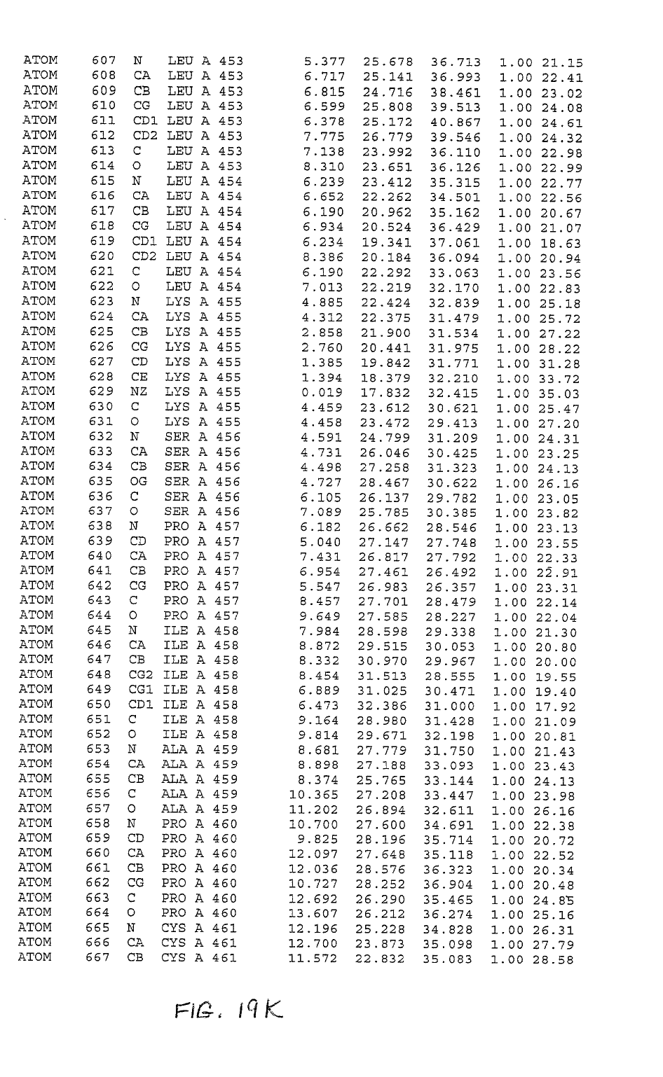

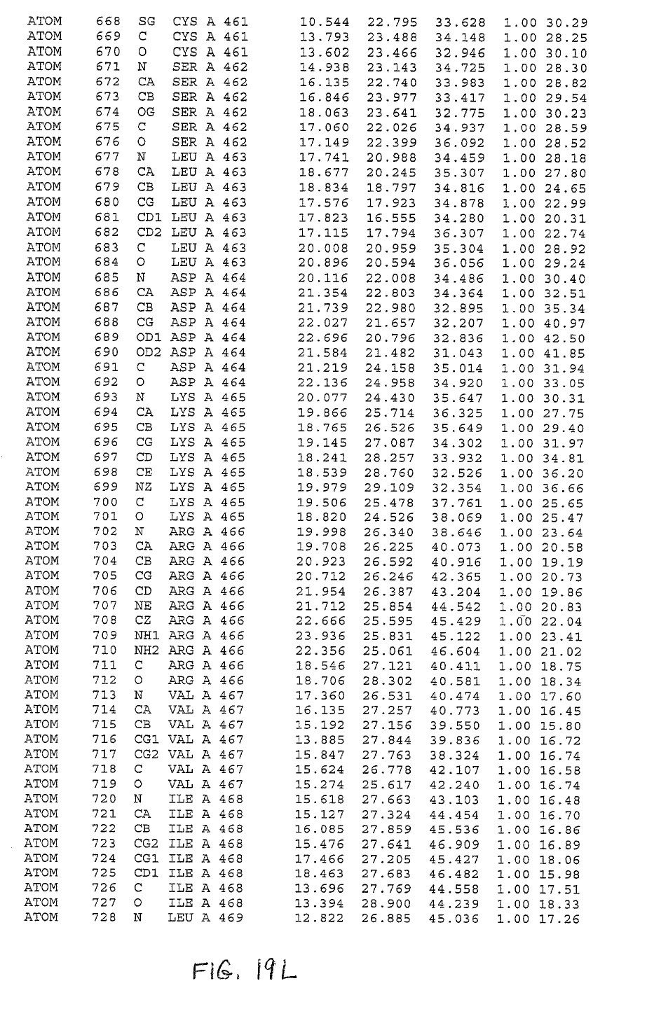

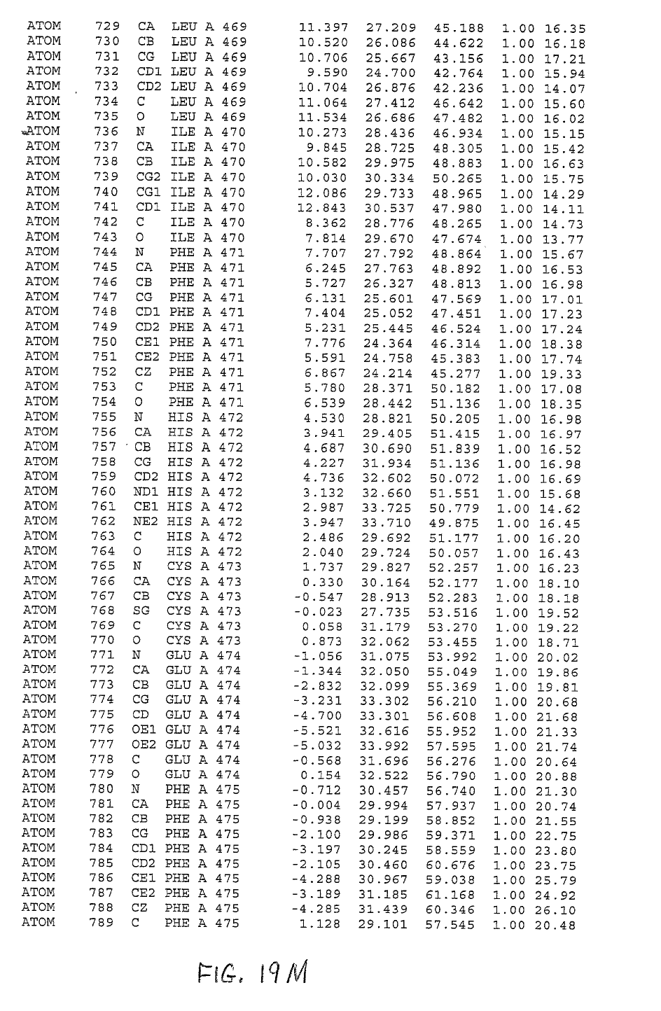

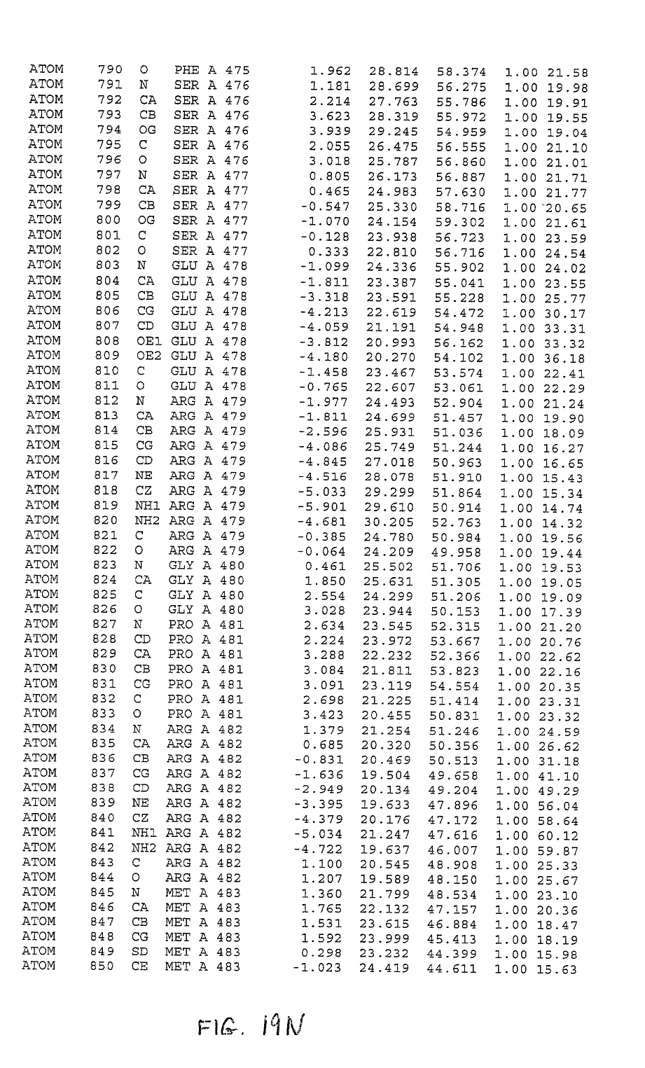

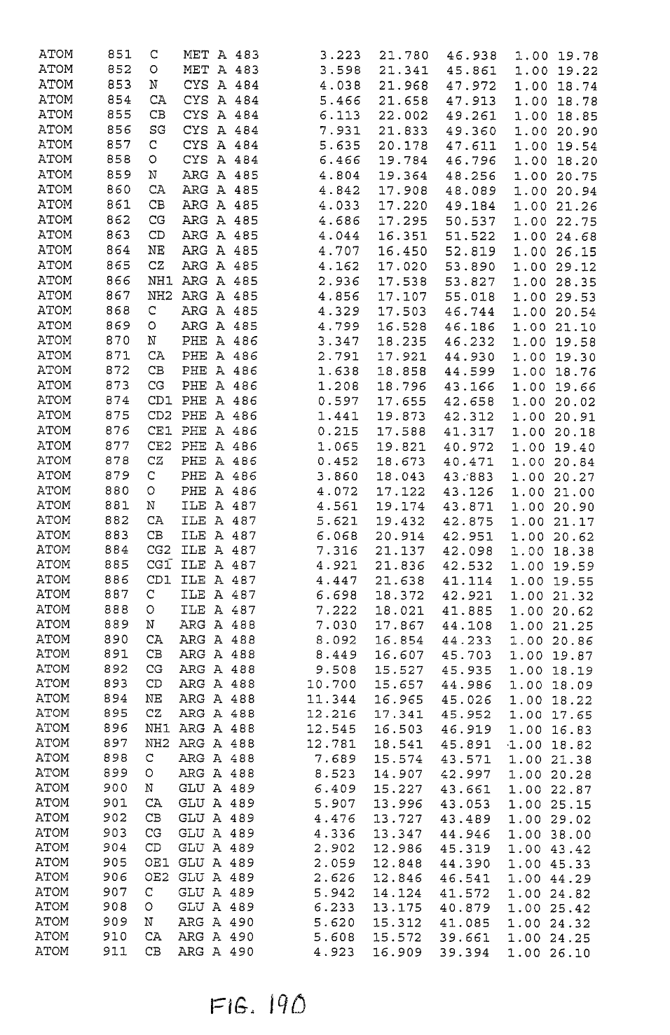

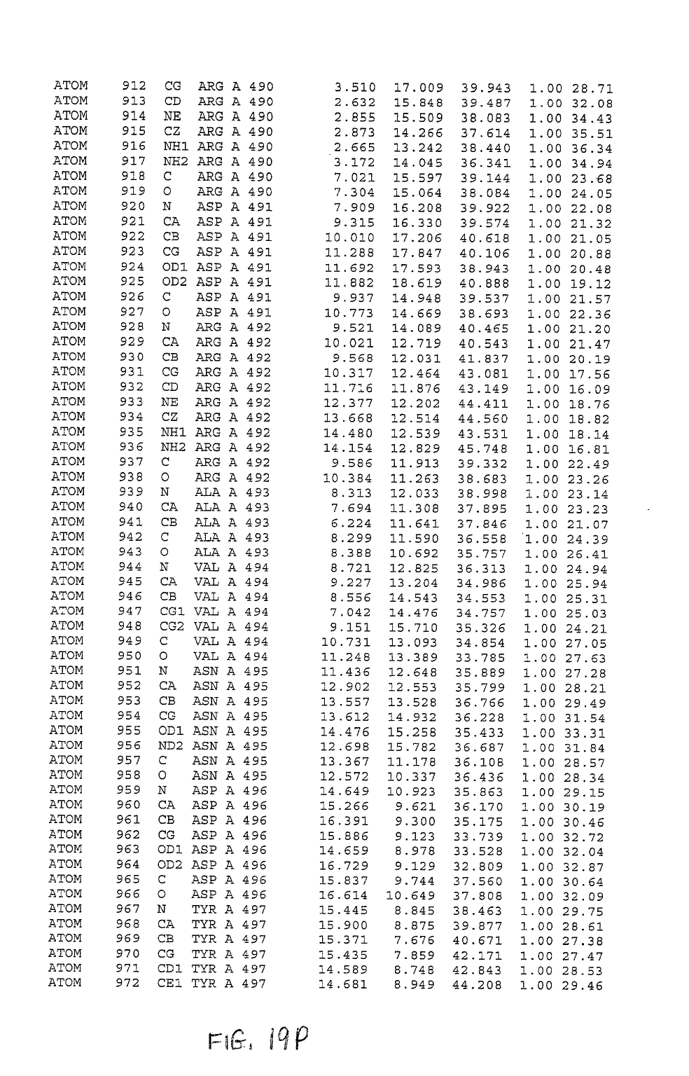

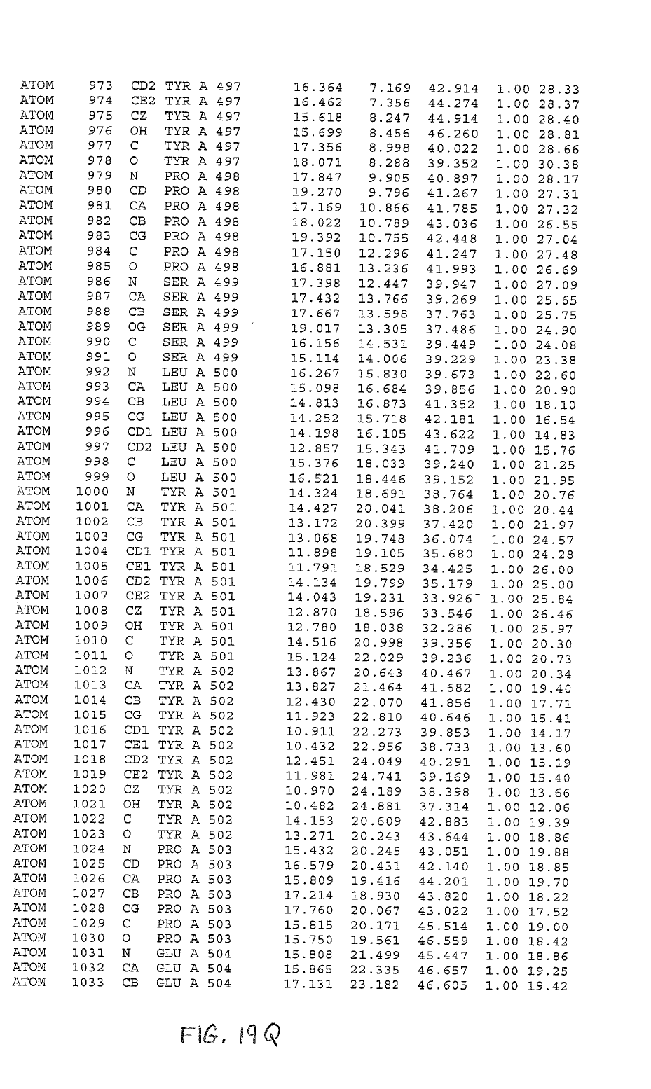

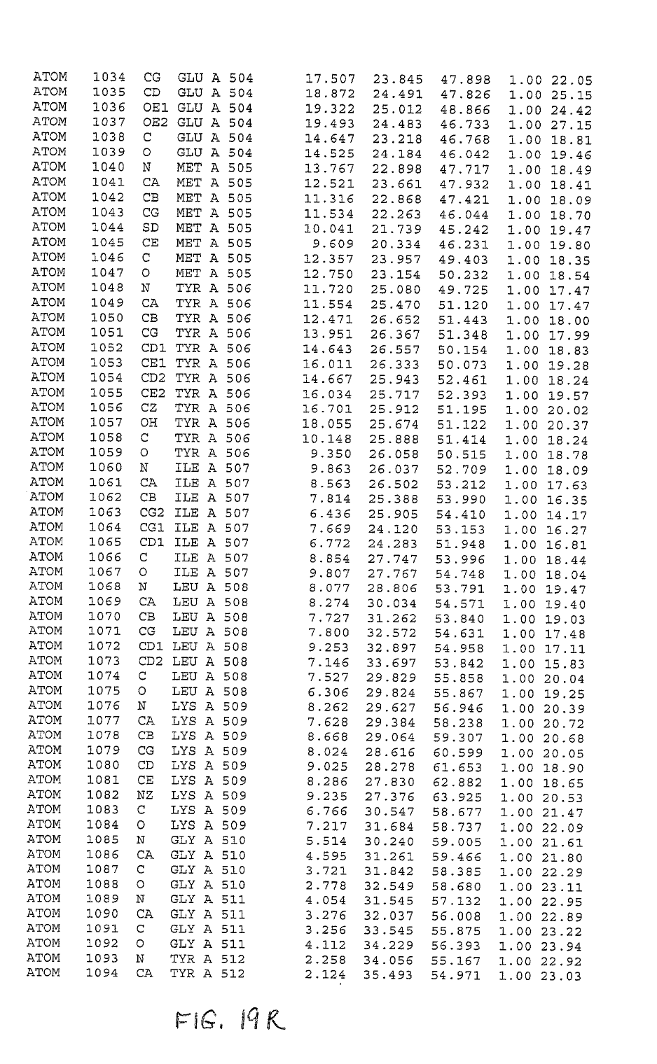

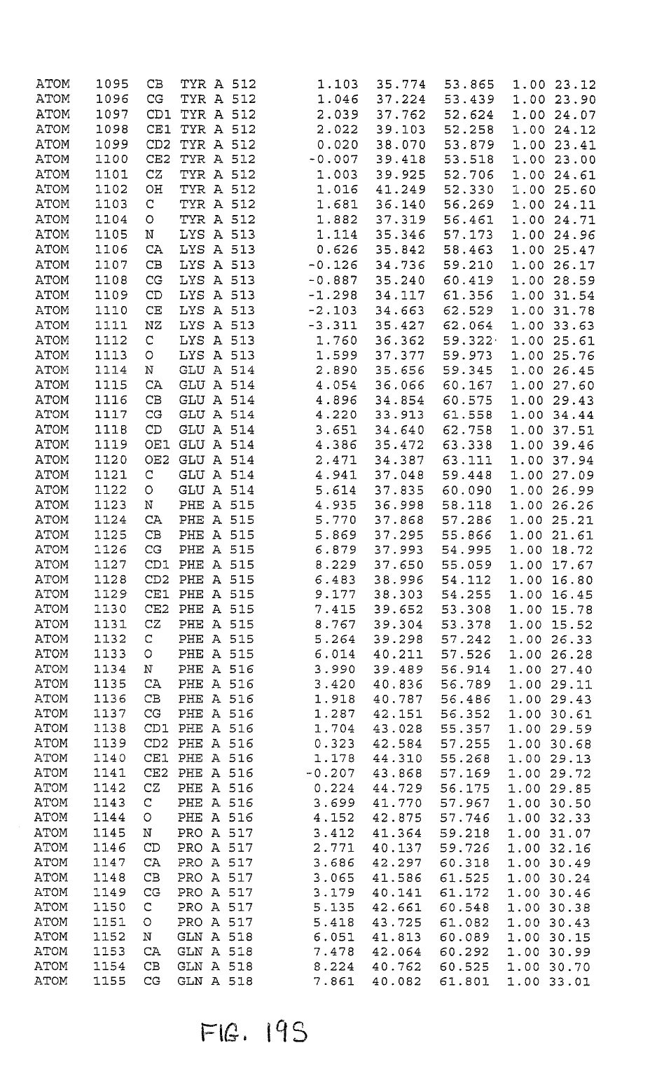

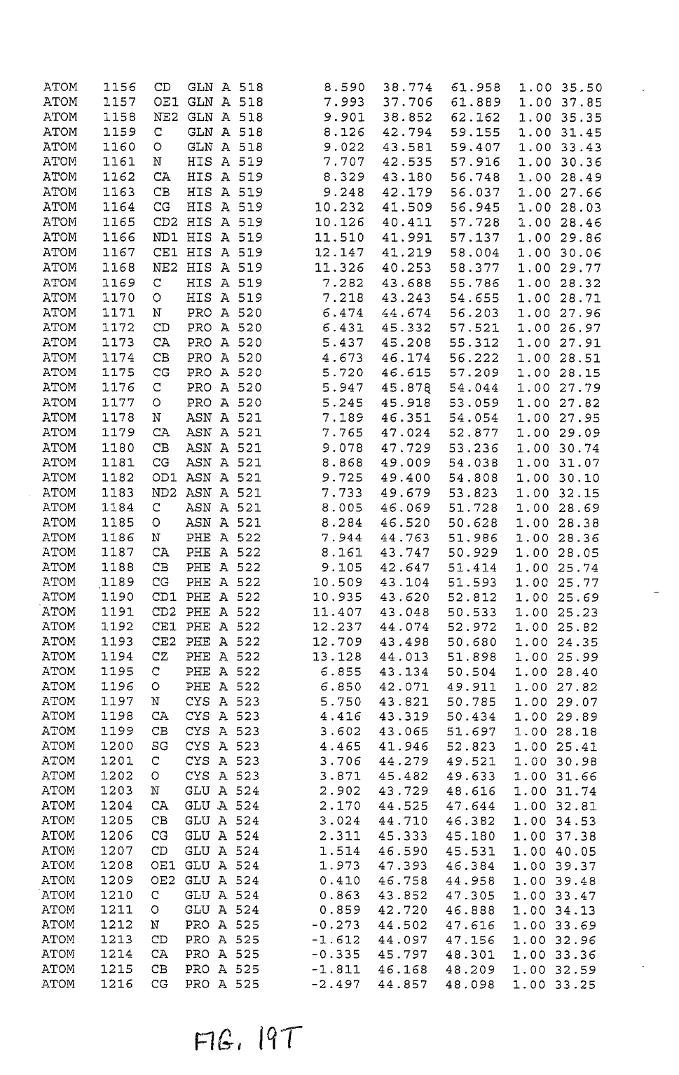

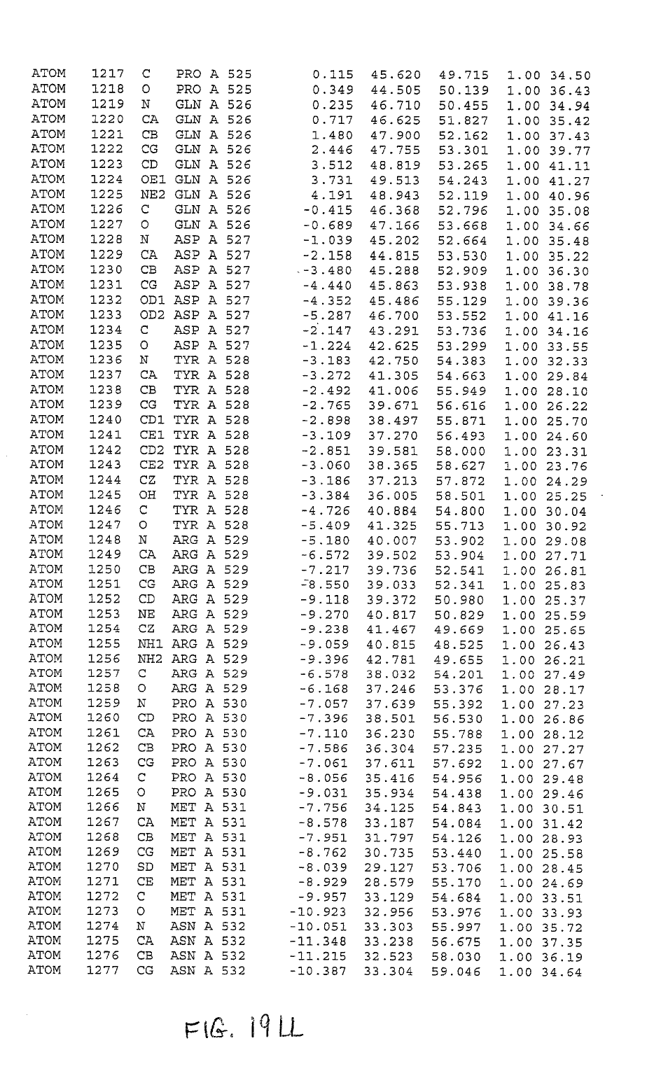

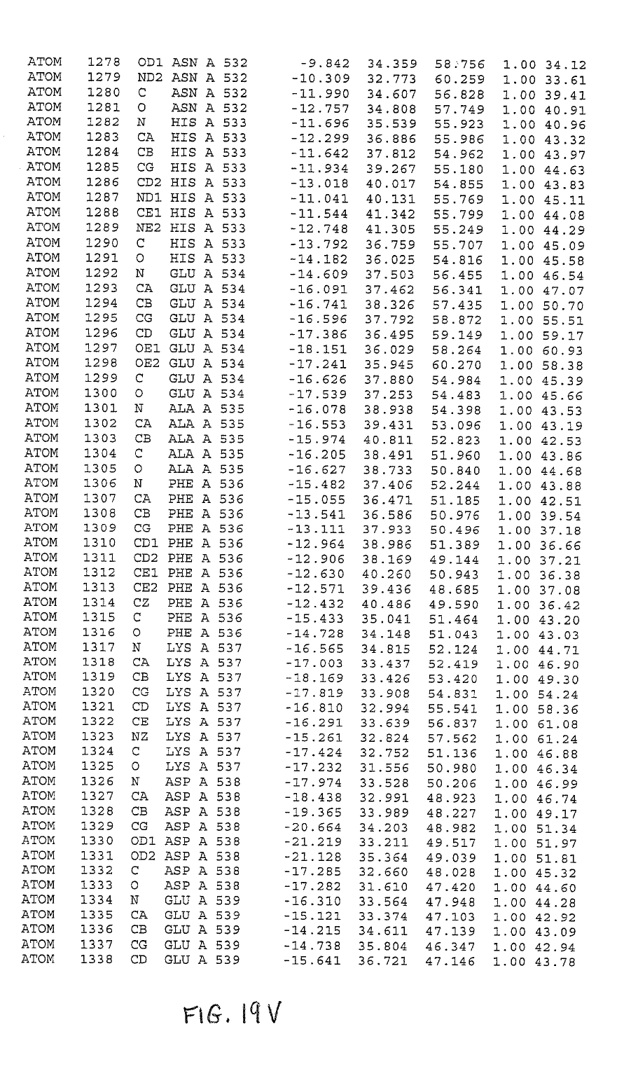

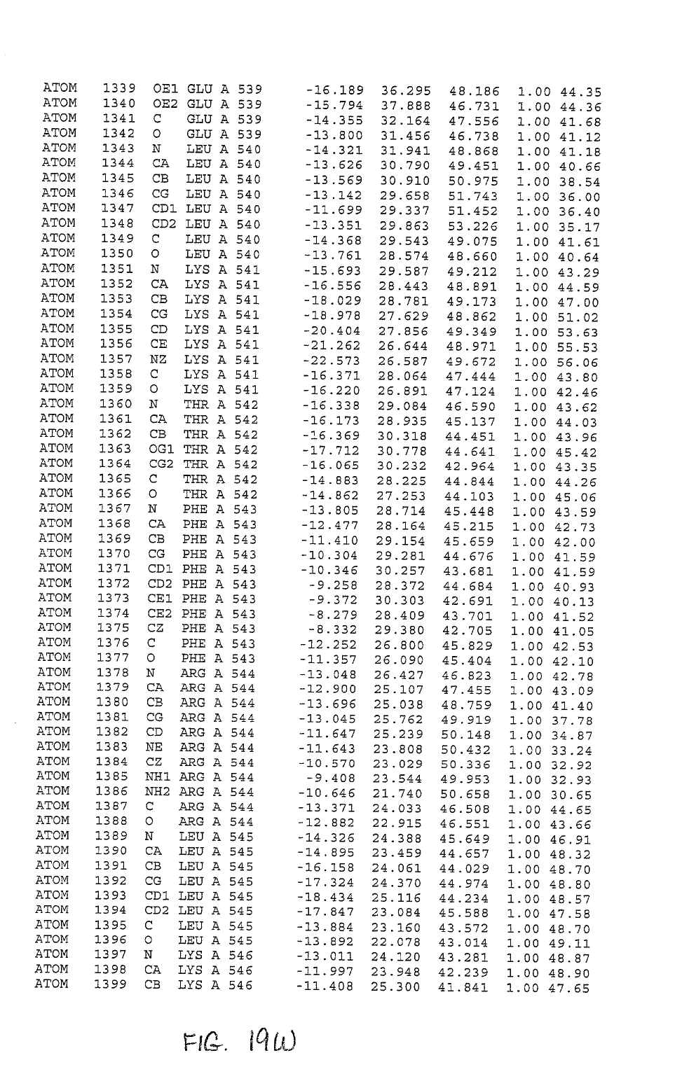

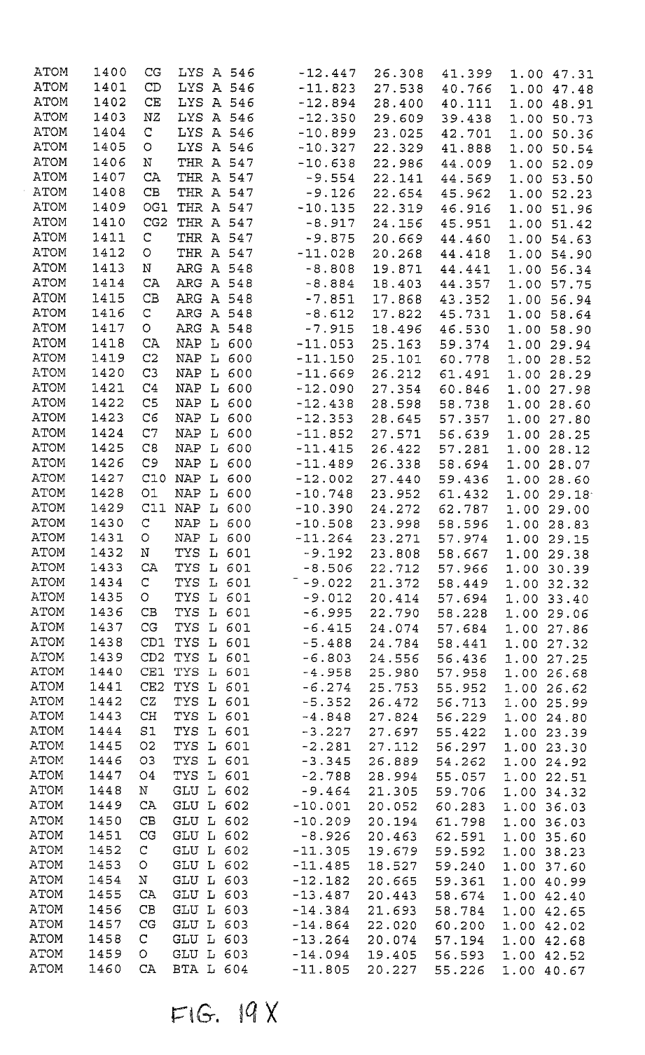

39. The method of claim 32 wherein the crystal structure coordinates are set forth in FIGS. 19A to 19I.

40. The method of claim 31 wherein the compound interacts with one or more of subsites 1 to 16.

41. The method of claim 40 wherein the compound interacts with two or more of subsites 1 to 16.

42. The method of claim 41 wherein the compound interacts with three or more of subsites 1 to 16.

43. The method of claim 41 wherein the compound interacts with a set of subsites comprising subsite 1 and subsite 2.

44. The method of claim 42 wherein the compound interacts with a set of subsites comprising subsite 1, subsite 2 and subsite 3.

45. The method of claim 41 wherein the compound interacts with a set of subsites comprising subsite 1 and subsite 5.

46. The method of claim 41 wherein the compound interacts with a set of subsites comprising subsite 1 and subsite 3.

47. The method of claim 42 wherein the compound interacts with a set of subsites comprising subsite 1, subsite 4 and subsite 5.

48. The method of claim 42 wherein the compound interacts with a set of subsites comprising subsite 1, subsite 5 and subsite 6.

49. The method of claim 42 wherein the compound interacts with a set of subsites comprising subsite 1, subsite 7 and subsite 8.

50. The method of claim 42 wherein the compound interacts with a set of subsites comprising subsite 1, subsite 2 and subsite 9.

51. The method of claim 42 wherein the compound interacts with a set of subsites comprising subsite 1, subsite 2, subsite 4 and subsite 9.

52. The method of claim 42 wherein the compound interacts with a set of subsites comprising subsite 1, subsite 3 and subsite 9.

53. The method of claim 42 wherein the compound interacts with a set of subsites comprising subsite 1, subsite 3, subsite 4 and subsite 9.

54. A Cdc25 inhibitor comprising two or more of the following:
(a) a negatively charged functional group positioned to interact with Arg 479 of human Cdc25B;
(b) a hydrogen bond donor or positively charged functional group positioned to interact with one or more of Cys 426, Tyr 428, Pro 444, Glu 446 and Thr 547 of human Cdc25B;
(c) a hydrogen bond acceptor or a negatively charged functional group positioned to interact with one or more of Tyr 428, Arg 482 and Arg 544 of human Cdc25B;
(d) a hydrophobic moiety positioned to interact with one or more of Leu 445, Glu 446, Arg 479, Met 483, Thr 547 and Arg 548;
(e) a negatively charged functional group positioned to interact with one or more of Arg 482 and Arg 544 of human Cdc25B;
(f) a hydrophobic moiety positioned to interact with one or more of Glu 478, Arg 479, Met 531 and Arg 544 of human Cdc25B;
(g) a hydrophobic moiety positioned to interact with one or more of Tyr 428, Glu 478, Arg 479, Met 531, Leu 540, and Arg 544 of human Cdc25B;
(h) a hydrophobic moiety positioned to interact with one or more of Phe 475, Met 531, Asn 532 and Leu 540 of human Cdc25B;
(i) a hydrophobic moiety positioned to interact with one or more of Phe 475 and Ser 477 of human Cdc25B;
(j) a hydrophobic moiety positioned to interact with one or more of Glu 474, Phe 475, Met 531 and Asn 532 of human Cdc25B;
(k) a hydrophobic moiety positioned to interact with one or more of Tyr 428, Met 531, Lys 537, Lys 541, Leu 540 and Arg 544 of human Cdc25B;
(l) a hydrogen bond donor or hydrogen bond acceptor positioned to interact with Ser 477 of human Cdc25B;
(m) a hydrogen bond donor or positively charged functional group positioned to interact with Glu 478 of human Cdc25B;
(n) a negatively charged functional group positioned to interact with Lys 394 of human Cdc25B;
(o) a negatively charged functional group positioned to interact with Arg 482 of human Cdc25B;
(p) a negatively charged functional group positioned to interact with Arg 544 of human Cdc25B; and
(q) a hydrophobic moiety and a hydrogen bond donor or hydrogen bond acceptor positioned to interact with Asn 532 of human Cdc25B.

55. The Cdc25 inhibitor of claim 54 comprising (a) and (e).

56. The Cdc25 inhibitor of claim 54 comprising (a) and at least one of (b), (c) and (d).

57. The Cdc25 inhibitor of claim 56 further comprising (e).

58. The Cdc25 inhibitor of claim 54 comprising (a), (e) and (f).

59. The Cdc25 inhibitor of claim 54 comprising (a) and (g).

60. The Cdc25 inhibitor of claim 54 comprising (a), (f) and (g).

61. The Cdc25 inhibitor of claim 54 comprising (a), (g) and (h).

62. The Cdc25 inhibitor of claim 54 comprising (a) and at least one of (i) and (j).

63. The Cdc25 inhibitor of claim 54 comprising (a), (k) and at least one of (b), (c) and (d).

64. The Cdc25 inhibitor of claim 63 further comprising (f).

65. The Cdc25 inhibitor of claim 55 further comprising (k).

66. The Cdc25 inhibitor of claim 61 further comprising (f).

67. A method of treating a Cdc25-mediated condition in a patient comprising the step of administering to the patient a therapeutically effective amount of a Cdc25 inhibitor of claim 54.

68. The method of claim 67 wherein the patient is a human.

69. The method of claim 67 wherein the Cdc25-mediated condition is characterized by excessive cellular proliferation.

70. The method of claim 69 wherein the Cdc25-mediated condition is cancer, restenosis, reocclusion of a coronary artery and inflammation.

71. A compound of Formula I,
R₁—A1A2-A3-A4-R₂ (I)
or a pharmaceutically acceptable salt thereof, a pharmaceutically acceptable prodrug thereof, or a combination thereof, wherein
R₁ is R₃—CO; R₄R₅N—CO; R₆—SO₂; R₇R₈NSO₂, wherein R₃, R_{4,} R₅, R₆, R₇, R₈, are, independently of each other, hydrogen, substituted or unsubstituted alkyl, substituted or unsubstituted aryl, substituted or unsubstituted cycloalkyl, substituted or unsubstituted heterocycloalkyl, E- or Z-aryl-C₂-C₄-alkenyl or aryl-C₂-C₄-alkinyl; or R₄ and R₅, together with the nitrogen atom, form a four to seven-membered heterocyclic ring; or or R₇ and R₈, together with the nitrogen atom, form a four to seven-membered heterocyclic ring; or
R₃—CO is an amino acid residue of the formula R₉—CO—A6, wherein
R₉ is hydrogen, C₁₋₆alkyl, phenyl, benzyl, naphthyl, benzyloxy or C₁₋₆alkoxy; and
A6 is aspartyl, asparagyl, prolyl, alanyl, valyl, lysyl, glycyl, arginyl, isoleucyl, seryl, threonyl, leucyl, tryptophanyl, cysteinyl, tyrosyl, methionyl, glycyl, glutamyl, phenylalanyl or histidyl;
A1 is an amino acid residue of Formula II 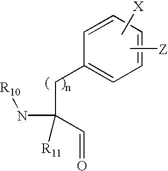
wherein R₁₀ and R₁₁ are each, independently, hydrogen or C₁₋₆alkyl;
n is 0, 1 or 2;
X is SO₃H, SO₂NR₁₂R₁₃, CH₂—SO₃H, CF₂—SO₃H, CH₂—SO₂NR₁₂R₁₃, CF₂—SO₂NR₁₂R₁₃, wherein R₁₂ and R₁₃ are each, independently, hydrogen, C₁₋₆alkyl, or substituted or unsubstituted phenyl, benzyl, furanyl, thiophenyl, thiazolyl, isothiazolyl, pyrazolyl, isoxazolyl or oxazolyl; or, R₁₂ is hydrogen and R₁₃ is hydroxy, C₁₋₆alkoxy, C₁₋₆alkylcarbonyl or substituted or unsubstituted benzoyl; or
X is PO₃H₂, OCH₂PO₃H₂, CH₂PO₃H₂, CF₂PO₃H₂, COOH, CH₂—COOH, CF₂—CO₂H, OCH₂CO₂H, OCF₂CO₂H, OCH(CO₂H)₂, OCF(CO₂H)₂; or
X is NH—SO₂—R₁₄, wherein R₁₄ is C₁₋₆alkyl, benzyl, phenyl; or
X is NH—CO—COO—R₁₅, wherein R₁₅ is C₁₋₆alkyl, benzyl, or phenyl;
Z is hydrogen, C₁₋₆alkyl, halo, hydroxy, C₁₋₆alkyl amino, di-C₁₋₆alkyl amino, C₁₋₆alkoxy, C₁₋₆alkythio, C₁₋₆alkylcarbonyl, halogen-substituted C₁₋₆alkylcarbonyl, formyl-, phenylcarbonyl, benzylcarbonyl, C₁₋₆alkyl-sulfonyl, C₁₋₆alkyl-sulfonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl, oxytrifluoromethyl; or
Z is —(CH₂)ₘ-NR₁₆R₁₇, wherein m is 0, 1 or 2 and R₁₆ and R₁₇ are each independently selected from the group consisting of hydrogen, C₁₋₆alkyl, C₁₋₆-alkyl-carbonyl, amino-C₂₋₆alkyl, C₁₋₆alkyl-amino-C₂₋₆alkyl, di-C₁₋₆alkyl-amino-C₂₋₆alkyl, hydroxy-C₁₋₆alkyl, C₁₋₆alkoxy-C₁₋₆alkyl, aryl-C₁₋₆alkyl, C₃₋₈cycloalkyl-C₁₋₆alkyl and heterocycloalklyl-C₁₋₆alkyl;
A2 is an amino acid residue of Formula III 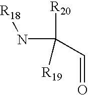
wherein R₁₈ and R₁₉ are each, independently, hydrogen or C₁₋₆alkyl; or
R₂₀ is the side chain of an amino acid selected from the group consisting of glycine, alanine, valine, leucine, isoleucine, norvaline, norleucine, aspartic acid, glutamic acid, lysine, asparagine, glutamine, phenylalanine, histidine, homoleucine, C₁₋₆alkyl-glutamic acid, C₁₋₆alkyl-aspartic acid, and lysine-(Boc); or
R₂₀ is —(CH₂)ₒ—COOR₂₁ wherein o is from about 3 to about 5 and R₂₁ is hydrogen or C₁₋₆alkyl; or
R₁₉ and R₂₀, together with the α-carbon, form a three to seven-membered carbocyclic ring system; or
R₁₈ and R₂₀, together with the α-carbon atom and the nitrogen atom, form a substituted or unsubstituted four- to seven-membered heterocyclic ring system; or
R₁₈ and R₂₀, together with the α-carbon atom and the nitrogen atom, form a eight to twelve-membered heterobicyclic ring system;
A3 is an amino acid of Formula IV, 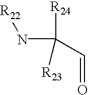
wherein R₂₂ and R₂₃ are each, independently, hydrogen or C₁₋₆alkyl; or
R₂₄ is the side chain of an amino acid selected from the group consisting of glycine, alanine, valine, leucine, isoleucine, norvaline, norleucine, aspartic acid, glutamic acid, lysine, asparagine, glutamine, phenylalanine, histidine, homoleucine, C₁₋₆alkyl-glutamic acid, C₁₋₆alkyl-aspartic acid, and lysine-(Boc); or
R₂₄ is —(CH₂)ₒ—COOR₂₁ wherein o is about 3 to about 5 and R₂₁ is hydrogen or C₁₋₆alkyl;
R₂₃ and R₂₄ can form together a three to seven-membered carbocyclic ring system; or
R₂₂ and R₂₄, together with the α-carbon atom and the nitrogen atom, form a substituted or unsubstituted four- to seven-membered heterocyclic ring system; or
R₂₂ and R₂₄, together with the α-carbon atom and the nitrogen atom, form a eight to twelve-membered heterobicyclic ring system; or
A2 and A3 together are a residue selected from the group consisting of 6-amino-5-oxoperhydropyrido[2,1-b][1,3]thiazole-3-carboxylic acid; 6-amino-5-oxoperhydro-3-indolizinecarboxylic acid; (S, R)-6-amino-5-oxoperhydro-8a-indolizinecarboxylic acid; (R,R)-6-amino-5-oxoperhydro-8a-indolizinecarboxylic acid; (R, S)-6-amino-5-oxoperhydro-8a-indolizinecarboxylic acid; (S,S)-6-amino-5-oxoperhydro-8a-indolizinecarboxylic acid; 2-(3-amino-2-oxo-1,2-dihydro-1-pyridinyl)acetic acid; 2-(3-amino-2-oxo-6-phenyl-1,2dihydro-1-pyridinyl)acetic acid; 3-amino benzoic acid; 4-aminobenzoic acid; 3-aminomethyl benzoic acid; (S)-3-(1-aminoethyl)benzoic acid; (R)-3-(1-aminoethyl)benzoic acid; (S)-3-(1-aminopropyl) benzoic acid; (R)-3-(1-aminopropyl) benzoic acid; (S)-3-(1-aminobutyl) benzoic acid; (R)-3-(1-aminobutyl) benzoic acid; 2-(3-amino-2-oxo-1-azepanyl)acetic acid; 2-[8-(aminomethyl)-3,6-dimethyl-9,10,10-trioxo-9,10-dihydro-10 ⁶-thioxanthen-1-yl]acetic acid; 2-(2-oxopiperazino)acetic acid; 2-[8-(aminomethyl)-2-oxo-5-phenyl-2,3-dihydro-1H-1,4-benzodiazepin-1-yl]acetic acid; 2-[8-(aminomethyl)-2-oxo-5-methyl-2,3-dihydro-1H-1,4-benzodiazepin-1-yl]acetic acid; 3-aminopropanoic acid; 4-aminobutanoic acid; 5-aminopentanoic acid; 2-[2-(2-aminoethoxy)ethoxy]acetic acid; and 2-(3-amino-2-oxo-2,3,4,5-tetrahydro-1H-1-benzazepin-1-yl)acetic acid;
A4 is an amino acid of Formula V 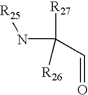
wherein
R₂₅ is hydrogen or C₁₋₆alkyl;
R₂₆ is hydrogen or C₁₋₆alkyl; and
R₂₇ is —(CH₂)ₚ—(CH(R₂₈))_{q}-aryl, wherein p is 0, 1 or 2; q is 0, 1 or 2; and R₂₈ is hydrogen or methyl; and
R₂ is NR₃₂R₃₃, wherein
R₃₂ is hydrogen or C₁₋₆alkyl; and
R₃₃ is (CH₂)_{w}W—(CH₂)ₓ—V, wherein
W is a single bond and the sum of w and x is 1 to 6; or
W is substituted or unsubstituted aryl or aryl-T, wherein T is O, S or NH; w is 0, 1, 2 or 3 and x is 0, 1, 2 or 3; or
W is C₃₋₈cycloalkyl; w is 0, 1, 2 or 3 and x is 0, 1, 2 or 3; and
V is COOR₃₄ wherein R₃₄ is hydrogen and C₁₋₆alkyl; or
V is COC₁₋₆alkyl, CONH₂, SO₃H or NO₂; or
R₂ is an amino acid A5 of Formula VI 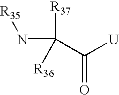
wherein
R₃₅ and R₃₆ are each, independently, hydrogen or C₁₋₆alkyl;
R₃₇ is the side chain of aspartic acid, asparagine, glutamic acid, glutamine, Aspartyl-C₁₋₆alkyl ester, glutamyl-C₁₋₆alkyl ester; or
R₃₇ is (CH₂)_{y}—COOR₄₂, wherein y is 3, 4 or 5, and R₄₂ is hydrogen or C₁₋₆alkyl; or
R₃₇ is (CH₂)_{z}—CONR₄₀R₄₁, wherein z is 1 to 5 and R₄₀ and R₄₁ are independently, hydrogen or C₁₋₆-alkyl, or R₄₀, R₄₁ and the nitrogen atom together form a 5- to 8-member heterocycle; or
R₃₇ is (CH₂)ₐ—SO₃H, wherein a is 1, 2, 3, 4 or 5; or (CH₂)_{b}-tetrazolyl, wherein b is 1, 2, 3, 4 or5; or
R₃₇ is (CH₂)_{d}-phenyl-(CH₂)ₑ—COOR₄₃ wherein d is 0 to 2, e is 0 to 2 and R₄₃ is hydrogen or C₁₋₆alkyl; or
R₃₇ is (CH₂)_{d}-phenyl-(CH₂)ₑ—CONR₄₄R₄₅ wherein d is 0 to 2, e is 0 to 2 and R₄₄ and R₄₅ are independently hydrogen, C₁₋₆alkyl or R₄₄ and R₄₅ and the nitrogen atom together form a 5 to 8-member heterocyclic ring; and
U is hydroxy, C₁₋₆alkoxy or NR₃₈R₃₉, wherein R₃₈ and R₃₉ are each, independently, hydrogen; substituted or unsubstituted C₁₋₁₀-alkyl, substituted or unsubstituted aryl or substituted or unsubstituted cycloalkyl or bicycloalkyl; or
R₃₈ and R₃₉, together with the nitrogen atom, form a four- to seven-membered heterocyclic ring.

72. The compound of claim 71 wherein R₃, at least one of R₄ and R₅, R₆, or at least one of R₇ and R₈, is an alkyl which is substituted by one or more substituents independently selected from the group consisting of hydroxy, C₁₋₆alkoxy, phenoxy, benzyloxy, halogen, amino, C₁₋₆alkylamino, di-C₁₋₆alkylamino, C₁₋₆alkyl-CO—NH, substituted and unsubstituted aryl, and substituted and unsubstituted cycloalkyl.

73. The compound of claim 72 wherein R₃, at least one of R₄ and R₅, R₆, or at least one of R₇ and R₈, is an alkyl which is substituted by at least one aryl group selected from the group consisting of substituted and unsubstituted phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzothienyl, pyrazolyl, indolyl, purinyl, isoxazolyl, oxazolyl, dibenzofuranyl.

74. The compound of claim 73 wherein the aryl group is substituted by one or more substituents independently selected from the group consisting of C₁₋₆alkyl, halo, hydroxy, C₁₋₆alkyl amino, di-C₁₋₆alkyl amino, C₁₋₆alkoxy, C₁₋₆alkylthio, C₁₋₆alkylcarbonyl, phenylcarbonyl, benzylcarbonyl, C₁₋₆alkyl-sulfonyl, C₁₋₆alkyl-sulfonyl-amino, C₁₋₆alkyl-carbonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl and oxytrifluoromethyl.

75. The compound of claim 72 wherein R₃, at least one of R₄ and R₅, R₆, or at least one of R₇ and R₈, is an alkyl which is substituted by at least one cycloalkyl selected from the group consisting of C₃₋₈-cycloalkyl, adamantyl and bicyclooct[3.3.0]-yl.

76. The compound of claim 75 wherein the cycloalkyl group is substituted by one or more substituents independently selected from the group consisting of C₁₋₆alkyl, halogen, hydroxy, C₁₋₆alkyl amino, di-C₁₋₆alkyl amino, C₁₋₆alkoxy, C₁₋₆alkylthio, and C₁₋₆alkylcarbonyl.

77. The compound of claim 71 wherein R₃, at least one of R₄ and R₅, R₆, or at least one of R₇ and R₈, is a cycloalkyl group selected from the group consisting of substituted and unsubstituted C₃₋₈-cycloalkyl, adamantyl and bicyclooct[3.3.0]-yl.

78. The compound of claim 77 wherein the cycloalkyl group is substituted by one or more substituents independently selected from the group consisting of C₁₋₆alkyl, halo, hydroxy, C₁₋₆alkyl amino, di-C₁₋₆alkyl amino, C₁₋₆alkoxy, C₁₋₆alkylthio and C₁₋₆alkylcarbonyl.

79. The compound of claim 71 wherein R₃, at least one of R₄ and R₅, R₆, or at least one of R₇ and R₈, is an aryl-E-C₂₋₄-alkenyl, aryl-Z-C₂₋₄-alkenyl or aryl-C₂₋₄-alkinyl group, wherein the aryl group is selected from the group consisting of substituted and unsubstituted phenyl, naphthyl, anthracenyl, phenantluenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzothienyl, pyrazolyl, indolyl, purinyl, isoxazolyl, oxazolyl, and dibenzofuranyl.

80. The compound of claim 79 wherein the aryl group is substituted by one or more substituents independently selected from the group consisting of C₁₋₆alkyl, halo, hydroxy, C₁₋₆-alkyl amino, di-C₁₋₆alkyl amino, C₁₋₆alkoxy, C₁₋₆alkylthio, C₁₋₆alkylcarbonyl, phenylcarbonyl, benzylcarbonyl, C₁₋₆alkyl-sulfonyl, C₁₋₆alkyl-sulfonyl-amino, C₁₋₆alkyl-carbonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl, oxytrifluoromethyl, substituted and unsubstituted cycloalkyl and substituted and unsubstituted heterocycloalkyl.

81. The compound of claim 71 wherein R₃, at least one of R₄ and R₅, R₆, or at least one of R₇ and R₈, is a substituted or unsubstituted heterocycloalkyl group selected from the group consisting of substituted and unsubstituted pyrrolidinyl, piperazinyl, tetrahydropyranyl, tetrahydrofuranyl, pyrrolidinonyl and morpholinyl.

82. The compound of claim 71 wherein R₃, at least one of R₄ and R₅, R₆, or at least one of R₇ and R₈, is an aryl group selected from the group consisting of substituted and unsubstituted phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzothienyl, pyrazolyl, indolyl, purinyl, isoxazolyl, oxazolyl, and dibenzofuranyl.

83. The compound of claim 71 wherein the aryl group is substituted by one or more substituents independently selected from the group consisting of C₁₋₆alkyl, halo, hydroxy, C₁₋₆alkyl amino, di-C₁₋₆alkyl amino, C₁₋₆alkoxy, C₁₋₆alkylthio, C₁₋₆alkylcarbonyl, phenylcarbonyl, benzylcarbonyl, C₁₋₆alkyl-sulfonyl, C₁₋₆alkyl-sulfonyl-amino, C₁₋₆alkyl-carbonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl, oxytrifluoromethyl, substituted and unsubstituted cycloalkyl and substituted and unsubstituted heterocycloalkyl.

84. The compound of claim 71 wherein W is aryl or aryl-T, wherein the aryl group is selected from the group consisting of substituted and unsubstituted phenyl, naphthyl, pyridyl, furanyl, thienyl and pyrimidyl.

85. The compound of claim 71 wherein R₂ is of Formula VI and U is NR₃₈R₃₉, wherein at least one of R₃₈ and R₃₉ is a C₁₋₆-alkyl group substituted with one or more substituents independently selected from the group consisting of hydroxy, halogen, substituted and unsubstituted aryl and substituted and unsubstituted cycloalkyl.

86. The compound of claim 85 wherein at least one of R₃₈ and R₃₉ is a C₁₋₆-alkyl group substituted with one or more aryl groups independently selected from the group consisting of substituted and unsubstituted phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzothienyl, pyrazolyl, indolyl, purinyl, isoxazolyl, oxazolyl, dibenzofuranyl.

87. The compound of claim 86 wherein at least one of R₃₈ and R₃₉ is a C₁₋₆-alkyl group substituted with one or more aryl groups, at least one of said aryl groups being substituted with one or more substituents selected from the group consisting of C₁₋₆alkyl, halo, hydroxy, C₁₋₆alkyl amino, di-C₁₋₆alkyl amino, C₁₋₆alkoxy, C₁₋₆alkylthio, C₁₋₆alkylcarbonyl, phenylcarbonyl, benzylcarbonyl, C₁₋₆alkyl-sulfonyl, C₁₋₆alkyl-sulfonyl-amino, C₁₋₆alkyl-carbonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl and oxytrifluoromethyl.

88. The compound of claim 87 wherein at least one of R₃₈ and R₃₉ is a C₁₋₆-alkyl group substituted with one or more cycloalkyl groups independently selected from the group consisting of substituted and unsubstituted C₃₋₈-cycloalkyl, adamantyl and bicyclooctyl.

89. The compound of claim 71 wherein R₂ is of Formula VI and U is NR₃₈R₃₉, wherein at least one of R₃₈ and R₃₉ is an aryl group selected from the group consisting of substituted and unsubstituted phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzothienyl, pyrazolyl, indolyl, purinyl, isoxazolyl, oxazolyl, dibenzofuranyl.

90. The compound of claim 89 wherein at least one of R₃₈ and R₃₉ is an aryl group substituted with one or more substituents independently selected from the group consisting of C₁₋₆alkyl, halo, hydroxy, C₁₋₆alkyl amino, di-C₁₋₆alkyl amino, C₁₋₆alkoxy, C₁₋₆alkylthio, C₁₋₆alkylcarbonyl, phenylcarbonyl, benzylcarbonyl, C₁₋₆alkyl-sulfonyl, C₁₋₆alkyl-sulfonyl-amino, C₁₋₆alkyl-carbonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl and oxytrifluoromethyl.

91. The compound of claim 71 wherein R₂ is of Formula VI and U is NR₃₈R₃₉, wherein at least one of R₃₈ and R₃₉ is a cycloalkyl group selected from the group consisting of substituted and unsubstituted C₃₋₈-cycloalkyl, adamantyl and bicyclooctyl.

92. The compound of claim 91 wherein the cycloalkyl group is substituted by one or more substituents independently selected from the group consisting of C₁₋₆alkyl, halo, hydroxy, C₁₋₆alkyl amino, di-C₁₋₆alkyl amino, C₁₋₆alkoxy, C₁₋₆alkylthio and C₁₋₆alkylcarbonyl.

93. The compound of claim 71 wherein at least one of R₁₆ and R₁₇ is an aryl-C₁₁₋₆alkyl group, wherein said aryl group is selected from the group consisting of substituted and unsubstituted phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzothienyl, pyrazolyl, indolyl, purinyl, isoxazolyl, oxazolyl, dibenzofuranyl.

94. The compound of claim 93 wherein at least one of R₁₆ and R₁₇ is an aryl-C₁₋₆alkyl group wherein said aryl group is substituted with one or more substituents independently selected from the group consisting of C₁₋₆alkyl, halo, hydroxy, C₁₋₆alkyl amino, di-C₁₋₆alkyl amino, C₁₋₆alkoxy, C₁₋₆alkylthio, C₁₋₆alkylcarbonyl, phenylcarbonyl, benzylcarbonyl, C₁₋₆alkyl-sulfonyl, C₁₋₆alkyl-sulfonyl-amino, C₁₋₆alkyl-carbonyl-amino, carboxyl, O-C₁₋₆alkyl carboxyl, carboxylalkenyl, O-C₁₋₆alkyl carboxyl alkenyl, C₁₋₆alkylcarbamoyl, cyano, nitro, trifluoromethyl and oxytrifluoromethyl.

95. The compound of claim 71 wherein at least one of R₁₆ and R₁₇ is an heterocycloalkyl-C₁₋₆alkyl group, wherein said heterocycloalkyl group is selected from the group consisting of substituted and unsubstituted pyrrolidinyl, piperazinyl, tetrahydropyranyl, tetrahydrofuranyl, pyrrolidinonyl and morpholinyl.

96. The compound of claim 71 wherein R₂₇ is —(CH₂)ₚ—(CH(R₂₈))_{q}-aryl, wherein the aryl group is selected from the group consisting of substituted and unsubstituted phenyl, naphthyl, anthracenyl, phenanthrenyl, fluorenyl, pyridyl, pyridazinyl, pyridinonyl, furanyl, thienyl, thiazolyl, isothiazolyl, imidazolyl, triazolyl, pyrrolyl, tetrazolyl, benzimidazolyl, pyrazinyl, pyrimidyl, quinolyl, isoquinolyl, benzofuranyl, benzothienyl, pyrazolyl, indolyl, purinyl, isoxazolyl, oxazolyl, tetrahydronaphthyl, benzodihydrofuranyl, quinazoline and dibenzofuranyl.

97. The compound of claim 96 wherein the aryl group is substituted by one or more substituents independently selected from the group consisting of C₁₋₆alkyl, halo, hydroxy, C₁₋₆alkyl amino, di-C₁₋₆alkyl amino, C₁₋₆alkoxy, C₁₋₆alkylthio, C₁₋₆alkylcarbonyl, halogen-substituted C₁₋₆alkylcarbonyl, formyl, phenylcarbonyl, benzylcarbonyl, C₁₋₆alkyl-sulfonyl, C₁₋₆alkyl-sulfonylamino, carboxyl, O-C₁₋₆alkyl carboxyl, carboxylalkenyl, O-C₁₋₆alkylcarboxyalkenyl, C₁₋₆alkylcarbamoyl, cyano, nitro, trifluoromethyl, oxytrifluoromethyl, aryl; Y—(CH₂)ₛ-C₃₋₈-cycloalkyl and Y—(CH₂)ₛ-aryl, wherein Y is O, S or NH and s is 0, 1, 2 or 3; Y—(CH₂)ₜ-heterocycloalkyl, wherein t is 1 to 6; Y—(CH₂)ᵤ—R₂₉ where Y is O, NH, or S, u is 2 to 6 and R₂₉ is OH, CH₂—OH, NH₂ or NH(C═NH)NH₂; Y—(CH₂)ᵥ—R₃₀ where Y is O, NH or S, v is 1-6 and R₃₀ is COC₁₋₆alkyl, COOH or CONH₂; and Y—(CH═CH)—R₃₁, wherein R₃₁ is COC₁₋₆alkyl, COOH, CONH₂ or phenyl.

98. The compound of claim 97 wherein the aryl groups in the aryl substituents are selected from the group consisting of substituted and unsubstituted phenyl, pyridyl, furanyl, thienyl, thiazolyl, isothiazolyl, imidazolyl, pyrazinyl, pyrimidyl, pyrazolyl, isoxazolyl and oxazolyl, which can be independently substituted by one or more of hydroxy, amino, carboxyl, carboxamide, halo, hydroxy, C₁₋₆alkyl amino, di-C₁₋₆alkyl amino, C₁₋₆alkoxy, C₁₋₆alkylthio, C₁₋₆-alkylcarbonyl.

99. The compound of claim 97 wherein the aryl group is substituted by Y—(CH₂)ₜ-heterocycloalkyl, wherein the heterocycloalkyl group is selected from the group consisting of morpholinyl, pyrrolidinyl, piperazinyl, N-substituted piperazinyl, piperidinyl, tetrahydropyranyl, tetrahydrofuranyl, and pyrrolidinonyl.

100. The compound of claim 71, wherein substituent X in Formula II is at position 3 or position 4 of the phenyl ring.

101. The compound of claim 71, wherein A2 is aspartyl or an ester thereof; glutamyl or an ester thereof; alpha-aminoadipic acid or an ester thereof; valyl, norvalyl or leucyl.

102. The compound of claim 71 wherein A3 is is aspartyl or an ester thereof; glutamyl or an ester thereof; alpha-aminoadipic acid or an ester thereof; valyl, norvalyl,leucyl, prolyl, or thiaprolyl; or
R₂₃ and R₂₄ together form a three to seven-membered ring;
or R₂₂ and R₂₄, together with the nitrogen, form a substituted or unsubstituted heterocycle.

103. The compound of claim 71, wherein R₂₇ is substituted or unsubstituted phenyl, naphthyl or benzothienyl.

104. The compound of claim 71, wherein R₂ is (CH₂)_{w}—W—(CH₂)ₓ—COOR₃₄, wherein W is a single bond, phenyl or C₆-cycloalkyl.

105. The compound of claim 71, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

106. The compound of claim 105, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

107. The compound of claim 100, wherein A2 is aspartyl or an ester thereof; glutamyl or an ester thereof; alpha-aminoadipic acid or an ester thereof; valyl, norvalyl or leucyl.

108. The compound of claim 100 wherein A3 is is aspartyl or an ester thereof; glutamyl or an ester thereof; alpha-aminoadipic acid or an ester thereof; valyl, norvalyl, leucyl, prolyl, or thiaprolyl; or
R₂₃ and R₂₄ together form a three to seven-membered ring;
or R₂₂ and R₂₄, together with the nitrogen, form a substituted or unsubstituted heterocycle.

109. The compound of claim 100, wherein R₂₇ is substituted or unsubstituted phenyl, naphthyl or benzothienyl.

110. The compound of claim 100, wherein R₂ is (CH₂)_{w}—W—(CH₂)ₓ—COOR₃₄, wherein W is a single bond, phenyl or C₆-cycloalkyl.

111. The compound of claim 100, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

112. The compound of claim 111, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

113. The compound of claim 107 wherein A3 is is aspartyl or an ester thereof; glutamyl or an ester thereof; alpha-aminoadipic acid or an ester thereof; valyl, norvalyl,leucyl, prolyl, or thiaprolyl; or
R₂₃ and R₂₄ together form a three to seven-membered ring;
or R₂₂ and R₂₄, together with the nitrogen, form a substituted or unsubstituted heterocycle.

114. The compound of claim 107, wherein R₂₇ is substituted or unsubstituted phenyl, naphthyl or benzothienyl.

115. The compound of claim 107, wherein R₂ is (CH₂)_{w}—W—(CH₂)ₓ—COOR₃₄, wherein W is a single bond, phenyl or C₆-cycloalkyl.

116. The compound of claim 107, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

117. The compound of claim 107, wherein U is OH or NHR_{38,} wherein R₃8 is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

118. The compound of claim 113, wherein R₂₇ is substituted or unsubstituted phenyl, naphthyl or benzothienyl.

119. The compound of claim 113, wherein R₂ is (CH₂)_{w}—W—(CH₂)ₓ—COOR₃₄, wherein W is a single bond, phenyl or C₆-cycloalkyl.

120. The compound of claim 113, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

121. The compound of claim 120, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring

122. The compound of claim 118, wherein R₂ is (CH₂)_{w}—W—(CH₂)ₓ—COOR₃₄, wherein W is a single bond, phenyl or C₆-cycloalkyl.

123. The compound of claim 118, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

124. The compound of claim 123, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

125. The compound of claim 114, wherein R₂ is (CH₂)_{w}—W—(CH₂)ₓ—COOR₃₄, wherein W is a single bond, phenyl or C₆-cycloalkyl.

126. The compound of claim 114, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

127. The compound of claim 126, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

128. The compound of claim 109, wherein R₂ is (CH₂)_{w}—W—(CH₂)ₓ—COOR₃₄, wherein W is a single bond, phenyl or C₆-cycloalkyl.

129. The compound of claim 109, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

130. The compound of claim 129, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

131. The compound of claim 71 wherein A2 and A3 together are a residue selected from the group consisting of 6-amino-5-oxoperhydropyrido[2,1-b][1,3]thiazole-3-carboxylic acid; 6-amino-5-oxoperhydro-3-indolizinecarboxylic acid; (S, R)-6-amino-5-oxoperhydro-8a-indolizinecarboxylic acid; (R,R)-6-amino-5-oxoperhydro-8a-indolizinecarboxylic acid; (R, S)-6-amino-5-oxoperhydro-8a-indolizinecarboxylic acid; (S,S)-6-amino-5-oxoperhydro-8a-indolizinecarboxylic acid; 2-(3-amino-2-oxo-1,2-dihydro-1-pyridinyl)acetic acid; 2-(3-amino-2-oxo-6-phenyl-1,2-dihydro-1-pyridinyl)acetic acid; 3-amino benzoic acid; 4-aminobenzoic acid; 3-aminomethyl benzoic acid; (S)-3-(1-aminoethyl)benzoic acid; (R)-3-(1-aminoethyl)benzoic acid; (S)-3-(1-aminopropyl) benzoic acid; (R)-3-(1-aminopropyl) benzoic acid; (S)-3-(1-aminobutyl) benzoic acid; (R)-3-(1-aminobutyl) benzoic acid; 2-(3-amino-2-oxo-1-azepanyl)acetic acid; 2-[8-(aminomethyl)-3,6-dimethyl-9,10,10-trioxo-9,10-dihydro-10⁶-thioxanthen-1-yl]acetic acid; 2-(2-oxopiperazino)acetic acid; 2-[8-(aminomethyl)-2-oxo-5-phenyl-2,3-dihydro-1H-1,4-benzodiazepin-1-yl]acetic acid; 2-[8-(aminomethyl)-2-oxo-5-methyl-2,3-dihydro-1H-1,4-benzodiazepin-1-yl]acetic acid; 3-aminopropanoic acid; 4-aminobutanoic acid; 5-aminopentanoic acid; 2-[2-(2-aminoethoxy)ethoxy]acetic acid; and 2-(3-amino-2-oxo-2,3,4,5-tetrahydro-1H-1-benzazepin-1-yl)acetic acid.

132. The compound of claim 131 wherein substituent X in Formula II is at position 3 or position 4 of the phenyl ring.

133. The compound of claim 131 wherein R₂₇ is substituted or unsubstituted phenyl, naphthyl or benzothienyl.

134. The compound of claim 131 wherein R₂ is (CH₂)_{w}—W—(CH₂)ₓ—COOR₃₄, wherein W is a single bond, phenyl or C₆-cycloalkyl.

135. The compound of claim 131, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

136. The compound of claim 135, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

137. The compound of claim 132, wherein R₂₇ is phenyl, naphthyl or benzothienyl.

138. The compound of claim 132, wherein R₂ is (CH₂)_{w}—W—(CH₂)ₓ—COOR₃₄, wherein W is a single bond, phenyl or C₆-cycloalkyl.

139. The compound of claim 132, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

140. The compound of claim 139, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

141. The compound of claim 137, wherein R₂ is (CH₂)_{w}—W—(CH₂)ₓ—COOR₃₄, wherein W is a single bond, phenyl or C₆-cycloalkyl.

142. The compound of claim 137, wherein R₃₇ is the side chain of aspartic acid or glutamic acid;
(CH₂)_{y}—COOR₄₂ wherein y is 3 to 5 and R₄₂ is hydrogen or C₁₋₆alkyl; or -phenyl-(CH₂)ₑ—COOR₄₃, wherein e is 0, 1 or 2 and R₄₃ is hydrogen or C₁₋₆alkyl.

143. The compound of claim 142, wherein U is OH or NHR₃₈, wherein R₃₈ is tert.butyl, isopropyl, 2,4-dimethylpent-3-yl, cyclopentyl, cyclohexyl, or bicyclooct[3.3.0]yl; or R₃₈ and R₃₉, together with the nitrogen atom, form a pyrrolidinyl or piperazinyl ring.

144. A method of treating a Cdc25-mediated condition in a patient comprising the step of administering to the patient a therapeutically effective amount of a Cdc25 inhibitor of claim 71.

145. The method of claim 144 wherein the patient is a human.

146. The method of claim 145 wherein the Cdc25-mediated condition is characterized by excessive cellular proliferation.

147. The method of claim 146 wherein the Cdc25-mediated condition is cancer, restenosis, reocclusion of a coronary artery and inflammation.
